(19) United States
(12) Patent Application Publication
(10) Pub. No.: US 2010/0162432 A1
(43) Pub. Date: Jun. 24, 2010

(54) PLANT CELLS AND PLANTS WITH INCREASED TOLERANCE AND/OR RESISTANCE TO ENVIRONMENTAL STRESS AND INCREASED BIOMASS PRODUCTION-KO

(75) Inventors: Piotr Puzio, Mariakerke (BE); Oliver Bläsing, Potsdam (DE); Oliver Thimm, Berlin (DE)

Correspondence Address: CONNOLLY BOVE LODGE & HUTZ, LLP, P O BOX 2207, WILMINGTON, DE, 19899, US

(73) Assignee: BASF Plant Science GmbH, Limburgerhof (DE)

(21) Appl. No.: 12/601,053

(22) PCT Filed: May 19, 2008
(86) PCT No.: PCT/EP08/56091

§ 371 Date: Nov. 20, 2009

(30) Foreign Application Priority Data
May 22, 2007 (EP) 07108576.5
May 22, 2007 (EP) 07108577.3

(51) Int. Cl. A01H 1/06; A01H 1/00; C07H 21/00; C07H 21/02; C07K 16/00; C07K 2/00; C12N 15/63; C12N 5/04; A01H 5/00; C07K 1/00; C12Q 1/02

(52) U.S. Cl. 800/276; 800/278; 536/23.1; 536/23.5; 530/387.1; 536/24.33; 530/300; 435/320.1; 435/419; 800/298; 435/412; 435/416; 435/417; 435/414; 435/411; 800/320.1; 800/320.3; 800/320; 800/320.2; 800/314; 800/322; 800/317; 800/317.2; 800/317.1; 800/317.3; 800/317.4; 530/333; 435/29

(57) ABSTRACT

The invention relates generally to transformed plant cells and plants comprising an in-activated or down-regulated gene resulting in increased tolerance and/or resistance to environmental stress and increased biomass production as compared to non-transformed wild type cells and methods of producing such plant cells or plants.

This invention relates generally to transformed plant cells and plants comprising an inactivated or down-regulated gene resulting in increased tolerance and/or resistance to environmental stress and increased biomass production as compared to non-transformed wild type cells and methods of producing such plant cells or plants.

In particular, this invention relates to plants tailored to grow under conditions of water deficiency.

The invention also deals with methods of producing and screening for and breeding such plant cells or plants.

Under field conditions, plant performance in terms of growth, development, biomass accumulation and yield depends on acclimation ability to the environmental changes and stresses. Abiotic environmental stresses such as drought stress, salinity stress, heat stress and cold stress, are major limiting factors of plant growth and productivity (Boyer. 1982. Science 218, 443-448). Plants exposed to heat and/or low water or drought conditions typically have low yields of plant material, seeds, fruit and other edible products. Crop losses and crop yield losses of major crops such as rice, maize (corn) and wheat caused by these stresses represent a significant economic and political factor and contribute to food shortages in many underdeveloped countries.

Drought, heat, cold and salt stress have a common theme important for plant growth and that is water availability. Plants are typically exposed during their life cycle to conditions of reduced environmental water content. Most plants have evolved strategies to protect themselves against these conditions of low water or desiccation. However, if the severity and duration of the drought conditions are too great, the effects on plant development, growth and yield of most crop plants are profound. Continuous exposure to drought causes major alterations in the plant metabolism. These great changes in metabolism ultimately lead to cell death and consequently yield losses.

Developing stress-tolerant and/or resistant plants is a strategy that has the potential to solve or mediate at least some of these problems (McKersie and Leshem, 1994. Stress and Stress Coping in Cultivated Plants, Kluwer Academic Publishers). However, traditional plant breeding strategies to develop new lines of plants that exhibit resistance (tolerance) to these types of stress are relatively slow and require specific resistant lines for crossing with the desired line. Limited germplasm resources for stress tolerance and incompatibility in crosses between distantly related plant species represent significant problems encountered in conventional breeding. Additionally, the cellular processes leading to drought, cold and salt tolerance and/or resistance are complex in nature and involve multiple mechanisms of cellular adaptation and numerous metabolic pathways (McKersie and Leshem, 1994. Stress and Stress Coping in Cultivated Plants, Kluwer Academic Publishers). This multicomponent nature of stress tolerance and/or resistance has not only made breeding for tolerance and/or resistance largely unsuccessful, but has also limited the ability to genetically engineer stress tolerance plants using biotechnological methods.

Plants are exposed during their life cycle also to heat, cold and salt stress. The protection strategies are similar to those of drought resistance. Since high salt content in some soils results in less available water for cell intake, its effect is similar to those observed under drought conditions. Likewise, under freezing temperatures, plant cells loose water as a result of ice formation that starts in the apoplast and withdraws water from the symplast (McKersie and Leshem, 1994. Stress and Stress Coping in Cultivated Plants, Kluwer Academic Publishers). Physiologically these stresses are also interconnected and may induce similar cellular damage. For example drought and salt stress are manifested primarily as osmotic stress, leading to the disruption of homeostasis and ion distribution in the cell (Serrano et al., 1999; Zhu, 2001a; Wang et al., 2003). Oxidative stress, which frequently accompanies high temperature, salinity or drought stress, may cause denaturation of functional or structural proteins (Smirnoff, 1998). As a consequence these abiotic stresses often activate similar signaling pathways (Shinozaki and Ymaguchi-Shinozaki, 2000; Knight and Knight, 2001; Zhu 2001b, 2002) and cellular responses, e.g. the production of certain stress proteins, antioxidants and compatible solutes (Vierling and Kimpel, 1992; Zhu et al., 1997; .Cushman and Bohnert, 2000).

Plant with increased resistance to abiotic stress by gene knock-out are known from WO 2004/092349 A and WO 2006/032707.

Generally the transformed and stress resistant plants exhibit slower growth and reduced biomass due to a decreased growth rate (Serrano et al.), due to an imbalance in development and physiology of the plant, thus having significant fitness cost (Kasuga et al., 1999, Danby and Gehring et al., 2005). Despite maintaining basic metabolic function this leads to severe biomass and yield loss. Sometimes the root/shoot dry weight ratio increase as plant water stress develops. The increase is mostly due to a relative reduction in shoot dry weight. The ratio of seed yield to above-ground dry weight is relatively stable under many environmental conditions and so a robust correlation between plant size and grain yield can often be obtained. These processes are intrinsically linked because the majority of grain biomass is dependent on current stored photosynthetic productivity by the leaves and stem of the plant. Therefore selecting for plant size, even at early stages of development, has been used as an indicator for future potential.

In some cases (US20060037108) an increased biomass, mainly a greater shoot biomass was observed after a drought treatment by withholding water for 6 to 8 days.

The results of current research indicate that drought tolerance and/or resistance is a complex quantitative trait and that no real diagnostic marker is available yet. This lack of a mechanistic understanding makes it difficult to design a transgenic approach to improve water stress tolerance and/or resistance.

Plant stress-regulated genes, whose expression are dependent on plant growth under different stress conditions, where identified by using the micro array technology and are disclosed in WO 03/000898 A1, WO 02/16655 A, WO 02/22675 A2 and WO 03/008540 A2. It is believed that the identification of these plant genes will be helpful in conferring plants with a selective advantage, for example better propagation, development, growth, survival by increasing the resistance to bacterial or fungal pathogen infection, the herbicidal resistance, insect resistance, environmental or stress resistance and disease resistance. Additionally also the grain composition or quality respectively could be enhanced, for example several limiting amino acids, oil content, starch content, pigmentation, vitamins. Nevertheless, no practical approach of deleting (or knocking out respectively) the genes was made. Accordingly the disclosure refers to the consequence of stress conditions on plants but there is no proof for involvement in plant's stress resistance.

According to WO 03/020015 A2 an increased salt resistance is exhibited in A. thaliana C24 by deleting or by inactivation (mutagenesis or anti-sense) but also by overexpression of the nced3 gene which encodes for 9-cis-epoxycarotenoid dioxygenase. In contrast to their salt resistance the mutant plants were much more sensitive to soil dessication than wild type plants.

At the moment many genetical and biotechnological approaches are known in order to obtain plants growing under conditions of low water availability.

There is still a need to identify genes expressed in stress tolerant plants that have the capacity to confer stress resistance to its host plant and to other plant species, specially to confer increased tolerance and/or resistance to environmental stress, preferably under conditions of water deficiency and confers increased biomass production. It is a object of this invention to identify new methods to confer stress tolerance and/or resistance in plants or plant cells. Complex traits of abiotic stress phenomena make genetic optimisation difficult. However, the modification of a single gene e.g. transcription factors or antiporters resulted in several cases in a significant increase in stress tolerance (Wang et al., 2003).

It is further a object of this invention to put plants at disposal, which are drought resistant for a period of at least 1.0, preferably 1.5 days of water deficiency as compared to a corresponding non-transformed wild type plant, and exhibit additionally under conditions of low water or desiccation an equal, preferably an increased biomass production.

Summarized, the present invention relates to a method for producing a transgenic plant with increased tolerance and/or resistance to environmental stress and increased biomass production as compared to a corresponding non-transformed wild type plant, which comprises the following steps:
a) Reducing, repressing or deleting of one or more activities selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme., in a plant cell, a plant or a part thereof, and
b) generating a transformed plant with increased tolerance and/or resistance to environmental stress and increased biomass production as compared to a corresponding non-transformed wild type plant and growing under conditions which permit the development of the plant.

Further, in another embodiment, the present invention relates to a method for producing a transgenic plant with increased tolerance and/or resistance to environmental stress and increased biomass production as compared to a corresponding non-transformed wild type plant, which comprises the following steps:
a) reduction, repression or deletion of the activity of
   (i) a polypeptide comprising a polypeptide, a consensus sequence or at least one polypeptide motif as depicted in column 5 or 7 of Table II or of Table IV, respectively; or
   (ii) an expression product of a nucleic acid molecule comprising a polynucleotide as depicted in column 5 or 7 of Table I,
   (iii) or a functional equivalent of (i) or (ii);
in a plant cell, a plant or a part thereof, and
b) generating a transformed plant with increased tolerance and/or resistance to environmental stress and increased biomass production as compared to a corresponding non-transformed wild type plant and growing under conditions which permit the development of the plant.

Preferably, the process of the invention further comprises reducing, decreasing or deleting the expression or activity of at least one nucleic acid molecule having or encoding the activity of at least one nucleic acid molecule represented by the nucleic acid molecule as depicted in column 5 of Table I, and comprising a nucleic acid molecule which is selected from the group consisting of:
a) an isolated nucleic acid molecule encoding the polypeptide as depicted in column 5 or 7 of Table II;
b) an isolated nucleic acid molecule as depicted in column 5 or 7 of Table I;
c) an isolated nucleic acid molecule, which, as a result of the degeneracy of the genetic code, can be derived from a polypeptide sequence as depicted in column 5 or 7 of Table II;
d) an isolated nucleic acid molecule having at least 30% identity with the nucleic acid molecule sequence of a polynucleotide comprising the nucleic acid molecule as depicted in column 5 or 7 of Table I;
e) an isolated nucleic acid molecule encoding a polypeptide having at least 30% identity with the amino acid sequence of the polypeptide encoded by the nucleic acid molecule of (a) to (c) and having the activity represented by a nucleic acid molecule comprising a polynucleotide as depicted in column 5 of Table I;
f) an isolated nucleic acid molecule encoding a polypeptide which can be isolated with the aid of monoclonal or polyclonal antibodies made against a polypeptide encoded by one of the nucleic acid molecules of (a) to (e) and having the activity represented by the nucleic acid molecule comprising a polynucleotide as depicted in column 5 of Table I;
g) an isolated nucleic acid molecule encoding a polypeptide comprising the consensus sequence or one or more polypeptide motifs as depicted in column 7 of Table IV and preferably having the activity represented by a nucleic acid molecule comprising a polynucleotide as depicted incolumn 5 of Table II or IV;
h) an isolated nucleic acid molecule encoding a polypeptide having the activity represented by a protein as depicted in column 5 of Table II;
i) an isolated nucleic acid molecule which comprises a polynucleotide, which is obtained by amplifying a cDNA library or a genomic library using the primers as depicted in column 7 of Table III which do not start at their 5′-end with the nucleotides ATA and preferably having the activity represented by a nucleic acid molecule comprising a polynucleotide as depicted in column 5 of Table II or IV;
j) an isolated nucleic acid molecule encoding a polypeptide, the polypeptide being derived by substituting, deleting and/or adding one or more amino acids of the amino acid sequence of the polypeptide encoded by the nucleic acid molecules (a) to (d); and
k) an isolated nucleic acid molecule which is obtainable by screening a suitable nucleic acid library under stringent hybridization conditions with a probe comprising a complementary sequence of a nucleic acid molecule of (a) or (b) or with a fragment thereof, having at least 15 nt, preferably 20 nt, 30 nt, 50 nt, 100 nt, 200 nt or 500 nt of a nucleic acid molecule complementary to a nucleic acid molecule sequence characterized in (a) to (d) and encoding a polypeptide having the activity represented by a protein comprising a polypeptide as depicted in column 5 of Table II;
   or which comprises a sequence which is complementary thereto;

Preferably, the process of the invention comprises further reducing, repressing, decreasing or deleting of an expression product of a nucleic acid molecule comprising a nucleic acid molecule as depicted in (a) to (j) above, e.g. a polypeptide comprising a polypeptide as depicted in column 5 or 7 of Table II or of a protein encoded by said nucleic acid molecule.

Preferably, the process of the invention comprises further the reduction of the activity or expression of a polypeptide comprising a polypeptide encoded by the nucleic acid molecule characterized above in a plant or part thereof.

Preferably, the process of the invention comprises further at least one step selected from the group consisting of:
a) introducing of a nucleic acid molecule encoding a ribonucleic acid sequence, which is able to form a double-stranded ribonucleic acid molecule, whereby a fragment of at least 17 nt of said double-stranded ribonucleic acid molecule has a homology of at least 50% to a nucleic acid molecule selected from the group of
   aa) an isolated nucleic acid molecule as characterized above;
   ab) an isolated nucleic acid molecule as depicted in column 5 or 7 of Table I or encoding a polypeptide as depicted in column 5 or 7 of Table II, and
   ac) an isolated nucleic acid molecule encoding a polypeptide having the activity of polypeptide depicted in column 5 of Table II or encoding the expression product of a polynucleotide comprising a nucleic acid molecule as depicted in column 5 or 7 of Table I;
b) introducing an RNAi, snRNA, dsRNA, siRNA, miRNA, ta-siRNA, cosuppression molecule, ribozyme, or antisense nucleic acid molecule, whereby the RNAi, snRNA, dsRNA, siRNA, miRNA, ta-siRNA, cosuppression molecule, ribozyme, or antisense nucleic acid molecule comprises a fragment of at least 17 nt with a homology of at least 50% to a nucleic acid molecule selected from the group defined in section (a) of this claim.
c) introducing of a ribozyme which specifically cleaves a nucleic acid molecule selected from the group defined in section (a) of this claim;
d) introducing of the RNAi, snRNA, dsRNA, siRNA, miRNA, ta-siRNA, cosuppression molecule, ribozyme, or antisense nucleic acid molecule characterized in (b) and the ribozyme characterized in (c);
e) introducing of a sense nucleic acid molecule conferring the expression of a nucleic acid molecule comprising a nucleic acid molecule selected from the group defined herein above or defined in section (ab) or (ac) above or a nucleic acid molecule encoding a polypeptide having at least 50% identity with the amino acid sequence of the polypeptide encoded by the nucleic acid molecule mentioned in section (a) to (c) and having the activity represented by a protein comprising a polypeptide as depicted in column 5 of Table II for inducing a cosuppression of the endogenous expression product;
f) introducing a nucleic acid molecule conferring the expression of a dominant-negative mutant of a protein having the activity of a protein as depicted in column 5 or 7 of Table II or comprising a polypeptide being encoded by a nucleic acid molecule as characterized herein above;
g) introducing a nucleic acid molecule encoding a factor, which binds to a nucleic acid molecule comprising a nucleic acid molecule selected from the group defined herein above or defined in section (ab) or (ac) of this claim conferring the expression of a protein having the activity of a protein encoded by a nucleic acid molecule as characterized herein above;
h) introducing a viral nucleic acid molecule conferring the decline of a RNA molecule comprising a nucleic acid molecule selected from the group defined herein above or defined in section (ab) or (ac) of this claim conferring the expression of a protein encoded by a nucleic acid molecule as characterized herein above;
i) introducing a nucleic acid construct capable to recombine with and silence, inactivate, repress or reduces the activity of an endogenous gene comprising a nucleic acid molecule selected from the group defined herein above or defined in section (ab) or (ac) of this claim conferring the expression of a protein encoded by a nucleic acid molecule as characterized herein above;
j) introducing a non-silent mutation in an endogenous gene comprising a nucleic acid molecule selected from the group defined herein above or defined in section (ab) or (ac) of this claim; and
k) introducing an expression construct conferring the expression of nucleic acid molecule characterized in any one of (a) to (i).

Preferably, in the process of the invention, a fragment of at least 17 by of a 3′- or 5′-nucleic acid sequence of a sequences comprising a nucleic acid molecule selected from the group defined herein above or defined in section (ab) or (ac) above with an identity of at least 50% is used for the reduction of the nucleic acid molecule characterized above or the polypeptide encoded by said nucleic acid molecule.

Preferably, in the process of the invention, the reduction or deletion is caused by applying a chemical compound to the non-human-organism.

Preferably, in the process of the invention, the plant is selected from the group consisting of Anacardiaceae, Asteraceae, Apiaceae, Betulaceae, Boraginaceae, Brassicaceae, Bromeliaceae, Caricaceae, Cannabaceae, Convolvulaceae, Chenopodiaceae, Cucurbitaceae, Elaeagnaceae, Ericaceae, Euphorbiaceae, Fabaceae, Geraniaceae, Gramineae, Juglandaceae, Lauraceae, Leguminosae, Linaceae, perennial grass, fodder crops, vegetables and ornamentals.

Preferably, the process of the invention further comprises the step, introduction of a RNAi, snRNA, dsRNA, siRNA, miRNA, ta-siRNA, cosuppression molecule, ribozyme, antibody and/or antisense nucleic that has been designed to target the expression product of a gene comprising the nucleic acid molecule as characterized herein above to induce a breakdown of the mRNA of the said gene of interest and thereby silence the gene expression, or of an expression cassette ensuring the expression of the former.

Further, in another embodiment, the present invention relates to an isolated nucleic acid molecule, which comprises a nucleic acid molecule selected from the group consisting of:
a) an isolated nucleic acid molecule which encodes a polypeptide comprising the polypeptide as depicted in column 5 or 7 of Table II B or;
b) an isolated nucleic acid molecule which comprising a polynucleotide as depicted in column 5 or 7 of Table I B or;
c) an isolated nucleic acid molecule comprising a nucleic acid sequence, which, as a result of the degeneracy of the genetic code, can be derived from a polypeptide sequence as depicted in column 5 or 7 of Table II B and having the activity represented by the protein as depicted in column 5 of Table II;
d) an isolated nucleic acid molecule encoding a polypeptide having at least 50% identity with the amino acid sequence of a polypeptide encoded by the nucleic acid molecule of (a) or (c) and having the activity represented by the protein as depicted in column 5 of Table II ;
e) an isolated nucleic acid molecule encoding a polypeptide, which is isolated with the aid of monoclonal antibodies against a polypeptide encoded by one of the nucleic acid molecules of (a) to (c) and having the activity represented by the protein as depicted in column 5 of Table II;
f) an isolated nucleic acid molecule encoding a polypeptide comprising the consensus sequence or a poylpeptide motif as depicted in column 7 of Table IV and having the biological activity represented by the protein as depicted in column 5 of Table II;
g) an isolated nucleic acid molecule encoding a polypeptide having the activity represented by a protein as depicted in column 5 of Table II;
h) an isolated nucleic acid molecule which comprises a polynucleotide, which is obtained by amplifying a cDNA library or a genomic library using the primers as depicted in column 7 of Table III which do not start at their 5′-end with the nucleotides ATA; and
i) an isolated nucleic acid molecule which is obtainable by screening a suitable library under stringent hybridization conditions with a probe comprising one of the sequences of the nucleic acid molecule of (a) to (c) or with a fragment of at least 17 nt of the nucleic acid molecule characterized in any one of (a) to (h) and encoding a polypeptide having the activity represented by the protein as depicted in column 5 of Table II;
   or which comprises a sequence which is complementary thereto;

whereby the nucleic acid molecule according to (a) to (i) is at least in one or more nucleotides different from the sequence as depicted in column 5 or 7 of Table I A and preferably which encodes a protein which differs at least in one or more amino acids from the protein sequences as depicted in column 5 or 7 of Table II A.

Further, in another embodiment, the present invention relates to an RNAi, snRNA, dsRNA, siRNA, miRNA, ta-siRNA, cosuppression molecule, ribozyme, antibody or antisense nucleic acid molecule for the reduction of the activity characterized above or of the activity or expression of a nucleic acid molecule as characterized herein above or a polypeptide encoded by said nucleic acid molecule.

Preferably, the RNAi, snRNA, dsRNA, siRNA, miRNA, ta-siRNA, cosuppression molecule, ribozyme, or antisense nucleic acid molecule of the invention comprises a fragment of at least 17 nt of the nucleic acid molecule defined herein above.

Further, in another embodiment, the present invention relates to a double-stranded RNA (dsRNA), RNAi, snRNA, siRNA, miRNA, antisense or to-siRNA molecule or ribozyme, which is able to form a double-stranded ribonucleic acid molecule, whereby a fragment of at least 17 nt of said double-stranded ribonucleic acid molecule has a homology of at least 50% to a nucleic acid molecule selected from the group consisting of
aa) an isolated nucleic acid molecule as characterized herein above;
ab) an isolated nucleic acid molecule as depicted in column 5 or 7 of Table I or encoding a polypeptide as depicted in column 5 or 7 of Table II, and
ac) an isolated nucleic acid molecule encoding a polypeptide having the activity of polypeptide as depicted in column 5 or 7 of Table II or encoding the expression product of a polynucleotide comprising a nucleic acid molecule as depicted in column 5 or 7 of Table I.

Preferably, in the dsRNA molecule of the invention, the sense strand and the antisense strand are covalently bound to each other and the antisense strand is essentially the complement of the “sense”-RNA strand.

Further, in another embodiment, the present invention relates to a viral nucleic acid molecule conferring the decline of an RNA molecule conferring the expression of a protein having the activity characterized above or of the activity or expression of a nucleic acid molecule as characterized herein above or a polypeptide encoded by said nucleic acid molecule.

Further, in another embodiment, the present invention relates to a TILLING primer for the identification of a knock out of a gene comprising a nucleic acid sequence of a nucleic acid molecule as depicted in any one column 5 or 7 of Table I.

Further, in another embodiment, the present invention relates to a dominant-negative mutant of polypeptide comprising a polypeptide as depicted in column 5 or 7 of Table

Further, in another embodiment, the present invention relates to a nucleic acid molecute encoding the dominant negative mutant defined above.

Further, in another embodiment, the present invention relates to a nucleic acid construct conferring the expression of the RNAi, snRNA, dsRNA, siRNA, miRNA, ta-siRNA, cosuppression molecule, ribozyme, antibody or antisense nucleic acid molecule of the invention, the viral nucleic acid molecule of the invention or the nucleic acid molecule of the invention.

Further, in another embodiment, the present invention relates to a nucleic acid construct comprising the isolated nucleic acid molecule of the invention or the RNAi, snRNA, dsRNA, siRNA, miRNA, ta-siRNA, cosuppression molecule, ribozyme, or antisense nucleic acid molecule of the invention, or the viral nucleic acid molecule of the invention, wherein the nucleic acid molecule is functionally linked to one or more regulatory signals.

Further, in another embodiment, the present invention relates to a vector comprising the nucleic acid molecule of the invention or the RNAi, snRNA, dsRNA, siRNA, miRNA, ta-siRNA, cosuppression molecule, ribozyme, or antisense nucleic acid molecule of the invention, or the viral nucleic acid molecule of the invention, or the nucleic acid construct of the invention.

Preferably, in the vector of the invention, the nucleic acid molecule is in operable linkage with regulatory sequences for the expression in a plant host.

Further, in another embodiment, the present invention relates to a transgenic plant host cell, which has been transformed stably or transiently with the vector of the invention, or the nucleic acid molecule of the invention or the nucleic acid construct of the invention.

Further, in another embodiment, the present invention relates to a plant cell, a plant or a part thereof, wherein the activity of a protein comprising a polypeptide, a consensus sequence or a polypeptide motif as depicted in column 5 or 7 of Table II, preferably Table II B, or IV or a nucleic acid molecule comprising a nucleic acid molecule as depicted in column 5 or 7 of Table I, preferably Table I B, is reduced.

Further, in another embodiment, the present invention relates to a process for producing a polypeptide encoded by a nucleic acid sequence of the invention, the polypeptide being expressed in a plant cell, a plant or a part thereof, of the invention.

Preferably, in the process for producing a polypeptide of the invention or in the host cell of the invention, the host cell is a plant cell selected from the group consisting of Anacardiaceae, Asteraceae, Apiaceae, Betulaceae, Boraginaceae, Brassicaceae, Bromeliaceae, Caricaceae, Cannabaceae, Convolvulaceae, Chenopodiaceae, Cucurbitaceae, Elaeagnaceae, Ericaceae, Euphorbiaceae, Fabaceae, Geraniaceae, Gramineae, Juglandaceae, Lauraceae, Leguminosae, Linaceae, perennial grass, fodder crops, vegetables and ornamentals or is a microorganism as defined above.

Further, in another embodiment, the present invention relates to an isolated polypeptide encoded by a nucleic acid molecule of the invention or comprising the polypeptide as depicted in column 7 of Table II B.

Further, in another embodiment, the present invention relates to an antibody, which specifically binds to the polypeptide of the invention.

Further, in another embodiment, the present invention relates to a plant tissue, plant, harvested plant material or propagation material of a plant comprising the plant cell of the invention.

Further, in another embodiment, the present invention relates to a method for screening of an antagonist of an activity as characterized in the process of the invention above or being represented by the polypeptide encoded by the nucleic acid molecule characterized for the process of the invention above:
a) contacting an organism, its cells, tissues or parts, which express the polypeptide with a chemical compound or a sample comprising a plurality of chemical compounds under conditions which permit the reduction or deletion of the expression of the nucleic acid molecule encoding the activity represented by the protein or which permit the reduction or deletion of the activity of the protein;
b) assaying the level of the activity of the protein or the polypeptide expression level in the plant, its cells, tissues or parts wherein the plant, its cells, tissues or parts is cultured or maintained in; and
c) identifying an antagonist by comparing the measured level of the activity of the protein or the polypeptide expression level with a standard level of the activity of the protein or the polypeptide expression level measured in the absence of said chemical compound or a sample comprising said plurality of chemical compounds, whereby an decreased level in comparison to the standard indicates that the chemical compound or the sample comprising said plurality of chemical compounds is an antagonist.

Further, in another embodiment, the present invention relates to a process for the identification of a compound conferring increased tolerance and/or resistance to environmental stress and increased biomass production as compared to a corresponding non-transformed wild type plant in a plant, comprising the steps:
a) culturing or maintaining a plant, plant cell or their tissues or a part thereof expressing the polypeptide having the activity characterized in the process of the invention above or the polypeptide encoded by the nucleic acid molecule characterized in the process of the invention above or a polynucleotide encoding said polypeptide and a readout system capable of interacting with the polypeptide under suitable conditions which permit the interaction of the polypeptide with this readout system in the presence of a chemical compound or a sample comprising a plurality of chemical compounds and capable of providing a detectable signal in response to the binding of a chemical compound to said polypeptide under conditions which permit the depression of said readout system and of said polypeptide; and
b) identifying if the chemical compound is an effective antagonist by detecting the presence or absence or decrease or increase of a signal produced by said readout system.

Further, in another embodiment, the present invention relates to a method for the production of an agricultural composition comprising the steps of the process for the identification of a compound conferring increased tolerance and/or resistance to environmental stress and increased biomass production as compared to a corresponding nontransformed wild type plant in a plant; in a plant cell or part thereof, of the invention and formulating the compound identified said claims in a form acceptable for an application in agriculture.

Further, in another embodiment, the present invention relates to a composition comprising the protein of the invention, the nucleic acid molecule of the invention, the nucleic acid construct of the invention, the vector of the invention, the antagonist identified according to the method for identification of an antagonist of the invention, the antibody of the invention, the host cell of the invention, the nucleic acid molecule characterized in the process of the invention, the RNAi, snRNA, dsRNA, siRNA, miRNA, ta-siRNA, cosuppression molecule, ribozyme, or antisense nucleic acid molecule of the invention and optionally a agricultural acceptable carrier.

Further, in another embodiment, the present invention relates to a food or feed comprising the protein of the invention, the nucleic acid molecule of the invention, the nucleic acid construct of the invention, the vector of the invention, the antagonist identified according to the method for identification of an antagonist of the invention, the antibody of the invention, the host cell of the invention, the nucleic acid molecule characterized in the process of the invention, the RNAi, snRNA, dsRNA, siRNA, miRNA, ta-siRNA, cosuppression molecule, ribozyme, or antisense nucleic acid molecule of the invention, the plant, plant tissue, the harvested plant material or propagation material of a plant of the invention.

Further, in another embodiment, the present invention relates to use of the protein of the invention, the nucleic acid molecule of the invention, the nucleic acid construct of the invention, the vector of the invention, the antagonist identified according to the method for identification of an antagonist of the invention, the antibody of the invention, the host cell of the invention, the nucleic acid molecule characterized in the process of the invention, the RNAi, snRNA, dsRNA, siRNA, miRNA, ta-siRNA, cosuppression molecule, ribozyme, or antisense nucleic acid molecule of the invention, for producing a transgenic plant with increased tolerance and/or resistance to environmental stress and increased biomass production as compared to a corresponding non-transformed wild type plant.

Table I shows the SEQ ID NOs. of relevant polynucleotides. Table II shows the SEQ ID NOs. of relevant polypeptides. Table IV shows the SEQ ID NOs. of relevant consensus sequences and relevant polypeptide motifs. In all these tables the abbreviation “A. th.” was used for the organism “Arabidopsis thaliana”.

In the following, the term “polypeptide as depicted in Table II or IV” also relates to a polypeptide comprising the consensus sequence or at least one polypeptide motif as depicted in Table IV.

The molecule which activity is to be reduced according to the process of the invention to provide the increase of tolerance and/or resistance to environmental stress and increased biomass production as compared to a corresponding non-transformed wild type plant, e.g. the molecule of I. and/or II. above, is in the following the molecule “which activity is to be reduced in the process of the invention”. The molecule can for example be a polypeptide or a nucleic acid molecule.

Accordingly, in other words, the invention relates to a method for producing a transgenic plant with increased tolerance and/or resistance to environmental stress and increased biomass production as compared to a corresponding non-transformed wild type plant, which comprises the following steps:
a) reduction, repression or deletion of the activity of
   I) at least one polypeptide comprising a polypeptide selected from the group consisting of SEQ ID NOs 28, 105, 191, 411, 513, 674, 730, 814, 924, 1026, 1084, 1386, 1419, 1465, 1552, 1594, and 1651 or a homologue thereof as depicted in column 7 of Table II, preferably as depicted in Table II B, or comprising, a consensus sequence or at least one polypeptide motif of Table IV, Or
   II) at least one expression product of a nucleic acid molecule comprising a polynucleotide selected from the group consisting of SEQ ID NOs 27, 104, 190, 410, 512, 673, 729, 813, 923, 1025, 1083, 1385, 1418, 1464, 1551, 1593, and 1650 or a homologue thereof as depicted in column 7 of Table I, preferably as depicted in in column 5 or 7 of Table I B,
   III) or a functional equivalent of (I) or (II)
   in a plant or a part thereof; and
b) generating a transformed plant with increased tolerance and/or resistance to environmental stress and increased biomass production as compared to a corresponding non-transformed wild type plant and growing under conditions which permit the development of the plant.

In one embodiment the invention relates to a method for producing a transgenic plant with increased tolerance and/or resistance to environmental stress and increased biomass production as compared to a corresponding non-transformed wild type plant, which comprises the following steps:
a) reduction, repression or deletion of the activity of
   I) at least one polypeptide comprising a polypeptide selected from the group consisting of SEQ ID NOs 28, 105, 191, 411, 513, 674, 730, 814, 924, 1026, 1084, 1386, 1419, 1465, 1552, 1594, and 1651 or a homologue thereof as depicted in column 7 of Table II, preferably as depicted in Table II B, or comprising, a consensus sequence or at least one polypeptide motif of Table IV, or
   II) at least one expression product of a nucleic acid molecule comprising a polynucleotide selected from the group consisting of SEQ ID NOs 27, 104, 190, 410, 512, 673, 729, 813, 923, 1025, 1083, 1385, 1418, 1464, 1551, 1593, and 1650 or a homologue thereof as depicted in column 7 of Table I, preferably as depicted in in column 5 or 7 of Table I B,
   III) or a functional equivalent of (I) or (II)
   in a plant or a part thereof; and
b) generating a transformed plant with increased tolerance and/or resistance to environmental stress and increased biomass production as compared to a corresponding non-transformed wild type plant and growing under conditions which permit the development of the plant,
c) imposing drought by withholding water,
d) after the non-transformed wild type plants show visual symptoms of injury selecting the plant with increased tolerance and/or resistance to environmental stress and increased biomass production as compared to a corresponding non-transformed wild type plant.

Surprisingly, it was observed that a knock out of at least one gene conferring an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme or of a gene comprising a nucleic acid sequence described in column 5 of Table I in Arabidopsis thaliana conferred an increase in tolerance and/or resistance to environmental stress and increased biomass production as compared to a corresponding non-transformed wild type plant in the transformed plants.

In particular, it was observed that the knock out of a gene comprising the nucleic acid sequence SEQ ID NO.: 1418 in Arabidopsis thaliana conferred an increased drought resistance by surviving longer than the wild type control without showing any symptoms of injury for a period between 2.7 and 5 days as shown in the Examples, table 1. It was further observed that reducing the activity of a gene product with the activity of a “ubiquitin conjugating enzyme/ubiquitin-like activating enzyme” encoded by a gene comprising the nucleic acid sequence SEQ ID NO.: 1418 in Arabidopsis thaliana conferred an increased biomass production compared with the wild type control without showing any symptoms of injury for a period between 0.6 and 2 days as shown in the Examples.

In particular, it was observed that the knock out of a gene comprising the nucleic acid sequence SEQ ID NO.: 1025 in Arabidopsis thaliana conferred an increased drought resistance by surviving longer than the wild type control without showing any symptoms of injury for a period between 4.7 and 5 days as shown in the Examples, table 1. It was further observed that reducing the activity of a gene product with the activity of a “methyltransferase” encoded by a gene comprising the nucleic acid sequence SEQ ID NO.: 1025 in Arabidopsis thaliana conferred an increased biomass production compared with the wild type control without showing any symptoms of injury for a period between 0.5 and 5 days as shown in the Examples.

In particular, it was observed that the knock out of a gene comprising the nucleic acid sequence SEQ ID NO.: 729 in Arabidopsis thaliana conferred an increased drought resistance by surviving longer than the wild type control without showing any symptoms of injury for a period between 1.8 and 4 days as shown in the Examples, table 1. It was further observed that reducing the activity of a gene product with the activity of a “transcription factor” encoded by a gene comprising the nucleic acid sequence SEQ ID NO.: 729 in Arabidopsis thaliana conferred an increased biomass production compared with the wild type control without showing any symptoms of injury for a period between 1.2 and 3 days as shown in the Examples.

In particular, it was observed that the knock out of a gene comprising the nucleic acid sequence SEQ ID NO.: 27 in Arabidopsis thaliana conferred an increased drought resistance by surviving longer than the wild type control without showing any symptoms of injury for a period between 3 and 5 days as shown in the Examples, table 1. It was further observed that reducing the activity of a gene product with the activity of a “nitrate/chlorate transporter (NRT1.1)” encoded by a gene comprising the nucleic acid sequence SEQ ID NO.: 27 in Arabidopsis thaliana conferred an increased biomass production compared with the wild type control without showing any symptoms of injury for a period between 1.8 and 3 days as shown in the Examples.

In particular, it was observed that the knock out of a gene comprising the nucleic acid sequence SEQ ID NO.: 104 in Arabidopsis thaliana conferred an increased drought resistance by surviving longer than the wild type control without showing any symptoms of injury for a period between 2.9 and 5 days as shown in the Examples, table 1. It was further observed that reducing the activity of a gene product with the activity of a “metalloexopeptidase (MAP1 C)” encoded by a gene comprising the nucleic acid sequence SEQ ID NO.: 104 in Arabidopsis thaliana conferred an increased biomass production compared with the wild type control without showing any symptoms of injury for a period between 1.4 and 3 days as shown in the Examples.

In particular, it was observed that the knock out of a gene comprising the nucleic acid sequence SEQ ID NO.: 190 in Arabidopsis thaliana conferred an increased drought resistance by surviving longer than the wild type control without showing any symptoms of injury for a period between 2.9 and 5 days as shown in the Examples, table 1. It was further observed that reducing the activity of a gene product with the activity of a “proton-dependent oligopeptide transport protein” encoded by a gene comprising the nucleic acid sequence SEQ ID NO.: 190 in Arabidopsis thaliana conferred an increased biomass production compared with the wild type control without showing any symptoms of injury for a period between 1.3 and 2 days as shown in the Examples.

In particular, it was observed that the knock out of a gene comprising the nucleic acid sequence SEQ ID NO.: 512 in Arabidopsis thaliana conferred an increased drought resistance by surviving longer than the wild type control without showing any symptoms of injury for a period between 2.5 and 4 days as shown in the Examples, table 1. It was further observed that reducing the activity of a gene product with the activity of a “amino acid permease (AAP1)” encoded by a gene comprising the nucleic acid sequence SEQ ID NO.: 512 in Arabidopsis thaliana conferred an increased biomass production compared with the wild type control without showing any symptoms of injury for a period between 1 and 2 days as shown in the Examples.

In particular, it was observed that the knock out of a gene comprising the nucleic acid sequence SEQ ID NO.: 1464 in Arabidopsis thaliana conferred an increased drought resistance by surviving longer than the wild type control without showing any symptoms of injury for a period between 2.4 and 5 days as shown in the Examples, table 1. It was further observed that reducing the activity of a gene product with the activity of a “nitrate transporter (ATNRT2.3)” encoded by a gene comprising the nucleic acid sequence SEQ ID NO.: 1464 in Arabidopsis thaliana conferred an increased biomass production compared with the wild type control without showing any symptoms of injury for a period between 1.3 and 3 days as shown in the Examples.

In particular, it was observed that the knock out of a gene comprising the nucleic acid sequence SEQ ID NO.: 813 in Arabidopsis thaliana conferred an increased drought resistance by surviving longer than the wild type control without showing any symptoms of injury for a period between 2.7 and 4 days as shown in the Examples, table 1. It was further observed that reducing the activity of a gene product with the activity of a “pectate lyase protein/powdery mildew susceptibility protein (PMR6)” encoded by a gene comprising the nucleic acid sequence SEQ ID NO.: 813 in Arabidopsis thaliana conferred an increased biomass production compared with the wild type control without showing any symptoms of injury for a period between 1 and 3 days as shown in the Examples.

In particular, it was observed that the knock out of a gene comprising the nucleic acid sequence SEQ ID NO.: 673 in Arabidopsis thaliana conferred an increased drought resistance by surviving longer than the wild type control without showing any symptoms of injury for a period between 2.8 and 5 days as shown in the Examples, table 1. It was further observed that reducing the activity of a gene product with the activity of a “ATP-dependent peptidase/ATPase/nucleoside-triphosphatase/serine-type endopeptidase” encoded by a gene comprising the nucleic acid sequence SEQ ID NO.: 673 in Arabidopsis thaliana conferred an increased biomass production compared with the wild type control without showing any symptoms of injury for a period between 1.5 and 4 days as shown in the Examples.

In particular, it was observed that the knock out of a gene comprising the nucleic acid sequence SEQ ID NO.: 27 in Arabidopsis thaliana conferred an increased drought resistance by surviving longer than the wild type control without showing any symptoms of injury for a period between 2.7 and 5 days as shown in the Examples, table 1. It was further observed that reducing the activity of a gene product with the activity of a “nitrate/chlorate transporter (NRT1.1)” encoded by a gene comprising the nucleic acid sequence SEQ ID NO.: 27 in Arabidopsis thaliana conferred an increased biomass production compared with the wild type control without showing any symptoms of injury for a period between 1.7 and 4 days as shown in the Examples.

In particular, it was observed that the knock out of a gene comprising the nucleic acid sequence SEQ ID NO.: 512 in Arabidopsis thaliana conferred an increased drought resistance by surviving longer than the wild type control without showing any symptoms of injury for a period between 2.9 and 5 days as shown in the Examples, table 1. It was further observed that reducing the activity of a gene product with the activity of a “amino acid permease (AAP1)” encoded by a gene comprising the nucleic acid sequence SEQ ID NO.: 512 in Arabidopsis thaliana conferred an increased biomass production compared with the wild type control without showing any symptoms of injury for a period between 1.4 and 2 days as shown in the Examples.

In particular, it was observed that the knock out of a gene comprising the nucleic acid sequence SEQ ID NO.: 1385 in Arabidopsis thaliana conferred an increased drought resistance by surviving longer than the wild type control without showing any symptoms of injury for a period between 2.5 and 5 days as shown in the Examples, table 1. It was further observed that reducing the activity of a gene product with the activity of a “At5g40590-protein” encoded by a gene comprising the nucleic acid sequence SEQ ID NO.: 1385 in Arabidopsis thaliana conferred an increased biomass production compared with the wild type control without showing any symptoms of injury for a period between 0.9 and 2 days as shown in the Examples.

Further, it was observed that the knock out of a gene comprising the nucleic acid sequence SEQ ID NO.: 1385 in Arabidopsis thaliana conferred an increased cold resistance”, particulary low temperature tolerance, by increasing the biomass production under low temperature conditions compared with the wild type control for 5% to 100% or even more, preferably 10% to 50%, 15% to 40%, more preferably 20% to 30%, 22% to 25%, 23% as shown in the Examples.

In particular, it was observed that the knock out of a gene comprising the nucleic acid sequence SEQ ID NO.: 410 in Arabidopsis thaliana conferred an increased drought resistance by surviving longer than the wild type control without showing any symptoms of injury for a period between 2.7 and 5 days as shown in the Examples, table 1. It was further observed that reducing the activity of a gene product with the activity of a “DNA binding protein/transcription factor” encoded by a gene comprising the nucleic acid sequence SEQ ID NO.: 410 in Arabidopsis thaliana conferred an increased biomass production compared with the wild type control without showing any symptoms of injury for a period between 1.2 and 4 days as shown in the Examples.

In particular, it was observed that the knock out of a gene comprising the nucleic acid sequence SEQ ID NO.: 923 in Arabidopsis thaliana conferred an increased drought resistance by surviving longer than the wild type control without showing any symptoms of injury for a period between 2.5 and 5 days as shown in the Examples, table 1. It was further observed that reducing the activity of a gene product with the activity of a “hydro-lyase/aconitate hydratase” encoded by a gene comprising the nucleic acid sequence SEQ ID NO.: 923 in Arabidopsis thaliana conferred an increased biomass production compared with the wild type control without showing any symptoms of injury for a period between 1.3 and 4 days as shown in the Examples.

In particular, it was observed that the knock out of a gene comprising the nucleic acid sequence SEQ ID NO.: 1593 in Arabidopsis thaliana conferred an increased drought resistance by surviving longer than the wild type control without showing any symptoms of injury for a period between 4 and 5 days as shown in the Examples, table 1. It was further observed that reducing the activity of a gene product with the activity of a “peptidase/ubiquitin-protein ligase/zinc ion binding protein (JR700)” encoded by a gene comprising the nucleic acid sequence SEQ ID NO.: 1593 in Arabidopsis thaliana conferred an increased biomass production compared with the wild type control without showing any symptoms of injury for a period between 0.1 and 0.1 days as shown in the Examples.

In particular, it was observed that the knock out of a gene comprising the nucleic acid sequence SEQ ID NO.: 1083 in Arabidopsis thaliana conferred an increased drought resistance by surviving longer than the wild type control without showing any symptoms of injury for a period between 2.2 and 4 days as shown in the Examples, table 1. It was further observed that reducing the activity of a gene product with the activity of a “DC1 domain-containing protein/protein-binding protein/zinc ion binding protein” encoded by a gene comprising the nucleic acid sequence SEQ ID NO.: 1083 in Arabidopsis thaliana conferred an increased biomass production compared with the wild type control without showing any symptoms of injury for a period between 1 and 3 days as shown in the Examples.

In particular, it was observed that the knock out of a gene comprising the nucleic acid sequence SEQ ID NO.: 1551 in Arabidopsis thaliana conferred an increased drought resistance by surviving longer than the wild type control without showing any symptoms of injury for a period between 2.3 and 4 days as shown in the Examples, table 1. It was further observed that reducing the activity of a gene product with the activity of a “1-phosphatidylinositol 4-kinase” encoded by a gene comprising the nucleic acid sequence SEQ ID NO.: 1551 in Arabidopsis thaliana conferred an increased biomass production compared with the wild type control without showing any symptoms of injury for a period between 0.7 and 2 days as shown in the Examples.

In particular, it was observed that the knock out of a gene comprising the nucleic acid sequence SEQ ID NO.: 1650 in Arabidopsis thaliana conferred an increased drought resistance by surviving longer than the wild type control without showing any symptoms of injury for a period between 4.5 and 5 days as shown in the Examples, table 1. It was further observed that reducing the activity of a gene product with the activity of a “At3g55990-protein” encoded by a gene comprising the nucleic acid sequence SEQ ID NO.: 1650 in Arabidopsis thaliana conferred an increased biomass production compared with the wild type control without showing any symptoms of injury for a period between 2.2 and 4 days as shown in the Examples.

Thus, according to the method of the invention for an increased tolerance and/or resistance to environmental stress and increased biomass production in a plant cell, plant or a part thereof compared to a control or wild type can be achieved.

Accordingly, in one embodiment, in case the activity of the A. thaliana nucleic acid molecule or a polypeptide comprising the nucleic acid SEQ ID NO.: 1418 or polypeptide SEQ ID NO.: 1419, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 1418 or polypeptide SEQ ID NO.: 1419, respectively is reduced or if the activity “ubiquitin conjugating enzyme/ubiquitin-like activating enzyme” is reduced in a plant cell, a plant or a part thereof, preferably

an increased drought resistance by surviving longer than the wild type control without showing any symptoms of injury for a period between 2.7 and 5 days or more and an increased biomass production compared with the wild type control without showing any symptoms of injury for a period between 0.6 and 2 days is conferred.

Accordingly, in one embodiment, in case the activity of the A. thaliana nucleic acid molecule or a polypeptide comprising the nucleic acid SEQ ID NO.: 1025 or polypeptide SEQ ID NO.: 1026, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 1025 or polypeptide SEQ ID NO.: 1026, respectively is reduced or if the activity “methyltransferase” is reduced in a plant cell, a plant or a part thereof, preferably an increased drought resistance by surviving longer than the wild type control without showing any symptoms of injury for a period between 4.7 and 5 days or more and an increased biomass production compared with the wild type control without showing any symptoms of injury for a period between 0.5 and 5 days is conferred.

Accordingly, in one embodiment, in case the activity of the A. thaliana nucleic acid molecule or a polypeptide comprising the nucleic acid SEQ ID NO.: 729 or polypeptide SEQ ID NO.: 730, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 729 or polypeptide SEQ ID NO.: 730, respectively is reduced or if the activity “transcription factor” is reduced in a plant cell, a plant or a part thereof, preferably an increased drought resistance by surviving longer than the wild type control without showing any symptoms of injury for a period between 1.8 and 4 days or more and an increased biomass production compared with the wild type control without showing any symptoms of injury for a period between 1.2 and 3 days is conferred.

Accordingly, in one embodiment, in case the activity of the A. thaliana nucleic acid molecule or a polypeptide comprising the nucleic acid SEQ ID NO.: 27 or polypeptide SEQ ID NO.: 28, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 27 or polypeptide SEQ ID NO.: 28, respectively is reduced or if the activity “nitrate/chlorate transporter (NRT1.1)” is reduced in a plant cell, a plant or a part thereof, preferably an increased drought resistance by surviving longer than the wild type control without showing any symptoms of injury for a period between 3 and 5 days or more and an increased biomass production compared with the wild type control without showing any symptoms of injury for a period between 1.8 and 3 days is conferred.

Accordingly, in one embodiment, in case the activity of the A. thaliana nucleic acid molecule or a polypeptide comprising the nucleic acid SEQ ID NO.: 104 or polypeptide SEQ ID NO.: 105, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 104 or polypeptide SEQ ID NO.: 105, respectively is reduced or if the activity “metalloexopeptidase (MAP1C)” is reduced in a plant cell, a plant or a part thereof, preferably an increased drought resistance by surviving longer than the wild type control without showing any symptoms of injury for a period between 2.9 and 5 days or more and an increased biomass production compared with the wild type control without showing any symptoms of injury for a period between 1.4 and 3 days is conferred.

Accordingly, in one embodiment, in case the activity of the A. thaliana nucleic acid molecule or a polypeptide comprising the nucleic acid SEQ ID NO.: 190 or polypeptide SEQ ID NO.: 191, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 190 or polypeptide SEQ ID NO.: 191, respectively is reduced or if the activity “proton-dependent oligopeptide transport protein” is reduced in a plant cell, a plant or a part thereof, preferably

an increased drought resistance by surviving longer than the wild type control without showing any symptoms of injury for a period between 2.9 and 5 days or more and an increased biomass production compared with the wild type control without showing any symptoms of injury for a period between 1.3 and 2 days is conferred.

Accordingly, in one embodiment, in case the activity of the A. thaliana nucleic acid molecule or a polypeptide comprising the nucleic acid SEQ ID NO.: 512 or polypeptide SEQ ID NO.: 513, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 512 or polypeptide SEQ ID NO.: 513, respectively is reduced or if the activity “amino acid permease (AAP1)” is reduced in a plant cell, a plant or a part thereof, preferably an increased drought resistance by surviving longer than the wild type control without showing any symptoms of injury for a period between 2.5 and 4 days or more and an increased biomass production compared with the wild type control without showing any symptoms of injury for a period between 1 and 2 days is conferred.

Accordingly, in one embodiment, in case the activity of the A. thaliana nucleic acid molecule or a polypeptide comprising the nucleic acid SEQ ID NO.: 1464 or polypeptide SEQ ID NO.: 1465, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 1464 or polypeptide SEQ ID NO.: 1465, respectively is reduced or if the activity “nitrate transporter (ATNRT2.3)” is reduced in a plant cell, a plant or a part thereof, preferably an increased drought resistance by surviving longer than the wild type control without showing any symptoms of injury for a period between 2.4 and 5 days or more and an increased biomass production compared with the wild type control without showing any symptoms of injury for a period between 1.3 and 3 days is conferred.

Accordingly, in one embodiment, in case the activity of the A. thaliana nucleic acid molecule or a polypeptide comprising the nucleic acid SEQ ID NO.: 813 or polypeptide SEQ ID NO.: 814, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 813 or polypeptide SEQ ID NO.: 814, respectively is reduced or if the activity “pectate lyase protein/powdery mildew susceptibility protein (PMR6)” is reduced in a plant cell, a plant or a part thereof, preferably

an increased drought resistance by surviving longer than the wild type control without showing any symptoms of injury for a period between 2.7 and 4 days or more and an increased biomass production compared with the wild type control without showing any symptoms of injury for a period between 1 and 3 days is conferred.

Accordingly, in one embodiment, in case the activity of the A. thaliana nucleic acid molecule or a polypeptide comprising the nucleic acid SEQ ID NO.: 673 or polypeptide SEQ ID NO.: 674, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 673 or polypeptide SEQ ID NO.: 674, respectively is reduced or if the activity “ATP-dependent peptidase/ATPase/nucleoside-triphosphatase/serine-type endopeptidase” is reduced in a plant cell, a plant or a part thereof, preferably an increased drought resistance by surviving longer than the wild type control without showing any symptoms of injury for a period between 2.8 and 5 days or more and an increased biomass production compared with the wild type control without showing any symptoms of injury for a period between 1.5 and 4 days is conferred.

Accordingly, in one embodiment, in case the activity of the A. thaliana nucleic acid molecule or a polypeptide comprising the nucleic acid SEQ ID NO.: 27 or polypeptide SEQ ID NO.: 28, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 27 or polypeptide SEQ ID NO.: 28, respectively is reduced or if the activity “nitrate/chlorate transporter (NRT1.1)” is reduced in a plant cell, a plant or a part thereof, preferably an increased drought resistance by surviving longer than the wild type control without showing any symptoms of injury for a period between 2.7 and 5 days or more and an increased biomass production compared with the wild type control without showing any symptoms of injury for a period between 1.7 and 4 days is conferred.

Accordingly, in one embodiment, in case the activity of the A. thaliana nucleic acid molecule or a polypeptide comprising the nucleic acid SEQ ID NO.: 512 or polypeptide SEQ ID NO.: 513, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 512 or polypeptide SEQ ID NO.: 513, respectively is reduced or if the activity “amino acid permease (AAP1)” is reduced in a plant cell, a plant or a part thereof, preferably an increased drought resistance by surviving longer than the wild type control without showing any symptoms of injury for a period between 2.9 and 5 days or more and an increased biomass production compared with the wild type control without showing any symptoms of injury for a period between 1.4 and 2 days is conferred.

Accordingly, in one embodiment, in case the activity of the A. thaliana nucleic acid molecule or a polypeptide comprising the nucleic acid SEQ ID NO.: 1385 or polypeptide SEQ ID NO.: 1386, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 1385 or polypeptide SEQ ID NO.: 1386, respectively is reduced or if the activity “At5g40590-protein” is reduced in a plant cell, a plant or a part thereof, preferably an increased drought resistance by surviving longer than the wild type control without showing any symptoms of injury for a period between 2.5 and 5 days or more and an increased biomass production compared with the wild type control without showing any symptoms of injury for a period between 0.9 and 2 days is conferred. Accordingly, in one embodiment, in case the activity of the A. thaliana nucleic acid molecule or a polypeptide comprising the nucleic acid SEQ ID NO.: 1385 or polypeptide SEQ ID NO.: 1386, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 1385 or polypeptide SEQ ID NO.: 1386, respectively is reduced or if the activity “At5g40590-protein” is reduced in a plant cell, a plant or a part thereof, preferably an increased biomass production under low temperature conditions compared with the wild type control for 5% to 100% or even more, preferably 10% to 50%, 15% to 40%, more preferably 20% to 30%, 22% to 25%, 23% is conferred.

Accordingly, in one embodiment, in case the activity of the A. thaliana nucleic acid molecule or a polypeptide comprising the nucleic acid SEQ ID NO.: 410 or polypeptide SEQ ID NO.: 411, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 410 or polypeptide SEQ ID NO.: 411, respectively is reduced or if the activity “DNA binding protein/transcription factor” is reduced in a plant cell, a plant or a part thereof, preferably

an increased drought resistance by surviving longer than the wild type control without showing any symptoms of injury for a period between 2.7 and 5 days or more and an increased biomass production compared with the wild type control without showing any symptoms of injury for a period between 1.2 and 4 days is conferred. Accordingly, in one embodiment, in case the activity of the A. thaliana nucleic acid molecule or a polypeptide comprising the nucleic acid SEQ ID NO.: 923 or polypeptide SEQ ID NO.: 924, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 923 or polypeptide SEQ ID NO.: 924, respectively is reduced or if the activity “hydrolyase/aconitate hydratase” is reduced in a plant cell, a plant or a part thereof, preferably

an increased drought resistance by surviving longer than the wild type control without showing any symptoms of injury for a period between 2.5 and 5 days or more and an increased biomass production compared with the wild type control without showing any symptoms of injury for a period between 1.3 and 4 days is conferred.

Accordingly, in one embodiment, in case the activity of the A. thaliana nucleic acid molecule or a polypeptide comprising the nucleic acid SEQ ID NO.: 1593 or polypeptide SEQ ID NO.: 1594, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 1593 or polypeptide SEQ ID NO.: 1594, respectively is reduced or if the activity “peptidase/ubiquitin-protein ligase/zinc ion binding protein (JR700)” is reduced in a plant cell, a plant or a part thereof, preferably

an increased drought resistance by surviving longer than the wild type control without showing any symptoms of injury for a period between 4 and 5 days or more and an increased biomass production compared with the wild type control without showing any symptoms of injury for a period between 0.1 and 0.1 days is conferred.

Accordingly, in one embodiment, in case the activity of the A. thaliana nucleic acid molecule or a polypeptide comprising the nucleic acid SEQ ID NO.: 1083 or polypeptide SEQ ID NO.: 1084, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 1083 or polypeptide SEQ ID NO.: 1084, respectively is reduced or if the activity “DC1 domain-containing protein/protein-binding protein/zinc ion binding protein” is reduced in a plant cell, a plant or a part thereof, preferably

an increased drought resistance by surviving longer than the wild type control without showing any symptoms of injury for a period between 2.2 and 4 days or more and an increased biomass production compared with the wild type control without showing any symptoms of injury for a period between 1 and 3 days is conferred.

Accordingly, in one embodiment, in case the activity of the A. thaliana nucleic acid molecule or a polypeptide comprising the nucleic acid SEQ ID NO.: 1551 or polypeptide SEQ ID NO.: 1552, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 1551 or polypeptide SEQ ID NO.: 1552, respectively is reduced or if the activity “1-phosphatidylinositol 4-kinase” is reduced in a plant cell, a plant or a part thereof, preferably

an increased drought resistance by surviving longer than the wild type control without showing any symptoms of injury for a period between 2.3 and 4 days or more and an increased biomass production compared with the wild type control without showing any symptoms of injury for a period between 0.7 and 2 days is conferred.

Accordingly, in one embodiment, in case the activity of the A. thaliana nucleic acid molecule or a polypeptide comprising the nucleic acid SEQ ID NO.: 1650 or polypeptide SEQ ID NO.: 1651, respectively is reduced or in case in an other organism the activity of the native homolog of said nucleic acid molecule or polypeptide is reduced, e.g. if the activity of a nucleic acid molecule or a polypeptide comprising the nucleic acid or polypeptide or the consensus sequence or the polypeptide motif, as depicted in Table I, II or IV, column 7 in the respective same line as the nucleic acid molecule SEQ ID NO.: 1650 or polypeptide SEQ ID NO.: 1651, respectively is reduced or if the activity “At3g55990-protein” is reduced in a plant cell, a plant or a part thereof, preferably an increased drought resistance by surviving longer than the wild type control without showing any symptoms of injury for a period between 4.5 and 5 days or more and an increased biomass production compared with the wild type control without showing any symptoms of injury for a period between 2.2 and 4 days is conferred.

For the purposes of the invention, as a rule the plural is intended to encompass the singular and vice versa.

Unless otherwise specified, the terms “polynucleotides”, “nucleic acid” and “nucleic acid molecule” are interchangeably in the present context. Unless otherwise specified, the terms “peptide”, “polypeptide” and “protein” are interchangeably in the present context. The term “sequence” may relate to polynucleotides, nucleic acids, nucleic acid molecules, peptides, polypeptides and proteins, depending on the context in which the term “sequence” is used. The terms “gene(s)”, “polynucleotide”, “nucleic acid sequence”, “nucleotide sequence”, or “nucleic acid molecule(s)” as used herein refers to a polymeric form of nucleotides of any length, either ribonucleotides or deoxyribonucleotides. The terms refer only to the primary structure of the molecule.

Thus, the terms “gene(s)”, “polynucleotide”, “nucleic acid sequence”, “nucleotide sequence”, or “nucleic acid molecule(s)” as used herein include double- and single-stranded DNA and/or RNA. They also include known types of modifications, for example, methylation, “caps”, substitutions of one or more of the naturally occurring nucleotides with an analog. Preferably, the DNA or RNA sequence comprises a coding sequence encoding the herein defined polypeptide.

A “coding sequence” is a nucleotide sequence, which is transcribed into an RNA, e.g. a regulatory RNA, such as a miRNA, a ta-siRNA, cosuppression molecule, an RNAi, a ribozyme, etc. or into a mRNA which is translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

As used in the present context a nucleic acid molecule may also encompass the untranslated sequence located at the 3′ and at the 5′ end of the coding gene region, for example at least 500, preferably 200, especially preferably 100, nucleotides of the sequence upstream of the 5′ end of the coding region and at least 100, preferably 50, especially preferably 20, nucleotides of the sequence downstream of the 3′ end of the coding gene region. In the event for example the antisense, RNAi, snRNA, dsRNA, siRNA, miRNA, ta-siRNA, cosuppression molecule, ribozyme etc. technology is used coding regions as well as the 5′- and/or 3′-regions can advantageously be used.

However, it is often advantageous only to choose the coding region for cloning and expression purposes.

“Polypeptide” refers to a polymer of amino acid (amino acid sequence) and does not refer to a specific length of the molecule. Thus peptides and oligopeptides are included within the definition of polypeptide. This term does also refer to or include posttranslational modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring.

The term “Table I” used in this specification is to be taken to specify the content of Table I A and Table I B. The term “Table II” used in this specification is to be taken to specify the content of Table II A and Table II B. The term “Table I A” used in this specification is to be taken to specify the content of Table I A. The term “Table I B” used in this specification is to be taken to specify the content of Table I B. The term “Table II A” used in this specification is to be taken to specify the content of Table II A. The term “Table II B” used in this specification is to be taken to specify the content of Table II B. In one preferred embodiment, the term “Table I” means Table I B. In one preferred embodiment, the term “Table II” means Table II B.

The terms “comprise” or “comprising” and grammatical variations thereof when used in this specification are to be taken to specify the presence of stated features, integers, steps or components or groups thereof, but not to preclude the presence or addition of one or more other features, integers, steps, components or groups thereof.

In accordance with the invention, the term “organism” as understood herein relates always to a non-human organism, in particular to a plant organism, the whole organism, tissues, organs or cell(s) thereof.

The terms “reduction”, “repression”, “decrease” or “deletion” relate to a corresponding change of a property in an organism, a part of an organism such as a tissue, seed, root, tuber, fruit, leave, flower etc. or in a cell. Under “change of a property” it is understood that the activity, expression level or amount of a gene product or a metabolite content is changed in a specific volume or in a specific amount of protein relative to a corresponding volume or amount of protein of a control, reference or wild type. Preferably, the overall activity in the volume is reduced, decreased or deleted in cases if the reduction, decrease or deletion is related to the reduction, decrease or deletion of an activity of a gene product, independent whether the amount of gene product or the specific activity of the gene product or both is reduced, decreased or deleted or whether the amount, stability or translation efficacy of the nucleic acid sequence or gene encoding for the gene product is reduced, decreased or deleted.

The terms “reduction”, “repression”, “decrease” or “deletion” include the change of said property in only parts of the subject of the present invention, for example, the modification can be found in compartment of a cell, like an organelle, or in a part of a plant, including but not limited to tissue, seed, root, leave, tuber, fruit, flower etc. but is not detectable if the overall subject, i.e. complete cell or plant, is tested. Preferably, the “reduction”, “repression”, “decrease” or “deletion” is found cellular, thus the term “reduction, decrease or deletion of an activity” or “reduction, decrease or deletion of a metabolite content” relates to the cellular reduction, decrease or deletion compared to the wild type cell. In addition the terms “reduction”, “repression”, “decrease” or “deletion” include the change of said property only during different growth phases of the organism used in the inventive process, for example the reduction, repression, decrease or deletion takes place only during the seed growth or during blooming. Furthermore the terms include a transitional reduction, decrease or deletion for example because the used method, e.g. the antisense, RNAi, snRNA, dsRNA, siRNA, miRNA, ta-siRNA, cosuppression molecule, or ribozyme, is not stable integrated in the genome of the organism or the reduction, decrease, repression or deletion is under control of a regulatory or inducible element, e.g. a chemical or otherwise inducible promoter, and has therefore only a transient effect.

Accordingly, the term “reduction”, “repression”, “decrease” or “deletion” means that the specific activity of a gene product, an enzyme or other protein or a regulatory RNA as well as the amount of a compound or metabolite, e.g. of a polypeptide, a nucleic acid molecule, or an encoding mRNA or DNA, can be reduced, decreased or deleted in a specific volume. The terms “reduction”, “repression”, “decrease” or “deletion” include that the reason for said “reduction”, “repression”, “decrease” or “deletion” could be a chemical compound that is administered to the organism or part thereof.

Throughout the specification a deletion of the activity or of the expression of an expression product, e.g. of a protein as depicted in Table II means a total loss of the activity. The terms “reduction”, “repression”, or “decrease” are interchangeable. The term “reduction” shall include the terms “repression”, “decrease” or “deletion” if not otherwise specified.

The term “reducing”, “repressing”, “decreasing” or “deleting” as used herein also comprises the term “debasing”, “depleting”, diminishing” or “bringing down”.

Reduction is also understood as meaning the modification of the substrate specificity as can be expressed for example, by the kcat/Km value. In this context, the function or activity, e.g. the enzymatic activity or the “biological activity”, is reduced by at least 10%, advantageously 20%, preferably 30%, especially preferably 40%, 50% or 60%, very especially preferably 70%, 80%, 85% or 90% or more, very especially preferably are 95%, more preferably are 99% or more in comparison to the control, reference or wild type. Most preferably the reduction, decrease or deletion in activity amounts to essentially 100%. Thus, a particularly advantageous embodiment is the inactivation of the function of a compound, e.g. a polypeptide or a nucleic acid molecule.

The reduction, repression or deletion of the expression level or of the activity leads to an increased tolerance and/or resistance to environmental stress and increased biomass production as compared to a corresponding non-transformed wild type plant of 10%, 20%, 30%, 40%, 50%, 100%, 150% or 200% or more, preferably of 250% or 300% or more, particularly preferably of 350% or 400% or more, most particularly preferably of 500% or 600% w/w, or more, expressed in the time the transgenic plant survives longer under conditions of dessication and/or without watering and/or expressed in the time the transgenic plant shows a higher biomass production in comparison to the reference or wild type.

The term “activity” of a compound refers to the function of a compound in a biological system such as a cell, an organ or an organism. For example, the term “activity” of a compound refers to the enzymatic function, regulatory function or its function as binding partner, transporter, regulator, or carrier, etc of a compound.

In one embodiment, the term “biological activity” refers to an activity selected from the group consisting of:
1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme, according to the corresponding context.

The terms “enhance”, “increase”, “decrease”, “repress” or “reduce” or similar terms include the change or the modulation of said property in only one or some parts as well as in all parts of the subject of the present invention. For example, the modification can be found in compartment of a cell, like an organelle, or preferably in a part of a plant, like a tissue, seed, root, leave, fruit, tuber, flower etc. but is not detectable if the overall subject, i.e. complete cell or plant, is tested.

More preferred is the finding that a change or a modulation of said property is found in more than one part of an organism, particularly of a plant.

Thus, in one embodiment, the change or the modulation of said property is found in a tissue, seed, root, fruit, tuber, leave and/or flower of a plant produced according to the process of the present invention.

However, the terms “enhance”, “increase”, “decrease”, “repress” or “reduce” or similar terms as used herein also include the change or modulation of said property in the whole organism as mentioned.

The terms “enhanced” or “increase” mean a 10%, 20%, 30%, 40% or 50% or higher, preferably at least a 60%, 70%, 80%, 90% or 100% or higher, more preferably 150%, 200%, 300%, 400% or 500% or higher tolerance and/or resistance to environmental stress and increased biomass production as compared to a corresponding non-transformed wild type plant. In one embodiment, the increase is calculated as in the examples shown.

As used herein, the term “environmental stress” refers to any suboptimal growing condition and includes, but is not limited to, sub-optimal conditions associated with drought, cold or salinity or combinations thereof. In preferred embodiments, environmental stress is drought and low water content. Wherein drought stress means any environmental stress which leads to a lack of water in plants or reduction of water supply to plants.

In one embodiment of the invention the term “increased tolerance and/or resistance to environmental stress” relates to an increased resistance to water stress, which is produced as a secondary stress by cold, salt, and of course, as a primary stress during drought.

In one embodiment of the invention the term “increased tolerance and/or resistance to environmental stress” relates to an increased cold resistance.

In one embodiment of the invention the term “increased cold resistance” relates to low temperature tolerance, comprising freezing tolerance and/or chilling tolerance.

Further, improved or enhanced “chilling tolerance” or variations thereof refers to improved adaptation to low but non-freezing temperatures around 10° C., preferably temperatures between 1 to 18° C., more preferably 4-14° C., and most preferred 8 to 12° C.; hereinafter called “chilling temperature.

Improved or enhanced “freezing tolerance” or variations thereof refers to improved adaptation to temperatures near or below zero, namely preferably temperatures below 4° C., more preferably below 3 or 2° C., and particularly preferred at or below 0 (zero) ° C. or below −4° C., or even extremely low temperatures down to -10° C. or lower; hereinafter called “freezing temperature.

More generally, “improved adaptation” to environmental stress like low temperatures e.g. freezing and/or chilling temperatures refers to increased biomass production as compared to a corresponding non-transformed wild type plant.

Accordingly, for the purposes of the description of the present invention, the term “low temperature” with respect to low temperature stress on a plant, and preferably a crop plant, refers to any of the low temperature conditions as described herein, preferably chilling and/or freezing temperatures as defined above, as the context requires. It is understood that a skilled artisan will be able to recognize from the particular context in the present description which temperature or temperature range is meant by “low temperature”.

In the present invention, enhanced tolerance to low temperature may, for example and preferably, be determined according to the following method:

Transformed plants are grown in pots in a growth chamber (e.g. York, Mannheim, Germany). In case the plants are Arabidopsis thaliana seeds thereof are sown in pots containing a 3.5:1 (v:v) mixture of nutrient rich soil (GS90, Tantau, Wansdorf, Germany). Plants are grown under standard growth conditions. In case the plants are Arabidopsis thaliana, the standard growth conditions are: photoperiod of 16 h light and 8 h dark, 20° C., 60% relative humidity, and a photon flux density of 200 pmol/m²s. Plants are grown and cultured. In case the plants are Arabidopsis thaliana they are watered every second day. After 12 to 13 days the plants are individualized. Cold (e.g. chilling at 11-12° C.) is applied 14 days after sowing until the end of the experiment. For measuring biomass performance, plant fresh weight was determined at harvest time (29-30 days after sowing) by cutting shoots and weighing them. Beside weighing, phenotypic information was added in case of plants that differ from the wild type control.

In one embodiment of the invention the term “ increased tolerance and/or resistance to environmental stress” relates to an increased salt resistance.

In a preferred embodiment of the invention the term “ increased tolerance and/or resistance to environmental stress” relates to an increased drought resistance.

In an other preferred embodiment of the invention the term “ increased tolerance and/or resistance to environmental stress” relates to an increased resistance to water stress, e.g. drought, cold and salt resistance. Water stress relates to conditions of low water or desiccation.

In one embodiment of the invention the term “increased tolerance and/or resistance to environmental stress” is defined as survival of plants under drought conditions longer than non-transformed wild type plant.

Drought conditions means under conditions of water deficiency, in other words the plants survives and growth under conditions of water deficiency in Arabidopsis for a period of at least 10, preferably 11, 12, more preferably 13 day or more without showing any symptoms of injury, such as wilting and leaf browning and/or rolling, on the other hand the plants being visually turgid and healthy green in color.

In one embodiment of the invention the term “increased biomass production” means that the plants exhibit an increased growth rate from the starting of withholding water as compared to a corresponding non-transformed wild type plant. An increased growth rate comprises an increased in biomass production of the whole plant, an increase in biomass of the visible part of the plant, e.g. of stem and leaves and florescence, visible higher and larger stem.

In one embodiment increased biomass production includes higher seed yield, higher photosynthesis and/or higher dry matter production.

In one embodiment of the invention the term “increased biomass production” means that the plants exhibit an prolonged growth from the starting of withholding water as compared to a corresponding non-transformed wild type plant. An prolonged growth comprises survival and/or continued growth of the whole plant at the moment when the non-transformed wild type plants show visual symptoms of injury.

In one embodiment of the invention the term “increased biomass production” means that the plants exhibit an increased growth rate and prolonged growth from the starting of withholding water as compared to a corresponding non-transformed wild type plant.

In one embodiment of the invention the increased drought resistance is determinated and quantified according to the following method:

Transformed plants are grown individually in pots in a growth chamber (York Industriekälte GmbH, Mannheim, Germany).

Germination is induced. In case the plants are Arabidopsis thaliana sown seeds are kept at 4° C., in the dark, for 3 days in order to induce germination. Subsequently conditions are changed for 3 days to 20° C./6° C. day/night temperature with a 16/8 h day-night cycle at 150 μE/m²s.

Subsequently the plants are grown under standard growth conditions. In case the plants are Arabidopsis thaliana, the standard growth conditions are: photoperiod of 16 h light and 8 h dark, 20° C., 60% relative humidity, and a photon flux density of 200 μE. Plants are grown and cultured until they develop leaves. In case the plants are Arabidopsis thaliana they are watered daily until they were approximately 3 weeks old. Starting at that time drought was imposed by withholding water.

After the non-transformed wild type plants show visual symptoms of injury, the evaluation starts and plants are scored for symptoms of drought symptoms and biomass production comparison to wild type and neighboring plants for 5-6 days in succession.

Visual symptoms of injury stating for one or any combination of two, three or more of the following features:
a) wilting
b) leaf browning
c) loss of turgor, which results in drooping of leaves or needles stems, and flowers,
d) drooping and/or shedding of leaves or needles,
e) the leaves are green but leaf angled slightly toward the ground compared with controls,
f) leaf blades begun to fold (curl) inward,
g) premature senescence of leaves or needles,
h) loss of chlorophyll in leaves or needles and/or yellowing.

The term “reference”, “control” or “wild type” mean an organism without the aforementioned modification of the expression or activity of an expression product of a nucleic acid molecule comprising a polynucleotide indicated in Table I, column 5 or 7 or of the activity of a protein having the activity of a polypeptide comprising a polypeptide indicated in Table II or IV, column 5 or 7, or of the activity of a protein encoded by nucleic acid molecule comprising a nucleic acid molecule indicated in Table I, column 5 or 7.

In other words wild type denotes (a) the organism which carries the unaltered (usually the “normal”) form of a gene or allele; (b) the laboratory stock from which mutants are derived. The adjective “wild-type” may refer to the phenotype or genotype.

A “reference”, “control” or “wild type” is in particular a cell, a tissue, an organ, a plant, or a part thereof, which was not produced according to the process of the invention.

Accordingly, the terms “wild type”, “control” or “reference” are exchangeable and can be a cell or a part of organisms such as an organelle or tissue, or an organism, in particular a plant, which was not modified or treated according to the herein described process according to the invention. Accordingly, the cell or a part of organisms such as an organelle or a tissue, or an organism, in particular a plant used as wild type, control or reference corresponds to the cell, organism or part thereof as much as possible and is in any other property but in the result of the process of the invention as identical to the subject matter of the invention as possible. Thus, the wild type, control or reference is treated identically or as identical as possible, saying that only conditions or properties might be different which do not influence the quality of the tested property.

Preferably, any comparison is carried out under analogous conditions. The term “analogous conditions” means that all conditions such as, for example, culture or growing conditions, assay conditions (such as buffer composition, temperature, substrates, pathogen strain, concentrations and the like) are kept identical between the experiments to be compared.

The “reference”, “control”, or “wild type” is preferably a subject, e.g. an organelle, a cell, a tissue, an organism, in particular a plant, which was not modified or treated according to the herein described process of the invention and is in any other property as similar to the subject matter of the invention as possible. The reference, control or wild type is in its genome, transcriptome, proteome or metabolome as similar as possible to the subject of the present invention. Preferably, the term “reference-” “control-” or “wild type-”-organelle, -cell, -tissue or -organism, in particular plant, relates to an organelle, cell, tissue or organism, in particular plant, which is nearly genetically identical to the organelle, cell, tissue or organism, in particular plant, of the present invention or a part thereof preferably 95%, more preferred are 98%, even more preferred are 99.00%, in particular 99.10%, 99.30%, 99.50%, 99.70%, 99.90%, 99.99%, 99.999% or more. Most preferable the “reference”, “control”, or “wild type” is preferably a subject, e.g. an organelle, a cell, a tissue, an organism, which is genetically identical to the organism, cell organelle used according to the process of the invention except that nucleic acid molecules or the gene product encoded by them are changed or modified according to the inventive process.

In case, a control, reference or wild type differing from the subject of the present invention only by not being subject of the process of the invention can not be provided, a control, reference or wild type can be an organism in which the cause for the modulation of the activity conferring the increase of tolerance and/or resistance to environmental stress and increase of biomass production as described herein has been switched back or off, e.g. by complementation of responsible reduced gene product, e.g. by stable or transient (over)expression, by activation of an activator or agonist, by inactivation of an inhibitor or antagonist, by adding active compounds as e.g. hormones, by introducing enhancers etc.

Accordingly, preferred reference subject is the starting subject of the present process of the invention.

Preferably, the reference and the subject matter of the invention are compared after standardization and normalization, e.g. to the amount of total RNA, DNA, or protein or activity or expression of reference genes, like housekeeping genes, such as certain actin or ubiquitin genes.

Preferably, the reference, control or wild type differs form the subject of the present invention only in the cellular activity of the polypeptide or RNA used in the process of the invention, e.g. as result of a reduction, decrease or deletion in the level of the nucleic acid molecule of the present invention or a reduction, decrease or deletion of the specific activity of the polypeptide or RNA used in the process of the invention, e.g. by the expression level or activity of protein or RNA, that means by reduction or inhibition of its biological activity and/or of its biochemical or genetical causes.

The term “expression” refers to the transcription and/or translation of a codogenic gene segment or gene. As a rule, the resulting product is a mRNA or a protein. However, expression products can also include functional RNAs such as, for example, antisense, tRNAs, snRNAs, rRNAs, dsRNAs, siRNAs, miRNAs, ta-siRNA, cosuppression molecules, ribozymes etc. Expression may be systemic, local or temporal, for example limited to certain cell types, tissues organs or time periods.

The term “expression” means the transcription of a gene into an RNA (e.g. rRNA, tRNA, miRNA, dsRNA, snRNA, ta-siRNA, sRNA) or messenger RNA (mRNA). Thus, term “expression” means the expression of a gene with or without the subsequent translation of the latter into a protein. Experimentally, expression on RNA level can be detected by methods well known, e.g. Northern blotting, array hybridizations, qRT PCR, transcriptional run-on assays. Further, experimentally, expression on polypeptide level can be detected by methods well known, e.g. Western blotting or other immuno assays.

The term “functional equivalent” of a polypeptide as depicted in column 5 or 7 of Table II is a polypeptide which confers essentially the activity of a polypeptide as depicted in column 5 Table II.

The term “functional equivalent” of a nucleic acid molecule as depicted in column 5 or 7 of Table I is a polynucleotide which confers essentially the activity of a nucleic acid molecule as depicted in column 5 of Table I.

In accordance with the invention, a protein or polypeptide has the activity of a polypeptide as depicted in column 5 of Table II if the reduction, repression, decrease or deletion of its activity mediates the increased tolerance and/or resistance to environmental stress and increased biomass production as compared to a corresponding non-transformed wild type plant.

In particular, a protein or polypeptide has the activity of a polypeptide as depicted in column 5 of Table II if the reduction, repression, decrease or deletion of its activity mediates the increased tolerance and/or resistance to environmental stress and increased biomass production as compared to a corresponding non-transformed wild type plant.

In accordance with the invention, a nucleic acid molecule or polynucleotide has the activity of a nucleic acid molecule as depicted in column 5 of Table I″ if the reduction, repression, decrease or deletion of its expression mediates the increased tolerance and/or resistance to environmental stress and increased biomass production as compared to a corresponding non-transformed wild type plant.

That means for example that the reduction, repression or deletion of an expression, like the expression of a gene product, or of an activity like an enzymatic activity, is somehow related to the increased tolerance and/or resistance to environmental stress and increased biomass production as compared to a corresponding nontransformed wild type plant.

Throughout the specification the reduction, repression or deletion of the activity of such an aforementioned protein or polypeptide or of the expression product of such an aforementioned nucleic acid molecule or sequence means a reduction of the translation, transcription or expression level or activity of the gene product or the polypeptide, for example the enzymatic or biological activity of the polypeptide, of at least 10% preferably 20%, 30%, 40% or 50%, particularly preferably 60% 70% or 80%, most particularly preferably 90%, 91%, 92%, 93%, 94%, 95%, 96%, 97%, 98% or 99% in comparison to the original endogenous expression level of the expression product or to the original endogenous activity of an expression product or polypeptide comprising or being encoded by a nucleic acid molecule as indicated in column 5 or 7 of Table I or comprising a polypeptide as indicated in column 5 or 7 of Table II or IV or the endogenous homologue or equivalent thereof.

Further, the person skilled in the art can determine whether a polypeptide has the “activity of a polypeptide as depicted in column 5 of Table II” in a complementation assay.

Further, the person skilled in the art can determine whether a nucleic acid molecule has the “activity of a nucleic acid molecule as depicted in column 5 of Table I” in a complementation assay.

The specific activity of a polypeptide or a nucleic acid molecule as described herein for use in the process of the present invention can be tested as described in the examples or in the state of the art. In particular, determination whether the expression of a polynucleotide or a polypeptide in question is reduced, decreased or deleted in a plant cell and the detection of an increased tolerance and/or resistance to environmental stress and increased biomass production as compared to a corresponding non-transformed wild type plant is an easy test and can be performed as described in the examples or in the state of the art.

In order to test whether a nucleic acid molecule, e.g. a gene, is a functional homologue of a nucleic acid molecule depicted in columns 5 or 7, in particular depicted in column 5, a complementation assay in a microorganism or a plant can be performed. For example, a plant lacking the activity of the gene, e.g. a Arabidopsis thaliana strain in which a nucleic acid molecule comprising the nucleic acid molecule has been knocked out, in particular deleted or interrupted, can be transformed with the respective nucleic acid molecule in question, e.g. a gene or homologue, under control of a suitable promoter, e.g. in a suitable vector. The promoter may either confer constitutive or transient or tissue or development specific or inducible expression. Preferably the promoter may be similar or identical in spatial and temporal activity to the promoter of the gene, which has been knock out, deleted or interrupted. The nucleic acid molecule in question, e.g. the gene or the homologue to be tested preferably comprises the complete coding region either with or without introns(s). In addition, it might be preferable to add 5′and 3′ UTR or other features to the sequence in order to increase stability or translation of the transcript.

Transformed plants are analyzed for the presence of the respective construct and the expression of the nucleic acid molecule in question, e.g. the gene or homologue, or its expression product. Plants exhibiting expression of the gene or homologue are compared to wild type plants. The transgenic plant, comprising a knockout mutation and expressing the respective gene or homologue is essentially identical to wild type controls with regard to the change in the tolerance and/or resistance to environmental stress and biomass production as compared to a corresponding non-transformed wild type plant.

A qualified complementation assay is for example described in Iba K (1993) Journal of Biological Chemistry 268 (32) pp 24099-24105, Bonaventure G et al (2003) Plant Growth. Plant Cell 15 pp 1020-1033, or in Gachotte D et al (1995) Plant Journal 8 (3) pp 407-416.

The sequence of At5g50870 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as ubiquitin conjugating enzyme/ubiquitin-like activating enzyme.

Accordingly, in one embodiment, the process of the present invention comprises the reduction of a gene product with the activity of a “ubiquitin conjugating enzyme/ubiquitin-like activating enzyme” from Arabidopsis thaliana or its functional equivalent or its homolog, e.g. the reduction of
a) a gene product of a gene comprising the nucleic acid molecule as shown in column 5 of Table I and being depicted in the same respective line as said At5g50870 or a functional equivalent or a homologue thereof as depicted in column 7 of Table I, preferably a homologue or functional equivalent as depicted in column 7 of Table I B, and being depicted in the same respective line as said At5g50870; or
b) a polypeptide comprising a polypeptide, a consensus sequence or a polypeptide motif as depicted in column 5 of Table II or column 7 of table IV respectively, and being depicted in the same respective line as said At5g50870 or a functional equivalent or a homologue thereof as depicted in column 7 of Table II, preferably a homologue or functional equivalent as depicted in column 7 of Table II B, and being depicted in the same respective line as said At5g50870,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “ubiquitin conjugating enzyme/ubiquitin-like activating enzyme”, preferably it is the molecule of section (a) or (b) of this paragraph [0024.1.1.1].

The sequence of At4g31120 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as methyltransferase.

Accordingly, in one embodiment, the process of the present invention comprises the reduction of a gene product with the activity of a “methyltransferase” from Arabidopsis thaliana or its functional equivalent or its homolog, e.g. the reduction of
a) a gene product of a gene comprising the nucleic acid molecule as shown in column 5 of Table I and being depicted in the same respective line as said At4g31120 or a functional equivalent or a homologue thereof as depicted in column 7 of Table I, preferably a homologue or functional equivalent as depicted in column 7 of Table I B, and being depicted in the same respective line as said At4g31120; or
b) a polypeptide comprising a polypeptide, a consensus sequence or a polypeptide motif as depicted in column 5 of Table II or column 7 of table IV respectively, and being depicted in the same respective line as said At4g31120 or a functional equivalent or a homologue thereof as depicted in column 7 of Table II, preferably a homologue or functional equivalent as depicted in column 7 of Table II B, and being depicted in the same respective line as said At4g31120,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “methyltransferase”, preferably it is the molecule of section (a) or (b) of this paragraph [0024.1.1.1].

The sequence of At3g14230 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as transcription factor.

Accordingly, in one embodiment, the process of the present invention comprises the reduction of a gene product with the activity of a “transcription factor” from Arabidopsis thaliana or its functional equivalent or its homolog, e.g. the reduction of
a) a gene product of a gene comprising the nucleic acid molecule as shown in column 5 of Table I and being depicted in the same respective line as said At3g14230 or a functional equivalent or a homologue thereof as depicted in column 7 of Table I, preferably a homologue or functional equivalent as depicted in column 7 of Table I B, and being depicted in the same respective line as said At3g14230; or
b) a polypeptide comprising a polypeptide, a consensus sequence or a polypeptide motif as depicted in column 5 of Table II or column 7 of table IV respectively, and being depicted in the same respective line as said At3g14230 or a functional equivalent or a homologue thereof as depicted in column 7 of Table II, preferably a homologue or functional equivalent as depicted in column 7 of Table II B, and being depicted in the same respective line as said At3g14230,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “transcription factor”, preferably it is the molecule of section (a) or (b) of this paragraph [0024.1.1.1].

The sequence of At1g12110 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as nitrate/chlorate transporter (NRT1.1).

Accordingly, in one embodiment, the process of the present invention comprises the reduction of a gene product with the activity of a “nitrate/chlorate transporter (NRT1.1)” from Arabidopsis thaliana or its functional equivalent or its homolog, e.g. the reduction of
a) a gene product of a gene comprising the nucleic acid molecule as shown in column 5 of Table I and being depicted in the same respective line as said At1g12110 or a functional equivalent or a homologue thereof as depicted in column 7 of Table I, preferably a homologue or functional equivalent as depicted in column 7 of Table I B, and being depicted in the same respective line as said At1g12110; or
b) a polypeptide comprising a polypeptide, a consensus sequence or a polypeptide motif as depicted in column 5 of Table II or column 7 of table IV respectively, and being depicted in the same respective line as said At1g12110 or a functional equivalent or a homologue thereof as depicted in column 7 of Table II, preferably a homologue or functional equivalent as depicted in column 7 of Table II B, and being depicted in the same respective line as said At1g12110,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “nitrate/chlorate transporter (NRT1.1)”, preferably it is the molecule of section (a) or (b) of this paragraph [0024.1.1.1].

The sequence of At1g13270 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as metalloexopeptidase (MAP1C).

Accordingly, in one embodiment, the process of the present invention comprises the reduction of a gene product with the activity of a “metalloexopeptidase (MAP1C)” from Arabidopsis thaliana or its functional equivalent or its homolog, e.g. the reduction of
a) a gene product of a gene comprising the nucleic acid molecule as shown in column 5 of Table I and being depicted in the same respective line as said At1g13270 or a functional equivalent or a homologue thereof as depicted in column 7 of Table I, preferably a homologue or functional equivalent as depicted in column 7 of Table I B, and being depicted in the same respective line as said At1g13270; or
b) a polypeptide comprising a polypeptide, a consensus sequence or a polypeptide motif as depicted in column 5 of Table II or column 7 of table IV respectively, and being depicted in the same respective line as said At1g13270 or a functional equivalent or a homologue thereof as depicted in column 7 of Table II, preferably a homologue or functional equivalent as depicted in column 7 of Table II B, and being depicted in the same respective line as said At1g13270,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “metalloexopeptidase (MAP1 C)”, preferably it is the molecule of section (a) or (b) of this paragraph [0024.1.1.1].

The sequence of At1g27080 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as proton-dependent oligopeptide transport protein.

Accordingly, in one embodiment, the process of the present invention comprises the reduction of a gene product with the activity of a “proton-dependent oligopeptide transport protein” from Arabidopsis thaliana or its functional equivalent or its homolog, e.g. the reduction of
a) a gene product of a gene comprising the nucleic acid molecule as shown in column 5 of Table I and being depicted in the same respective line as said At1g27080 or a functional equivalent or a homologue thereof as depicted in column 7 of Table I, preferably a homologue or functional equivalent as depicted in column 7 of Table I B, and being depicted in the same respective line as said At1g27080; or
b) a polypeptide comprising a polypeptide, a consensus sequence or a polypeptide motif as depicted in column 5 of Table II or column 7 of table IV respectively, and being depicted in the same respective line as said At1g27080 or a functional equivalent or a homologue thereof as depicted in column 7 of Table II, preferably a homologue or functional equivalent as depicted in column 7 of Table II B, and being depicted in the same respective line as said At1g27080,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “proton-dependent oligopeptide transport protein”, preferably it is the molecule of section (a) or (b) of this paragraph [0024.1.1.1].

The sequence of AT1G58360 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as amino acid permease (AAP1).

Accordingly, in one embodiment, the process of the present invention comprises the reduction of a gene product with the activity of a “amino acid permease (AAP1)” from Arabidopsis thaliana or its functional equivalent or its homolog, e.g. the reduction of
a) a gene product of a gene comprising the nucleic acid molecule as shown in column 5 of Table I and being depicted in the same respective line as said AT1G58360 or a functional equivalent or a homologue thereof as depicted in column 7 of Table I, preferably a homologue or functional equivalent as depicted in column 7 of Table I B, and being depicted in the same respective line as said AT1G58360; or
b) a polypeptide comprising a polypeptide, a consensus sequence or a polypeptide motif as depicted in column 5 of Table II or column 7 of table IV respectively, and being depicted in the same respective line as said AT1G58360 or a functional equivalent or a homologue thereof as depicted in column 7 of Table II, preferably a homologue or functional equivalent as depicted in column 7 of Table II B, and being depicted in the same respective line as said AT1G58360,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “amino acid permease (AAP1)”, preferably it is the molecule of section (a) or (b) of this paragraph [0024.1.1.1].

The sequence of At5g60780 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as nitrate transporter (ATNRT2.3).

Accordingly, in one embodiment, the process of the present invention comprises the reduction of a gene product with the activity of a “nitrate transporter (ATNRT2.3)” from Arabidopsis thaliana or its functional equivalent or its homolog, e.g. the reduction of
a) a gene product of a gene comprising the nucleic acid molecule as shown in column 5 of Table I and being depicted in the same respective line as said At5g60780 or a functional equivalent or a homologue thereof as depicted in column 7 of Table I, preferably a homologue or functional equivalent as depicted in column 7 of Table I B, and being depicted in the same respective line as said At5g60780; or
b) a polypeptide comprising a polypeptide, a consensus sequence or a polypeptide motif as depicted in column 5 of Table II or column 7 of table IV respectively, and being depicted in the same respective line as said At5g60780 or a functional equivalent or a homologue thereof as depicted in column 7 of Table II , preferably a homologue or functional equivalent as depicted in column 7 of Table II B, and being depicted in the same respective line as said At5g60780,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “nitrate transporter (ATNRT2.3)”, preferably it is the molecule of section (a) or (b) of this paragraph [0024.1.1.1].

The sequence of At3g54920 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as pectate lyase protein/powdery mildew susceptibility protein (PMR6).

Accordingly, in one embodiment, the process of the present invention comprises the reduction of a gene product with the activity of a “pectate lyase protein/powdery mildew susceptibility protein (PMR6)” from Arabidopsis thaliana or its functional equivalent or its homolog, e.g. the reduction of
a) a gene product of a gene comprising the nucleic acid molecule as shown in column 5 of Table I and being depicted in the same respective line as said At3g54920 or a functional equivalent or a homologue thereof as depicted in column 7 of Table I, preferably a homologue or functional equivalent as depicted in column 7 of Table I B, and being depicted in the same respective line as said At3g54920; or
b) a polypeptide comprising a polypeptide, a consensus sequence or a polypeptide motif as depicted in column 5 of Table II or column 7 of table IV respectively, and being depicted in the same respective line as said At3g54920 or a functional equivalent or a homologue thereof as depicted in column 7 of Table II, preferably a homologue or functional equivalent as depicted in column 7 of Table II B, and being depicted in the same respective line as said At3g54920,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “pectate lyase protein/powdery mildew susceptibility protein (PMR6)”, preferably it is the molecule of section (a) or (b) of this paragraph [0024.1.1.1].

The sequence of AT2G03670 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as ATP-dependent peptidase/ATPase/nucleoside-triphosphatase/serine-type endopeptidase.

Accordingly, in one embodiment, the process of the present invention comprises the reduction of a gene product with the activity of a “ATP-dependent peptidase/ATPase/nucleoside-triphosphatase/serine-type endopeptidase” from Arabidopsis thaliana or its functional equivalent or its homolog, e.g. the reduction of
a) a gene product of a gene comprising the nucleic acid molecule as shown in column 5 of Table I and being depicted in the same respective line as said AT2G03670 or a functional equivalent or a homologue thereof as depicted in column 7 of Table I, preferably a homologue or functional equivalent as depicted in column 7 of Table I B, and being depicted in the same respective line as said AT2G03670; or
b) a polypeptide comprising a polypeptide, a consensus sequence or a polypeptide motif as depicted in column 5 of Table II or column 7 of table IV respectively, and being depicted in the same respective line as said AT2G03670 or a functional equivalent or a homologue thereof as depicted in column 7 of Table II, preferably a homologue or functional equivalent as depicted in column 7 of Table II B, and being depicted in the same respective line as said AT2G03670,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “ATP-dependent peptidase/ATPase/nucleoside-triphosphatase/serine-type endopeptidase”, preferably it is the molecule of section (a) or (b) of this paragraph [0024.1.1.1].

The sequence of At1g12110 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as nitrate/chlorate transporter (NRT1.1).

Accordingly, in one embodiment, the process of the present invention comprises the reduction of a gene product with the activity of a “nitrate/chlorate transporter (NRT1.1)” from Arabidopsis thaliana or its functional equivalent or its homolog, e.g. the reduction of
a) a gene product of a gene comprising the nucleic acid molecule as shown in column 5 of Table I and being depicted in the same respective line as said At1g12110 or a functional equivalent or a homologue thereof as depicted in column 7 of Table I, preferably a homologue or functional equivalent as depicted in column 7 of Table I B, and being depicted in the same respective line as said At1g12110; or
b) a polypeptide comprising a polypeptide, a consensus sequence or a polypeptide motif as depicted in column 5 of Table II or column 7 of table IV respectively, and being depicted in the same respective line as said At1g12110 or a functional equivalent or a homologue thereof as depicted in column 7 of Table II, preferably a homologue or functional equivalent as depicted in column 7 of Table II B, and being depicted in the same respective line as said At1g12110,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “nitrate/chlorate transporter (NRT1.1)”, preferably it is the molecule of section (a) or (b) of this paragraph [0024.1.1.1].

The sequence of AT1G58360 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as amino acid permease (AAP1).

Accordingly, in one embodiment, the process of the present invention comprises the reduction of a gene product with the activity of a “amino acid permease (AAP1)” from Arabidopsis thaliana or its functional equivalent or its homolog, e.g. the reduction of
a) a gene product of a gene comprising the nucleic acid molecule as shown in column 5 of Table I and being depicted in the same respective line as said AT1G58360 or a functional equivalent or a homologue thereof as depicted in column 7 of Table I, preferably a homologue or functional equivalent as depicted in column 7 of Table I B, and being depicted in the same respective line as said AT1G58360; or
b) a polypeptide comprising a polypeptide, a consensus sequence or a polypeptide motif as depicted in column 5 of Table II or column 7 of table IV respectively, and being depicted in the same respective line as said AT1G58360 or a functional equivalent or a homologue thereof as depicted in column 7 of Table II preferably a homologue or functional equivalent as depicted in column 7 of Table II B, and being depicted in the same respective line as said AT1G58360,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “amino acid permease (AAP1)”, preferably it is the molecule of section (a) or (b) of this paragraph [0024.1.1.1].

The sequence of At5g40590 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as At5g40590-protein.

Accordingly, in one embodiment, the process of the present invention comprises the reduction of a gene product with the activity of a “At5g40590-protein” from Arabidopsis thaliana or its functional equivalent or its homolog, e.g. the reduction of
a) a gene product of a gene comprising the nucleic acid molecule as shown in column 5 of Table I and being depicted in the same respective line as said At5g40590 or a functional equivalent or a homologue thereof as depicted in column 7 of Table I, preferably a homologue or functional equivalent as depicted in column 7 of Table I B, and being depicted in the same respective line as said At5g40590; or
b) a polypeptide comprising a polypeptide, a consensus sequence or a polypeptide motif as depicted in column 5 of Table II or column 7 of table IV respectively, and being depicted in the same respective line as said At5g40590 or a functional equivalent or a homologue thereof as depicted in column 7 of Table II, preferably a homologue or functional equivalent as depicted in column 7 of Table II B, and being depicted in the same respective line as said At5g40590,
   as mentioned herein, for the increased tolerance and/or resistance to environmental stress and increased biomass production as compared to a corresponding non-transformed wild type plant.

In one embodiment of the invention the term “increased tolerance and/or resistance to environmental stress” relates to an increased cold resistance, meaning to low temperature tolerance, comprising freezing tolerance and/or chilling tolerance.

Accordingly, in one embodiment, the molecule which activity is to be reduced in the process of the invention is the gene product with an activity described as “At5g40590-protein”, preferably it is the molecule of section (a) or (b) of this paragraph [0024.1.1.1].

The sequence of At1g33760 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as DNA binding protein/transcription factor.

Accordingly, in one embodiment, the process of the present invention comprises the reduction of a gene product with the activity of a “DNA binding protein/transcription factor” from Arabidopsis thaliana or its functional equivalent or its homolog, e.g. the reduction of
a) a gene product of a gene comprising the nucleic acid molecule as shown in column 5 of Table I and being depicted in the same respective line as said At1g33760 or a functional equivalent or a homologue thereof as depicted in column 7 of Table I, preferably a homologue or functional equivalent as depicted in column 7 of Table I B, and being depicted in the same respective line as said At1g33760; or
b) a polypeptide comprising a polypeptide, a consensus sequence or a polypeptide motif as depicted in column 5 of Table II or column 7 of table IV respectively, and being depicted in the same respective line as said At1g33760 or a functional equivalent or a homologue thereof as depicted in column 7 of Table II , preferably a homologue or functional equivalent as depicted in column 7 of Table II B, and being depicted in the same respective line as said At1g33760,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “DNA binding protein/transcription factor”, preferably it is the molecule of section (a) or (b) of this paragraph [0024.1.1.1].

The sequence of At4g13430 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as hydro-lyase/aconitate hydratase.

Accordingly, in one embodiment, the process of the present invention comprises the reduction of a gene product with the activity of a “hydro-lyase/aconitate hydratase” from Arabidopsis thaliana or its functional equivalent or its homolog, e.g. the reduction of
a) a gene product of a gene comprising the nucleic acid molecule as shown in column 5 of Table I and being depicted in the same respective line as said At4g13430 or a functional equivalent or a homologue thereof as depicted in column 7 of Table I, preferably a homologue or functional equivalent as depicted in column 7 of Table I B, and being depicted in the same respective line as said At4g13430; or
b) a polypeptide comprising a polypeptide, a consensus sequence or a polypeptide motif as depicted in column 5 of Table II or column 7 of table IV respectively, and being depicted in the same respective line as said At4g13430 or a functional equivalent or a homologue thereof as depicted in column 7 of Table II , preferably a homologue or functional equivalent as depicted in column 7 of Table II B, and being depicted in the same respective line as said At4g13430,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “hydro-lyase/aconitate hydratase”, preferably it is the molecule of section (a) or (b) of this paragraph [0024.1.1.1].

The sequence of At5g66160 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as peptidase/ubiquitin-protein ligase/zinc ion binding protein (JR700).

Accordingly, in one embodiment, the process of the present invention comprises the reduction of a gene product with the activity of a “peptidase/ubiquitin-protein ligase/zinc ion binding protein (JR700)” from Arabidopsis thaliana or its functional equivalent or its homolog, e.g. the reduction of
a) a gene product of a gene comprising the nucleic acid molecule as shown in column 5 of Table I and being depicted in the same respective line as said At5g66160 or a functional equivalent or a homologue thereof as depicted in column 7 of Table I, preferably a homologue or functional equivalent as depicted in column 7 of Table I B, and being depicted in the same respective line as said At5g66160; or
b) a polypeptide comprising a polypeptide, a consensus sequence or a polypeptide motif as depicted in column 5 of Table II or column 7 of table IV respectively, and being depicted in the same respective line as said At5g66160 or a functional equivalent or a homologue thereof as depicted in column 7 of Table II , preferably a homologue or functional equivalent as depicted in column 7 of Table II B, and being depicted in the same respective line as said At5g66160,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “peptidase/ubiquitin-protein ligase/zinc ion binding protein (JR700)”, preferably it is the molecule of section (a) or (b) of this paragraph [0024.1.1.1].

The sequence of At5g02330 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as DC1 domain-containing protein/protein-binding protein/zinc ion binding protein.

Accordingly, in one embodiment, the process of the present invention comprises the reduction of a gene product with the activity of a “DC1 domain-containing protein/protein-binding protein/zinc ion binding protein” from Arabidopsis thaliana or its functional equivalent or its homolog, e.g. the reduction of
a) a gene product of a gene comprising the nucleic acid molecule as shown in column 5 of Table I and being depicted in the same respective line as said At5g02330 or a functional equivalent or a homologue thereof as depicted in column 7 of Table I, preferably a homologue or functional equivalent as depicted in column 7 of Table I B, and being depicted in the same respective line as said At5g02330; or
b) a polypeptide comprising a polypeptide, a consensus sequence or a polypeptide motif as depicted in column 5 of Table II or column 7 of table IV respectively, and being depicted in the same respective line as said At5g02330 or a functional equivalent or a homologue thereof as depicted in column 7 of Table II , preferably a homologue or functional equivalent as depicted in column 7 of Table II B, and being depicted in the same respective line as said At5g02330,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “DC1 domain-containing protein/protein-binding protein/zinc ion binding protein”, preferably it is the molecule of section (a) or (b) of this paragraph [0024.1.1.1].

The sequence of At5g64070 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as 1-phosphatidylinositol 4-kinase.

Accordingly, in one embodiment, the process of the present invention comprises the reduction of a gene product with the activity of a “1-phosphatidylinositol 4-kinase” from Arabidopsis thaliana or its functional equivalent or its homolog, e.g. the reduction of
a) a gene product of a gene comprising the nucleic acid molecule as shown in column 5 of Table I and being depicted in the same respective line as said At5g64070 or a functional equivalent or a homologue thereof as depicted in column 7 of Table I, preferably a homologue or functional equivalent as depicted in column 7 of Table I B, and being depicted in the same respective line as said At5g64070; or
b) a polypeptide comprising a polypeptide, a consensus sequence or a polypeptide motif as depicted in column 5 of Table II or column 7 of table IV respectively, and being depicted in the same respective line as said At5g64070 or a functional equivalent or a homologue thereof as depicted in column 7 of Table II , preferably a homologue or functional equivalent as depicted in column 7 of Table II B, and being depicted in the same respective line as said At5g64070,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “1-phosphatidylinositol 4-kinase”, preferably it is the molecule of section (a) or (b) of this paragraph [0024.1.1.1].

The sequence of At3g55990 from Arabidopsis thaliana, e.g. as shown incolumn 5 of Table I, has been published in the TAIR database http://www.arabidopsis.org (Huala, E. et al., Nucleic Acids Res. 2001 Vol. 29(1), 102-5), and its activity is described as At3g55990-protein.

Accordingly, in one embodiment, the process of the present invention comprises the reduction of a gene product with the activity of a “At3g55990-protein” from Arabidopsis thaliana or its functional equivalent or its homolog, e.g. the reduction of
a) a gene product of a gene comprising the nucleic acid molecule as shown in column 5 of Table I and being depicted in the same respective line as said At3g55990 or a functional equivalent or a homologue thereof as depicted in column 7 of Table I, preferably a homologue or functional equivalent as depicted in column 7 of Table I B, and being depicted in the same respective line as said At3g55990; or
b) a polypeptide comprising a polypeptide, a consensus sequence or a polypeptide motif as depicted in column 5 of Table II or column 7 of table IV respectively, and being depicted in the same respective line as said At3g55990 or a functional equivalent or a homologue thereof as depicted in column 7 of Table II, preferably a homologue or functional equivalent as depicted in column 7 of Table II B, and being depicted in the same respective line as said At3g55990,
   as mentioned herein, for the increased tolerance and/or resistance to environmental stress and increased biomass production as compared to a corresponding non-transformed wild type plant.

Accordingly, in one embodiment, the molecule which activity is to be reduced in the process of the invention is the gene product with an activity described as “At3g55990-protein”, preferably it is the molecule of section (a) or (b) of this paragraph [0024.1.1.1].

Homologues (=homologs) of the present gene products, in particular homologues of a gene product which is encoded by or which is comprising a nucleic acid molecule as shown in column 7 of Table I, or a polypeptide comprising the polypeptide, a consensus sequence or a polypeptide motif as shown in column 7 of Table II or IV, can be derived from any organisms as long as the homologue confers the herein mentioned activity, i.e. it is a functional equivalent of said molecules. In particular, the homologue confers an increased tolerance and/or resistance to environmental stress and increased biomass production as compared to a corresponding non-transformed wild type plant after its reduction, repression and/or deletion.

Further, according to the present invention, the term “homologue” relates to the sequence of an organism having preferably the highest or essentially the highest sequence homology to the herein mentioned or listed sequences of all expressed sequences of said organism.

The person skilled in the art knows how to find, identify and confirm, that, preferably, a putative homologue has said the:“tolerance and/or resistance to environmental stress and/or biomass production increasing activity”, e.g. as described herein. If known, the biological function or activity in an organism essentially relates or corresponds to the activity or function as described for the genes mentioned in paragraph [0024.1.1.1], for example to at least one of the protein(s) indicated in Table II, Column 5.

Accordingly, in one embodiment, the homologue or the functional equivalent comprises the sequence of a polypeptide encoded by a nucleic acid molecule comprising a sequence indicated in Table I, Column 7 or a polypeptide sequence, a consensus sequence or a polypeptide motif indicated in Table II or IV, Column 7 or it is the expression product of a nucleic acid molecule comprising a polynucleotide indicated in Table I, Column 7.

The herein disclosed information about sequence, activity, consensus sequence, polypeptide motifs and tests leads the person skilled in the art to the respective homologous or functional equivalent expression product in an organism.

In one embodiment, throughout the specification the activity of a protein or polypeptide or a nucleic acid molecule or sequence encoding such protein or polypeptide, e.g. an activity selected from the group consisting of 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, or ubiquitin conjugating enzyme/ubiquitin-like activating enzyme , is an identical or similar activity according to the present invention if it has essentially the same activity or it has at least 10% of the original enzymatic or biological activity, preferably 30% or 40%, particularly preferably 50%, 60% or 70%, most particularly preferably 80%, 85%, 90%, 95% or more of the activity in comparison to a protein as shown in table II, column 5 or 7, more preferably as shown in table II, column 5.

In one embodiment, the homolog of any one of the polypeptides indicated in Table II, column 5 is derived from an Eukaryote and has a sequence identity of at least 50% and preferably has essentially the same or a similar activity as described in [0024.1.1.1], however its reduction, repression or deletion of expression or activity confers an increased tolerance and/or resistance to environmental stress and increased biomass production as compared to a corresponding non-transformed wild type plant, respectively, in the organisms or a part thereof.

In one embodiment, the homolog of any one of the polypeptides indicated in Table II, column 5 is derived from a plant, preferably from a plant selected from the group consisting of Nacardiaceae, Asteraceae, Apiaceae, Betulaceae, Boraginaceae, Brassicaceae, Bromeliaceae, Caricaceae, Cannabaceae, Convolvulaceae, Chenopodiaceae, Cucurbitaceae, Elaeagnaceae, Ericaceae, Euphorbiaceae, Fabaceae, Geraniaceae, Gramineae, Juglandaceae, Lauraceae, Leguminosae, Linaceae, perennial grass, fodder crops, vegetables and ornamentals and has a sequence identiy of at least 50% and preferably has essentially the same or a essentially similar activity as described in [0024.1.1.1], however at least its reduction of expression or activity confers an increased tolerance and/or resistance to environmental stress and increased biomass production as compared to a corresponding non-transformed wild type plant. In one embodiment, the homolog of any one of the polypeptides indicated in Table II, column 5 is derived from a crop plant and has a sequence identiy of at least 30% and preferably has essentially the same or a similar activity as described in [0024.1.1.1], however at least an reduction of expression or activity confers an increased tolerance and/or resistance to environmental stress and increased biomass production as compared to a corresponding non-transformed wild type plant.

Accordingly, in one embodiment, the molecule which activity is to be reduced in the process of the invention is the molecule of (a) or (b) of paragraph [0024.1.1.1], [0025.1.1.1] or of paragraph [00027.1.1.1].

Thus, a homolog or a functional equivalent of a polypeptide as indicated in Table II, column 3 or column 5 may be a polypeptide encoded by a nucleic acid molecule comprising a polynucleotide as indicated in Table I, column 7 in the same line, or may be a polypeptide comprising a polypeptide indicated in Table II, column 7, or one or more polypeptide motifs indicated in Table IV, column 7, or the consensus sequence as indicated in Table IV, column 7 in the same line as the polypeptide indicated in Table II, column 3 or column 5.

Thus, a homolog or a functional equivalent of a nucleic acid molecule as indicated in Table I, column 5 may be a nucleic acid molecule encoding a polypeptide comprising a polynucleotide as indicated in Table I, column 7 in the same line, or nucleic acid molecule encoding a polypeptide comprising a polypeptide indicated in Table II, column 7, or the consensus sequence or polypeptide motifs indicated in Table IV, column 7 in the same line as the nucleic acid molecule indicated in Table I, column 3 or column 5.

Further homologs or functional equivalents of said polypeptide which activity is to be reduced in the process of the present invention are described herein below.

As consequence of the reduction, repression, decrease or deletion of the translation, transcription and/or expression, e.g. as consequence of the reduced, repressed, decreased or deleted transcription of a gene, in particular of a gene as described herein (e.g. comprising a nucleic acid molecule indicated in column 5 or 7 of Table I), a related phenotypic trait appears such as the enhanced or increased tolerance and/or resistance to environmental stress and increased biomass production as compared to a corresponding non-transformed wild type plant.

A decreased, repressed or reduced activity of the molecule which activity is to be reduced in the process of the invention manifests itself in an increased tolerance and/or resistance to environmental stress and increased biomass production as compared to a corresponding non-transformed wild type plant.

In one embodiment, in the process of the invention for increasing the tolerance and/or resistance to environmental stress and increased biomass production as compared to a corresponding non-transformed wild type plant, the process comprises reducing, repressing or deleting the expression or activity of at least one nucleic acid molecule having or encoding a polypeptide having the activity of at least one protein encoded by the nucleic acid molecule as depicted in column 5 of Table I, and wherein the nucleic acid molecule comprises a nucleic acid molecule selected from the group consisting of:
a) an isolated nucleic acid molecule encoding the polypeptide as depicted in column 5 or 7 of Table II and/or containing a consensus sequence as depicted in column 7 of table IV;
b) an isolated nucleic acid molecule as depicted in column 5 or 7 of Table I;
c) an isolated nucleic acid sequence, which, as a result of the degeneracy of the genetic code, can be derived from a polypeptide sequence as depicted in column 5 or 7 of Table II or from a polypeptide containing a consensus sequence as depicted in column 7 of table IV;
d) an isolated nucleic acid molecule having at least 30%, 40%, 50%, 60%, 70%, 80%, 90%, 95%, 96%. 97%, 98%, 99%, 99.5% or 99.9% identity with the nucleic acid molecule sequence of a polynucleotide comprising the nucleic acid molecule as depicted in column 5 or 7 of Table I;
e) an isolated nucleic acid molecule encoding a polypeptide having at least 30%, 40%, 50%, 60%, 70%, 80%, 90%, 95%, 96%, 97%, 98%, 99%, 99.5% or 99.9% identity with the amino acid sequence of the polypeptide encoded by the nucleic acid molecule of (a) to (c) and having the activity represented by a protein as depicted in column 5 of Table II;
f) an isolated nucleic acid molecule encoding a polypeptide which is isolated with the aid of monoclonal or polyclonal antibodies made against a polypeptide encoded by one of the nucleic acid molecules of (a) to (e) and having the activity represented by the protein as depicted in column 5 of Table II;
g) an isolated nucleic acid molecule encoding a polypeptide comprising the consensus sequence or one or more polypeptide motifs as depicted in the corresponding lane of column 7 of Table IV and preferably having the activity represented by a nucleic acid molecule encoding a polynucleotide as depicted in column 5 of Table I;
h) an isolated nucleic acid molecule which comprises a polynucleotide, which is obtained by amplifying a cDNA library or a genomic library using primers as depicted in column 7 of Table III and which primers do not start at their 5′-end with with the nucleotides ATA; and preferably said isolated nucleic acid molecule encoding a polypeptide having the activity represented by a polypeptide encoded by a nucleic acid molecule comprising a polynucleotide as depicted in column 5 of Table I;
i) an isolated nucleic acid molecule encoding a polypeptide having the activity represented by the protein as depicted in column 5 of Table II; and
j) an isolated nucleic acid molecule which is obtainable by screening a suitable nucleic acid library under stringent hybridization conditions with a probe comprising a complementary sequence of a nucleic acid molecule of (a) or (b) or with a fragment thereof having at least 15, 17, 19, 20, 21, 22, 23, 24, 25 nt or more of a nucleic acid molecule complementary to a nucleic acid molecule sequence characterized in (a) to (d) and encoding a polypeptide having the activity represented by a protein as depicted in column 5 of Table II;
   or which comprises a sequence which is complementary thereto;
   or of a protein encoded by said nucleic acid molecules.

Accordingly, in one embodiment, the term “molecule which activity is to be reduced in the process of the invention” refers to above nucleic acid molecules comprising at least one of said nucleic acid molecules a) to j) according to this paragraph.

In one embodiment, said nucleic acid molecule or said polypeptide as depicted in column 5 or 7 of Table I, II or IV is a novel nucleic acid molecule or a novel polypeptide as depicted in column 7 of Table I B or II B.

A series of mechanisms exists via which the molecule which activity is to be reduced in the process of the invention, e.g. a polypeptide or a nucleic acid molecule, in particular a nucleic acid molecule comprising the nucleic acid molecule as described in column 5 or 7 of Table I or a polypeptide comprising a polypeptide or a consensus sequence as described in column 5 or 7 of Table II or IV respectively, or a functional homolog of said nucleic acid molecule or polypeptide, can be manipulated to directly or indirectly affect the tolerance and/or resistance to environmental stress and biomass production as compared to a corresponding non-transformed wild type plant.

For example, the molecule number or the specific activity of the polypeptide which activity is to be reduced in the process of the invention or processed by polypeptide which activity is to be reduced in the process of the invention or the molecule number processed by or expressed by the nucleic acid molecule which activity is to be reduced in the process of the invention may be reduced, decreased or deleted.

However, it is known to the person skilled in the art to reduce, decrease, repress, or delete the expression of a gene which is naturally present in the organisms can be achieved by several ways, for example by modifying the regulation of the gene, or by reducing or decreasing the stability of the mRNA or of the gene product encoded by the nucleic acid molecule which activity is to be reduced, repressed, decreased or deleted in the process of the invention, e.g. of a nucleic acid molecule comprising a polynucleotide as depicted in column 5 or 7 of Table I.

The term “reduction” of a biological function refers, for example, to the quantitative reduction in a binding capacity or binding strength of a protein to a substrate in an organism, a tissue, a cell or a cell compartment in comparison with the wild type of the same genus and species to which this method has not been applied, under otherwise identical conditions (such as, for example, culture conditions, age of the plants and the like).

Binding partners for the protein can be identified in the manner with which the skilled worker is familiar, for example by the yeast 2-hybrid system.

This also applies analogously to the combined reduction, repression, decrease or deletion of the expression of a gene or gene product of the nucleic acid molecule described in column 5 or 7, Table I together with the manipulation of further activities.

In one embodiment, the reduction, repression, decrease, deletion or modulation according to this invention can be conferred by the (e.g. transgenic) expression of a antisense nucleic acid molecule, an RNAi, a snRNA, a dsRNA, a siRNA, a miRNA, a ta-siRNA, a cosuppression molecule, a ribozyme or of an antibody, an inhibitor or of an other molecule inhibiting the expression or activity of the expression product of the nucleic acid molecule which activity is to be reduced, decreased or deleted in the process of the invention. E.g. the reduction, repression, decrease, deletion or modulation according to this invention can be conferred by the (e.g. transgenic) expression of a nucleic acid molecule comprising a polynucleotide encoding antisense nucleic acid molecule, RNAi, snRNA, dsRNA, siRNA, miRNA, ta-siRNA, a cosuppression molecule, ribozyme or of an antibody Against the nucleic acid molecule or the polypeptide which activity is to be reduced in the process of the invention.

In a further embodiment, the reduction, repression, decrease, deletion or modulation according to this invention can be to a stable mutation in the corresponding endogenous gene encoding the nucleic acid molecule to be reduced, decreased or deleted in the process of the invention, e.g. of a nucleic acid molecule comprising a polynucleotide as depicted in column 5 or 7 of Table I.

In another embodiment, the reduction, repression, decrease, deletion or modulation according to this invention can be a modulation of the expression or of the behaviour of a gene conferring the expression of the polypeptide to be reduced, decreased, repressed or deleted according to the process of the invention, e.g. of a polypeptide comprising a polypeptide, a consensus sequence or a polypeptide motif as depicted in column 5 or 7 of Table II or IV

Said expression may be constitutive, e.g. due to a stable, permanent, systemic, local or temporal expression, for example limited to certain cell types, tissues organs or time periods.

For example, the reduction, repression, decrease, deletion or modulation according to this invention can be transient, e.g. due to an transient transformation, a transiently active promoter or temporary addition of a modulator, such as an antagonist, inhibitor or inductor, e.g. after transformation with an inducible construct carrying the double-stranded RNA nucleic acid molecule (dsRNA), antisense, RNAi, snRNA, siRNA, miRNA, ta-siRNA, a cosuppression molecule, ribozyme, antibody etc. as described herein, for example under control of an inducible promoter combined with the application of a corresponding inducer, e.g. tetracycline or ecdysone.

The reduction, decrease or repression of the activity of the molecule which activity is reduced according to the process of the invention amounts preferably by at least 10%, preferably by at least 30% or at least 60%, especially preferably by at least 70%, 80%, 85%, 90% or more, very especially preferably are at least 95%, more preferably are at least 99% or more in comparison to the control, reference or wild type. Most preferably the reduction, decrease, repression or deletion in activity amounts to 100%.

Various strategies for reducing the quantity, the expression, the activity or the function of proteins encoded by the nucleic acids or the nucleic acid sequences themselves according to the invention are encompassed in accordance with the invention. The skilled worker will recognize that a series of different methods are available for influencing the quantity of a protein, the activity or the function in the desired manner.

Accordingly, in one embodiment, the process of the present invention comprises one or more of the following steps:
i) Inhibition, repression, inactivation or reduction of translation or transcription of,
ii) Destabilization of transcript stability or polypeptide stability of,
iii) Reduction of accumulation of,
iv) Inhibition, repression, inactivation or reduction of activity of transcript or polypeptide of, and/or
v) Reduction of the copy number of functional (e.g. expressed) genes of,
   a suitable compound, for example, of
a) a protein enabling, mediating or controlling the expression of a protein encoded by the nucleic acid molecule which activity is reduced in the process of invention or of the polypeptide which activity is reduced in the process of the invention, e.g. of a polypeptide comprising a polypeptide, a consensus sequence or a polypeptide motif as depicted in column 5 or 7, of Table II or IV or being enoded by a nucleic acid molecule comprising a polynucleotide as depicted in column 5 or 7, of Table I;
b) a mRNA molecule enabling, mediating or controlling the expression of a protein to be reduced in the process of the invention or being encoded by the nucleic acid molecule which activity is reduced in the process of the invention, e.g. enabling, mediating or controlling the expression of a polypeptide comprising a polypeptide, a consensus sequence or a polypeptide motif as depicted in column 5 or 7, of Table II or IV, or of a polypeptide being encoded by a nucleic acid molecule comprising a polynucleotide as depicted in column 5 or 7, of Table I,
c) an RNA molecule enabling, mediating or controlling the expression of a mRNA encoding a polypeptide which activity is reduced in the process of the invention, e.g. of a mRNA encoding a polypeptide comprising a polypeptide, a consensus sequence or a polypeptide motif as depicted in column 5 or 7, of Table II or IV, or of a mRNA comprising the nucleic acid molecule which activity is reduced in the process of the invention, e.g. comprising a polynucleotide as depicted in column 5 or 7, of Table I;
d) an RNA molecule enabling, mediating or controlling the expression of an expression product of a nucleic acid molecule comprising the polynucleotide which activity is reduced in the process of the invention; e.g. of a nucleic acid molecule comprising a polynucleotide as depicted in column 5 or 7, of Table I;
e) a mRNA encoding the polynucleotide or the polypeptide which activity is reduced in the process of the invention; e.g. of a nucleic acid molecule comprising a polynucleotide as depicted in column 5 or 7, of Table I or of a mRNA enabling, mediating or controlling the expression of a polypeptide which activity is reduced in the process of the invention, the polypeptide depicted in column 5 or 7, of Table II or IV;
f) a gene encoding an activator enabling the activation or increase of the expression of a nucleic acid molecule encoding a polypeptide encoded by the nucleic acid molecule which activity is reduced in the process of the invention or the polypeptide which activity is to be reduced in the process of the invention, e.g. a gene encoding an activator enabling the activation or increase of the expression of a polypeptide comprising a polypeptide, a consensus sequence or a polypeptide motif as depicted column 5 or 7, of Table II or IV or of a nucleic acid molecule comprising a polynucleotide as depicted in column 5 or 7, of Table I; or
g) an endogenous gene encoding the polypeptide or the nucleic acid molecule which activity is reduced in the process of the invention, for example an endogenous gene encoding a polypeptide comprising a polypeptide, a consensus sequence or a polypeptide motif as depicted column 5 or 7, of Table II or IV, or a nucleic acid molecule comprising a polynucleotide as depicted in column 5 or 7, of Table I;

Accordingly, the
i) Inhibition, repression, inactivation or reduction of translation or transcription,
ii) Destabilization transcript stability or polypeptide stability,
iii) Reduction of accumulation,
iv) Inhibition, repression, inactivation or reduction of activation of transcript or polypeptide, and/or
v) reducing the copy number of functional (e.g. expressed) genes,
   can for example be mediated e.g. by adding or expressing an antisense molecule, cosuppression molecule, an antibody, ribozyme, siRNA, microRNA, ta-siRNA, a cosuppression molecule, or RNAi, by mutation or deletion of a gene sequence, expressing or improving the activity of a negative expression element or by other methods known to the person skilled in the art or mentioned herein. A polynucleotide, which activity is to be reduced in the process of the invention or one or more fragments thereof, can for example be expressed in antisense orientation. In another embodiment, a hairpin RNAi constructs is expressed. It is also advantageous to express simultaneously a sense and antisense RNA molecule of the nucleic acid molecule or polypeptide which activity is to be reduced in the process of the invention.

For example, in an embodiment of the present invention, the present invention relates to a process, wherein the number of functional (e.g. expressed) copies of a gene encoding the polynucleotide or nucleic acid molecule of the invention is decreased.

Further, the endogenous level of the polypeptide of the invention can for example be decreased by modifying the transcriptional or translational regulation or efficiency of the polypeptide.

Details are described later in the description or in the examples

In one embodiment, the process of the present invention comprises for example one or more of the following steps
a) stabilizing a protein conferring the decreased expression of a protein of the nucleic acid molecule or polypeptide which activity is reduced in the process of the invention;
b) stabilizing a mRNA or functional RNA conferring the decreased expression of a of the nucleic acid molecule or polypeptide which activity is reduced in the process of the invention;
c) increasing or stimulating the specific activity of a protein conferring the decreased expression of a of the nucleic acid molecule or polypeptide which activity is reduced in the process of the invention;
d) decreasing the specific activity of a protein conferring the increased expression of a of the nucleic acid molecule or polypeptide which activity is reduced in the process of the invention;
e) expressing a transgenic gene encoding a protein conferring the decreased expression of a nucleic acids molecule or polypeptide which activity is reduced in the process of the invention,
f) generating or increasing the expression of an endogenous or artificial transcription factor repressing the expression of a protein conferring the increased expression of the nucleic acid molecule or polypeptide which activity is reduced in the process of the invention;
g) generating or increasing the expression of an endogenous or artificial transcription factor mediating the expression of a protein conferring the decreased expression of the nucleic acid molecule or polypeptide which activity is reduced in the process of the invention;
h) reducing, repressing or deleting the expression of an endogenous or artificial transcription factor repressing the expression of a protein conferring the decreased expression of the nucleic acid molecule or polypeptide which activity is reduced in the process of the invention;
i) reducing, repressing or deleting the expression of an endogenous or artificial transcription factor mediating the expression of a protein conferring the increased expression of the nucleic acid molecule or polypeptide which activity is reduced in the process of the invention;
j) increasing the number of functional copies or expression of a gene conferring the decreased expression of the nucleic acid molecule or polypeptide which activity is reduced in the process of the invention,
k) increasing the activity of a repressor protein or a repressor RNA
l) Increasing the activity of a protein or RNA leading to a dominant negative pheno-type of the protein which activity is reduced in the process of the invention;
m) expression of an antibody or aptamer, which binds to the nucleic acid molecule which activity is to be reduced in the process of the invention or the protein which activity is reduced in the process of the invention and thereby reducing, decreasing or deleting its activity;
n) expressing a repressor conferring the reduced, repressed, decreased or deleted expression of a protein encoded by the nucleic acid to be reduced in the process molecule of the invention or of the polypeptide which activity is reduced in the process of the invention, or increasing the inhibitory regulation of the polypeptide of the invention;
o) reducing or deleting the expression of the nucleic acid molecule which activity is reduced in the process of the invention or the polypeptide which activity is reduced in the process of the invention by adding one or more exogenous repression factors such as a inhibiting chemical compound to the organism or its medium or its feed, e.g. to the organism's water supply; or
p) modulating growth conditions of an organism in such a manner, that the expression or activity of a nucleic acid molecule encoding the protein which activity is reduced in the process of the invention or the protein itself is reduced, repressed, decreased or deleted. This can be achieved by e.g modulating light and/or nutrient conditions, which in terms modulated the expression of the gene or protein which activity is reduced in the process of the invention.

Others strategies and modifications and combinations of above strategies are well known to the person skilled in the art and are also embodiment of this invention. Above said can for example be achieved by adding positive expression or removing negative expression elements, e.g. homologous recombination can be used to either introduce positive or negative regulatory elements, like a 35S enhancer into a plant promoter, or to remove repressor elements from regulatory regions. Further gene conversion methods can be used to disrupt elements or to enhance the activity of repressor elements. Repressor elements can be randomly introduced in plants by T-DNA or transposon mutagenesis. Lines can be identified in which the repressor elements are integrated near to a gene encoding the nucleic acid molecule or polypeptide which activity is to be reduced in the process of the invention, the expression of which is thereby reduced, repressed or deleted. Furthermore mutations like point mutations can be introduced randomly by different mutagenesis methods and can be selected by specific methods such like TILLING (reviewed in Slade and Knauf, Transgenic Res. 2005, 14(2), 109-115).

For example, an increase of the activity of a protein or RNA leading to a dominant negative phenotype of the protein which activity is reduced in the process of the invention can be achieved through the expression of a nucleic acid molecule encoding a protein, which has lost its biological activity but which binds to another protein in a multimeric complex thereby decreasing, repressing or deleting the activity of said complex or which binds for example as a transcription factor to DNA and thereby decreasing or deleting the activity of the translated protein.

In general, the amount of mRNA, polynucleotide or nucleic acid molecule in a cell or a compartment of an organism correlates to the amount of encoded protein and thus with the overall activity of the encoded protein in said volume. Said correlation is not always linear, the activity in the volume is dependent on the stability of the molecules, the degradation of the molecules or the presence of activating or inhibiting co-factors. Further, product and educt inhibitions of enzymes are well known.

The activity of the abovementioned proteins and/or polypeptide encoded by the nucleic acid molecule to be reduced in the process of the present invention can be reduced, repressed, decreased or deleted in various ways.

For example, the activity in an organism or in a part thereof, like a cell, is reduced, repressed or decreased via reducing or decreasing the gene product number, e.g. by reducing, repressing or decreasing the expression rate, like mutating the natural promoter to a lower activity, or by reducing, repressing or decreasing the stability of the mRNA expressed, thus reducing, repressing or decreasing the translation rate, and/or reducing, repressing or decreasing the stability of the gene product, thus increasing the proteins decay. Further, the activity or turnover of enzymes or channels or carriers, transcription factors, and similar active proteins can be influenced in such a manner that a reduction of the reaction rate or a modification (reduction, repression, decrease or deletion) of the affinity to the substrate results, is reached.

A mutation in the catalytic centre of a polypeptide or nucleic acid molecule which activity is reduced in the process of the invention, e.g. of an enzyme or a catalytic or regulatory RNA, can modulate the turn over rate of the enzyme, e.g. a knock out of an essential amino acid can lead to a reduced or complete knock out of the activity of the enzyme, or the deletion of regulator binding sites can reduce a positive regulation.

The specific activity of an enzyme of the present invention can be decreased such that the turn over rate is decreased or the binding of a co-factor is reduced. Reducing the stability of the encoding mRNA or the protein can also decrease the activity of a gene product. The reduction of the activity is also under the scope of the term “reduced, repressed, decreased or deleted activity”. Besides this, advantageously the reduction of the activity in cis, eg. mutating the promoter including other cis-regulatory elements, or the transcribed or coding parts of the gene, inhibition can also be achieved in trans, eg. by transfactors like chimeric transcription factor, ribozymes, antisense RNAs, dsRNAs or dominant negative protein versions, which interfere with various stages of expression, eg the transcription, the translation or the activity of the protein or protein complex itself. Also epigenetic mechanisms like DNA modifications, DNA methylation, or DNA packaging might be recruited to inactivate or down regulate the nucleic acids of the invention or the encoded proteins.

Accordingly, the increase of tolerance and/or resistance to environmental stress and increased biomass production as compared to a corresponding non-transformed wild type plant in a non-human organism is in one embodiment achieved through the use of an RNA interference (dsRNAi), the introduction of an antisense nucleic acid, RNAi, snRNA, siRNA, miRNA, ta-siRNA, cosuppression molecule, or a ribozyme nucleic acid combined with an ribozyme, a nucleic acid encoding a cosuppressor, a nucleic acid encoding a dominant negative protein, DNA- or protein-binding factor or antibodies targeting said gene or -RNA or -proteins, RNA degradation inducing viral nucleic acids or a micro RNA molecule or combinations thereof against the nucleic acid molecule characterized in this paragraph.

The regulation of the abovementioned nucleic acid sequences may be modified so that gene expression is decreased. This reduction, repression, decrease or deletion (reduction, repression, decrease, deletion, inactivation or down-regulation shall be used as synonyms throughout the specification) can be achieved as mentioned above by all methods known to the skilled person, preferably by double-stranded RNA interference (dsRNAi), introduction of an antisense nucleic acid, a ribozyme, an antisense nucleic acid combined with a ribozyme, a nucleic acid encoding a cosuppressor, a nucleic acid encoding a dominant negative protein, DNA- or protein-binding factor or antibodies targeting said gene or -RNA or -proteins, RNA degradation inducing viral nucleic acids and expression systems, systems for inducing a homolog recombination of said genes, mutations in said genes or a combination of the above.

In general, an activity of a gene product in an organism or part thereof, in particular in a plant cell, a plant, or a plant tissue or a part thereof or in a microorganism can be decreased by decreasing the amount of the specific encoding mRNA or the corresponding protein in said organism or part thereof. “Amount of protein or mRNA” is understood as meaning the molecule number of polypeptides or mRNA molecules in an organism, a tissue, a cell or a cell compartment. “Decrease” in the amount of a protein means the quantitative decrease of the molecule number of said protein in an organism, a tissue, a cell or a cell compartment or part thereof—for example by one of the methods described herein below—in comparison to a wild type, control or reference.

In this context, “inactivation” means that the activity of the polypeptide encoded is essentially no longer detectable in the organism or in the cell such as, for example, within the plant or plant cell. For the purposes of the invention, downregulation (=reduction) means that its activity, e.g. the enzymatic or biological activity of the polypeptide encoded is partly or essentially completely reduced in comparison with the activity of the untreated organism. This can be achieved by different cell-biological mechanisms. In this context, the activity can be downregulated in the entire organism or, in the case of multi-celled organisms, in individual parts of the organism, in the case of plants for example in tissues such as the seed, the leaf, the root or other parts.

A modification, i.e. a decrease, can be caused by endogenous or exogenous factors. For example, a decrease in activity in an organism or a part thereof can be caused by adding a chemical compound such as an antagonist to the media, nutrition, soil of the plants or to the plants themselves.

In one embodiment the increase in the tolerance and/or resistance to environmental stress and increased biomass production as compared to a corresponding non-transformed wild type plant can be achieved by decreasing the level of the endogenous nucleic acid molecule or the endogenous polypeptide described herein, i.e. of the nucleic acid molecule or the polypeptide which activity is to be reduced according to the process of the invention, in particular of a polynucleotide or polypeptide described in the corresponding line of Table I or II, column 5 or 7, respectively.

Accordingly, in one further embodiment of the process of the invention the reduction, repression or deletion of the activity represented by the protein or nucleic acid molecule to be reduced in the process of the invention is achieved by at least one step selected from the group consisting of:
a) introducing a nucleic acid molecule comprising a polynucleotide encoding a ribonucleic acid sequence, which is able to form a double-stranded ribonucleic acid molecule, whereby a fragment of at least 17, 18, 19, 20, 21, 22, 23, 24 or 25 nucleotides (nt) or more, preferably of 25, 26, 27, 28, 29, 30, 31, 32, 33, 34, 35, 36, 37, 38, 39 or 40 nucleotides (nt) or more, more preferably of 50, 60, 70, 80, 90 or 100 nucleotides (nt) or more and whereby said double-stranded ribonucleic acid molecule has an identity of 50% or more, preferably an identity of 60, 65, 70, 75, 80, 85, 90, 95, 97, 98, 99 or most preferred of 100% to the nucleic acid molecule to be reduced according to the process of the invention or a nucleic acid molecule encoding the polypeptide to be reduced according to the process of the invention or selected from the group consisting of:
   aa) the nucleic acid molecule which activity is reduced in the process of the present invention;
   ab) a nucleic acid molecule comprising a polynucleotide as depicted in column 5 or 7 of Table I or encoding a polypeptide comprising a polypeptide as depicted in column 5 or 7 of Table II, preferably, a nucleic acid molecule as depicted in column 5 or 7 of Table I or encoding a polypeptide as depicted in column 5 or 7 of Table II, preferably a nucleic acid molecule as depicted in column 5 or 7 of Table I A or encoding a polypeptide as depicted in column 5 or 7 of Table II B, and
   ac) a nucleic acid molecule encoding a polypeptide having the activity of polypeptide depicted in column 5 of Table II or encoding the expression product of a polynucleotide comprising a nucleic acid molecule as depicted in column 5 or 7 of Table I; and
b) anyone of the steps disclosed in following paragraph:
a) introducing an RNAi, snRNA, dsRNA, siRNA, miRNA, ta-siRNA, cosuppression molecule, or an antisense nucleic acid molecule, whereby the RNAi, snRNA, dsRNA, siRNA, miRNA, ta-siRNA, cosuppression molecule,or antisense nucleic acid molecule comprises a fragment of 17, 18, 19, 20, 21, 22, 23, 24 or 25 nucleotides (nt) or more, preferably of 25, 26, 27, 28, 29, 30, 31, 32, 33, 34, 35, 36, 37, 38, 39 or 40 nucleotides (nt) or more, more preferably of 50, 60, 70, 80, 90 or 100 nucleotides (nt) or more with an identity of at least 30% or more, preferably of 40, 50, 60, 65, 70, 75, 80, 85, 90, 95, 97, 98, 99 or most preferably of 100% to the nucleic acid molecule to be reduced according to the process of the invention or a nucleic acid molecule encoding the polypeptide to be reduced according to the process of the invention or to a nucleic acid molecule selected from a group defined in section (aa) to (ac);
b) introducing of a ribozyme which specifically cleaves the nucleic acid molecule to be reduced according to the process of the invention or a nucleic acid molecule encoding the polypeptide to be reduced according to the process of the invention or a nucleic acid molecule selected from a group defined in section (aa) to (ac);
c) introducing the RNAi, snRNA, dsRNA, siRNA, miRNA, ta-siRNA, cosuppression molecule, ribozyme, antibody, antisense nucleic acid molecule characterized in (a) and the ribozyme characterized in (b);
d) introducing of a sense nucleic acid molecule conferring the expression of the nucleic acid molecule to be reduced according to the process of the invention or a nucleic acid molecule encoding the polypeptide to be reduced according to the process of the invention or of a nucleic acid molecule selected from a group defined in section (aa) to (ac) for inducing a co-suppression of the endogenous expression product of the nucleic acid molecule to be reduced according to the process of the invention or a nucleic acid molecule encoding the polypeptide to be reduced according to the process of the invention or of a nucleic acid molecule selected from a group defined in section (aa) to (ac);
e) introducing a nucleic acid molecule comprising a polynucleotide conferring the expression of a dominant-negative mutant of a protein having the activity of a protein to be reduced according to the process of the invention or of a protein encoded by a nucleic acid molecule to be reduced according to the process of the invention or of a protein encoded by a nucleic acid molecule selected from a group defined in section (aa) to (ac);
f) introducing a nucleic acid molecule comprising a polynucleotide encoding a factor, which binds to a nucleic acid molecule comprising the nucleic acid molecule to be reduced according to the process of the invention or comprising a nucleic acid molecule encoding the polypeptide to be reduced according to the process of the invention or comprising a nucleic acid molecule selected from a group defined in section (aa) to (ac);
g) introducing a viral nucleic acid molecule conferring the decline of a RNA molecule comprising the nucleic acid molecule to be reduced according to the process of the invention or comprising a nucleic acid molecule encoding the polypeptide to be reduced according to the process of the invention or comprising a nucleic acid molecule selected from a group defined in section (aa) to (ac);
h) introducing a nucleic acid construct capable to recombinate with and silence, inactivate, repress or reduce the activity of an endogenous gene comprising the nucleic acid molecule to be reduced according to the process of the invention or comprising a nucleic acid molecule encoding the polypeptide to be reduced according to the process of the invention or comprising a nucleic acid molecule selected from a group defined in section (aa) to (ac);
i) introducing a non-silent mutation in an endogenous gene comprising the nucleic acid molecule to be reduced according to the process of the invention or comprising a nucleic acid molecule encoding the polypeptide to be reduced according to the process of the invention or comprising a nucleic acid molecule selected from a group defined in section (aa) to (ac); and/or
j) introducing an expression construct conferring the expression of nucleic acid molecule characterized in any one of (a) to (i).

Accordingly, in one further embodiment of the process of the invention the reduction or deletion of the activity represented by the protein or nucleic acid molecule used in the process of the invention is achieved by at least one step selected from the group consisting of:
a) introducing of nucleic acid molecules encoding a ribonucleic acid molecule, which sequence is able to form a double-stranded ribonucleic acid molecule, whereby the sense strand of said double-stranded ribonucleic acid molecules has a identity of at least 30%, preferably of 60, 65, 70, 75, 80, 85, 90, 95, 97, 98, 99 or 100% to the nucleic acid molecule to be reduced according to the process of the invention or a nucleic acid molecule encoding the polypeptide to be reduced according to the process of the invention or to a nucleic acid molecule selected from the group consisting of:
   i) a nucleic acid molecule conferring the expression of a protein comprising a polypeptide, a consensus sequence or a polypeptide motif, as depicted in column 5 or 7 of Table II or IV or conferring the expression of nucleic acid molecule comprising a polynucleotide as depicted in column 5 or 7 of Table I;
   ii) a nucleic acid molecule encoding a protein having the activity of a protein to be reduced according to the process of the invention, e.g. comprising a polypeptide, a consensus sequence or a polypeptide motif as depicted in column 5 or 7 of Table II or IV or conferring the expression of nucleic acid molecule comprising a polynucleotide as depicted in column 5 or 7 of Table I; and
   iii) a nucleic acid molecule comprising a fragment of at least 17, 18, 19, 20, 21, 22, 23, 24 or 25 base pairs of a nucleic acid molecule with a homology of at least 50% preferably of 60, 65, 70, 75, 80, 85, 90, 95, 97, 98, 99 or 100% to a nucleic acid molecule of (i) or (ii);
b) introducing an antisense nucleic acid molecule, whereby the antisense nucleic acid molecule has an identity of at least 30% or more, preferably of 40, 50, 60, 65, 70, 75, 80, 85, 90, 95, 97, 98, 99 or 100% to a nucleic acid molecule antisense to the nucleic acid molecule to be reduced according to the process of the invention or a nucleic acid molecule encoding the polypeptide to be reduced according to the process of the invention or a nucleic acid molecule selected from the group consisting of (i) to (iii) above;
c) introducing of a ribozyme which specifically cleaves a nucleic acid molecule conferring the expression of a protein having the activity of a protein to be reduced according to the process of the invention, e.g. comprising a polypeptide, a consensus sequence or a polypeptide motif as depicted in column 5 or 7 of Table II or IV, or which specifically cleaves a nucleic acid molecule conferring the expression of the nucleic acid molecule to be reduced according to the process of the invention or the polypeptide to be reduced according to the process of the invention or a nucleic acid molecule encoding the polypeptide to be reduced according to the process of the invention or a nucleic acid molecule selected from the group consisting of (i) to (iii) above;
d) introducing of the antisense nucleic acid molecule characterized in (b) and the ribozyme characterized in (c);
e) introducing of a sense nucleic acid molecule conferring the expression of the nucleic acid molecule to be reduced according to the process of the invention or the polypeptide to be reduced according to the process of the invention or a nucleic acid molecule encoding the polypeptide to be reduced according to the process of the invention or a nucleic acid molecule selected from the group consisting of (i) to (iii) above for inducing a co-suppression of the endogenous the nucleic acid molecule to be reduced according to the process of the invention or a nucleic acid molecule encoding the polypeptide to be reduced according to the process of the invention or a nucleic acid molecule selected from the group consisting of (i) to (iii) above;
f) introducing a nucleic acid molecule conferring the expression of a dominant-negative mutant of a protein having the activity of a protein to be reduced according to the process of the invention, e.g. comprising a polypeptide, a consensus sequence or a polypeptide motif as depicted in column 5 or 7 of Table II or IV or of a dominant-negative mutant of a polypeptide encoded by a nucleic acid molecule selected from the group consisting of (i) to (iii) above, for example expressing said sequence leading to the dominant-negative mutant protein thereby the activity of the protein used in the inventive process is reduced, decreased or deleted;
g) introducing a nucleic acid molecule encoding a factor, which binds to a nucleic acid molecule conferring the expression of a protein having the activity of a polypeptide to be reduced according to the process of the invention, e.g. comprising a polypeptide, a consensus sequence or a polypeptide motif as depicted in column 5 or 7 of Table II or IV or being encoded by a nucleic acid molecule selected from the group consisting of (i) to (iii) above;
h) introducing a viral nucleic acid molecule conferring the decline of a RNA molecule conferring the expression of a protein having the activity of a protein used in the process of the invention, especially a polypeptide comprising a polypeptide, a consensus sequence or a polypeptide motif as depicted in column 5 or 7 of Table II or IV or being encoded by a nucleic acid molecule selected from the group consisting of (i) to (iii) above;
i) introducing a nucleic acid construct capable to recombinate with and mutate an endogenous gene conferring the expression of a protein having the activity of a protein used in the inventive process especially a polypeptide comprising a polypeptide, a consensus sequence or a polypeptide motif as depicted in column 5 or 7 of Table II or IV or being encoded by a nucleic acid molecule selected from the group consisting of (i) to (iii) above;
j) introducing a non-silent mutation in an endogenous gene conferring the expression of a protein having the activity of a protein used in the inventive process especially a polypeptide comprising a polypeptide, a consensus sequence or a polypeptide motif as depicted in column 5 or 7 of Table II or IV or being encoded by a nucleic acid molecule selected from the group consisting of (i) to (iii) above;
k) selecting of a non-silent mutation in a nucleic acid sequence encoding a protein having the activity of a protein used in the inventive process especially a polypeptide comprising a polypeptide, a consensus sequence or a polypeptide motif as depicted in column 5 or 7 of Table II or IV or being encoded by a nucleic acid molecule selected from the group consisting of (i) to (iii) above from a randomly mutagenized population of organisms used in the inventive process; and/or
l) introducing an expression construct conferring the expression of a nucleic acid molecule or polypeptide as characterized in any one of (a) to (k) or conferring the expression of a nucleic acid molecule or polypeptide characterized in any one of (a) to (k).

In one embodiment, the process of the present invention comprises the following step:
introducing into an endogenous nucleic acid molecule, e.g. into an endogenous gene, which confers the expression of a polypeptide comprising a polypeptide, a consensus sequence or a polypeptide motif as depicted in column 5 or 7 of Table II or IV or a polypeptide being encoded by a nucleic acid molecule selected from the group consisting of (i) to (iii) mentioned above, a mutation of a distinct amino acid shown in the consensus sequence depicted in column 7 of Table IV in the same line,

whereby the mutation confers a non-silent mutation in the polypeptide which activity is to be reduced in the process of the invention, in particular in a polypeptide comprising a polypeptide, a consensus sequence or a polypeptide motif as depicted in column 5 or 7 of Table II or IV or a polypeptide being encoded by a nucleic acid molecule selected from the group consisting of (i) to (iii), mentioned above.

The consensus sequence depicted in column 7 of Table IV indicates the amino acids which were found to be strongly conserved within the sequences of the polypeptides depicted in columns 5 and 7 of Table II. Thus, it is preferred to mutate one or more of the distinct conserved amino acids (not defined as X or Xaa) by a random mutation approach or by selectively introducing a mutation into such a amino acid or into a stretch of several conserved amino acids, for example via applying a chemical, physical or biological mutagens such as site directed mutagenesis or introducing a homologous recombination.

In one embodiment, the coding sequences of a nucleic acid molecule which activity is to be reduced in the process of the invention, in particular from the nucleic acid molecule mentioned under sections (a) to (i) of paragraph [0030.1.1.1], preferably of a nucleic acid molecule comprising a nucleic acid molecule as depicted in column 5 or 7 of Table I, is used for the reduction, repression, decrease or deletion of the nucleic acid sequences which activity is to be reduced in the process of the invention according to the different process steps (a) to (l) mentioned above in paragraphs [0052.1.1.1] to [0053.1.1.1], e.g. as described in Liu Q et al (2002) High-Stearic and High-Oleic Cottonseed Oils Produced by Hairpin RNA-Mediated Post-Transcriptional Gene Silencing. Plant Physiology 129 pp 1732-1743.

Preferably less than 1000 bp, 900 bp, 800 bp or 700 bp, particular preferably less than 600 bp, 500 bp, 400 bp, 300 bp, 200 bp or 100 bp of the coding region of the said nucleic acid sequence are used.

The skilled person knows that it is possible starting from the nucleic acid sequences disclosed herein as the nucleic acid molecule which activity is to be reduced in the process of the invention to reduce or delete the activity particularly of orthologs of the molecules disclosed herein. In particular, the skilled person knows how to isolate the complete gene, the coding region (CDR), the expressed regions (e.g. as cDNA), or fragments thereof of said nucleic acid sequences, in particular said regions of molecules as indicated in Table I, column 5 or 7, if not already disclosed herein, e.g. starting from the nucleic acid molecule mentioned under sections (a) to (j) of paragraph [0030.1.1.1] above, preferably starting from a nucleic acid molecule comprising a nucleic acid molecule as depicted in column 5 or 7 of Table I.

In one embodiment, the 5′- and/or 3′-sequences of a nucleic acid molecule which activity is to be reduced in the process of the invention, in particular from the nucleic acid molecule mentioned under sections (a) to (i) of paragraph [0030.1.1.1], preferably of a nucleic acid molecule comprising a nucleic acid molecule as depicted in column 5 or 7 of Table I, is used for the reduction, repression, decrease or deletion of the nucleic acid sequences which activity is to be reduced in the process of the invention according to the different process steps (a) to (j) mentioned above in paragraphs [0052.1.1.1] to [0053.1.1.], e.g. as described in Ifuku K et al (2003) Specific Interference in psbP Genes Encoded by a Multigene Family in Nicotiana tabacum with Short 3′-Untranslated Sequence. Biosci. Biotechnol. Biochem., 67 (1) pp 107-113.

Preferably less than 1000 bp, 900 bp, 800 bp or 700 bp, particular preferably less than 600 bp, 500 bp, 400 bp, 300 bp, 200 bp or 100 bp of the 5′- and/or 3′-region of the said nucleic acid sequence are used.

The skilled person knows that it is possible starting from the nucleic acid sequences disclosed herein as the nucleic acid molecule which activity is to be reduced in the process of the invention to isolate the UTRs of said molecules. In particular, the skilled person knows how to isolate the 5′- and/or 3′-regions of said nucleic acid sequences, in particular the 5′- and/or 3′-regions of the molecules indicated in Table I, column 5 or 7, if not already disclosed herein, e.g. starting from the nucleic acid molecule mentioned under sections (a) to (j) of paragraph [0030.1.1.1] above, preferably starting from a nucleic acid molecule comprising a nucleic acid molecule as depicted in column 5 or 7 of Table I.

5′- and 3′-regions can be isolated by different methods like RACE (Zang and Frohman (1997) Using rapid amplification of cDNA ends (RACE) to obtain full length cDNAs. Methods Mol Biol 1997; 69:61-87 or genomic walking PCR technologies (Mishra et al., 2002, Biotechniques 33(4): 830-832; Spertini et al 1999, Biotechniques 27(2), 308-314).

The aforementioned process steps of the reduction or deletion of the biological activity represented by the protein of the invention lead to an increase of tolerance and/or resistance to environmental stress and increased biomass production as compared to a corresponding non-transformed wild type plant.

A reduction in the activity or the function is preferably achieved by a reduced expression of a gene encoding the protein of the inventive process.

In a preferred embodiment of the process of the invention, said reduction of the activity or function of the activity of a gene product encoding the nucleic acid molecule or the polypeptide to be reduced according to the process of the invention, e.g. a polypeptide encoded by nucleic acid molecules comprising the nucleic acid molecules shown in column 5 or 7 of Table I or a polypeptide comprising the amino acid sequences, consensus sequences or polypeptide motifs shown in column 5 or 7 of Table II or in column 7 of Table IV or a nucleic acid molecule comprising the nucleic acid molecules shown in column 5 or 7 of Table I or encoding a polypeptide comprising the amino acid sequences, consensus sequences or polypeptide motifs shown in column 5 or 7 of Table II or IV can be achieved for example using the following methods:
a) introduction of a double-stranded RNA nucleic acid sequence (dsRNA) as described above or of an expression cassette, or more than one expression cassette, ensuring the expression of the latter;
b) introduction of an antisense nucleic acid sequence or of an expression cassette ensuring the expression of the latter. Encompassed are those methods in which the antisense nucleic acid sequence is directed against a gene (i.e. genomic DNA sequences including the promoter sequence) or a gene transcript (i.e. RNA sequences) including the 5′ and 3′ non-translated regions. Also encompassed are alpha-anomeric nucleic acid sequences;
c) introduction of an antisense nucleic acid sequence in combination with a ribozyme or of an expression cassette ensuring the expression of the former;
d) introduction of sense nucleic acid sequences for inducing cosuppression or of an expression cassette ensuring the expression of the former;
e) introduction of a nucleic acid sequence encoding dominant-negative protein or of an expression cassette ensuring the expression of the latter;
f) introduction of DNA-, RNA- or protein-binding factor or antibodies against genes, RNA's or proteins or of an expression cassette ensuring the expression of the latter;
g) introduction of viral nucleic acid sequences and expression constructs which bring about the degradation of RNA, or of an expression cassette ensuring the expression of the former;
h) introduction of constructs for inducing homologous recombination on endogenous genes, for example for generating knockout mutants;
i) introduction of mutations into endogenous genes for generating a loss of function (e.g. generation of stop codons, reading-frame shifts and the like);
j) introduction of a microRNA or micro-RNA (miRNA) that has been designed to target the gene of interest in order to induce a breakdown or translation inhibition of the mRNA of the gene of interest and thereby silence gene expression or of an expression cassette ensuring the expression of the former;
k) introduction of a ta-sRNA that has been designed to target the gene of interest in order to induce breakdown or translational inhibition of the mRNA of the gene of interest and thereby silence gene expression or of an expression cassette ensuring the expression of the former; and/or
l) identifying a non silent mutation, e.g. generation of stop codons, reading-frame shifts, inversions and the like in random mutagenized population, e.g. according to the so called TILLING method.

Each of these methods may bring about a reduction in the expression, the activity or the function for the purposes of the invention. A combined use is also feasible. Further methods are known to the skilled worker and may encompass all possible steps of gene expression, like hindering or preventing processing of the protein, transport of the protein or its mRNA, inhibition of ribosomal attachment, inhibition of RNA splicing, induction of an enzyme which degrades RNA or the protein of the invention and/or inhibition of translational elongation or termination.

Accordingly, the following paragraphs relate preferably to the repression, reduction, decrease or deletion of an activity selected from the group consisting of 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme, or an activity being represented by a nucleic acid molecule or polypeptide which activity is to be reduced in the process of the invention, in particular of a nucleic acid molecule comprising a polynucleotide as depicted in column 5 or 7, of Table I, preferably of column 5, or encoding a polypeptide comprising a polypeptide, a consensus sequence or polypeptide motif as depicted in column 5 or 7 of Table II or IV, preferably of column 5.

Thus, a reference to column 5 or 7 of Table I, Table I A, Table I B, Table II, Table II A, Table II B, Table III or Table IV as used herein refers preferably to column 5 or 7 of Table I, Table I A, Table I B, Table II, Table II A, Table II B, Table III or Table IV, respectively.

What follows is a brief description of the individual preferred methods.

a) Introduction of a double-stranded RNA nucleic acid sequence (dsRNA) e.g. for the reduction or deletion of activity of the nucleic acid molecule or polypeptide which activity is to be reduced in the process of the invention, in particular of a nucleic acid molecule comprising a polynucleotide as depicted in column 5 or 7, of Table I or encoding a polypeptide comprising a polypeptide, consensus sequence or polypeptide motif as depicted in column 5 or 7 of Table II or IV

The method of regulating genes by means of double-stranded RNA (“double-stranded RNA interference”; dsRNAi) has been described extensively for animal, yeast, fungi and plant organisms such as Neurospora, Zebrafish, Drosophila, mice, planaria, humans, Trypanosoma, petunia or Arabidopsis [for example Matzke M A et al. (2000) Plant Mol. Biol. 43: 401-415; Fire A. et al. (1998) Nature 391: 806-811; WO 99/32619; WO 99/53050; WO 00/68374; WO 00/44914; WO 00/44895; WO 00/49035; WO 00/63364]. In addition RNAi is also documented as an advantageously tool for the repression of genes in bacteria such as E. coli for example by Tchurikov et al. [J. Biol. Chem., 2000, 275 (34): 26523-26529]. Fire et al. named the phenomenon RNAi for RNA interference. The techniques and methods described in the above references are expressly referred to. Efficient gene suppression can also be observed in the case of transient expression or following transient transformation, for example as the consequence of a biolistic transformation [Schweizer P et al. (2000) Plant J 2000 24: 895-903]. dsRNAi methods are based on the phenomenon that the simultaneous introduction of complementary strand and counterstrand of a gene transcript brings about highly effective suppression of the expression of the gene in question. The resulting phenotype is very similar to that of an analogous knock-out mutant (Waterhouse P M et al. (1998) Proc. Natl. Acad. Sci. USA 95: 13959-64).

Tuschl et al., Gens Dev., 1999, 13 (24): 3191-3197, were able to show that the efficiency of the RNAi method is a function of the length of the duplex, the length of the 3′-end overhangs, and the sequence in these overhangs.

Accordingly, another embodiment of the invention is a double-stranded RNA molecule (dsRNA), which confers—after being introduced or expressed in a suitable organism, e.g. a plant, or a part thereof—the reduction, repression, decrease or deletion of the an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme.

Based on the work of Tuschl et al. and assuming that the underlining principles are conserved between different species the following guidelines can be given to the skilled worker. Accordingly, the dsRNA molecule of the invention or used in the process of the invention preferable fulfills at least one of the following principles:

to achieve good results the 5′ and 3′ untranslated regions of the used nucleic acid sequence and regions close to the start codon should be in general avoided as this regions are richer in regulatory protein binding sites and interactions between RNAi sequences and such regulatory proteins might lead to undesired interactions;

in plants the 5′ and 3′ untranslated regions of the used nucleic acid sequence and regions close to the start codon preferably 50 to 100 nt upstream of the start codon give good results and therefore should not be avoided;

preferably a region of the used mRNA is selected, which is 50 to 100 nt (=nucleotides or bases) downstream of the AUG start codon;

only dsRNA (=double-stranded RNA) sequences from exons are useful for the method, as sequences from introns have no effect;

the G/C content in this region should be greater than 30% and less than 70% ideally around 50%;

a possible secondary structure of the target mRNA is less important for the effect of the RNAi method.

The dsRNAi method can be particularly effective and advantageous for reducing the expression of the nucleic acid molecule which activity is to be reduced in the process of the invention, particular of a nucleic acid molecule comprising a polynucleotide as depicted in column 5 or 7, of Table I or encoding a polypeptide comprising a polypeptide, consensus sequence or polypeptide motif as depicted column 5 or 7 of Table II or IV and/or homologs thereof. As described inter alia in WO 99/32619, dsRNAi approaches are clearly superior to traditional antisense approaches.

Accordingly, the invention therefore furthermore relates to double-stranded RNA molecules (dsRNA molecules) which, when introduced into an organism, advantageously into a plant (or a cell, tissue, organ or seed derived therefrom), bring about altered metabolic activity by the reduction in the expression of the nucleic acid molecule which activity is reduced in the process of the invention, particular of a nucleic acid molecule comprising a polynucleotide as depicted in column 5 or 7, of Table I or encoding a polypeptide comprising a polypeptide, consensus sequence or polypeptide motif as depicted in column 5 or 7 of Table II or IV and/or homologs thereof.

In a double-stranded RNA molecule of the invention, e.g. a dsRNA for reducing the expression of a protein encoded by a nucleic acid molecule which activity is to be reduced in the process of the invention, particular of a nucleic acid molecule comprising a polynucleotide as depicted in column 5 or 7, of Table I and/or homologs thereof,
i) one of the two RNA strands is essentially identical to at least part of a nucleic acid sequence, and
ii) the respective other RNA strand is essentially identical to at least part of the complementary strand of a nucleic acid sequence.

The term “essentially identical” refers to the fact that the dsRNA sequence may also include insertions, deletions and individual point mutations in comparison to the target sequence while still bringing about an effective reduction in expression. Preferably, the identity as defined above amounts to at least 30%, preferably at least 40%, 50%, 60%, 70% or 80%, very especially preferably at least 90%, most preferably 100%, between the “sense” strand of an inhibitory dsRNA and a part-segment of a nucleic acid sequence of the invention including in a preferred embodiment of the invention their endogenous 5′- and 3′ untranslated regions or between the “antisense” strand and the complementary strand of a nucleic acid sequence, respectively. The part-segment amounts to at least 10 bases, preferably at least 17, 18, 19, 20, 21, 22, 23, 24, 25, 26, 27, 28, 29 or 30 bases, especially preferably at least 40, 50, 60, 70, 80 or 90 bases, very especially preferably at least 100, 200, 300 or 400 bases, most preferably at least 500, 600, 700, 800, 900 or more bases or at least 1000 or 2000 bases or more in length. In another preferred embodiment of the invention the part-segment amounts to 17, 18, 19, 20, 21, 22, 23, 24, 25, 26 or 27 bases, preferably to 20, 21, 22, 23, 24 or 25 bases. These short sequences are preferred in animals and plants. The longer sequences preferably between 200 and 800 bases are preferred in non-mammalian animals, preferably in invertebrates, in yeast, fungi or bacteria, but they are also usable in plants. Long double-stranded RNAs are processed in the organisms into many siRNAs (=small/short interfering RNAs) for example by the protein Dicer, which is a ds-specific Rnase III enzyme. As an alternative, an “essentially identical” dsRNA may also be defined as a nucleic acid sequence, which is capable of hybridizing with part of a gene transcript (for example in 400 mM NaCl, 40 mM PIPES pH 6.4, 1 mM EDTA at 50° C. or 70° C. for 12 to 16 h).

The dsRNA may consist of one or more strands of polymerized ribonucleotides. Modification of both the sugar-phosphate backbone and of the nucleosides may furthermore be present. For example, the phosphodiester bonds of the natural RNA can be modified in such a way that they encompass at least one nitrogen or sulfur heteroatom. Bases may undergo modification in such a way that the activity of, for example, adenosine deaminase is restricted. These and other modifications are described herein below in the methods for stabilizing antisense RNA.

The dsRNA can be prepared enzymatically; it may also be synthesized chemically, either in full or in part. Short dsRNA up to 30 bp, which effectively mediate RNA interference, can be for example efficiently generated by partial digestion of long dsRNA templates using E. coli ribonuclease III (RNase III). (Yang, D., et al. (2002) Proc. Natl. Acad. Sci. USA 99, 9942.)

The double-stranded structure can be formed starting from a single, self-complementary strand or starting from two complementary strands. In a single, self-complementary strand, “sense” and “antisense” sequence can be linked by a linking sequence (“linker”) and form for example a hairpin structure. Preferably, the linking sequence may take the form of an intron, which is spliced out following dsRNA synthesis. The nucleic acid sequence encoding a dsRNA may contain further elements such as, for example, transcription termination signals or polyadenylation signals. If the two strands of the dsRNA are to be combined in a cell or an organism advantageously in a plant, this can be brought about in a variety of ways:

a) transformation of the cell or of the organism, advantageously of a plant, with a vector encompassing the two expression cassettes;

b) cotransformation of the cell or of the organism, advantageously of a plant, with two vectors, one of which encompasses the expression cassettes with the “sense” strand while the other encompasses the expression cassettes with the “antisense” strand;

c) supertransformation of the cell or of the organism, advantageously of a plant, with a vector encompassing the expression cassettes with the “sense” strand, after the cell or the organism had already been transformed with a vector encompassing the expression cassettes with the “antisense” strand or vice versa;

d) hybridization e.g. crossing of two organisms, advantageously of plants, each of which has been transformed with one vector, one of which encompasses the expression cassette with the “sense” strand while the other encompasses the expression cassette with the “antisense” strand;

e) introduction of a construct comprising two promoters that lead to transcription of the desired sequence from both directions; and/or

f) infecting of the cell or of the organism, advantageously of a plant, with an engineered virus, which is able to produce the desired dsRNA molecule.

Formation of the RNA duplex can be initiated either outside the cell or within the cell. If the dsRNA is synthesized outside the target cell or organism it can be introduced into the organism or a cell of the organism by injection, microinjection, electroporation, high velocity particles, by laser beam or mediated by chemical compounds (DEAE-dextran, calciumphosphate, liposomes) or in case of animals it is also possible to feed bacteria such as E. coli strains engineered to express double-stranded RNAi to the animals.

Accordingly, in one embodiment, the present invention relates to a dsRNA whereby the sense strand of said double-stranded RNA nucleic acid molecule has an identity of at least 30%, 35%, 40%, 45%, 50%, 55% or 60%, preferably 65%, 70%, 75% or 80%, more preferably 85%, 90%, 95%, 96%, 97%, 98% or 99% or more preferably 95%, 96%, 97%, 98%, 99% or 100% to a nucleic acid molecule comprising a nucleic acid molecule as depicted in column 5 or 7 of Table I, preferably as depicted in Table I B, or encoding a polypeptide comprising a polypeptide as depicted in column 5 or 7 of Table II, preferably as depicted in Table II B, or of Table IV.

Another embodiment of the invention is a dsRNA molecule, comprising a fragment of at least 10 base paires (=bases, nt, nucleotides), preferably at least 17, 18, 19, 20, 21, 22, 23, 24, 25, 26, 27, 28, 29, 30, 35, 40, 45 or 50, especially preferably at least 55, 60, 70, 80 or 90 base pairs, very especially preferably at least 100, 200, 300 or 400 base pairs, most preferably at least 500, 600, 700, 800, 900 or more base pairs or at least 1000 or 2000 base pairs of a nucleic acid molecule with an identity of at least 50%, 60%, 70%, 80% or 90%, preferably 95%, 96%, 97%, 98%, 99% or 100% to a nucleic acid molecule as depicted in column 5 or 7 of Table I, preferably as depicted in Table I B, or to the nucleic acid molecule encoding a polypeptide protein comprising a polypeptide as depicted in column 5 or 7 of Table II, preferably as depicted in Table II B, or of Table IV.

In another preferred embodiment of the invention the encoded sequence or its part-segment of the dsRNA molecule amounts to 17, 18, 19, 20, 21, 22, 23, 24, 25, 26 or 27 bases, preferably to 20, 21, 22, 23, 24 or 25 bases, whereby the identity of the sequence is essentially 95%, 96%, 97%, 98%, or preferred 99% or 100%.

The expression of the dsRNA molecule of the invention confers the increase of tolerance and/or resistance to environmental stress and increased biomass production as compared to a corresponding non-transformed wild type plant in the organism or part thereof.

In a preferred embodiment of the invention the sense and antisense strand of the double-stranded RNA are covalently bound or are bound by other, e.g. weak chemical bonds such as hydrogen bonds to each other and the antisense strand is essentially the complement of the sense-RNA strand.

As shown in WO 99/53050, the dsRNA may also encompass a hairpin structure, by linking the “sense” and “antisense” strands by a “linker” (for example an intron), which is hereby incorporated by reference. The self-complementary dsRNA structures are preferred since they merely require the expression of a construct and always encompass the complementary strands in an equimolar ratio.

The expression cassettes encoding the “antisense” or the “sense” strand of the dsRNA or the self-complementary strand of the dsRNA are preferably inserted into a vector and stably inserted into the genome of a plant, using the methods described herein below (for example using selection markers), in order to ensure permanent expression of the dsRNA. Transient expression with bacterial or viral vectors are similar useful.

The dsRNA can be introduced using an amount which makes possible at least one copy per cell. A larger amount (for example at least 5, 10, 100, 500 or 1 000 copies per cell) may bring about more efficient reduction.

As has already been described, 100% sequence identity between the dsRNA and a gene transcript of a nucleic acid molecule to be reduced according to the process of the invention, e.g. of one of the molecules comprising a molecule as shown in column 5 or 7 of Table I or encoding a polypeptide encompassing a polypeptide, a consensus sequence or a polypeptide motif as shown in column 5 or 7 of Table II or IV or it's homolog is not necessarily required in order to bring about effective reduction in the expression. The advantage is, accordingly, that the method is tolerant with regard to sequence deviations as may be present as a consequence of genetic mutations, polymorphisms or evolutionary divergences. Thus, for example, using the dsRNA, which has been generated starting from a nucleic acid molecule to be reduced according to the process of the invention, e.g. of one of the molecules comprising a molecule as shown in column 5 or 7 of Table I or encoding a polypeptide encompassing a polypeptide, a consensus sequence or a motif as shown in column 5 or 7 of Table II or IV or homologs thereof of the one organism, may be used to suppress the corresponding expression in another organism.

The high degree of sequence homology or identity between nucleic acid molecules to be reduced according to the process of the invention, from various organisms (e.g. plants), e.g. of one of the molecules comprising a molecule as depicted in column 5 or 7 of Table I, preferably of Table I B or encoding a polypeptide encompassing a polypeptide, a consensus sequence, or a polypeptide motif as depicted in column 5 or 7 of Table II or IV, preferably II B, allows the conclusion that these proteins are likely conserved to a high degree within the evolution, for example also in other plants, and therefore it is optionally possible that the expression of a dsRNA derived from one of the disclosed nucleic acid molecule to be reduced according to the process of the invention, e.g. of one of the molecules comprising a molecule as depicted in column 5 or 7 of Table I or encoding a polypeptide encompassing a polypeptide, a consensus sequence or a polypeptide motif as depicted in column 5 or 7 of Table II or IV or homologs thereof should also have an advantageous effect in other plant species.

The dsRNA can be synthesized either in vivo or in vitro. To this end, a DNA sequence encoding a dsRNA can be introduced into an expression cassette under the control of at least one genetic control element (such as, for example, promoter, enhancer, silencer, splice donor or splice acceptor or polyadenylation signal). Suitable advantageous constructs are described herein below. Polyadenylation is not required, nor do elements for initiating translation have to be present.

A dsRNA can be synthesized chemically or enzymatically. Cellular RNA polymerases or bacteriophage RNA polymerases (such as, for example T3, T7 or SP6 RNA polymerase) can be used for this purpose. Suitable methods for the in-vitro expression of RNA are described (WO 97/32016; U.S. Pat. No. 5,593,874; U.S. Pat. No. 5,698,425, U.S. Pat. No. 5,712,135, U.S. Pat. No. 5,789,214, U.S. Pat. No. 5,804,693). Prior to introduction into a cell, tissue or organism, a dsRNA which has been synthesized in vitro either chemically or enzymatically can be isolated to a higher or lesser degree from the reaction mixture, for example by extraction, precipitation, electrophoresis, chromatography or combinations of these methods. The dsRNA can be introduced directly into the cell or else be applied extracellularly (for example into the interstitial space). In one embodiment of the invention the RNAi method leads to only a partial loss of gene function and therefore enables the skilled worker to study a gene dose effect in the desired organism and to fine tune the process of the invention. In another preferred embodiment it leads to a total loss of function and therefore increases the tolerance and/or resistance to environmental stress and increased biomass production as compared to a corresponding non-transformed wild type plant. Furthermore it enables a person skilled in the art to study multiple functions of a gene.

Stable transformation of the plant with an expression construct, which brings about the expression of the dsRNA is preferred, however. Suitable methods are described herein below.

b) Introduction of an antisense nucleic acid sequence, e.g. for the reduction, repression or deletion of the nucleic acid molecule or polypeptide which activity is to be reduced in the process of the invention, in particular of a nucleic acid molecule comprising a polynucleotide as depicted in column 5 or 7, of Table I or encoding a polypeptide comprising a polypeptide, a consensus sequence or a polypeptide motif as depicted in column 5 or 7 of Table II or IV

Methods for suppressing a specific protein by preventing the accumulation of its mRNA by means of “antisense” technology can be used widely and has been described extensively, including for plants; Sheehy et al. (1988) Proc. Natl. Acad. Sci. USA 85: 8805-8809; U.S. Pat. No. 4,801,34100; Mol J N et al. (1990) FEBS Lett 268(2): 427-430. The antisense nucleic acid molecule hybridizes with, or binds to, the cellular mRNA and/or the genomic DNA encoding the target protein to be suppressed. This process suppresses the transcription and/or translation of the target protein. Hybridization can be brought about in the conventional manner via the formation of a stable duplex or, in the case of genomic DNA, by the antisense nucleic acid molecule binding to the duplex of the genomic DNA by specific interaction in the large groove of the DNA helix.

In one embodiment, an “antisense” nucleic acid molecule comprises a nucleotide sequence, which is at least in part complementary to a “sense” nucleic acid molecule encoding a protein, e.g., complementary to the coding strand of a double-stranded cDNA molecule or complementary to an encoding mRNA sequence. Accordingly, an antisense nucleic acid molecule can bind via hydrogen bonds to a sense nucleic acid molecule. The antisense nucleic acid molecule can be complementary to an entire coding strand of a nucleic acid molecule conferring the expression of the polypeptide to be reduced in the process of the invention or comprising the nucleic acid molecule which activity is to be reduced in the process of the invention or to only a portion thereof. Accordingly, an antisense nucleic acid molecule can be antisense to a “coding region” of the coding strand of a nucleotide sequence of a nucleic acid molecule of the present invention.

The term “coding region” refers to the region of the nucleotide sequence comprising codons, which are translated into amino acid residues.

In another embodiment, the antisense nucleic acid molecule is antisense to a “noncoding region” of the mRNA flanking the coding region of a nucleotide sequence. The term “noncoding region” refers to 5′ and 3′ sequences which flank the coding region that are not translated into a polypeptide, i.e., also referred to as 5′ and 3′ untranslated regions (5′-UTR or 3′-UTR). Advantageously the noncoding region is in the area of 50 bp, 100 bp, 200 bp or 300 bp, preferably 400 bp, 500 bp, 600 bp, 700 bp, 800 bp, 900 bp or 1000 by up- and/or downstream from the coding region.

Given the coding strand sequences encoding the polypeptide or the nucleic acid molecule to be reduced in the process of the invention, e.g. having above mentioned activity, e.g. the activity of a polypeptide with the activity of the protein which activity is to be reduced in the process of the invention as disclosed herein, antisense nucleic acid molecules can be designed according to the rules of Watson and Crick base pairing.

Accordingly, yet another embodiment of the invention is an antisense nucleic acid molecule, which confers—after being expressed in a suitable organism, e.g. a plant, or a part thereof—the reduction, repression, or deletion of the an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme.

Accordingly, in another embodiment, the invention relates to an antisense nucleic acid molecule, whereby the antisense nucleic acid molecule has an identity of at least 30% to a nucleic acid molecule antisense to a nucleic acid molecule encoding the protein as shown in column 5 or 7 of Table II, preferably as depicted in Table II B, or encoding a protein encompassing a consensus sequence or a polypeptide motif as depicted in of Table IV or being encoded by a nucleic acid molecule comprising a polynucleotide as depicted in column 5 or 7 of Table I, preferably as depicted in Table I B or a homologue thereof as described herein and which confers the increase of tolerance and/or resistance to environmental stress and increased biomass production as compared to a corresponding non-transformed wild type plant, respectively after its expression.

Thus in another embodiment, the antisense nucleic acid molecule of the invention comprises a fragment of at least 15, 16, 17, 18, 19, 20, 21, 22, 23, 24, 25, 26, 27, 28, 29, 30, 35, 40, 45 or 50, especially preferably at least 60, 70, 80 or 90 base pairs, very especially preferably at least 100, 200, 300 or 400 base pairs, most preferably at least 500, 600, 700, 800, 900 or more base pairs or at least the entire sequence of a nucleic acid molecule with an identity of at least 50% 60%, 70%, 80% or 90%, preferably 100% to an antisense nucleic acid molecule to a nucleic acid molecule conferring the expression of a protein as depicted in column 5 or 7 of Table II, preferably as depicted in Table II B, or encoding a protein encompassing a consensus sequence or a polypeptide motif as depicted in Table IV or being encoded by a nucleic acid molecule comprising a polynucleotide as depicted in column 5 or 7 of Table I, preferably as depicted in Table I B, or a homologue thereof as described herein and which confers after its expression the increase of tolerance and/or resistance to environmental stress and increased biomass production as compared to a corresponding non-transformed wild type plant.

An antisense nucleic acid sequence which is suitable for reducing the activity of a protein can be deduced using the nucleic acid sequence encoding this protein, for example the nucleic acid sequence which activity is to be reduced in the process of the invention, e.g. comprising a nucleic acid molecule as depicted in column 5 or 7 of Table I or a nucleic acid molecule encoding a polypeptide comprising a polypeptide, a consensus sequence or a polypeptide as depicted in column 5 or 7 of Table II or IV (or homologs, analogs, paralogs, orthologs thereof), by applying the base-pair rules of Watson and Crick. The antisense nucleic acid sequence can be complementary to all of the transcribed mRNA of the protein; it may be limited to the coding region, or it may only consist of one oligonucleotide, which is complementary to part of the coding or noncoding sequence of the mRNA. Thus, for example, the oligonucleotide can be complementary to the nucleic acid region, which encompasses the translation start for the protein. Antisense nucleic acid sequences may have an advantageous length of, for example, 5, 10, 15, 20, 25, 30, 35, 40, 45 or 50 nucleotides but they may also be longer and encompass at least 100, 200, 500, 1000, 2000 or 5000 nucleotides. A particular preferred length is between 15 and 30 nucleotides such as 15, 20, 25 or 30 nucleotides. Antisense nucleic acid sequences can be expressed recombinantly or synthesized chemically or enzymatically using methods known to the skilled worker. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of substances which can be used are phosphorothioate derivatives and acridine-substituted nucleotides such as 5-fluorouracil, 5-bromouracil, 5-chlorouracil, 5-iodouracil, hypoxanthin, xanthin, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, methyl uracil-5-oxyacetate, uracil-5-oxyacetic acid, 5-methyl-2-thiouracil, 3-(3-amino-3-N-2-carboxypropyl)uracil and 2,6-diaminopurine. Alternatively, the antisense nucleic acid can be produced biologically using an expression vector into which a nucleic acid molecule has been subcloned in an antisense orientation (i.e., RNA transcribed from the inserted nucleic acid molecule will be of an antisense orientation to a target nucleic acid molecule of interest, described further in the following subsection).

In a further preferred embodiment, the expression of a protein which activity is to be reduced in the process of the invention, e.g. encoded by a nucleic acid molecule comprising a nucleic acid molecule as depicted in column 5 or 7 of Table I or of a polypeptide comprising a polypeptide, a consensus sequence or a polypeptide motif as depicted in column 5 or 7 of Table II or IV or homologs, analogs, paralogs, orthologs thereof can be inhibited by nucleotide sequences which are complementary to the regulatory region of a gene (for example a promoter and/or enhancer) and which may form triplex structures with the DNA double helix in this region so that the transcription of the gene is reduced. Such methods have been described (Helene C (1991) Anticancer Drug Res. 6(6): 569-84; Helene C et al. (1992) Ann. NY Acad. Sci. 660: 27-36; Maher L J (1992) Bioassays 14(12): 807-815).

In a further embodiment, the antisense nucleic acid molecule can be an alpha-anomeric nucleic acid. Such alpha-anomeric nucleic acid molecules form specific double-stranded hybrids with complementary RNA in which—as opposed to the conventional b-nucleic acids—the two strands run in parallel with one another (Gautier C et al. (1987) Nucleic Acids Res. 15: 6625-6641). Furthermore, the antisense nucleic acid molecule can also comprise 2′-O-methylribonucleotides (Inoue et al. (1987) Nucleic Acids Res. 15: 6131-6148), or chimeric RNA-DNA analogs (Inoue et al. (1987) FEBS Lett 215: 327-330).

The antisense nucleic acid molecules of the invention are typically administered to a cell or generated in situ such that they hybridize with or bind to cellular mRNA and/or genomic DNA encoding a polypeptide having the activity of protein which activity is to be reduced in the process of the invention or encoding a nucleic acid molecule having the activity of the nucleic acid molecule which activity is to be reduced in the process of the invention and thereby inhibit expression of the protein, e.g., by inhibiting transcription and/or translation and leading to the aforementioned increased tolerance and/or resistance to environmental stress and increased biomass production as compared to a corresponding non-transformed wild type plant.

The antisense molecule of the present invention comprises also a nucleic acid molecule comprising a nucleotide sequences complementary to the regulatory region of an nucleotide sequence encoding the natural occurring polypeptide of the invention, e.g. the polypeptide sequences shown in the sequence listing, or identified according to the methods described herein, e.g., its promoter and/or enhancers, e.g. to form triple helical structures that prevent transcription of the gene in target cells. See generally, Helene, C. (1991) Anticancer Drug Des. 6(6): 569-84; Helene, C. et al. (1992) Ann. N.Y. Acad. Sci. 660: 27-36; and Maher, L. J. (1992) Bioassays 14(12): 807-15.

c) Introduction of an antisense nucleic acid sequence combined with a ribozyme, e.g. for the reduction or deletion of activity of the nucleic acid molecule or polypeptide which activity is to be reduced in the process of the invention, in particular of a nucleic acid molecule comprising a polynucleotide as depicted in column 5 or 7, of Table I or encoding a polypeptide comprising a polypeptide as depicted in column 5 or 7 of Table II or IV

Yet another embodiment of the invention is a ribozyme, which confers—after being expressed in a suitable organism, e.g. a plant, or a part thereof—the reduction, repression, decrease or deletion of the activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme.

Thus, in a further embodiment, the invention relates to a ribozyme, which specifically cleaves a nucleic acid molecule conferring expression of a protein as depicted in column 5 or 7 of Table II, preferably as depicted in Table II B, or comprising a consensus sequence or a polypeptide motif as depicted in Table IV or being encoded by a nucleic acid molecule comprising a polynucleotide as depicted in column 5 or 7 of Table I, preferably as depicted in Table I B, or a homologue thereof as described herein, and which confers after its expression the increase of tolerance and/or resistance to environmental stress and increase of biomass production as compared to a corresponding non-transformed wild type plant.

It is advantageous to combine the above-described antisense strategy with a ribozyme method. Catalytic RNA molecules or ribozymes can be adapted to any target RNA and cleave the phosphodiester backbone at specific positions, thus functionally deactivating the target RNA (Tanner N K (1999) FEMS Microbiol. Rev. 23(3): 257-275). The ribozyme per se is not modified thereby, but is capable of cleaving further target RNA molecules in an analogous manner, thus acquiring the properties of an enzyme. The incorporation of ribozyme sequences into “antisense” RNAs imparts this enzyme-like RNA-cleaving property to precisely these “antisense” RNAs and thus increases their efficiency when inactivating the target RNA. The preparation and the use of suitable ribozyme “antisense” RNA molecules is described, for example, by Haseloff et al. (1988) Nature 33410: 585-591.

Further the antisense nucleic acid molecule of the invention can be also a ribozyme. Ribozymes are catalytic RNA molecules with ribonuclease activity, which are capable of cleaving a single-stranded nucleic acid, such as an mRNA, to which they have a complementary region. In this manner, ribozymes [for example “Hammer-head” ribozymes; Haselhoff and Gerlach (1988) Nature 33410: 585-591] can be used to catalytically cleave the mRNA of an enzyme to be suppressed and to prevent translation. The ribozyme technology can increase the efficacy of an antisense strategy. Methods for expressing ribozymes for reducing specific proteins are described in (EP 0 291 533, EP 0 321 201, EP 0 360 257). Ribozyme expression has also been described for plant cells [Steinecke P et al. (1992) EMBO J 11(4): 1525-1530; de Feyter R et al. (1996) Mol. Gen. Genet. 250(3): 329-338]. Suitable target sequences and ribozymes can be identified for example as described by Steinecke P, Ribozymes, Methods in Cell Biology 50, Galbraith et al. eds, Academic Press, Inc. (1995), pp. 449-460 by calculating the secondary structures of ribozyme RNA and target RNA and by their interaction [Bayley C C et al. (1992) Plant Mol. Biol. 18(2): 353-361; Lloyd A M and Davis R W et al. (1994) Mol. Gen. Genet. 242(6): 653-657]. For example, derivatives of the tetrahymena L-19 IVS RNA, which have complementary regions to the mRNA of the protein to be suppressed, can be constructed (see also U.S. Pat. No. 4,987,071 and U.S. Pat. No. 5,116,742). As an alternative, such ribozymes can also be identified from a library of a variety of ribozymes via a selection process [Bartel D and Szostak J W (1993) Science 261: 1411-1418].

d) Introduction of a (sense) nucleic acid sequence for inducing cosuppression, e.g. for the reduction, repression or deletion of activity of the nucleic acid molecule or polypeptide which activity is to be reduced in the process of the invention, in particular of a nucleic acid molecule comprising a polynucleotide as depicted in column 5 or 7, of Table I or encoding a polypeptide comprising a polypeptide, a consensus sequence or a polypeptide motif as depicted in column 5 or 7 of Table II or IV

Accordingly, yet another embodiment of the invention is a coexpression construct, which confers—after being expressed in a suitable organism, e.g. a plant, or a part thereof—the reduction, repression, or deletion of an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme.

Yet another embodiments of the invention is a coexpression construct conferring the decline or inactivation of a molecule conferring the expression of a protein as shown in column 5 or 7 of Table II, preferably as depicted in Table II B, or comprising a consensus sequence or a polypeptide motif as shown in Table IV or being encoded by a nucleic acid molecule comprising a polynucleotide as depicted in column 5 or 7 of Table I, preferably as depicted in Table I B, or a homologue thereof as described herein, e.g. conferring the decline or inactivation of the nucleic acid molecule or the polypeptide of the invention, with the result that the tolerance and/or resistance to environmental stress and the biomass production as compared to a corresponding non-transformed wild type plant are increased.

The expression of a nucleic acid sequence in sense orientation can lead to cosuppression of the corresponding homologous, endogenous genes. The expression of sense RNA with homology to an endogenous gene can reduce or indeed eliminate the expression of the endogenous gene, in a similar manner as has been described for the following antisense approaches: Jorgensen et al. (1996) Plant Mol. Biol. 31(5): 957-973, Goring et al. (1991) Proc. Natl. Acad. Sci. USA 88: 1770-1774], Smith et al. (1990) Mol. Gen. Genet. 224: 447-481, Napoli et al. (1990) Plant Cell 2: 279-289 or Van der Krol et al. (1990) Plant Cell 2: 291-99. In this context, the construct introduced may represent the homologous gene to be reduced either in full or only in part. The application of this technique to plants has been described for example by Napoli et al. (1990) The Plant Cell 2: 279-289 and in U.S. Pat. No. 5,03410,323. Furthermore the above described cosuppression strategy can advantageously be combined with the RNAi method as described by Brummell et al., 2003, Plant J. 33, pp 793-800. At least in plants it is advantageously to use strong or very strong promoters in cosuppression approaches. Recent work for example by Schubert et al., (Plant Journal 2004, 16, 2561-2572) has indicated that cosuppression effects are dependent on a gene specific threshold level, above which cosuppression occurs.

e) Introduction of nucleic acid sequences encoding a dominant-negative protein, e.g. for the reduction or deletion of activity of the polypeptide which activity is reduced in the process of the invention, in particular of a polypeptide encoded by a nucleic acid molecule comprising a polynucleotide as depicted in column 5 or 7, of Table I or of a polypeptide comprising a polypeptide, or a consensus sequence or a polypeptide motif as depicted in column 5 or 7 of Table II or IV

Accordingly, yet another embodiment of the invention is a dominant negative mutant, which confers—after being expressed in a suitable organism, e.g. a plant, or a part thereof—the reduction, repression, or deletion of an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme.

Yet another embodiment of the invention is a dominate negative mutant conferring the decline or inactivation of a polypeptide conferring the expression of a protein as depicted in column 5 or 7 of Table II, preferably as depicted in Table II B, or of a polypeptide comprising a consensus sequence or a polypeptide motif as depicted in Table IV or being encoded by a nucleic acid molecule comprising a polynucleotide as depicted in column 5 or 7 of Table I, preferably as depicted in Table I B, or a homologue thereof as described herein, e.g. conferring the decline or inactivation of the nucleic acid molecule or the polypeptide of the invention, with the result that the tolerance and/or resistance to environmental stress and the biomass production as compared to a corresponding non-transformed wild type plant are increased.

The function or activity of a protein can efficiently also be reduced by expressing a dominant-negative variant of said protein. The skilled worker is familiar with methods for reducing the function or activity of a protein by means of coexpression of its dominant-negative form [Lagna G and Hemmati-Brivanlou A (1998) Current Topics in Developmental Biology 36: 75-98; Perlmutter R M and Alberola-Ila J (1996) Current Opinion in Immunology 8(2): 285-90; Sheppard D (1994) American Journal of Respiratory Cell & Molecular Biology 11(1): 1-6; Herskowitz I (1987) Nature 329 (6136): 219-22].

A dominant-negative variant can be realized for example by changing of an amino acid of a polypeptide encoded by a nucleic acid molecule comprising a polynucleotide as depicted in column 5 or 7, of Table I or of a polypeptide comprising a polypeptide or a consensus sequence or a polypeptide motif as depicted in column 5 or 7 of Table II or IV or homologs thereof.

This change can be determined for example by computer-aided comparison (“alignment”). These mutations for achieving a dominant-negative variant are preferably carried out at the level of the nucleic acid sequences. A corresponding mutation can be performed for example by PCR-mediated in-vitro mutagenesis using suitable oligonucleotide primers by means of which the desired mutation is introduced. To this end, methods are used with which the skilled worker is familiar. For example, the “LA PCR in vitro Mutagenesis Kit” (Takara Shuzo, Kyoto) can be used for this purpose. It is also possible and known to those skilled in the art that deleting or changing of functional domains, e.g. TF or other signaling components which can bind but not activate may achieve the reduction of protein activity.

f) Introduction of DNA- or protein-binding factor against genes RNAs or proteins, e.g. for the reduction, repression or deletion of activity of the nucleic acid molecule or polypeptide which activity is reduced in the process of the invention, in particular of a nucleic acid molecule comprising a polynucleotide as depicted in column 5 or 7, of Table I or encoding a polypeptide comprising a polypeptide or a consensus sequence or a polypeptide motif as depicted in column 5 or 7 of Table II or IV

Accordingly, yet another embodiment of the invention is a DNA- or protein-binding factor against genes RNAs or proteins, which confers—after being expressed in a suitable organism, e.g. a plant, or a part thereof—the reduction, repression, or deletion of an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme.

Yet another embodiment of the invention is a DNA- or protein-binding factor against genes RNAs or proteins conferring the decline or inactivation of a molecule conferring the expression of a protein as depicted in column 5 or 7 of Table II, preferably as depicted in Table II B, or of a polypeptide comprising a consensus sequence or a polypeptide motif as depicted in Table IV or being encoded by a nucleic acid molecule comprising a polynucleotide as depicted in column 5 or 7 of Table I, preferably as depicted in Table I B, or a homologue thereof as described herein, e.g. conferring the decline or inactivation of the nucleic acid molecule or the polypeptide of the invention, with the result that the tolerance and/or resistance to environmental stress and increased biomass production as compared to a corresponding non-transformed wild type plant are increased.

A reduction in the expression of a gene encoding the nucleic acid molecule or the polypeptide which activity is reduced in the process of the invention, in particular comprising a nucleic acid molecule comprising a polynucleotide as depicted in column 5 or 7, of Table I or encoding a polypeptide comprising a polypeptide, a consensus sequence or a polypeptide motif as depicted in column 5 or 7 of Table II or IV or homologs thereof according to the invention can also be achieved with specific DNA-binding factors, for example factors of the zinc finger transcription factor type. These factors attach to the genomic sequence of the endogenous target gene, preferably in the regulatory regions, and bring about repression of the endogenous gene. The use of such a method makes possible the reduction in the expression of an endogenous gene without it being necessary to recombinantly manipulate the sequence of the latter. Such methods for the preparation of relevant factors are described in Dreier B et al. (2001) J. Biol. Chem. 276(31): 29466-78 and (2000) J. Mol. Biol. 303(4): 489-502, Beerli R R et al. (1998) Proc. Natl. Acad. Sci. USA 95(25): 14628-14633; (2000) Proc. Natl. Acad. Sci. USA 97(4): 1495-1500 and (2000) J. Biol. Chem. 275(42): 32617-32627), Segal D J and Barbas C F, 3rd (2000) Curr. Opin. Chem. Biol. 4(1): 3410-39, Kang J S and Kim J S (2000) J. Biol. Chem. 275(12): 8742-8748, Kim J S et al. (1997) Proc. Natl. Acad. Sci. USA 94(8): 3616-3620, Klug A (1999) J. Mol. Biol. 293(2): 215-218, Tsai S Y et al. (1998) Adv. Drug Deliv. Rev. 30(1-3): 23-31, Mapp A K et al. (2000) Proc. Natl. Acad. Sci. USA 97(8): 3930-3935, Sharrocks A D et al. (1997) Int. J. Biochem. Cell Biol. 29(12): 1371-1387 and Zhang L et al. (2000) J. Biol. Chem. 275(43): 33850-33860. Examples for the application of this technology in plants have been described in WO 01/52620, Ordiz M I et al., (Proc. Natl. Acad. Sci. USA, Vol. 99, Issue 20, 13290-13295, 2002) or Guan et al., (Proc. Natl. Acad. Sci. USA, Vol. 99, Issue 20, 13296-13301, 2002)

These factors can be selected using any portion of a gene. This segment is preferably located in the promoter region. For the purposes of gene suppression, however, it may also be located in the region of the coding exons or introns. The skilled worker can obtain the relevant segments from Genbank by database search or starting from a cDNA whose gene is not present in Genbank by screening a genomic library for corresponding genomic clones.

It is also possible to first identify sequences in a target crop, which encompass the nucleic acid molecule or which encode the polypeptide which activity is reduced in the process of the invention, in particular of a nucleic acid molecule comprising a polynucleotide as depicted in column 5 or 7, of Table I B or encoding a polypeptide comprising a polypeptide, a consensus sequence or a polypeptide motif as depicted in column 5 or 7 of Table II B or homologs thereof, then find the promoter and reduce expression by the use of the abovementioned factors.

The skilled worker is familiar with the methods required for doing so.

Furthermore, factors which are introduced into a cell may also be those which themselves inhibit the target protein. The protein-binding factors can, for example, be aptamers [Famulok M and Mayer G (1999) Curr. Top Microbiol. Immunol. 243: 123-36] or antibodies or antibody fragments or single-chain antibodies. Obtaining these factors has been described, and the skilled worker is familiar therewith. For example, a cytoplasmic scFv antibody has been employed for modulating activity of the phytochrome A protein in genetically modified tobacco plants [Owen M et al. (1992) Biotechnology (NY) 10(7): 790-794; Franken E et al. (1997) Curr. Opin. Biotechnol. 8(4): 411-416; Whitelam (1996) Trend Plant Sci. 1: 286-272].

Gene expression may also be suppressed by tailor-made low-molecular-weight synthetic compounds, for example of the polyamide type Dervan P B and Bürli R W (1999) Current Opinion in Chemical Biology 3: 688-693; Gottesfeld J M et al. (2000) Gene Expr. 9(1-2): 77-91. These oligomers consist of the units 3-(dimethylamino)propylamine, N-methyl-3-hydroxypyrrole, N-methylimidazole and N-methylpyrroles; they can be adapted to each portion of double-stranded DNA in such a way that they bind sequence-specifically to the large groove and block the expression of the gene sequences located in this position. Suitable methods have been described in Bremer R E et al. [(2001) Bioorg. Med. Chem. 9 (8): 2093-103], Ansari A Z et al. [(2001) Chem. Biol. 8(6): 583-92], Gottesfeld J M et al. [(2001) J. Mol. Biol. 309(3): 615-29], Wurtz N R et al. [(2001) Org. Lett 3(8): 1201-3], Wang C C et al. [(2001) Bioorg. Med. Chem. 9(3): 653-7], Urbach A R and Dervan P B [(2001) Proc. Natl. Acad. Sci. USA 98(8): 434103-8] and Chiang S Y et al. [(2000) J. Biol. Chem. 275(32): 24246-54].

g) Introduction of viral nucleic acid sequences and expression constructs which bring about the degradation of RNA, e.g. for the reduction, repression or deletion of activity of the nucleic acid molecule or polypeptide which activity is to be reduced in the process of the invention, in particular of a nucleic acid molecule comprising a polynucleotide as depicted in column 5 or 7, of Table I or encoding a polypeptide comprising a polypeptide, a consensus sequence or a polypeptide motif as depicted in column 5 or 7 of Table II or IV.

Accordingly, yet another embodiment of the invention is a viral nucleic acid molecule, which confers—after being expressed in a suitable organism, e.g. a plant, or a part thereof—the reduction, repression, or deletion of an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme.

Yet another embodiment of the invention is a viral nucleic acid molecule conferring the decline or inactivation of a RNA molecule conferring the expression of a protein as depicted in column 5 or 7 of Table II, preferably as depicted in Table II B, or a polypeptide comprising a consensus sequence or a polypeptide motif of Table IV or being encoded by a nucleic acid molecule comprising a polynucleotide as depicted in column 5 or 7 of Table I, preferably as depicted in Table I B, or a homologue thereof as described herein, e.g. conferring the decline or inactivation of the nucleic acid molecule or the polypeptide of the invention, with the result that the tolerance and/or resistance to environmental stress and the biomass production as compared to a corresponding non-transformed wild type plant are increased.

Inactivation or downregulation can also be efficiently brought about by inducing specific RNA degradation by the organism, advantageously in the plant, with the aid of a viral expression system (Amplikon) (Angell, S M et al. (1999) Plant J. 20(3): 357-362). Nucleic acid sequences with homology to the transcripts to be suppressed are introduced into the plant by these systems—also referred to as “VIGS” (viral induced gene silencing) with the aid of viral vectors. Then, transcription is switched off, presumably mediated by plant defense mechanisms against viruses. Suitable techniques and methods are described in Ratcliff F et al. (2001) Plant J. 25(2): 237-45, Fagard M and Vaucheret H (2000) Plant Mol. Biol. 43(2-3): 285-93, Anandalakshmi Ret al. (1998) Proc. Natl. Acad. Sci. USA 95(22): 13079-84 and Ruiz M T (1998) Plant Cell 10(6): 937-46.

h) Introduction of constructs for inducing a homologous recombination on endogenous genes, for example for generating knock-out mutants e.g. for the reduction, repression or deletion of activity of the nucleic acid molecule or polypeptide which activity is reduced in the process of the invention, in particular of a nucleic acid molecule comprising a polynucleotide as depicted in column 5 or 7, of Table I or encoding a polypeptide comprising a polypeptide, a consensus sequence or a polypeptide motif as depicted in column 5 or 7 of Table II or IV.

Accordingly, yet another embodiment of the invention is a construct for inducing a homologous recombination on endogenous genes, which confers—after being introduced in a suitable organism, e.g. a plant, or a part thereof—the reduction, repression, or deletion of an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme.

Yet another embodiment of the invention is a construct for inducing homologous recombination on endogenous genes conferring the decline or inactivation of a molecule conferring the expression of a protein as depicted in column 5 or 7 of Table II, preferably as depicted in Table II B, or of a polypeptide comprising a consensus sequence or a polypeptide motif as depicted in Table IV or being encoded by a nucleic acid molecule comprising a polynucleotide as depicted in column 5 or 7 of Table I, preferably as depicted in Table I B, or a homologue thereof as described herein, e.g. conferring the decline or inactivation of the nucleic acid molecule or the polypeptide of the invention, with the result that the tolerance and/or resistance to environmental stress and the biomass production as compared to a corresponding non-transformed wild type plant are increased.

To generate a homologously-recombinant organism with reduced activity, a nucleic acid construct is used which, for example, comprises at least part of an endogenous gene which is modified by a deletion, addition or substitution of at least one nucleotide in such a way that the functionality is reduced or completely eliminated. The modification may also affect the regulatory elements (for example the promoter) of the gene so that the coding sequence remains unmodified, but expression (transcription and/or translation) does not take place or is reduced.

In the case of conventional homologous recombination, the modified region is flanked at its 5′ and 3′ end by further nucleic acid sequences, which must be sufficiently long for allowing recombination. Their length is, as a rule, in a range of from one hundred bases up to several kilobases [Thomas K R and Capecchi M R (1987) Cell 51: 503; Strepp et al. (1998) Proc. Natl. Acad. Sci. USA 95(8): 4368-4373]. In the case of homologous recombination, the host organism—for example a plant—is transformed with the recombination construct using the methods described herein below, and clones, which have successfully undergone recombination are selected using for example a resistance to antibiotics or herbicides. Using the cotransformation technique, the resistance to antibiotics or herbicides can subsequently advantageously be re-eliminated by performing crosses. An example for an efficient homologous recombination system in plants has been published in Nat. Biotechnol. 2002 October; 20(10): 1030-4, Terada R et al.: Efficient gene targeting by homologous recombination in rice.

Homologous recombination is a relatively rare event in higher eukaryotes, especially in plants. Random integrations into the host genome predominate. One possibility of removing the randomly integrated sequences and thus increasing the number of cell clones with a correct homologous recombination is the use of a sequence-specific recombination system as described in U.S. Pat. No. 6,110,736, by means of which unspecifically integrated sequences can be deleted again, which simplifies the selection of events which have integrated successfully via homologous recombination. A multiplicity of sequence-specific recombination systems may be used, examples which may be mentioned being Cre/lox system of bacteriophage P1, the FLP/FRT system from yeast, the Gin recombinase of phage Mu, the Pin recombinase from E. coli and the R/RS system of the pSR1 plasmid. The bacteriophage P1 Cre/lox system and the yeast FLP/FRT system are preferred. The FLP/FRT and the cre/lox recombinase system have already been applied to plant systems [Odell et al. (1990) Mol. Gen. Genet. 223: 369-378].

I) Introduction of mutations into endogenous genes for bringing about a loss of function (for example generation of stop codons, reading-frame shifts and the like) e.g. for the reduction, repression or deletion of activity of the nucleic acid molecule or polypeptide which activity is reduced in the process of the invention, in particular of a nucleic acid molecule comprising a polynucleotide as depicted in column 5 or 7, of Table I or encoding a polypeptide comprising a polypeptide, a consensus sequence or a polypeptide motif as depicted in column 5 or 7 of Table II or IV

Accordingly, yet another embodiment of the invention is a mutated homologue of the nucleic acid molecule which activity is reduced in the process of the invention and, which confers—after being expressed in a suitable organism, e.g. a plant, or a part thereof—the reduction, repression, or deletion of an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme.

Further suitable methods for reducing activity are the introduction of nonsense, deletion or integration mutations into endogenous genes, for example by introducing RNA/DNA oligonucleotides into the plant [Zhu et al. (2000) Nat. Biotechnol. 18(5): 555-558], and the generation of knock-out mutants with the aid of, for example, T-DNA mutagenesis [Koncz et al. (1992) Plant Mol. Biol. 20(5): 963-976], ENU-(N-ethyl-N-nitrosourea)—mutagenesis or homologous recombination [Hohn B and Puchta (1999) H. Proc. Natl. Acad. Sci. USA 96: 8321-8323]. Point mutations may also be generated by means of DNA-RNA hybrids also known as “chimeraplasty” [Cole-Strauss et al. (1999) Nucl. Acids Res. 27(5): 1323-1330; Kmiec (1999) Gene Therapy American Scientist 87(3): 240-247]. The mutation sites may be specifically targeted or randomly selected. If the mutations have been created randomly e.g. by Transposon-Tagging or chemical mutagenesis, the skilled worked is able to specifically enrich selected mutation events in the inventive nucleic acids, especially by different PCR methods know to the person skilled in the art. Mutations can also be introduced by the introduction of so-called homing endonucleases which can be designed to set double strand breaks in specific sequences within the genome. The repair of said double strand breaks often leads to the desired non-functional mutations. [Arnould et al (2006) Engineering of large numbers of highly specific homing endonucleases that induce recombination on novel DNA targets. Journal of Molecular Biology 355(3): 443-458]

j) Introduction of a microRNA (or micro-RNA) that has been designed to target the gene of interest in order to induce a breakdown or translational inhibition of the mRNA of the gene of interest and thereby silence gene expression or of an expression cassette ensuring the expression of the former, e.g. for the reduction, repression or deletion of activity of the nucleic acid molecule or polypeptide which activity is reduced in the process of the invention, in particular of a nucleic acid molecule comprising a polynucleotide as depicted in column 5 or 7, of Table I or encoding a polypeptide comprising a polypeptide, a consensus sequence or a polypeptide motif as depicted in column 5 or 7 of Table II or IV

Accordingly, yet another embodiment of the invention is a miRNA molecule, which confers—after being expressed in a suitable organism, e.g. a plant, or a part thereof—the reduction, repression, or deletion of an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme.

Yet another embodiment of the invention is a miRNA molecule conferring the decline or inactivation of a molecule conferring the expression of a protein as depicted in column 5 or 7 of Table II, preferably as depicted in Table II B, or a polypeptide comprising a consensus sequence or a polypeptide motif as depicted in Table IV or being encoded by a nucleic acid molecule comprising a polynucleotide as depicted in column 5 or 7 of Table I, preferably as depicted in Table I B, or a homologue thereof as described herein, e.g. conferring the decline or inactivation of the nucleic acid molecule or the polypeptide of the invention, with the result that the tolerance and/or resistance to environmental stress and the biomass production as compared to a corresponding non-transformed wild type plant are increased.

MicroRNAs (miRNAs) have emerged as evolutionarily conserved, RNA-based regulators of gene expression in plants and animals. MiRNAs (˜21 to 25 nt) arise from larger precursors with a stem loop structure that are transcribed from non-protein-coding genes. miRNA targets a specific mRNA to suppress gene expression at post-transcriptional (i.e. degrades mRNA) or translational levels (i.e. inhibits protein synthesis) (Bartel D 2004, Cell 116, 281-297). MiRNAs can be efficiently designed to specifically target and down regulated selected genes. Determinants of target selection of natural plant miRNAs have been analysed by Schwab and coworkers (Schwab et al. 2005, 2005 Dev. Cell 8, 517-527). This work has been extended to the design and use of artificial miRNAs (amiRNAs) to efficiently down regulate target genes, resulting in concepts and rules for the design of effective amiRNAs for directed gene silencing [Highly Specific Gene Silencing by Artificial microRNAs in Arabidopsis, Schwab et al., Plant Cell 2006 18 (4)] and a web based tool for efficient amiRNA design (http://wmd.weigelworld.org).

k) Introduction of a transacting small interfering RNA (ta-siRNA) or of an expression cassette ensuring the expression of the former, e.g. for the reduction, repression or deletion of activity of the nucleic acid molecule or polypeptide which activity is reduced in the process of the invention, in particular of a nucleic acid molecule comprising a polynucleotide as depicted in column 5 or 7, of Table I or encoding a polypeptide comprising a polypeptide, a consensus sequence or a polypeptide motif as depicted in column 5 or 7 of Table II or IV

Accordingly, yet another embodiment of the invention is a ta-siRNA, which confers—after being expressed in a suitable organism, e.g. a plant, or a part thereof—the reduction, repression, or deletion of an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme.

Yet another embodiment of the invention is a ta-siRNA conferring the decline or inactivation of a molecule conferring the expression of a protein as depicted in column 5 or 7 of Table II, preferably as depicted in Table II B, or a polypeptide comprising a consensus sequence or a polypeptide motif as depicted in Table IV or being encoded by a nucleic acid molecule comprising a polynucleotide as depicted in column 5 or 7 of Table I, preferably as depicted in Table I B, or a homologue thereof as described herein, e.g. conferring the decline or inactivation of the nucleic acid molecule or the polypeptide of the invention, with the result that the tolerance and/or resistance to environmental stress and the biomass production as compared to a corresponding non-transformed wild type plant are increased.

A transacting small interfering RNA (ta-siRNA) can be designed to target the gene of interest in order to induce a breakdown of the mRNA of the gene of interest and thereby silence gene expression.

Methods employing ta-siRNAs useful for the repression or inactivation of a gene product according to the process of the present invention are described in U.S. 60/672,976 and 60/718,645.

Nucleic acid sequences as described in item B) to K) are expressed in the cell or organism by transformation/transfection of the cell or organism or are introduced in the cell or organism by known methods, for example as disclosed in item A).

l) Identifying a non silent mutation, e.g. generation of stop codons, reading-frame shifts, integrations, inversions and the like in random mutagenized population according to different approaches like reverse screening or the so called TILLING (Targeting Induced Local Lesions IN Genomes) method, e.g. for the reduction, repression or deletion of activity of the nucleic acid molecule or polypeptide which activity is reduced in the process of the invention, in particular of a nucleic acid molecule comprising a polynucleotide as depicted in column 5 or 7, of Table I or encoding a polypeptide comprising a polypeptide, a consensus sequence or a polypeptide motif, as depicted in column 5 or 7 of Table II or IV.

Accordingly, yet another embodiment of the invention is a TLLING or severse screening primer or a heteroduplex between a mutated DNA and a wild type DNA, which can be used to a identify mutation which confers—after being expressed in a suitable organism, e.g. a plant, or a part thereof—the reduction, repression, or deletion of an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme.

Yet another embodiment of the invention is a TLLING or reverse screening primer for identifying a mutation conferring the decline or inactivation of a molecule conferring the expression of a protein as depicted in column 5 or 7 of Table II, preferably as depicted in Table II B, or of a polypeptide comprising a consensus sequence or a polypeptide motif as depicted in Table IV or being encoded by a nucleic acid molecule comprising a polynucleotide as depicted in column 5 or 7 of Table I, preferably as depicted in Table I B, or a homologue thereof as described herein, e.g. conferring the decline or inactivation of the nucleic acid molecule or the polypeptide of the invention, with the result that the tolerance and/or resistance to environmental stress and the biomass production as compared to a corresponding non-transformed wild type plant are increased.

Particular preferred is a TILLING or a reverse screening primer for the identification of a mutation in a nucleic acid molecule which is a homologue of a nucleic acid molecule as depicted in column 5 or 7 of Table I, preferably as depicted in Table I B, such as a nucleic acid molecule comprising a nucleic acid molecule as depicted in column 5 or 7 of Table I, preferably as depicted in Table I B but which is mutated in one or more nucleotides.

In one embodiment, the TILLING or reverse screening primer comprises a fragment of at least 17 nucleotides (nt), preferably of 18, 19, 20, 21, 22, 23, 24, 25, 27, 30 nt of a nucleic acid molecule as depicted in column 5 or 7 of Table I, preferably as depicted in Table I B.

In one embodiment, the TILLING or reverse screening primer comprises a fragment of at least 17 nucleotides (nt), preferably of 18, 19, 20, 21, 22, 23, 24, 25, 27, 30 nt and which is at least 70%, 75%, 80%, 90%, more preferred at least 95%, most preferred 100% homologue to a nucleic acid molecule as depicted in column 5 or 7 of Table I, preferably as depicted in Table I B.

For the TILLING, mutations are induced by treatment with a chemical mutagen (EMS). DNAs are prepared from individuals and arrayed in pools for initial screening. These pools become templates for PCR using primers that amplify a region of interest. Heteroduplexes are formed between wild-type and mutant fragments in the pool by denaturing and reannealing PCR products. These heteroduplexes are the substrate for cleavage by the nuclease CEL I. After digestion, the resulting products are visualized using standard fluorescent sequencing slab gel electrophoresis. Positive pools are then rescreened as individual DNAs, thus identifying the mutant plant and the approximate position of the mutation along the sequence. This positional information increases the efficiency of sequence analysis, as heterozygous mutations may be otherwise difficult to identify.

High-throughput TILLING is for example described in Colbert et al. (2001) Plant Physiology 126: 480-484 and has recently been applied to crops [reviewed in Slade and Knauf, Transgenic Res. 2005 April; 14(2): 109-15].

Other reverse screening methods aims to identify individuals in populations, mutated through the random integration of nucleic acids, like transposons or T-DNAs have been described severals times, eg. Krysan et al., 1999 (Plant Cell 1999, 11, 2283-2290); Sessions et al., 2002 (Plant Cell 2002, 14, 2985-2994); Young et al., 2001, (Plant Physiol. 2001, 125, 513-518); Koprek et al., 2000 (Plant J. 2000, 24, 253-263); Jeon et al., 2000 (Plant J. 2000, 22, 561-570); Tissier et al., 1999 (Plant Cell 1999, 11, 1841-1852); Speulmann et al., 1999 (Plant Cell 1999, 11, 1853-1866),

In one further embodiment of the process according to the invention, organisms are used in which one of the abovementioned genes, or one of the above-mentioned nucleic acids, is mutated in such a manner that the activity of the encoded gene products is influenced by cellular factors to a greater extent than in the reference organism, as compared with the unmutated proteins. This kind of mutation could lead to a change in the metabolic activity of the organism, which than causes in a higher tolerance and/or resistance to environmental stress and higher biomass production as compared to a corresponding non-transformed wild type plant. The reason for this higher productivity can be due to a change in regulation mechanism of enzymic activity such as substrate inhibition or feed back regulation. In a further embodiment the process according to the invention, organisms are grown under such conditions, that the expression of the nucleic acids of the invention is reduced or repressed leading to an enhanced tolerance and/or resistance to environmental stress and higher biomass production as compared to a corresponding non-transformed wild type plant according to the invention.

In one embodiment the tolerance and/or resistance to environmental stress and the biomass production as compared to a corresponding non-transformed wild type plant in the organism or part thereof can be increased by targeted or random mutagenesis of the endogenous genes comprising or encoding the molecule which activity is to be reduced in the process of the invention, e.g. comprising a polynucleotide as depicted in column 5 or 7 of Table I or encoding an polypeptide comprising a polypeptide, a consensus sequence or a polypeptide motif as depicted in column 5 or 7 of Table II or IV.

For example homologous recombination can be used to either introduce negative regulatory elements or to remove, interrupt or delete enhancer elements form regulatory regions. In addition gene conversion like methods described by Kochevenko and Willmitzer (Plant Physiol. 2003 May; 132(1): 174-84) and citations therein may be modified to disrupt enhancer elements or to enhance to activity of negative regulatory elements. Furthermore mutations or repressing elements can be randomly introduced in (plant) genomes by T-DNA or transposon mutagenesis and lines can be screened for, in which repressing or interrupting elements have be integrated near to a gene of the invention, the expression of which is thereby repressed, reduced or deleted. The inactivation of plant genes by random integrations of enhancer elements has been described.

Reverse genetic strategies to identify insertions (which eventually carrying the inactivation elements) near in genes of interest have been described for various cases eg. Krysan et al., 1999 (Plant Cell 1999, 11, 2283-2290); Sessions et al., 2002 (Plant Cell 2002, 14, 2985-2994); Young et al., 2001, (Plant Physiol. 2001, 125, 513-518); Koprek et al., 2000 (Plant J. 2000, 24, 253-263); Jeon et al., 2000 (Plant J. 2000, 22, 561-570); Tissier et al., 1999 (Plant Cell 1999, 11, 1841-1852); Speulmann et al., 1999 (Plant Cell 1999, 11, 1853-1866).

The enhancement of negative regulatory elements or the disruption or weaking of enhancing or activating regulatory elements can also be achieved through common mutagenesis techniques: The production of chemically or radiation mutated populations is a common technique and known to the skilled worker.

Accordingly, the expression level can be increased if the endogenous genes encoding a polypeptide or a nucleic acid molecule conferring the activity described herein, in particular genes comprising the nucleic acid molecule of the present invention, are modified by a mutagenesis approach via homologous recombination with optional identification by TILLING or other reverse screening approaches, or gene conversion.

In one embodiment of the invention, the applicable modification of the nucleic acid molecules described herein for the use in the process of the invention, i.e. the reduction, repression or deletion of its activity and being itself encoded by the host organism can for example be achieved by random mutagenesis with chemicals, radiation or UV-light or side directed mutagenesis in such a manner that the tolerance and/or resistance to environmental stress and the biomass production as compared to a corresponding non-transformed wild type plant are increased. This embodiment of the invention shall be deemed as transgenic in the sense of the invention.

Using the herein mentioned cloning vectors and transformation methods such as those which are published and cited in: Plant Molecular Biology and Biotechnology (CRC Press, Boca Raton, Fla.), chapter 6/7, pp. 71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus, Annu. Rev. Plant Physiol. Plant Molec. Biol. 42 (1991), 205-225)) and further cited below, nucleic acid molecule derived from the polynucleotides described herein for the use in the process of the invention as described herein may be used for the recombinant modification of a wide range of organisms, in particular plants, so that they become a better and more efficient due to the deletion or reduction the activity of genes comprising nucleic acid molecule of the invention or of the expression product of said genes according to the process of the invention.

The improved tolerance and/or resistance to environmental stress and the biomass production as compared to a corresponding non-transformed wild type plant can be brought about by a direct effect of the manipulation or by an indirect effect of this manipulation.

In order to improve the introduction of a nucleic acid molecule for reduction, repression, decrease or deletion of the expression or activity of the molecules to be reduced in the process of the invention in an organisms, the nucleic acid molecules disclosed herein or derivates thereof can be incorporated into a nucleic acid construct and/or a vector in such a manner that their introduction into an organism, e.g. a cell, confers an reduced or deleted endogenous or cellulary activity either on the nucleic acid sequence expression level or on the level of the polypeptide encoded by said sequences.

Accordingly, in order to improve the introduction of a nucleic acid molecule and to confer or improve the reduction, repression, decrease or deletion of the expression or activity of the molecules to be reduced in the process of the invention in an organisms, e.g. in a transgenic plant or microorganism nucleic acid molecules encoding the herein disclosed antisense nucleic acid molecule, RNAi, snRNA, dsRNA, siRNA, miRNA, ta-siRNA, cosuppression molecule, ribozyme, antibodies or other molecule inhibiting the expression or activity of an expression product of the nucleic acid molecule to be reduced, repressed or deleted in the process of the invention can be incorporated into a nucleic acid construct and/or a vector.

After the above-described reducing, repressing, decreasing or deleting (which as defined above also encompasses the generating of an activity in an organism, i.e. a de novo activity), for example after the introduction and the expression of the RNAi, snRNA, dsRNA, siRNA, miRNA, ta-siRNA, cosuppression molecule, ribozyme, antibody or antisense molecule or ribozyme or other molecule inhibiting the expression or activity, as described in the methods or processes according to the invention, the organism according to the invention, advantageously, a plant, plant tissue or plant cell, is grown and subsequently harvested.

Examples can be transgenic or non-transgenic plants, cells or protoplasts thereof. Examples of preferred suitable organisms are described in the following paragraphs.

Suitable host organisms (transgenic organism) for generating the nucleic acid molecule used according to the invention or for the use in the process of the invention, e.g. to be transformed with the nucleic acid construct or the vector (both as described below) of the invention, e.g. conferring the expression of an RNAi, snRNA, dsRNA, siRNA, miRNA, ta-siRNA, ribozyme, or antisense molecule or ribozyme or an other molecule inhibiting the expression or activity, are, in principle, all plants which are suitable for the repression, reduction or deletion of genes, in particular of a nucleic acid molecule comprising a polynucleotide as depicted in column 5 or 7, of Table I or encoding a polypeptide comprising a polypeptide, a consensus sequence or a polypeptide motif as depicted in column 5 or 7 of Table II or IV.

In the event that the (transgenic) host organism is a plant, plant tissue or plant cell such as plants selected from the group consisting of the families Anacardiaceae, Asteraceae, Apiaceae, Betulaceae, Boraginaceae, Brassicaceae, Bromeliaceae, Caricaceae, Cannabaceae, Convolvulaceae, Chenopodiaceae, Cucurbitaceae, Elaeagnaceae, Ericaceae, Euphorbiaceae, Fabaceae, Geraniaceae, Gramineae, Juglandaceae, Lauraceae, Leguminosae, Linaceae or perennial grass, fodder crops, vegetables, ornamentals and Arabidopsis thaliana, this plant is for example either grown on a solid medium or as cells in an, e.g. liquid, medium, which is known to the skilled worker and suits the organism. Furthermore such plants can be grown in soil or therelike.

In one embodiment, the nucleic acid molecule used in the process of the invention, in particular the nucleic acid molecule of the invention, or the production or source organism is or originates from a plant, such as a plant selected from the families Aceraceae, Anacardiaceae, Apiaceae, Asteraceae, Brassicaceae, Cactaceae, Cucurbitaceae, Euphorbiaceae, Fabaceae, Malvaceae, Nymphaeaceae, Papaveraceae, Rosaceae, Salicaceae, Solanaceae, Arecaceae, Bromeliaceae, Cyperaceae, Iridaceae, Liliaceae, Orchidaceae, Gentianaceae, Labiaceae, Magnoliaceae, Ranunculaceae, Carifolaceae, Rubiaceae, Scrophulariaceae, Caryophyllaceae, Ericaceae, Polygonaceae, Violaceae, Juncaceae or Poaceae and preferably from a plant selected from the group of the families Apiaceae, Asteraceae, Brassicaceae, Cucurbitaceae, Fabaceae, Papaveraceae, Rosaceae, Solanaceae, Liliaceae or Poaceae.

Preferred plants are selected from the group consisting of Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrata, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabidopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa] Glycine max, Dolichos soja, Glycine gracilis, Glycine hispida, Phaseolus max, Soja hispida or Soja max [soybean]; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminata, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, long-headed poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea].

All abovementioned host organisms are also usable as source organisms for the nucleic acid molecule used in the process of the invention, e.g. the nucleic acid molecule of the invention.

Preferred are crop plants and in particular plants mentioned herein as host plants such as the families and genera mentioned above for example preferred the species Anacardium occidentale, Calendula officinalis, Carthamus tinctorius, Cichorium intybus, Cynara scolymus, Helianthus annus, Tagetes lucida, Tagetes erecta, Tagetes tenuifolia; Daucus carota; Corylus avellana, Corylus colurna, Borago officinalis; Brassica napus, Brassica rapa ssp., Sinapis arvensis, Brassica juncea, Brassica juncea var. juncea, Brassica juncea var. crispifolia, Brassica juncea var. foliosa, Brassica nigra, Brassica sinapioides, Melanosinapis communis, Brassica oleracea, Arabidopsis thaliana, Anana comosus, Ananas ananas, Bromelia comosa, Carica papaya, Cannabis sative, Ipomoea batatus, Ipomoea pandurata, Convolvulus batatas, Convolvulus tiliaceus, Ipomoea fastigiata, Ipomoea tiliacea, Ipomoea triloba, Convolvulus panduratus, Beta vulgaris, Beta vulgaris var. altissima, Beta vulgaris var. vulgaris, Beta maritima, Beta vulgaris var. perennis, Beta vulgaris var. conditiva, Beta vulgaris var. esculenta, Cucurbita maxima, Cucurbita mixta, Cucurbita pepo, Cucurbita moschata, Olea europaea, Manihot utilissima, Janipha manihot, Jatropha manihot, Manihot aipil, Manihot dulcis, Manihot manihot, Manihot melanobasis, Manihot esculenta, Ricinus communis, Pisum sativum, Pisum arvense, Pisum humile, Medicago sativa, Medicago falcata, Medicago varia, Glycine max, Dolichos soja, Glycine gracilis, Glycine hispida, Phaseolus max, Soja hispida, Soja max, Cocos nucifera, Pelargonium grossularioides, Oleum cocoas, Laurus nobilis, Persea americana, Arachis hypogaea, Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense, Linum trigynum, Punica granatum, Gossypium hirsutum, Gossypium arboreum, Gossypium barbadense, Gossypium herbaceum, Gossypium thurberi, Musa nana, Musa acuminata, Musa paradisiaca, Musa spp., Elaeis guineensis, Papaver orientale, Papaver rhoeas, Papaver dubium, Sesamum indicum, Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata, Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum, Avena sativa, Avena fatua, Avena byzantina, Avena fatua var. sativa, Avena hybrida, Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum, Zea mays, Triticum aestivum, Triticum durum, Triticum turgidum, Triticum hybernum, Triticum macha, Triticum sativum or Triticum vulgare, Cofea spp., Coffea arabica, Coffea canephora, Coffea liberica, Capsicum annuum, Capsicum annuum var. glabriusculum, Capsicum frutescens, Capsicum annuum, Nicotiana tabacum, Solanum tuberosum, Solanum melongena, Lycopersicon esculentum, Lycopersicon lycopersicum, Lycopersicon pyriforme, Solanum integrifolium, Solanum lycopersicum, Theobroma cacao or Camellia sinensis.

Particular preferred plants are plants selected from the group consisting of maize, soja, canola, wheat, barley, triticale, rice, linseed, sunflower, hemp, borage, oil palm, coconut, evening primrose, peanut, sufflower, potato and Arabidopsis.

Other preferred plants are a non-transformed from plants selected from the group consisting of rye, oat, soybean, cotton, rapeseed, manihot, pepper, sunflower, flax, safflower, primrose, rapeseed, turnip rape, tagetes, solanaceous plants, tobacco, eggplant, tomato, Vicia species, pea, alfalfa, coffee, cacao, tea, Salix species, perennial grass and forage crops.

More preferred plants are a non-transformed Linum plant cell, preferably Linum usitatissimum, more preferably the variety Brigitta, Golda, Gold Merchant, Helle, Juliel, Olpina, Livia, Marlin, Maedgold, Sporpion, Serenade, Linus, Taunus, Lifax or Liviola, a non-transformed Heliantus plant cell, preferably Heliantus annuus, more preferably the variety Aurasol, Capella, Flavia, Flores, Jazzy, Palulo, Pegasol, PIR64A54, Rigasol, Sariuca, Sideral, Sunny, Alenka, Candisol or Floyd, or a non-transformed Brassica plant cell, preferably Brassica napus, more preferably the variety Dorothy, Evita, Heros, Hyola, Kimbar, Lambada, Licolly, Liconira, Licosmos, Lisonne, Mistral, Passat, Serator, Siapula, Sponsor, Star, Caviar, Hybridol, Baical, Olga, Lara, Doublol, Karola, Falcon, Spirit, Olymp, Zeus, Libero, Kyola, Licord, Lion, Lirajet, Lisbeth, Magnum, Maja, Mendel, Mica, Mohican, Olpop, Ontarion, Panthar, Prinoe, Pronio, Susanna, Talani, Titan, Transfer, Wiking, Woltan, Zeniah, Artus, Contact or Smart.

In one embodiment of the invention transgenic plants are selected from the group comprising corn, soy, oil seed rape (including canola and winter oil seed reap), cotton, wheat and rice.

All abovementioned host plants are also usable as source organisms for isolation or identification of the nucleic acid molecule or polypeptide which activity is to be reduced in the process of the invention or of a functional equivalent thereof. Maize, soja, canola, hemp, borage, oil palm, coconut, evening primrose, peanut, sufflower, wheat, barley, triticale, rice, linseed, sunflower, potato and Arabidopsis are preferred source plants.

The increase of tolerance and/or resistance to environmental stress and increase in biomass production as compared to a corresponding non-transformed wild type plant are in a plant used in the process of the invention may be increased according to the process of the invention by at least a factor of 1.1, preferably at least a factor of 1.5; 2; or 5, especially preferably by at least a factor of 10 or 30, very especially preferably by at least a factor of 50, in comparison with the wild type, control or reference.

In a preferred embodiment, the present invention relates to a process for increasing the tolerance and/or resistance to environmental stress and the biomass production as compared to a corresponding non-transformed wild type plant comprising the reducing, repressing, decreasing or deleting of the activity of a nucleic acid molecule comprising a polynucleotide having the nucleotide sequence as depicted in column 5 or 7 of Table I or of a homolog thereof or comprising the reducing, repressing, decreasing or deleting of the activity of a polypeptide comprising a polypeptide having the amino acid sequence as depicted in column 5 or 7 of Table II or comprising a consensus sequence or a polypeptide motif as depicted in column 7 of table IV or of a homolog thereof as described herein.

Accordingly, in another preferred embodiment, the present invention relates to a process for increasing the tolerance and/or resistance to environmental stress and the biomass production as compared to a corresponding non-transformed wild type plant comprising reducing, repressing, decreasing or deleting the activity or expression of at least one nucleic acid molecule, comprising a nucleic acid molecule which is selected from the group consisting of:
a) an isolated nucleic acid molecule encoding the polypeptide as depicted in column 5 or 7 of Table II or comprising a consensus sequence or polypeptide motif as depicted in column 7 of Table IV;
b) an isolated nucleic acid molecule as depicted in column 5 or 7 of Table I;
c) an isolated nucleic acid molecule, which, as a result of the degeneracy of the genetic code, can be derived from a polypeptide sequence as depicted in column 5 or 7 of Table II or comprising a consensus sequence or polypeptide motif as depicted in column 7 of Table IV;
d) an isolated nucleic acid molecule having at least 30% identity with the nucleic acid molecule sequence of a polynucleotide comprising the nucleic acid molecule as depicted in column 5 or 7 of Table I;
e) an isolated nucleic acid molecule encoding a polypeptide having at least 30% identity with the amino acid sequence of the polypeptide encoded by the nucleic acid molecule of (a) to (c) and having the activity represented by a protein as depicted in column 5 Table II;
f) an isolated nucleic acid molecule encoding a polypeptide which is isolated with the aid of monoclonal or polyclonal antibodies made against a polypeptide encoded by one of the nucleic acid molecules of (a) to (e) and having the activity represented by the protein as depicted in column 5 of Table II;
g) an isolated nucleic acid molecule encoding a polypeptide comprising the consensus sequence or the polypeptide motif as depicted in column 7 of Table IV and preferably having the activity represented by a protein as depicted in column 5 Table II;
h) an isolated nucleic acid molecule encoding a polypeptide having the activity represented by the protein as depicted in column 5 of Table II;
i) an isolated nucleic acid molecule encoding a polypeptide, the polypeptide being derived by substituting, deleting and/or adding one or more amino acids of the amino acid sequence of the polypeptide encoded by the nucleic acid molecules (a) to (c);
j) an isolated nucleic acid molecule which is obtainable by screening a suitable nucleic acid library, e.g. a library derived from a cDNA or a genomic library, under stringent hybridization conditions with a probe comprising a complementary sequence of a nucleic acid molecule of (a) or (b) or with a fragment thereof, having at least 15 nt, preferably 20 nt, 30 nt, 50 nt, 100 nt, 200 nt or 500 nt of a nucleic acid molecule complementary to a nucleic acid molecule sequence characterized in (a) to (d) and encoding a polypeptide having the activity represented by a protein as depicted in column 5 of Table II; and

or which comprises a sequence which is complementary thereto;

or reducing, repressing, decreasing or deleting of a expression product of a nucleic acid molecule comprising a nucleic acid molecule as depicted in (a) to (j), e.g. a polypeptide comprising a polypeptide as depicted in column 5 or 7 of Table II or comprising a consensus sequence or polypeptide motif as depicted in column 7 of Table IV;

and whereby in a preferred embodiment said nucleic acid molecule or polypeptide confers at least one of the activities shown in [0024.1.1.1].

In one embodiment, the nucleic acid molecule used in the process distinguishes over the sequence as depicted in column 5 or 7 of Table I A or B by at least one or more nucleotides or does not consist of the sequence as depicted in column 5 or 7 of Table I A or B.

In one embodiment, the nucleic acid molecule of the present invention is less than 100%, 99.999%, 99.99%, 99.9% or 99% identical to the sequence as depicted in column 5 or 7 of Table I A or B. In another embodiment, the nucleic acid molecule does not consist of the sequence as depicted in column 5 or 7 of Table I A or B.

Nucleic acid molecules, which are advantageous for the process according to the invention and which encode nucleic acid molecules with the activity represented by an expression product of a nucleic acid molecule comprising a nucleic acid molecule as indicated in column 5 or 7 of Table I, preferable represented by a protein as indicated in column 5 or 7 of Table I B, more preferred represented by the protein as indicated in column 5 of Table I B and conferring the increase in the tolerance and/or resistance to environmental stress and in the biomass production as compared to a corresponding non-transformed wild type plant after reducing or deleting their activity, can be determined from generally accessible databases.

As well, nucleic acid molecules, which are advantageous for the process according to the invention and which encode polypeptides with the activity represented by the protein comprising a polypeptide as indicated in column 5 or 7 of Table II or a consensus sequence or a polypeptide as motif indicated in column 7 of Table IV, preferable represented by the protein as indicated in column 5 or 7 of Table II B or comprising a consensus sequence or a polypeptide motif as indicated in column 7 of Table IV, more preferred by the protein indicated in column 5 of Table II B and conferring the increased tolerance and/or resistance to environmental stress and increased biomass production as compared to a corresponding non-transformed wild type plant can be determined from generally accessible databases.

Those databases, which must be mentioned, in particular in this context are general gene databases such as the EMBL database (Stoesser G. et al., Nucleic Acids Res 2001, Vol. 29, 17-21), the GenBank database (Benson D. A. et al., Nucleic Acids Res 2000, Vol. 28, 15-18), or the PIR database (Barker W. C. et al., Nucleic Acids Res. 1999, Vol. 27, 39-43). It is furthermore possible to use organism-specific gene databases for determining advantageous sequences, in the case of yeast for example advantageously the SGD database (Cherry J. M. et al., Nucleic Acids Res. 1998, Vol. 26, 73-80) or the MIPS database (Mewes H. W. et al., Nucleic Acids Res. 1999, Vol. 27, 44-48), in the case of E. coli the GenProtEC database (http://web.bham.ac.uk/bcm4ght6/res.html), and in the case of Arabidopsis the TAIR-database (Huala, E. et al., Nucleic Acids Res. 2001 Vol. 29(1), 102-5) or the MIPS database.

Further, in another embodiment of the present invention, the molecule to be reduced in the process of the invention is novel. Thus, the present invention also relates to the novel nucleic acid molecule, the “nucleic acid molecule of the invention” or the “polynucleotide of the invention”

The nucleic acid molecules used in the process according to the invention take the form of isolated nucleic acid sequences, which encode polypeptides with the activity of a protein as indicated in column 5 or 7 of Table II A or B, preferable represented by a novel protein as indicated in column 7 of Table II B, and enabling the increase in tolerance and/or resistance to environmental stress and increase in biomass production as compared to a corresponding non-transformed wild type plant by reducing, repressing, decreasing or deleting their activity.

Accordingly, in one embodiment, the invention relates to an isolated nucleic acid molecule conferring the expression of a product, the reduction, repression or deletion of which results in an increase of tolerance and/or resistance to environmental stress and increase of biomass production as compared to a corresponding non-transformed wild type plant and which comprises a nucleic acid molecule selected from the group consisting of:
a) an isolated nucleic acid molecule encoding the polypeptide as depicted in column 5 or 7 of Table II, preferably of Table II B or comprising the consensus sequence or the polypeptide motif, as depicted in column 7 Table IV;
b) an isolated nucleic acid molecule as depicted in column 5 or 7 of Table I, preferably of Table I B;
c) an isolated nucleic acid molecule, which, as a result of the degeneracy of the genetic code, can be derived from a polypeptide sequence as depicted in column 5 or 7 of Table II, preferably of Table II B or from a polypeptide comprising the consensus sequence or the polypeptide motif, as depicted in column 7 Table IV;
d) an isolated nucleic acid molecule having at least 30% identity with the nucleic acid molecule sequence of a polynucleotide comprising the nucleic acid molecule as depicted in column 5 or 7 of Table I, preferably of Table I B;
e) an isolated nucleic acid molecule encoding a polypeptide having at least 30% identity with the amino acid sequence of the polypeptide encoded by the nucleic acid molecule of (a) to (c) and having the activity represented by a protein as depicted in column 5 of Table II;
f) an isolated nucleic acid molecule encoding a polypeptide which is isolated with the aid of monoclonal or polyclonal antibodies directed against a polypeptide encoded by one of the nucleic acid molecules of (a) to (e) and having the activity represented by the protein as depicted in column 5 of Table II;
g) an isolated nucleic acid molecule encoding a polypeptide comprising the consensus sequence or a polypeptide motif as depicted in column 7 of Table IV;
h) an isolated nucleic acid molecule encoding a polypeptide having the activity represented by the protein as depicted in column 5 of Table II;
i) an isolated nucleic acid molecule which comprises a polynucleotide, which is obtained by amplifying a cDNA library or a genomic library using the primers as depicted in column 7 of Table III, which do not start at their 5 prime end with the nucleotides ATA;
j) an isolated nucleic acid molecule encoding a polypeptide, the polypeptide being derived by substituting, deleting and/or adding one or more amino acids of the amino acid sequence of the polypeptide encoded by the nucleic acid molecules (a) to (c); and
k) an isolated nucleic acid molecule which is obtainable by screening a suitable nucleic acid library under stringent hybridization conditions with a probe comprising a complementary sequence of a nucleic acid molecule of (a) or (b) or with a fragment thereof, having at least 15 nt, preferably 20 nt, 30 nt, 50 nt, 100 nt, 200 nt or 500 nt of a nucleic acid molecule complementary to a nucleic acid molecule sequence characterized in (a) to (d) and encoding a polypeptide having the activity represented by a protein as depicted in column 5 of Table II; or which comprises a sequence which is complementary thereto;

whereby the nucleic acid molecule according to (a) to (k) differs at least in one, five, ten, 20, 50, 100 or more nucleotides from the sequence as depicted in column 5 or 7 of Table I A and/or which encodes a protein which differs at least in one, five, ten, 20, 30, 50 or more amino acids from the polypeptide sequences as depicted in column 5 or 7 of Table II A.

Accordingly, in another embodiment, the nucleic acid molecule of the invention does not consist of the sequence as depicted in column 5 or 7 of Table I A.

In a further embodiment, the nucleic acid molecule of the present invention is at least 30% identical to the nucleic acid sequence as depicted in column 5 or 7 of Table I A or B and less than 100%, preferably less than 99.999%, 99.99% or 99.9%, more preferably less than 99%, 98%, 97%, 96% or 95% identical to the sequence as depicted in column 5 or 7 of Table I A

As used herein, the term “the nucleic acid molecule of the invention” refers to said nucleic acid molecule as described in this paragraph.

In one embodiment, the present invention also relates to a novel polypeptide, thus to the “the polypeptide of the invention” or the “protein of the invention”.

Preferably, the polypeptide does not comprise a polypeptide as depicted in column 5 or 7 of Table II A. Preferably, the polypeptide of the inventions protein differs at least in one, five, ten, 20, 30, 50 or more amino acids from the polypeptide sequences as depicted in column 5 or 7 of Table II A. In a further embodiment, the polypeptide of the present invention is at least 30% identical to protein sequence as depicted in column 5 or 7 of Table II A or B and less than 100%, preferably less than 99.999%, 99.99% or 99.9%, more preferably less than 99%, 98%, 97%, 96% or 95% identical to the sequence as depicted in column 5 or 7 of Table II A.

As used herein, the terms “the molecule to be reduced in the process of the present invention”, “the nucleic acid molecule to be reduced in the process of the present invention” or “the polypeptide to be reduced in the process of the present invention” comprise the terms “the nucleic acid molecule of the invention” or “the polypeptide of the invention”, respectively.

In one embodiment, the nucleic acid molecule originates advantageously from a plant.

As mentioned, in one embodiment, crop plants are preferred, e.g. above host plants.

However, it is also possible to use artificial sequences, which differ preferably in one or more bases from the nucleic acid sequences found in organisms, or in one or more amino acid molecules from polypeptide sequences found in organisms, to carry out the invention, e.g. to repress, inactive or down regulate an activity selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme, e.g. to repress, inactive or down regulate an activity of the nucleic acid molecule or polypeptide, conferring above-mentioned activity, e.g. conferring the increased tolerance and/or resistance to environmental stress and increased biomass production as compared to a corresponding non-transformed wild type plant after reducing, repressing, decreasing or deleting its expression or activity.

In the process according to the invention nucleic acid molecules can be used, which, if appropriate, contain synthetic, non-natural or modified nucleotide bases, which can be incorporated into DNA or RNA. Said synthetic, non-natural or modified bases can for example increase the stability of the nucleic acid molecule outside or inside a cell. The nucleic acid molecules used in the process of the invention can contain the same modifications as aforementioned.

As used in the present context the nucleic acid molecule can also encompass the untranslated sequence located at the 3′ and at the 5′ end of the coding gene region, for example at least 500, preferably 200, especially preferably 100, nucleotides of the sequence upstream of the 5′ end of the coding region and at least 100, preferably 50, especially preferably 20, nucleotides of the sequence downstream of the 3′ end of the coding gene region. In the event for example the RNAi or antisense technology is used also the 5′- and/or 3′-regions can advantageously be used.

In one embodiment, it is advantageous to choose the coding region for cloning and expression of repression constructs, like antisense, RNAi oder cosuppression constructs, in order to target several or all of the orthologous genes, which otherwise could compensate for each other.

In another embodiment, it is advantageous to use very gene specific sequences originating from the 3′or 5′ prime region for the construction of repression constructs, with the aim to specifically reduce the activity or expression level of only the target gene and, thus, to avoid side effects by repressing other non-target genes (so called off-targets)

The person skilled in the art is familiar with analyzing the actual genomic situation in his target organism. The necessary information can be achieved by search in relevant sequence databases or performing genomic southern blottings disclosing the genomic structure of the target organism and eventually combining these results with informations about expression levels of the target genes disclosed herein, e.g. obtained by array experiments, northern blottings, or RT qPCR experiments.

Preferably, the nucleic acid molecule used in the process according to the invention or the nucleic acid molecule of the invention is an isolated nucleic acid molecule.

An “isolated” polynucleotide or nucleic acid molecule is separated from other polynucleotides or nucleic acid molecules, which are present in the natural source of the nucleic acid molecule. An isolated nucleic acid molecule may be a chromosomal fragment of several kb, or preferably, a molecule only comprising the coding region of the gene. Accordingly, an isolated nucleic acid molecule may comprise chromosomal regions, which are adjacent 5′ and 3′ or further adjacent chromosomal regions, but preferably comprises no such sequences which naturally flank the nucleic acid molecule sequence in the genomic or chromosomal context in the organism from which the nucleic acid molecule originates (for example sequences which are adjacent to the regions encoding the 5′- and 3′-UTRs of the nucleic acid molecule). In various embodiments, the isolated nucleic acid molecule used in the process according to the invention may, for example comprise less than approximately 5 kb, 4 kb, 3 kb, 2 kb, 1 kb, 0.5 kb or 0.1 kb nucleotide sequences which naturally flank the nucleic acid molecule in the genomic DNA of the cell from which the nucleic acid molecule originates.

The nucleic acid molecules used in the process or a part thereof can be isolated using molecular-biological standard techniques and the sequence information provided herein. Also, for example a homologous sequence or homologous, conserved sequence regions at the DNA or amino acid level can be identified with the aid of comparison algorithms. The former can be used as hybridization probes under standard hybridization techniques (for example those described in Sambrook et al., Molecular Cloning: A Laboratory Manual. 2nd Ed., Cold Spring Harbor Laboratory, Cold Spring Harbor Laboratory Press, Cold Spring Harbor, N.Y., 1989) for isolating further nucleic acid sequences useful in this process.

A nucleic acid molecule encompassing a complete sequence of a molecule which activity is to be reduced in the process of the present invention, e.g. as disclosed in column 5 or 7 of Table I, or a part thereof may additionally be isolated by polymerase chain reaction, oligonucleotide primers based on this sequence or on parts thereof being used. For example, a nucleic acid molecule comprising the complete sequence or part thereof can be isolated by polymerase chain reaction using oligonucleotide primers, which have been generated on the basis of the disclosed sequences. For example, mRNA can be isolated from cells, for example by means of the guanidinium thiocyanate extraction method of Chirgwin et al. (1979) Biochemistry 18:5294-5299, and cDNA can be generated by means of reverse transcriptase (for example Moloney MLV reverse transcriptase, available from Gibco/BRL, Bethesda, Md., or AMV reverse transcriptase, obtainable from Seikagaku America, Inc., St. Petersburg, Fla.).

Synthetic oligonucleotide primers for the amplification by means of polymerase chain reaction can be generated on the basis of a sequences shown herein, for example from the molecules comprising the molecules as depicted in column 5 or 7 of Table I or derived from the molecule as depicted in column 5 or 7 of Table I or II. Such primers can be used to amplify nucleic acids sequences for example from cDNA libraries or from genomic libraries and identify nucleic acid molecules, which are useful in the inventive process. For example, the primers as depicted in column 7 of Table III, which do not start at their 5 prime end with the nucleotides ATA, are used.

Moreover, it is possible to identify conserved regions from various organisms by carrying out protein sequence alignments with the polypeptide encoded by the nucleic acid molecule to be reduced according to the process of the invention, in particular with the sequences encoded by the nucleic acid molecule as depicted in column 5 or 7 of Table II, from which conserved regions, and in turn, degenerate primers can be derived.

Conserved regions are those, which show a very little variation in the amino acid in one particular position of several homologs from different origin. The consensus sequence and polypeptide motifs as depicted in column 7 of Table IV are derived from said alignments. Moreover, it is possible to identify conserved regions from various organisms by carrying out protein sequence alignments with the polypeptide encoded by the nucleic acid molecule to be reduced according to the process of the invention, in particular with the sequences encoded by the polypeptide molecule as depicted in column 5 or 7 of Table II, from which conserved regions, and in turn, degenerate primers can be derived.

Conserved regions are those, which show a very little variation in the amino acid in one particular position of several homologs from different origin. The consensus sequences and polypeptide motifs as depicted in column 7 of Table IV are derived from said alignments. In one advantageous embodiment, in the method of the present invention the activity of a polypeptide is decreased comprising or consisting of a consensus sequence or a polypeptide motif as depicted in table IV, column 7 and in one another embodiment, the present invention relates to a polypeptide comprising or consisting of a consensus sequence or a polypeptide motif as depicted in table IV, columns 7 whereby 20 or less, preferably 15 or 10, preferably 9, 8, 7, or 6, more preferred 5 or 4, even more preferred 3, even more preferred 2, even more preferred 1, most preferred 0 of the amino acids positions indicated can be replaced by any amino acid. In one embodiment not more than 15%, preferably 10%, even more preferred 5%, 4%, 3%, or 2%, most preferred 1% or 0% of the amino acid position indicated by a letter are/is replaced by another amino acid. In one embodiment 20 or less, preferably 15 or 10, preferably 9, 8, 7, or 6, more preferred 5 or 4, even more preferred 3, even more preferred 2, even more preferred 1, most preferred 0 amino acids are inserted into a consensus sequence or protein motif.

The consensus sequence was derived from a multiple alignment of the sequences as listed in table II. The letters represent the one letter amino acid code and indicate that the amino acids are conserved in at least 80% of the aligned proteins. The letter X stands for amino acids, which are not conserved in at least 80% of the aligned sequences. The consensus sequence starts with the first conserved amino acid in the alignment, and ends with the last conserved amino acid in the alignment of the investigated sequences. The number of given X indicates the distances between conserved amino acid residues, e.g. Y-x(21,23)-F means that conserved tyrosine and phenylalanine residues are separated from each other by minimum 21 and maximum 23 amino acid residues in all investigated sequences.

Conserved domains were identified from all sequences and are described using a subset of the standard Prosite notation, e.g. the pattern Y-x(21,23)-[FW] means that a conserved tyrosine is separated by minimum 21 and maximum 23 amino acid residues from either a phenylalanine or tryptophane. Patterns had to match at least 80% of the investigated proteins.

Conserved patterns were identified with the software tool MEME version 3.5.1 or manually. MEME was developed by Timothy L. Bailey and Charles Elkan, Dept. of Computer Science and Engineering, University of California, San Diego, USA and is described by Timothy L. Bailey and Charles Elkan [Fitting a mixture model by expectation maximization to discover motifs in biopolymers, Proceedings of the Second International Conference on Intelligent Systems for Molecular Biology, pp. 28-36, AAAI Press, Menlo Park, Calif., 1994]. The source code for the stand-alone program is public available from the San Diego Supercomputer center (http://meme.sdsc.edu).

For identifying common motifs in all sequences with the software tool MEME, the following settings were used: -maxsize 500000, -nmotifs 15, -evt 0.001, -maxw 60, -distance 1e-3, -minsites number of sequences used for the analysis. Input sequences for MEME were non-aligned sequences in Fasta format. Other parameters were used in the default settings in this software version.

Prosite patterns for conserved domains were generated with the software tool Pratt version 2.1 or manually. Pratt was developed by Inge Jonassen, Dept. of Informatics, University of Bergen, Norway and is described by Jonassen et al. [I. Jonassen, J. F. Collins and D. G. Higgins, Finding flexible patterns in unaligned protein sequences, Protein Science 4 (1995), pp. 1587-1595; I. Jonassen, Efficient discovery of conserved patterns using a pattern graph, Submitted to CABIOS February 1997]. The source code (ANSI C) for the stand-alone program is public available, e.g. at established Bioinformatic centers like EBI (European Bioinformatics Institute).

For generating patterns with the software tool Pratt, following settings were used: PL (max Pattern Length): 100, PN (max Nr of Pattern Symbols): 100, PX (max Nr of consecutive x's): 30, FN (max Nr of flexible spacers): 5, FL (max Flexibility): 30, FP (max Flex.Product): 10, ON (max number patterns): 50. Input sequences for Pratt were distinct regions of the protein sequences exhibiting high similarity as identified from software tool MEME. The minimum number of sequences, which have to match the generated patterns (CM, min Nr of Seqs to Match) was set to at least 80% of the provided sequences. Parameters not mentioned here were used in their default settings.

The Prosite patterns of the conserved domains can be used to search for protein sequences matching this pattern. Various established Bioinformatic centers provide public internet portals for using those patterns in database searches (e.g. PIR [Protein Information Resource, located at Georgetown University Medical Center] or ExPASy [Expert Protein Analysis System]). Alternatively, stand-alone software is available, like the program Fuzzpro, which is part of the EMBOSS software package. For example, the program Fuzzpro not only allows searching for an exact pattern-protein match but also allows to set various ambiguities in the performed search.

The alignment was performed with the software ClustalW (version 1.83) and is described by Thompson et al. [Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680]. The source code for the stand-alone program is public available from the European Molecular Biology Laboratory; Heidelberg, Germany. The analysis was performed using the default parameters of ClustalW v1.83 (gap open penalty: 10.0; gap extension penalty: 0.2; protein matrix: Gonnet; pprotein/DNA endgap: −1; protein/DNA gapdist: 4).

Degenerate primers, designed as described above, can then be utilized by PCR for the amplification of fragments of novel coding regions coding for proteins having above-mentioned activity, e.g. conferring the increase of the tolerance and/or resistance to environmental stress and the biomass production as compared to a corresponding non-transformed wild type plant after reducing, repressing, decreasing or deleting the expression or activity of the respective nucleic acid sequence or the protein encoded by said sequence, e.g. which having the activity of a protein encoded by a nucleic acid which activity is to be reduced or deleted in the process of the invention or further functional equivalent or homologues from other organisms.

These fragments can then be utilized as hybridization probe for isolating the complete gene sequence. As an alternative, the missing 5′ and 3′ sequences can be isolated by means of RACE-PCR. A nucleic acid molecule according to the invention can be amplified using cDNA or, as an alternative, genomic DNA as template and suitable oligonucleotide primers, following standard PCR amplification techniques. The nucleic acid molecule amplified thus can be cloned into a suitable vector and characterized by means of DNA sequence analysis. Oligonucleotides, which correspond to one of the nucleic acid molecules used in the process, can be generated by standard synthesis methods, for example using an automatic DNA synthesizer.

Nucleic acid molecules which are advantageously for the process according to the invention can be isolated based on their homology to the nucleic acid molecules disclosed herein using the sequences or part thereof as hybridization probe and following standard hybridization techniques under stringent hybridization conditions.

In this context, it is possible to use, for example, isolated nucleic acid molecules of at least 15, 20, 25, 30, 35, 40, 50, 60 or more nucleotides, preferably of at least 15, 20 or 25 nucleotides in length which hybridize under stringent conditions with the above-described nucleic acid molecules, in particular with those which encompass a nucleotide sequence as depicted in column 5 or 7 of Table I. Nucleic acid molecules with 30, 50, 100, 250 or more nucleotides may also be used.

The term “homology” means that the respective nucleic acid molecules or encoded proteins are functionally and/or structurally equivalent. The nucleic acid molecules that are homologous to the nucleic acid molecules described above and that are derivatives of said nucleic acid molecules are, for example, variations of said nucleic acid molecules which represent modifications having the same biological function, in particular encoding proteins with the same or substantially the same biological function. They may be naturally occurring variations, such as sequences from other plant varieties or species, or mutations. These mutations may occur naturally or may be obtained by mutagenesis techniques. The allelic variations may be naturally occurring allelic variants as well as synthetically produced or genetically engineered variants. Structurally equivalents can for example be identified by testing the binding of said polypeptide to antibodies or computer based predictions. Structurally equivalent have the similar immunological characteristic, e.g. comprise similar epitopes.

By “hybridizing” it is meant that such nucleic acid molecules hybridize under conventional hybridization conditions, preferably under stringent conditions such as described by, e.g., Sambrook (Molecular Cloning; A Laboratory Manual, 2^{nd} Edition, Cold Spring Harbor Laboratory Press, Cold Spring Harbor, N.Y. (1989)) or in Current Protocols in Molecular Biology, John Wiley & Sons, N. Y. (1989), 6.3.1-6.3.6.

According to the invention, DNA as well as RNA molecules of the nucleic acid of the invention can be used as probes. Further, as template for the identification of functional homologues Northern blot assays as well as Southern blot assays can be performed. The Northern blot assay advantageously provides further informations about the expressed gene product: e.g. expression pattern, occurrence of processing steps, like splicing and capping, etc. The Southern blot assay provides additional information about the chromosomal localization and organization of the gene encoding the nucleic acid molecule of the invention.

A preferred, nonlimiting example of stringent Southern blot hydridization conditions are hybridizations in 6×sodium chloride/sodium citrate (=SSC) at approximately 45° C., followed by one or more wash steps in 0.2×SSC, 0.1% SDS at 50 to 65° C., for example at 50° C., 55° C. or 60° C. The skilled worker knows that these hybridization conditions differ as a function of the type of the nucleic acid and, for example when organic solvents are present, with regard to the temperature and concentration of the buffer. The temperature under “standard hybridization conditions” differs for example as a function of the type of the nucleic acid between 42° C. and 58° C., preferably between 45° C. and 50° C. in an aqueous buffer with a concentration of 0.1×0.5×, 1×, 2×, 3×, 4× or 5×SSC (pH 7.2). If organic solvent(s) is/are present in the abovementioned buffer, for example 50% formamide, the temperature under standard conditions is approximately 40° C., 42° C. or 45° C. The hybridization conditions for DNA:DNA hybrids are preferably for example 0.1×SSC and 20° C., 25° C., 30° C., 35° C., 40° C. or 45° C., preferably between 30° C. and 45° C. The hybridization conditions for DNA:RNA hybrids are preferably for example 0.1×SSC and 30° C., 35° C., 40° C., 45° C., 50° C. or 55° C., preferably between 45° C. and 55° C. The abovementioned hybridization temperatures are determined for example for a nucleic acid approximately 100 bp (=base pairs) in length and a G+C content of 50% in the absence of formamide. The skilled worker knows to determine the hybridization conditions required with the aid of textbooks, for example the ones mentioned above, or from the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford.

A further example of one such stringent hybridization condition is hybridization at 4×SSC at 65° C., followed by a washing in 0.1×SSC at 65° C. for one hour. Alternatively, an exemplary stringent hybridization condition is in 50% formamide, 4×SSC at 42° C. Further, the conditions during the wash step can be selected from the range of conditions delimited by low-stringency conditions (approximately 2×SSC at 50° C.) and high-stringency conditions (approximately 0.2×SSC at 50° C., preferably at 65° C.) (20×SSC: 0.3M sodium citrate, 3M NaCl, pH 7.0). In addition, the temperature during the wash step can be raised from low-stringency conditions at room temperature, approximately 22° C., to higher-stringency conditions at approximately 65° C.

Both of the parameters salt concentration and temperature can be varied simultaneously, or else one of the two parameters can be kept constant while only the other is varied. Denaturants, for example formamide or SDS, may also be employed during the hybridization. In the presence of 50% formamide, hybridization is preferably effected at 42° C. Relevant factors like i) length of treatment, ii) salt conditions, iii) detergent conditions, iv) competitor DNAs, v) temperature and vi) probe selection can combined case by case so that not all possibilities can be mentioned herein.

Some examples of conditions for DNA hybridization (Southern blot assays) and wash step are shown hereinbelow:
(1) Hybridization conditions can be selected, for example, from the following conditions:
a) 4×SSC at 65° C.,
b) 6×SSC at 45° C.,
c) 6×SSC, 100 mg/ml denatured fragmented fish sperm DNA at 68° C.,
d) 6×SSC, 0.5% SDS, 100 mg/ml denatured salmon sperm DNA at 68° C.,
e) 6×SSC, 0.5% SDS, 100 mg/ml denatured fragmented salmon sperm DNA, 50% formamide at 42° C.,
f) 50% formamide, 4×SSC at 42° C.,
g) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C.,
h) 2× or 4×SSC at 50° C. (low-stringency condition), or
i) 30 to 40% formamide, 2× or 4×SSC at 42° C. (low-stringency condition).
(2) Wash steps can be selected, for example, from the following conditions:
a) 0.015 M NaCl/0.0015 M sodium citrate/0.1% SDS at 50° C.
b) 0.1×SSC at 65° C.
c) 0.1×SSC, 0.5% SDS at 68° C.
d) 0.1×SSC, 0.5% SDS, 50% formamide at 42° C.
e) 0.2×SSC, 0.1% SDS at 42° C.
f) 2×SSC at 65° C. (low-stringency condition).
g) 0.2×SSC, 0.1% SDS at 60° C. (medium-high stringency conditions), or
h) 0.1×SSC, 0.1% SDS at 60° C. (medium-high stringency conditions), or
i) 0.2×SSC, 0.1% SDS at 65° C. (high stringency conditions), or
j) 0.1×SSC, 0.1% SDS at 65° C. (high stringency conditions)

Polypeptides or nucleic acid molecules having above-mentioned activity, e.g. conferring the increased tolerance and/or resistance to environmental stress and increased biomass production as compared to a corresponding non-transformed wild type plant derived from other organisms, can be encoded by other DNA molecules, which hybridize to a molecule as depicted in column 5 or 7 of Table I, or comprising it, under relaxed hybridization conditions and which code on expression for peptides or nucleic acids which activity needs to reduced or deleted to confer an increase of the tolerance and/or resistance to environmental stress and the biomass production as compared to a corresponding non-transformed wild type plant.

Preferably, the polypeptides or polynucleotides have further biological activities of the protein or the nucleic acid molecule comprising a molecule as depicted in column 5 or 7 of Table I, II or IV, respectively.

Relaxed hybridization conditions can for example used in Southern Blotting experiments.

Some applications have to be performed at low stringency hybridization conditions, without any consequences for the specificity of the hybridization. For example, a Southern blot analysis of total DNA could be probed with a nucleic acid molecule of the present invention and washed at low stringency (55° C. in 2×SSPE, 0.1% SDS). The hybridisation analysis could reveal a simple pattern of only genes encoding polypeptides of the present invention, e.g. having herein-mentioned activity. A further example of such low-stringent hybridization conditions is 4×SSC at 50° C. or hybridization with 30 to 40% formamide at 42° C. Such molecules comprise those which are fragments, analogues or derivatives of the nucleic acid molecule to be reduced in the process of the invention or encoding the polypeptide to be reduced in the process of the invention and differ, for example, by way of amino acid and/or nucleotide deletion(s), insertion(s), substitution(s), addition(s) and/or recombination(s) or any other modification(s) known in the art either alone or in combination from the above-described amino acid sequences or said (underlying) nucleotide sequence(s).

However, it is preferred to use high stringency hybridisation conditions.

Hybridization should advantageously be carried out with fragments of at least 5, 10, 15, 20, 25, 30, 35 or 40 bp, advantageously at least 50, 60, 70 or 80 bp, preferably at least 90, 100 or 110 bp. Most preferably are fragments of at least 15, 20, 25 or 30 bp. Preferably are also hybridizations with at least 100 bp or 200, very especially preferably at least 400 bp in length. In an especially preferred embodiment, the hybridization should be carried out with the entire nucleic acid sequence with conditions described above.

The terms “fragment”, “fragment of a sequence” or “part of a sequence” mean a truncated sequence of the original sequence referred to. The truncated sequence (nucleic acid or protein sequence) can vary widely in length; the minimum size being a sequence of sufficient size to provide a sequence or sequence fragment with at least 15, 20, 21, 22, 23, 24, 25, 26, 27, 28, 29, 30 bp in length with at least 90, 91, 92, 93, 94, 95, 96, 97, 98 or 99% identity preferably 100% identity with a fragment of a nucleic acid molecule described herein for the use in the process of the invention, e.g. a fragment of the nucleic acid molecules which activity is to be reduced in the process of the invention. Said truncated sequences can as mentioned vary widely in length from 15 by up to 2 kb or more, advantageously the sequences have a minimal length of 15, 20, 25, 30, 35 or 40 bp, while the maximum size is not critical. 100, 200, 300, 400, 500 or more base pair fragments can be used. In some applications, the maximum size usually is not substantially greater than that required to provide the complete gene function(s) of the nucleic acid sequences. Such sequences can advantageously been used for the repression, reduction, decrease or deletion of the activity to be reduced in the process of the invention, by for example the antisense, RNAi, snRNA, dsRNA, siRNA, miRNA, ta-siRNA, cosuppression molecule, ribozyme etc.-technology.

For the reduction, decrease or deletion of the activity of a nucleic acid molecule comprising a nucleic acid molecule as depicted in column 5 or 7 of Table I and/or a polypeptide comprising a polypeptide as depicted in column 5 or 7 of Table II or a consensus sequence or a polypeptide motif as depicted in column 7 of Table IV also the promotor regions of the disclosed nucleic acid sequences can be used. The skilled worker knows how to clone said promotor regions.

Typically, the truncated amino acid molecule will range from about 5 to about 310 amino acids in length. More typically, however, the sequence will be a maximum of about 250 amino acid in length, preferably a maximum of about 200 or 100 amino acid. It is usually desirable to select sequences of at least about 10, 12 or 15 amino acid, up to a maximum of about 20 or 25 amino acids.

The term “one or several amino acid” relates to at least one amino acid but not more than that number of amino acid, which would result in a homology of below 50% identity. Preferably, the identity is more than 70% or 80%, more preferred are 85%, 90%, 91%, 92%, 93%, 94% or 95%, even more preferred are 96%, 97%, 98%, or 99% identity.

Further, the nucleic acid molecule used in the process of the invention comprises a nucleic acid molecule, which is a complement of one of the nucleotide sequences of above mentioned nucleic acid molecules or a portion thereof. A nucleic acid molecule which is complementary to one of the nucleotide sequences as depicted in column 5 or 7 of Table I or a nucleic acid molecule comprising said sequence is one which is sufficiently complementary to said nucleotide sequences such that it can hybridize to said nucleotide sequences, thereby forming a stable duplex.

Preferably, the hybridisation is performed under stringent hybrization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and vice versa. Modifications of the bases can influence the base-pairing partner.

The nucleic acid molecule which activity is to be reduced in the process of the invention, in particular the nucleic acid molecule of the invention comprises a nucleotide sequence which is at least about 30%, 35%, 40% or 45%, preferably at least about 50%, 55%, 60% or 65%, more preferably at least about 70%, 80%, or 90%, and even more preferably at least about 95%, 97%, 98%, 99% or more homologous to a nucleotide sequence comprising a nucleic acid molecule as depicted in column 5 or 7 of Table I, or a portion thereof and/or has the activity of the protein indicated in the same line in column 5 of Table II or the nucleic acid molecule encoding said protein.

The nucleic acid molecule which activity is to be reduced in the process of the invention, e.g. the nucleic acid molecule of the invention, comprises a nucleotide sequence which hybridizes, preferably hybridizes under stringent conditions as defined herein, to one of the nucleotide sequences as depicted in column 5 or 7 of Table I, or a portion thereof and encodes a protein having aforementioned activity, e.g. conferring the increased tolerance and/or resistance to environmental stress and increased biomass production as compared to a corresponding non-transformed wild type plant upon the reduction of deletion of its activity, and e.g. of the activity of the protein.

Moreover, the nucleic acid molecule which activity is reduced in the process of the invention, in particular the nucleic acid molecule of the invention, can comprise only a portion of the coding region of one of the sequences depicted in column 5 or 7 of Table I, for example a fragment which can be used as a probe or primer or a fragment encoding a biologically active portion of the nucleic acid molecule or polypeptide to be reduced in the process of the present invention or a fragment encoding a non active part of the nucleic acid molecule or the polypeptide which activity is reduced in the process of the invention but conferring an increased tolerance and/or resistance to environmental stress and increased biomass production as compared to a corresponding non-transformed wild type plant if its expression or activity is reduced or deleted.

The nucleotide sequences determined from the cloning of the gene encoding the molecule which activity is reduced in the process of the invention allows the generation of probes and primers designed for the use in identifying and/or cloning its homologues in other cell types and organisms. The probe/primer typically comprises substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth described for the use in the process of the invention, e.g., comprising the molecule as depicted in column 5 or 7 of Table I, an anti-sense sequence of one of said sequence or naturally occurring mutants thereof. Primers based on a nucleotide of invention can be used in PCR reactions to clone homologues of the nucleic acid molecule which activity is to be reduced according to the process of the invention, e.g. as primer pairs described in the examples of the present invention, for example primers as depicted in column 7 of Table III, which do not start at their 5 prime end with the nucleotides ATA. Said nucleic acid molecules, which are homologues of the nucleic acid molecules which activity is to be reduced in the process of the invention or the nucleic acid molecules of the invention themselves can be used to reduce, decrease or delete the activity to be reduced according to the process of the invention.

Primer sets are interchangable. The person skilled in the art knows to combine said primers to result in the desired product, e.g. in a full-length clone or a partial sequence. Probes based on the sequences of the nucleic acid molecule used in the process of the invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which contain, or express or do not contain or express a nucleic acid molecule which activity is reduced in the process of the invention, such as by measuring a level of an encoding nucleic acid molecule in a sample of cells, e.g., detecting mRNA levels or determining, whether a genomic gene comprising the sequence of the polynucleotide has been mutated or deleted.

In one embodiment, the nucleic acid molecule used in the process of the invention, preferably the polynucleotide of the invention, encodes a polypeptide or portion thereof which includes an amino acid sequence which is sufficiently homologous to the amino acid sequence as depicted in column 5 or 7 of Table II or which is sufficiently homologous to a polypeptide comprising a consensus sequence or a polypeptide motif as depicted in column 7 of Table IV.

As used herein, the language “sufficiently homologous” refers to polypeptides or portions thereof which have an amino acid sequence which includes a minimum number of identical or equivalent amino acid residues (e.g., an amino acid residue which has a similar side chain as the amino acid residue to which it is compared) compared to an amino acid sequence of an polypeptide which activity is reduced in the process of the present invention, in particular, the polypeptide is sufficiently homologous to a polypeptide comprising a polypeptide, a consensus sequence or a polypeptide motif as depicted in column 5 or 7 of Table II or IV or e.g. to a functional equivalent thereof.

Portions of the aforementioned amino acid sequence are at least 3, 5, 10, 20, 30, 40, 50 or more amino acid in length.

In one embodiment, the nucleic acid molecule used in the process of the present invention comprises a nucleic acid molecule that encodes at least a portion of the polypeptide which activity is reduced in the process of the present invention, e.g. of a polypeptide as depicted in column 5 or 7 of Table II A or B, or a homologue thereof.

In a further embodiment, the polypeptide which activity is reduced in the process of the invention, in particular the polypeptide of the invention, is at least about 30%, 35%, 40%, 45% or 50%, preferably at least about 55%, 60%, 65% or 70% and more preferably at least about 75%, 80%, 85%, 90%, 91%, 92%, 93% or 94% and most preferably at least about 95%, 97%, 98%, 99% or more homologous to an entire amino acid sequence of a polypeptide as depicted in column 5 or 7 of Table II or to a polypeptide comprising a consensus sequence or a polypeptide motif as depicted in column 7 of Table IV and having above-mentioned activity, e.g. conferring preferably the increased tolerance and/or resistance to environmental stress and increased biomass production as compared to a corresponding non-transformed wild type plantafter its activity has been reduced, repressed or deleted.

Portions of the protein are preferably in such a manner biologically active, that they are increasing the tolerance and/or resistance to environmental stress and the biomass production as compared to a corresponding non-transformed wild type plant by being in their activity reduced, repressed, decreased or deleted.

As mentioned herein, the term “biologically active portion” is intended to include a portion, e.g., a domain/motif or a epitope, that shows by introducing said portion or an encoding polynucleotide into an organism, or a part thereof, particulary into a cell, the same activity as its homologue as depicted in column 5 or 7 of Table II or IV.

In one embodiment, the portion of a polypeptide has the activity of a polypeptide as its homologue as depicted in column 5 or 7 of Table II if it is able to complementate a knock out mutant as described herein.

The invention further relates to nucleic acid molecules which as a result of degeneracy of the genetic code can be derived from a polypeptide as depicted in column 5 or 7 of Table II or from a polypeptide comprising a consensus sequence or a polypeptide motif as depicted in column 7 of Table IV and thus encodes a polypeptide to be reduced in the process of the present invention, in particular a polypeptide leading by reducing, repressing, decreasing or deleting its activity to an increase in the tolerance and/or resistance to environmental stress and in the biomass production as compared to a corresponding non-transformed wild type plant.

Advantageously, the nucleic acid molecule which activity is reduced in the process of the invention comprises or has a nucleotide sequence encoding a protein comprising or having an amino acid molecule, a consensus sequence or a polypeptide motif as depicted in column 5 or 7 of Table II or IV, and differs from the amino acid molecule's sequences as depicted in column 5 or 7 of Table II A, preferably in at least one or more amino acid.

Said above nucleic acid molecules, e.g. the nucleic acid molecules which as a result of the degeneracy of the genetic code can be derived from said polypeptide sequences, can be used for the production of a nucleic acid molecule, e.g. an antisense molecule, a tRNAs, a snRNAs, a dsRNAs, a siRNAs, a miRNAs, a ta-siRNA, cosuppression molecules, a ribozymes molecule, or a viral nucleic acid molecule, or another inhibitory or activity reducing molecule as described herein for the use in the process of the invention, e.g. for the repression, decrease or deletion of the activity of the polypeptide or the nucleic acid molecule for use in the process of the invention according to the disclosure herein.

In addition, it will be appreciated by those skilled in the art that DNA sequence polymorphisms that lead to changes in the amino acid sequences may exist within a population. Such genetic polymorphism in the gene, e.g. encoding the polypeptide of the invention or comprising the nucleic acid molecule of the invention may exist among individuals within a population due to natural variation.

As used herein, the terms “gene” and “recombinant gene” refer to nucleic acid molecules comprising an open reading frame encoding a polypeptide comprising the polypeptide which activity is reduced in the process or the invention or a to a nucleic acid molecule encoding a polypeptide molecule which activity is reduced in the process of the present invention. For example, the gene comprises a open reading frame encoding a polypeptide comprising the polypeptide, the consensus sequence or the polypeptide motif as depicted in column 5 or 7 of Table II or IV, such as the polypeptide of the invention, or encoding a nucleic acid molecule comprising a polynucleotide as depicted in column 5 or 7 of Table I, such as the nucleic acid molecule of the invention and being preferably derived from a crop plant.

The gene can also be a natural variation of said gene.

Such natural variations can typically result in 1-5% variance in the nucleotide sequence of the gene used in the inventive process.

Nucleic acid molecules corresponding to natural variant homologues of the nucleic acid molecule comprising a polynucleotide as depicted in column 5 or 7 of Table I, such as the nucleic acid molecule of the invention, and which can also be a cDNA, can be isolated based on their homology to the nucleic acid molecules disclosed herein using the nucleic acid molecule as depicted in column 5 or 7 of Table I, e.g. the nucleic acid molecule of the invention, or a fragment thereof, as a hybridization probe according to standard hybridization techniques under stringent hybridization conditions.

Accordingly, in another embodiment, the nucleic acid molecule which activity is reduced in the process of the invention, e.g. the nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention, e.g. comprising the sequence as depicted in column 5 or 7 of Table I. The nucleic acid molecule is preferably at least 20, 30, 50, 100, 250 or more nucleotides in length.

The term “hybridizes under stringent conditions” is defined above. In one embodiment, the term “hybridizes under stringent conditions” is intended to describe conditions for hybridization and washing under which nucleotide sequences of at least 30%, 40%, 50% or 65% identical to each other typically remain hybridized to each other. Preferably, the conditions are such that sequences of at least about 70%, more preferably at least about 75% or 80%, and even more preferably of at least about 85%, 90% or 95% or more identical to each other typically remain hybridized to each other.

In one emboliment the nucleic acid molecule of the invention hybridizes under stringent conditions to a sequence of column 7 of Table 1B and corresponds to a naturally-occurring nucleic acid molecule. As used herein, a “naturally-occurring” nucleic acid molecule refers to a RNA or DNA molecule having a nucleotide sequence that occurs in nature (e.g., encodes a natural protein). Preferably, the nucleic acid molecule encodes a natural protein conferring an increase of the tolerance and/or resistance to environmental stress and of the biomass production as compared to a corresponding non-transformed wild type plant after reducing, decreasing or deleting the expression or activity thereof.

In addition to naturally-occurring variants of the nucleic acid or protein sequence that may exist in the population, the skilled artisan will further appreciate that changes can be introduced by mutation into a nucleotide sequence of the nucleic acid molecule encoding the polypeptide, thereby leading to changes in the amino acid sequence of the encoded polypeptide and thereby altering the functional ability of the polypeptide, meaning preferably reducing, decreasing or deleting said activity. For example, nucleotide substitutions leading to amino acid substitutions at “essential” amino acid residues can be made in a sequence of the nucleic acid molecule to be reduced in the process of the invention, e.g. comprising the corresponding nucleic acid molecule as depicted in column 5 or 7 of Table I. An “essential” amino acid residue is a residue that if altered from the wild-type sequence of one of the polypeptide lead to an altered activity of said polypeptide, whereas a “non-essential” amino acid residue is not required for the activity of the protein for example for the activity as an enzyme. The alteration of “essential” residues often lead to a reduced decreased or deleted activity of the polypeptides. Preferably amino acid of the polypeptide are changed in such a manner that the activity is reduced, decreased or deleted that means preferably essential amino acid residues and/or more non-essential residues are changed and thereby the activity is reduced, which leads as mentioned above to an increase in tolerance and/or resistance to environmental stress and in biomass production as compared to a corresponding non-transformed wild type plant in a plant after decreasing the expression or activity of the polypeptide. Other amino acid residues, however, (e.g., those that are not conserved or only semi-conserved in the domain having said activity) may not be essential for activity and thus are likely to be amenable to alteration without altering said activity are less preferred.

A further embodiment of the invention relates to the specific search or selection of changes in a nucleic acid sequence which confer a reduced, repressed or deleted activity in a population, e.g. in a natural or artificial created population. It is often complex and expensive to search for an increase in tolerance and/or resistance to environmental stress and in biomass production as compared to a corresponding nontransformed wild type plant in a population, e.g. due to complex analytical procedures. It can therefore be advantageous to search for changes in a nucleic acid sequence which confer a reduced, repressed or deleted activity of the expression product in said population, thus, identifying candidates which bring about the desired increase in the tolerance and/or resistance to environmental stress and in biomass production as compared to a corresponding non-transformed wild type plantcontent. A typical example of a natural gene, the downregulation of which leads to the desired trait is the mlo locus (Pifanelli et al., Nature 2004 Aug. 19; 430(7002): 887-91. Barley plants carrying loss-of-function alleles (mlo) of the Mlo locus are resistant against all known isolates of the widespread powdery mildew fungus. The sole mlo resistance allele recovered so far from a natural habitat, mlo-11, was originally retrieved from Ethiopian landraces and nowadays controls mildew resistance in the majority of cultivated European spring barley elite varieties. Thus, one can search for natural alleles, which bring about the desired reduction, repression, deletion or decrease in the function of a nucleic acid molecule and can introduce such alleles into agronomical important crop varieties through crossing and marker assisted selection or related methods.

Further, a person skilled in the art knows that the codon usage between organisms can differ. Therefore, he will adapt the codon usage in the nucleic acid molecule of the present invention to the usage of the organism in which the polynucleotide or polypeptide is expressed, so that the expression of the nucleic acid molecule or the encoded protein is more likely reduced.

Accordingly, the invention relates to a homologues nucleic acid molecule of a nucleic acid molecules encoding a polypeptide having abovementioned activity in a plant or parts thereof after being reduced, decreases, repressed or deleted, that contain changes in its amino acid residues that are essential for its activity and thus reduce, decrease, repress or delete its activity.

Such polypeptides differ in the amino acid sequence from a sequence as depicted in column 5 or 7 of Table II or comprising a consensus sequence or a polypeptide motif as depicted in column 7 of Table IV yet and confer an increase the tolerance and/or resistance to environmental stress and the biomass production as compared to a corresponding non-transformed wild type plant. The nucleic acid molecule can comprise a nucleotide sequence encoding a polypeptide, wherein the polypeptide comprises an amino acid sequence at least about 50% identical to an amino acid sequence as depicted in column 5 or 7 of Table II or comprising a consensus sequence or a polypeptide motif as depicted in column 7 of Table IV and is capable of participation in the increase of the tolerance and/or resistance to environmental stress and of the biomass production as compared to a corresponding non-transformed wild type plant after decreasing its expression or its biological function.

Preferably, the protein encoded by the nucleic acid molecule is at least about 60%, 70% or 80% identical to the sequence in column 5 or 7 of Table II or to a sequence comprising a consensus sequence or a polypeptide motif as depicted in column 7 of Table IV, more preferably at least about 85% identical to one of the sequences in column 5 or 7 of Table II or to a sequence comprising a consensus sequence or a polypeptide motif as depicted in column 7 of Table IV, even more preferably at least about 90%, 91%, 92%, 93%, 94%, 95% homologous to the sequence in column 5 or 7 of Table II or to a sequence comprising a consensus sequence or a polypeptide motif as depicted in column 7 of Table IV, and most preferably at least about 96%, 97%, 98%, or 99% identical to the sequence in column 5 or 7 of Table II or to a sequence comprising a consensus sequence or a polypeptide motif as depicted in column 7 of Table IV.

To determine the percentage homology (=identity) of two amino acid sequences (for example of column 7 of Table II) or of two nucleic acid molecules (for example of column 5 or Table I), the sequences are written one underneath the other for an optimal comparison. Gaps may be inserted into the sequence of a protein or of a nucleic acid molecule in order to generate an optimal alignment with the other protein or the other nucleic acid. The amino acid residue or nucleotide at the corresponding amino acid position or nucleotide position is then compared between both polymers. If a position in one sequence is occupied by the same amino acid residue or the same nucleotide as in the corresponding position of the other sequence, the molecules are identical at this position. Amino acid or nucleotide “identity” as used in the present context corresponds to amino acid or nucleic acid “homology”. Generally the percentage homology between the two sequences is a function of the number of identical positions shared by the sequences (i.e. % homology=number of identical positions/total number of positions x 100). The terms “homology” and “identity” are thus to be considered as synonyms for this description.

For the determination of the percentage homology (=identity) of two or more amino acid or of two or more nucleotide sequences several computer software programs have been developed. The homology of two or more sequences can be calculated with for example the software fasta, which presently has been used in the version fasta 3 (W. R. Pearson and D. J. Lipman (1988), Improved Tools for Biological Sequence Comparison. PNAS 85:2444-2448; W. R. Pearson (1990) Rapid and Sensitive Sequence Comparison with FASTP and FASTA, Methods in Enzymology 183:63-98; W. R. Pearson and D. J. Lipman (1988) Improved Tools for Biological Sequence Comparison. PNAS 85:2444-2448; W. R. Pearson (1990); Rapid and Sensitive Sequence Comparison with FASTP and FASTAMethods in Enzymology 183:63-98). Another useful program for the calculation of homologies of different sequences is the standard blast program, which is included in the Biomax pedant software (Biomax, Munich, Federal Republic of Germany). This leads unfortunately sometimes to suboptimal results since blast does not always include complete sequences of the subject and the query. Nevertheless as this program is very efficient it can be used for the comparison of a huge number of sequences. The following settings are typically used for such a comparisons of sequences: -p Program Name [String]; -d Database [String]; default=nr; -i Query File [File In]; default=stdin; -e Expectation value (E) [Real]; default=10.0; -m alignment view options: 0=pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default=0;-o BLAST report Output File [File Out] Optional; default=stdout; -F Filter query sequence (DUST with blastn, SEG with others) [String]; default=T; -G Cost to open a gap (zero invokes default behavior) [Integer]; default=0;-E Cost to extend a gap (zero invokes default behavior) [Integer]; default=0;-X X dropoff value for gapped alignment (in bits) (zero invokes default behavior); blastn 30, megablast 20, tblastx 0, all others 15 [Integer]; default=0;-I Show GI's in deflines [T/F]; default=F; -q Penalty for a nucleotide mismatch (blastn only) [Integer]; default=-3;-r Reward for a nucleotide match (blastn only) [Integer]; default=1;-v Number of database sequences to show one-line descriptions for (V) [Integer]; default=500;-b Number of database sequence to show alignments for (B) [Integer]; default=250;-f Threshold for extending hits, default if zero; blastp 11, blastn 0, blastx 12, tblastn 13; tblastx 13, megablast 0 [Integer]; default=0;-g Perfom gapped alignment (not available with tblastx) [T/F]; default=T; -Q Query Genetic code to use [Integer]; default=1;-D DB Genetic code (for tblast[nx] only) [Integer]; default=1;-a Number of processors to use [Integer]; default=1;-O SegAlign file [File Out] Optional; -J Believe the query defline [T/F]; default=F; -M Matrix [String]; default=BLOSUM62;-W Word size, default if zero (blastn 11, megablast 28, all others 3) [Integer]; default=0;-z Effective length of the database (use zero for the real size) [Real]; default=0;-K Number of best hits from a region to keep (off by default, if used a value of 100 is recommended) [Integer]; default=0;-P 0 for multiple hit, 1 for single hit [Integer]; default=0;-Y Effective length of the search space (use zero for the real size) [Real]; default=0;-S Query strands to search against database (for blast[nx], and tblastx); 3 is both, 1 is top, 2 is bottom [Integer]; default=3;-T Produce HTML output [T/F]; default=F; -I Restrict search of database to list of GI's [String] Optional; -U Use lower case filtering of FASTA sequence [T/F] Optional; default=F; -y X dropoff value for ungapped extensions in bits (0.0 invokes default behavior); blastn 20, megablast 10, all others 7 [Real]; default=0.0;-Z X dropoff value for final gapped alignment in bits (0.0 invokes default behavior); blastn/megablast 50, tblastx 0, all others 25 [Integer]; default=0;-R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40 [Integer]; default=0;-w Frame shift penatty (OOF algorithm for blastx) [Integer]; default=0;-t Length of the largest intron allowed in tblastn for linking HSPs (0 disables linking) [Integer]; default=0.

Results of high quality are reached by using the algorithm of Needleman and Wunsch or Smith and Waterman. Therefore programs based on said algorithms are preferred. Advantageously the comparisons of sequences can be done with the program PileUp (J. Mol. Evolution., 25, 351 (1987), Higgins et al., CABIOS 5, 151 (1989)) or preferably with the programs “Gap” and “Needle”, which are both based on the algorithms of Needleman and Wunsch (J. Mol. Biol. 48; 443 (1970)), and “BestFit”, which is based on the algorithm of Smith and Waterman (Adv. Appl. Math. 2; 482 (1981)). “Gap” and “BestFit” are part of the GCG software-package (Genetics Computer Group, 575 Science Drive, Madison, Wis., USA 53711 (1991); Altschul et al., (Nucleic Acids Res. 25, 3389 (1997)), “Needle” is part of the The European Molecular Biology Open Software Suite (EMBOSS) (Trends in Genetics 16 (6), 276 (2000)). Therefore preferably the calculations to determine the percentages of sequence homology are done with the programs “Gap” or “Needle” over the whole range of the sequences. The following standard adjustments for the comparison of nucleic acid sequences were used for “Needle”: matrix: EDNAFULL, Gap penalty: 10.0, Extend penalty: 0.5. The following standard adjustments for the comparison of nucleic acid sequences were used for “Gap”: gap weight: 50, length weight: 3, average match: 10.000, average mismatch: 0.000.

For example a sequence which has a 80% homology with sequence depicted in SEQ ID NO.: 1025 at the nucleic acid level is understood as meaning a sequence which, upon comparison to the sequence SEQ ID NO: 1025 by the above Gap program algorithm with the above parameter set, has 80% homology.

Homology between two polypeptides is understood as meaning the identity of the amino acid sequence over in each case the entire sequence length which is calculated by comparison with the aid of the program algorithm “Needle” using Matrix: EBLOSUM62, Gap penalty: 8.0, Extend penalty: 2.0.

For example a sequence which has a 80% homology with sequence SEQ ID NO: 1026 at the protein level is understood as meaning a sequence which, upon comparison to the sequence SEQ ID NO: 1026 by the above program “Needle” with the above parameter set, has 80% identity.

Functional equivalents derived from one of the polypeptides as depicted column 5 or 7 of Table II or comprising the consensus sequence or the polypeptide motif as depicted in column 7 of Table IV according to the invention by substitution, insertion or deletion have at least 30%, 35%, 40%, 45% or 50%, preferably at least 55%, 60%, 65% or 70% by preference at least 80%, especially preferably at least 85% or 90%, 91%, 92%, 93% or 94%, very especially preferably at least 95%, 97%, 98% or 99% homology with one of the polypeptides as shown in column 5 or 7 of Table II or with one of the polypeptides comprising a consensus sequence or a polypeptide motif as depicted in column 7 of Table IV according to the invention and are distinguished by essentially the same properties as the polypeptide as depicted in column 5 or 7 of Table II preferably of the polypeptides of A. thaliana.

Functional equivalents derived from the nucleic acid sequence as depicted in column 5 or 7 of Table I according to the invention by substitution, insertion or deletion have at least 30%, 35%, 40%, 45% or 50%, preferably at least 55%, 60%, 65% or 70% by preference at least 80%, especially preferably at least 85% or 90%, 91%, 92%, 93% or 94%, very especially preferably at least 95%, 97%, 98% or 99% homology with one of the nucleic acids as depicted in column 5 or 7 of Table I according to the invention and encode polypeptides having essentially the same properties as the polypeptide as depicted in column 5 of Table II.

“Essentially the same properties” of a functional equivalent is above all understood as meaning that the functional equivalen has above mentioned activity, e.g conferring an increasing in the tolerance and/or resistance to environmental stress and in the biomass production as compared to a corresponding non-transformed wild type plant while decreasing the amount of protein, activity or function of said functional equivalent in an organism, e.g. a plant or in a plant tissue, plant cells or a part of the same.

A nucleic acid molecule encoding an homologous to a protein sequence shown herein can be created by introducing one or more nucleotide substitutions, additions or deletions into a nucleotide sequence of a nucleic acid molecule comprising the nucleic acid molecule as depicted in column 5 or 7 of Table I such that one or more amino acid substitutions, additions or deletions are introduced into the encoded protein. Mutations can be introduced into the sequences of, e.g. the sequences as depicted in column 5 or 7 of Table I, by standard techniques, such as site-directed mutagenesis and PCR-mediated mutagenesis.

Preferably, non-conservative amino acid substitutions are made at one or more predicted non-essential or preferably essential amino acid residues and thereby reducing, decreasing or deleting the activity of the respective protein. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methioninemethionine, tryptophan), beta-branched side chains (e.g., threonine, valine, isoleucine) and aromatic side chains (e.g., tyrosine, phenylalanine, tryptophan, histidine).

Thus, a predicted essential amino acid residue in a polypeptide used in the process or in the polypeptide of the invention, is preferably replaced with another amino acid residue from another family. Alternatively, in another embodiment, mutations can be introduced randomly along all or part of a coding sequence of a nucleic acid molecule coding for a polypeptide used in the process of the invention or a polynucleotide of the invention such as by saturation mutagenesis, and the resultant mutants can be screened for activity described herein to identify mutants that lost or have a decreased activity and conferring an increased tolerance and/or resistance to environmental stress and increased biomass production as compared to a corresponding non-transformed wild type plant.

Following mutagenesis of one of the sequences of column 5 or 7 of Table I, the encoded protein can be expressed recombinantly and the activity of the protein can be determined using, for example, assays described herein.

Essentially homologous polynucleotides of the nucleic acid molecule shown herein for the process according to the invention and being indicated in column 5 of Table I were found by BlastP database search with the corresponding polypeptide sequences. The SEQ ID No: of the found homologous sequences of a nucleic acid molecule indicated in column 5 of Table I are shown in column 7 of Table I in the respective same line. The SEQ ID No: of the found homologous sequences of a protein molecule indicated in column 5 of Table II are depicted in column 7 of Table II in the respective same line.

The protein sequence of a nucleic acid molecule depicted in column 5 and Table I were used to search protein databases using the tool BlastP. Homologous protein sequences were manually selected according to their similarity to the query protein sequence. The nucleotide sequence corresponding to the selected protein sequence is specified in the header section of the protein database entry in most cases and was used if present. If a protein database entry did not provide a direct cross-reference to the corresponding nucleotide database entry, the sequence search program TBlastN was used to identify nucleotide database entries from the same organism encoding exactly the same protein (100% identity). The expectation value was set to 0.001 in TBlastN and the blosum62 matrix was used; all other parameters were used in its default settings.

Further, the protein patterns defined for the protein sequences depicted in column 5 and 7 Table II were used to search protein databases. Protein sequences exhibiting all protein patterns depicted in column 7 of Table IV were aligned with the protein sequence depicted in column 5 and 7 Table II of the respective same line and selected as homologous proteins if significant similarity was observed.

Homologues of the nucleic acid sequences used, having or being derived from a sequence as depicted in column 5 or 7 of Table I or of the nucleic acid sequences derived from the sequences as depicted in column 5 or 7 of Table II or from the sequence comprising the consensus sequences or the polypeptide motifs as depicted in column 7 of Table IV comprise also allelic variants with at least approximately 30%, 35%, 40% or 45% homology, by preference at least approximately 50%, 60% or 70%, more preferably at least approximately 90%, 91%, 92%, 93%, 94% or 95% and even more preferably at least approximately 96%, 97%, 98%, 99% or more homology with one of the nucleotide sequences shown or the abovementioned derived nucleic acid sequences or their homologues, derivatives or analogues or parts of these. Allelic variants encompass in particular functional variants which can be obtained by deletion, insertion or substitution of nucleotides from the sequences shown or used in the process of the invention, preferably as depicted in column 5 or 7 of Table I, or from the derived nucleic acid sequences.

In one embodiment, however, the enzyme activity or the activity of the resulting proteins synthesized is advantageously lost or decreased, e.g. by mutation of sequence as described herein or by applying a method to reduce or inhibit or loose the biological activity as described herein.

In one embodiment of the present invention, the nucleic acid molecule used in the process of the invention or the nucleic acid molecule of the invention comprises a sequence as depicted in column 5 or 7 of Table I or its complementary sequence. It can be preferred that a homologue of a nucleic acid molecule as depicted in column 5 or 7 of Table I comprises as little as possible other nucleotides compared to the sequence as depicted in column 5 or 7 of Table I or its complementary sequence. In one embodiment, the nucleic acid molecule comprises less than 500, 400, 300, 200, 100, 90, 80, 70, 60, 50 or 40 further or other nucleotides. In a further embodiment, the nucleic acid molecule comprises less than 30, 20 or 10 further or other nucleotides. In one embodiment, the nucleic acid molecule use in the process of the invention is identical to the sequences as depicted in column 5 or 7 of Table I or its complementary sequence.

Also preferred is that the nucleic acid molecule used in the process of the invention encodes a polypeptide comprising the sequence, a consensus sequence or a polypeptide motif as depicted in column 5 or 7 of Table II or IV. In one embodiment, the nucleic acid molecule encodes less than 150, 130, 100, 80, 60, 50, 40 or 30 further or other amino acids. In a further embodiment, the encoded polypeptide comprises less than 20, 15, 10, 9, 8, 7, 6 or 5 further or other amino acids. In one embodiment used in the inventive process, the encoded polypeptide is identical to the sequences as depicted in column 5 or 7 of Table II.

In one embodiment, the nucleic acid molecule used in the process of invention encoding a polypeptide comprising a sequence, a consensus sequence or a polypeptide motif as depicted in column 5 or 7 of Table II or IV comprises less than 100 further or other nucleotides different from the sequence shown in column 5 or 7 of Table I. In a further embodiment, the nucleic acid molecule comprises less than 30 further or other nucleotides different from the sequence as depicted in column 5 or 7 of Table I. In one embodiment, the nucleic acid molecule is identical to a coding sequence of the sequences as depicted in column 5 or 7 of Table I

Homologues of sequences depicted in column 5 or 7 of Table I or of the derived sequences from the sequences as depicted in column 5 or 7 of Table II or from sequences comprising the consensus sequences or the polypeptide motifs as depicted in column 7 of Table IV also mean truncated sequences, cDNA, single-stranded DNA or RNA of the coding and noncoding DNA sequence. Homologues of the sequences as depicted in the column 5 or 7 of Table I or the derived sequences of the sequences as depicted in column 5 or 7 of Table II or from sequences comprising the consensus sequences or the polypeptide motifs as depicted in column 7 of Table IV are also understood as meaning derivatives which comprise noncoding regions such as, for example, UTRs, terminators, enhancers or promoter variants.

The regulatory sequences upstream or downstream of the nucleotide sequences stated can be modified by one or more nucleotide substitution(s), insertion(s) and/or deletion(s) with, however, preferably interfering with the functionality or activity either of the promoters, the open reading frame (=ORF) or with the 3′-regulatory region such as terminators or other 3′regulatory regions, which are far away from the ORF. It is furthermore possible that the activity of the promoters is decreased by modification of their sequence or their regulation, or that they are replaced completely by less active promoters and thereby the activity of the expressed nucleic acid sequence is reduced or deleted, even promoters from heterologous organisms can be used. Appropriate promoters are known to the person skilled in the art and are mentioned herein below. Modifying the regulatory sequences might be specifically advantageous in those cases were a complete elimination of the expression of the nucleic acid of the invention has negative side effects, such as reduced growth or yield. The person skilled in the art is able to modify the regulatory sequences of the nucleic acid of the invention in such a way that the reduction is sufficient to yield the increased tolerance and/or resistance to environmental stress and increased biomass production as compared to a corresponding non-transformed wild type plant without having unwanted side effects. In this context it might be further advantageously to modify the regulatory sequences in such a way that the reduction in expression occurs in a spatial or temporal manner. For example, it might be useful to inhibit, downregulate or repress the nucleic acids or the polypeptide of the invention only in the mature state of the plant, to achieve the desired increased tolerance and/or resistance to environmental stress and increased biomass production as compared to a corresponding non-transformed wild type plant without interfering with the growth or maturation of the organism. Further methods exists to modulate the promoters of the genes of the invention, e.g. by modifying the activity of transacting factors, meaning natural or artificial transcription factors, which can bind to the promoter and influence its activity. Furthermore it is possible to influence promoters of interest by modifying upstream signaling components like receptors or kinases, which are involved in the regulation of the promoter of interest.

In a further embodiment, the process according to the present invention comprises the following steps:
a) selecting an organism or a part thereof expressing the polypeptide or nucleic acid molecule which activity is reduced in the process of the invention, e.g. a polypeptide comprising a polypeptide, a consensus sequence or a polypeptide motif as depicted in column 5 or 7 of Table II or IV or a nucleic acid molecule comprising a nucleic acid molecule as depicted in column 5 or 7 of Table I;
b) mutagenizing the selected organism or the part thereof;
c) comparing the activity or the expression level of said polypeptide or nucleic acid molecule in the mutagenized organism or the part thereof with the activity or the expression of said polypeptide in the selected organisms or the part thereof;
d) selecting the mutagenized organisms or parts thereof, which comprise a decreased activity or expression level of said polypeptide compared to the selected organism (a) or the part thereof;
e) optionally, growing and cultivating the organisms or the parts thereof; and
f) testing, whether the organism or the part thereof has anincreased tolerance and/or resistance to environmental stress and increased biomass production as compared to a corresponding non-transformed wild type plant, such as a not mutagenized source or origin strain.

Advantageously the selected organisms were mutagenized according to the invention. According to the invention mutagenesis is any change of the genetic information in the genome of an organism, that means any structural or compositional change in the nucleic acid preferably DNA of an organism that is not caused by normal segregation or genetic recombiantion processes. Such mutations may occur spontaneously, or may be induced by mutagens as described below. Such change can be induced either randomly or selectively. In both cases the genetic information of the organism is modified. In general this leads to the situation that the activity of the gene product of the relevant genes inside the cells or inside the organism is reduced or repressed.

In case of the specific or so-called site directed mutagenesis a distinct gene is mutated and thereby its activity and/or the activity or the encoded gene product is repressed, reduced, decreased or deleted. In the event of a random mutagenesis one or more genes are mutated by chance and their activities and/or the activities of their gene products are repressed, reduced, decreased or deleted, preferably decreased or deleted. Nevertheless mutations in the gene of interest can be selected for by various methods know to the person skilled in the art.

For the purpose of a mutagenesis of a huge population of organisms, such population can be transformed with a DNA population or a DNA bank or constructs or elements, which are useful for the inhibition of as much as possible genes of an organism, preferably all genes. With this method it is possible to statistically mutagenize nearly all genes of an organism by the integration of an advantageously identified DNA-fragment. Afterwards the skilled worker can easely identify the knocked out event. For the mutagenesis of plants EMS, T-DNA and/or transposon mutagenesis is preferred.

In the event of a random mutagenesis a huge number of organisms are treated with a mutagenic agent. The amount of said agent and the intensity of the treatment is chosen in such a manner that statistically nearly every gene is mutated. The process for the random mutagensis as well as the respective agens is well known by the skilled person. Such methods are disclosed for example by A. M. van Harten [(1998), “Mutation breeding: theory and practical applications”, Cambridge University Press, Cambridge, UK], E Friedberg, G Walker, W Siede [(1995), “DNA Repair and Mutagenesis”, Blackwell Publishing], or K. Sankaranarayanan, J. M. Gentile, L. R. Ferguson [(2000) “Protocols in Mutagenesis”, Elsevier Health Sciences]. As the skilled worker knows the spontaneous mutation rate in the cells of an organism is very low and that a large number of chemical, physical or biological agents are available for the mutagenesis of organisms. These agents are named as mutagens or mutagenic agents. As mentioned before three different kinds of mutagens chemical, physical or biological agents are available.

There are different classes of chemical mutagens, which can be separated by their mode of action. For example base analogues such as 5-bromouracil, 2-amino purin. Other chemical mutagens are interacting with the DNA such as sulphuric acid, nitrous acid, hydroxylamine; or other alkylating agents such as monofunctional agents like ethyl methanesulfonate (=EMS), dimethylsulfate, methyl methanesulfonate, bifunctional like dichloroethyl sulphide, Mitomycin, Nitrosoguanidine—dialkylnitrosamine, N-Nitrosoguanidin derivatives, N-alkyl-N-nitro-N-nitroso-guanidine, intercalating dyes like Acridine, ethidium bromide.

Physical mutagens are for example ionizing irradiation (X-ray), UV irradiction. Different forms of irradiation are available and they are strong mutagens. Two main classes of irradiation can be distinguished: a) non-ionizing irradiation such as UV light or ionizing irradiation such as X-ray. Biological mutagens are for example transposable elements for example IS elements such as IS100, transposons such as Tn5, Tn10, Tn903, Tn916 or Tn1000 or phages like Mu^{amplac}, P1, T5, λplac etc. Methods for introducing this phage DNA into the appropriate microorganism are well known to the skilled worker (see Microbiology, Third Edition, Eds. Davis, B. D., Dulbecco, R., Eisen, H. N. and Ginsberg, H. S., Harper International Edition, 1980). The common procedure of a transposon mutagenesis is the insertion of a transposable element within a gene or nearby for example in the promotor or terminator region and thereby leading to a loss of the gene function. Procedures to localize the transposon within the genome of the organisms are well known by a person skilled in the art. For transposon mutagenesis in plants the maize transposon systems Activator-Dissociation (Ac/Ds) and Enhancer-Supressor mutator (En/Spm) are known to the worker skilled in the art but other transposon systems might be similar useful. The transposons can be brought into the plant genomes by different available standard techniques for plant transformations. Another type of biological mutagenesis in plants includes the T-DNA mutagenesis, meaning the random integration of T-DNA sequences into the plant genome [Feldmann, K. A. (1991) T-DNA insertion mutagenesis in Arabidopsis: Mutational spectrum. Plant J. 1, 71-82]. The event in which the gene of interest is mutated can later be searched by PCR- or other high throughput technologies [Krysan et al., (1999) T_DNA as an insertional mutagen in Arabidopsis, Plant Cell, 11, 2283-2290].

Biological methods are disclosed by Spee et al. (Nucleic Acids Research, Vol. 21, No. 3, 1993: 777-778). Spee et al. teaches a PCR method using dITP for the random mutagenesis. This method described by Spee et al. was further improved by Rellos et al. (Protein Expr. Purif., 5, 1994 : 270-277). The use of an in vitro recombination technique for molecular mutagenesis is described by Stemmer (Proc. Natl. Acad. Sci. USA, Vol. 91, 1994: 10747-10751). Moore et al. (Nature Biotechnology Vol. 14, 1996: 458-467) describe the combination of the PCR and recombination methods for increasing the enzymatic activity of an esterase toward a para-nitrobenzyl ester. Another route to the mutagenesis of enzymes is described by Greener et al. in Methods in Molecular Biology (Vol. 57, 1996: 375-385). Greener et al. use the specific Escherichia coli strain XL1-Red to generate Escherichia coli mutants, which have increased antibiotic resistance.

Preferably a chemical or biochemical procedure is used for the mutagenesis of the organisms. A preferred chemical method is the mutagenesis with N-methyl-N-nitro-nitrosoguanidine.

Other methods are for example the introduction of mutation with the aid of viruses such as bacteriophages such as P1, P22, T2, T3, T5, T7, Mu^{amplac}, Mu, Mu1, MuX, miniMu, λ, λplac or insertion elements such as IS3, IS 100, IS900 etc. Again the whole genome of the bacteria is randomly mutagenized. Mutants can be easily identifled.

Another method to disrupt the nucleic acid sequence used in the process of the invention and thereby reducing, decreasing or deleting the activity of the encoded polypeptide can be reached by homologous recombination with an little altered nucleic acid sequence of the invention described herein as usable for the process of the invention, e.g. derived from the sequence shown in column 5 or 7 of Table I. For example, the nucleic acid sequences used in the process of the invention can therefore be altered by one or more point mutations, deletions, insertions, or inversions. In another embodiment of the invention, one or more of the regulatory regions (e.g., a promoter, repressor, UTR, enhancer, or inducer) of the gene encoding the protein of the invention can be altered (e.g., by deletion, truncation, inversion, insertion, or point mutation) such that the expression of the corresponding gene is modulated that means reduced, decreased or deleted.

Accordingly, in one embodiment, the invention relates to an isolated nucleic acid molecule encoding an antisense, RNAi, snRNA, dsRNA, siRNA, miRNA, ta-siRNA, cosuppression molecule, or ribozyme molecule of the invention or the co-suppression nucleic acid molecule or the viral degradation nucleic acid molecule of the invention or encoding a DNA-, RNA- or protein-binding factor against genes, RNA's or proteins, a dominant negative mutant, or an antibody of the invention or the nucleic acid molecule for a recombination of the invention, in particular the nucleic acid molecule for a homologous recombination, comprising at least a fragment of 15, 16, 17, 18, 19, 20, 21, 25, 30, 35, 40, 50, 70, 100, 200, 300, 500, 1000, 2000 or more nucleotides of a nucleic acid molecule selected from the group consisting of:
a) a nucleic acid molecule encoding the polypeptide as depicted in column 5 or 7 of Table II, preferably of Table IIB or encompassing a consensus sequence or a polypeptide motif as depicted in column 7 Table IV;
b) a nucleic acid molecule as depicted in column 5 or 7 of Table I, preferably of Table IB;
c) a nucleic acid molecule, which, as a result of the degeneracy of the genetic code, can be derived from a polypeptide sequence as depicted in column 5 or 7 of Table II, preferably of Table IIB;
d) a nucleic acid molecule having at least 30% identity with the nucleic acid moleculesequence of a polynucleotide comprising the nucleic acid molecule as depicted in column 5 or 7 of Table I, preferably of Table IB;
e) a nucleic acid molecule encoding a polypeptide having at least 30% identity with the amino acid sequence of the polypeptide encoded by the nucleic acid molecule of (a) to (c) and having the activity represented by a protein as depicted in column 5 of Table II;
f) a nucleic acid molecule encoding a polypeptide which is isolated with the aid of monoclonal or polyclonal antibodies directed against a polypeptide encoded by one of the nucleic acid molecules of (a) to (e) and having the activity represented by the protein as depicted in column 5 7 of Table II;
g) a nucleic acid molecule encoding a polypeptide comprising the consensus sequence or polypeptide motif as depicted in column 7 of Table IV;
h) a nucleic acid molecule encoding a polypeptide having the activity represented by the protein as depicted in column 5 of Table II;
i) nucleic acid molecule which comprises a polynucleotide, which is obtained by amplifying a cDNA library or a genomic library using the primers as depicted in column 7 of Table III, which do not start at their 5 prime end with the nucleotides ATA;
j) nucleic acid molecule encoding a polypeptide, the polypeptide being derived by substituting, deleting and/or adding one or more amino acids of the amino acid sequence of the polypeptide encoded by the nucleic acid molecules (a) to (d); and
k) a nucleic acid molecule which is obtainable by screening a suitable nucleic acid library under stringent hybridization conditions with a probe comprising a complementary sequence of a nucleic acid molecule of (a) or (b) or with a fragment thereof, having at least 15 nt, preferably 20 nt, 30 nt, 50 nt, 100 nt, 200 nt or 500 nt of a nucleic acid molecule complementary to a nucleic acid molecule sequence characterized in (a) to (d) and encoding a polypeptide having the activity represented by a protein as depicted in column 5 Table II; or which comprises a sequence which is complementary thereto;

or which comprises a sequence which is complementary thereto;

whereby the antisense, RNAi, snRNA, dsRNA, siRNA, miRNA, ta-siRNA, cosuppression molecule, or ribozyme nucleic acid molecule differs at least in one, five, ten, 20, 50, 100 or more nucleotides from the sequence as depicted in column 5 or 7 of Table I A.

In a preferred embodiment, the term “the nucleic acid molecule used in the process of the invention” as used herein relates to said nucleic acid molecule which expression confers the reduction, repression or deletion of the activity selected from the group consisting of 1-phosphatidylinositol 4-kinase, amino acid permease (AAP1), At3g55990-protein, At5g40590-protein, ATP-dependent peptidase/ ATPase/ nucleo-side-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and/or ubiquitin conjugating enzyme/ubiquitin-like activating enzyme.

In a more preferred embodiment, the term “the nucleic acid molecule used in the process of the invention” as used herein relates to the nucleic acid molecule which expression confers the reduction, repression or deletion of the activity represented by a nucleic acid molecule comprising a nucleic acid molecule as depicted in column 5 or 7 of Table I or represented by a polypeptide comprising a polypeptide, a consensus sequence or a polypeptide motif as depicted in column 5 or 7 of Table II or IV.

In an even more preferred embodiment, the term “the nuleic acid molecule used in the process of the invention” relates to the antisense, RNAi, snRNA, dsRNA, siRNA, miRNA, ta-siRNA, cosuppression molecule, or ribozyme molecule of the invention or the cosuppression nucleic acid molecule or the viral degradation nucleic acid molecule of the invention or encoding a DNA-, RNA- or protein-binding factor against genes, RNA's or proteins, a dominant negative mutant, or an antibody of the invention or the nucleic acid molecule for a recombination of the invention, in particular the nucleic acid molecule for producing a homologous recombination event.

The nucleic acid sequences used in the process are advantageously introduced in a nucleic acid construct, preferably an expression cassette, which allows the reduction, depression etc. of the nucleic acid molecules in an organism, advantageously a plant or a microorganism.

Accordingly, the invention also relates to a nucleic acid construct, preferably to an expression construct, comprising the nucleic acid molecule used in the process of the present invention or a fragment thereof functionally linked to one or more regulatory elements or signals. Furthermore the invention also relates to a nucleic acid constructs for the production of homologous recombination events, comprising the nucleic acids molecule used in the process of the present invention or parts thereof.

As described herein, the nucleic acid construct can also comprise further genes, which are to be introduced into the organisms or cells. It is possible and advantageous to introduce into, and express in, the host organisms regulatory genes such as genes for inductors, repressors or enzymes, which, owing to their enzymatic activity, engage in the regulation of one or more genes of a biosynthetic pathway. These genes can be of heterologous or homologous origin. Moreover, further biosynthesis genes may advantageously be present, or else these genes may be located on one or more further nucleic acid constructs.

As described herein, regulator sequences or factors can have a positive effect on preferably the expression of the constructs introduced, thus increasing it. Thus, an enhancement of the regulator elements may advantageously take place at the transcriptional level by using strong transcription signals such as promoters and/or enhancers. In addition, however, an enhancement of translation is also possible, for example by increasing RNA stability. On the other hand the nucleic acid molecule described herein to be reduces according to the process of the invention and the gene products are reduced, decreased or deleted to increase the tolerance and/or resistance to environmental stress and the biomass production as compared to a corresponding non-transformed wild type plant.

In principle, the nucleic acid construct can comprise the herein described regulator sequences and further sequences relevant for the reduction of the expression of nucleic acid molecules to be reduced according to the process of the invention and on the other side for the expression of additional genes in the construct.

Thus, the nucleic acid construct of the invention can be used as expression cassette and thus can be used directly for introduction into the plant, or else they may be introduced into a vector. Accordingly in one embodiment the nucleic acid construct is an expression cassette comprising a microorganism promoter or a microorganism terminator or both. In another embodiment the expression cassette encompasses a plant promoter or a plant terminator or both.

Accordingly, in one embodiment, the process according to the invention comprises the following steps:
a) introduction of a nucleic acid construct comprising a nucleic acid molecule to be used in the process of the invention, e.g. which encodes an antisense, RNAi, snRNA, dsRNA, siRNA, miRNA, ta-siRNA, cosuppression molecule, or ribozyme molecule of the invention or the cosuppression nucleic acid molecule or the viral degradation nucleic acid molecule of the invention or encoding a DNA-, RNA- or protein-binding factor against genes, RNA's or proteins, a dominant negative mutant, or an antibody of the invention or which is suitable for a recombination, in particular a homologous recombination; or
b) introduction of a nucleic acid molecule, including regulatory sequences or factors, which expression increases the expression (a);
   in a cell, or an organism or a part thereof, preferably in a plant or plant cell, and
c) repressing, reducing or deleting the activity to be reduced in the process of the invention by the nucleic acid constructor the nucleic acid molecule mentioned under (a) or (b) in the cell or the organism.

After the introduction and expression of the nucleic acid construct the transgenic organism or cell is advantageously cultured and subsequently harvested. The transgenic organism or cell may be a eukaryotic organism such as a plant, a plant cell, a plant tissue, preferably a crop plant, or a part thereof.

To introduce a nucleic acid molecule for the reduction or repression of a polynucleotide or gene comprising a nucleic acid molecule shown in column 5 or 7 of Table I, or a homologue thereof, or a gene product of said polynucleotide, for e.g. which encodes an antisense, RNAi, snRNA, dsRNA, siRNA, miRNA, ta-siRNA, cosuppression molecule, or ribozyme molecule of the invention or the cosuppression nucleic acid molecule or the viral degradation nucleic acid molecule of the invention or encoding a DNA-, RNA- or protein-binding factor against genes, RNA's or proteins, a dominant negative mutant, or an antibody of the invention or which is suitable for a recombination, in particular a homologous recombination or a mutagenized nucleic acid sequence, into a nucleic acid construct, e.g. as part of an expression cassette, which leads to a reduced activity and/or expression of the respective gene, the codogenic gene segment or the untranslated regions are advantageously subjected to an amplification and ligation reaction in the manner known by a skilled person. It is preferred to follow a procedure similar to the protocol for the Pfu DNA polymerase or a Pfu/Taq DNA polymerase mixture. The primers are selected according to the sequence to be amplified. The specific cloning of antisense, RNAi, snRNA, dsRNA, siRNA, miRNA, ta-siRNA, cosuppression constructs, or ribozyme molecules of the invention or the cosuppression constructs or the viral degradation constructrs or constructs encoding a DNA-, RNA- or protein-binding factor against genes, RNAs or proteins, or constructs for a dominant negative mutant, or an antibody of the invention or of constructs which are suitable for a recombination, in particular a homologous recombination are know to the person skilled in the art. Suitable cloning vectors are generally known to the skilled worker [Cloning Vectors (Eds. Pouwels P. H. et al. Elsevier, Amsterdam-New York-Oxford, 1985, ISBN 0 444 904018)] and have been published, e.g. Earley et al., Plant J. 2006 February; 45 (4): 616-629; Lu et al., Nucleic Acids Res. 2004 Dec. 2; 32 (21): e171; Tao and Xhou, Plant J. 2004 June; 38 (5): 850-860; Miki and Shimamoto Plant Cell Physiol. 2004 April; 45(4):490-495; Akashi et al. , Methods Mol Biol. 2004; 252: 533-43; Wesley et al., Plant J. 2001 September; 27(6): 581-590.

They include, in particular, vectors which are capable of replication in easy to handle cloning systems like bacterial yeast or insect cell based (e.g. baculovirus expression) systems, that is to say especially vectors which ensure efficient cloning in E. coli, and which make possible the stable transformation of plants. Vectors, which must be mentioned, in particular are various binary and cointegrated vector systems, which are suitable for the T-DNA-mediated transformation. Such vector systems are generally characterized in that they contain at least the vir genes, which are required for the Agrobacterium-mediated transformation, and the T-DNA border sequences.

In general, vector systems preferably also comprise further cisregulatory regions such as promoters and terminators and/or selection markers by means of which suitably transformed organisms can be identified. While vir genes and T-DNA sequences are located on the same vector in the case of cointegrated vector systems, binary systems are based on at least two vectors, one of which bears vir genes, but no T-DNA, while a second one bears T-DNA, but no vir gene. Owing to this fact, the last-mentioned vectors are relatively small, easy to manipulate and capable of replication in E. coli and in Agrobacterium. These binary vectors include vectors from the series pBIB-HYG, pPZP, pBecks, pGreen. Those, which are preferably used in accordance with the invention, are Bin19, pB1101, pBinAR, pSun, pGPTV and pCAM-BIA. An overview of binary vectors and their use is given by Hellens et al, Trends in Plant Science (2000) 5, 446-451.

For a construct preparation, vectors may first be linearized using restriction endonuclease(s) and then be modified enzymatically in a suitable manner. Thereafter, the vector is purified, and an aliquot is employed in the cloning step. In the cloning step, the enzyme-cleaved and, if required, purified amplificate is cloned together with similarly prepared vector fragments, using ligase. Alternatively constructs can be prepared be recombination or ligation independent cloning procedure, know to the person skilled in the art. Generally, a specific nucleic acid construct, or vector or plasmid construct, may have one or else more nucleic acid fragments segments. The nucleic acid fragments in these constructs are preferably linked operably to regulatory sequences. The regulatory sequences include, in particular, plant sequences like the above-described promoters and terminators. The constructs can advantageously be propagated stably in microorganisms, in particular Escherichia coli and/or Agrobacterium tumefaciens, under selective conditions and enable the transfer of heterologous DNA into plants or other microorganisms. In accordance with a particular embodiment, the constructs are based on binary vectors (overview of a binary vector: Hellens et al., 2000). As a rule, they contain prokaryotic regulatory sequences, such as replication origin and selection markers, for the multiplication in microorganisms such as Escherichia coli and Agrobacterium tumefaciens. Vectors can further contain agrobacterial T-DNA sequences for the transfer of DNA into plant genomes or other eukaryotic regulatory sequences for transfer into other eukaryotic cells, e.g. Saccharomyces sp. or other prokaryotic regulatory sequences for the transfer into other prokaryotic cells, e.g. Corynebacterium sp. or Bacillus sp. For the transformation of plants, at least the right border sequence, which comprises approximately 25 base pairs, of the total agrobacterial T-DNA sequence is required. Usually, the plant transformation vector constructs according to the invention contain T-DNA sequences both from the right and from the left border region, which contain expedient recognition sites for site-specific acting enzymes, which, in turn, are encoded by some of the vir genes. Different types of repression constructs, e.g. antisense, cosuppression, RNAi, miRNA and so forth need different cloning strategies as described herein.

Advantageously preferred in accordance with the invention are plants host organisms. Preferred plants are selected from among the families Aceraceae, Anacardiaceae, Apiaceae, Asteraceae, Apiaceae, Betulaceae, Boraginaceae, Brassicaceae, Bromeliaceae, Cactaceae, Caricaceae, Caryophyllaceae, Cannabaceae, Convolvulaceae, Chenopodiaceae, Elaeagnaceae, Geraniaceae, Gramineae, Juglandaceae, Lauraceae, Leguminosae, Linaceae, Cucurbitaceae, Cyperaceae, Euphorbiaceae, Fabaceae, Malvaceae, Nymphaeaceae, Papaveraceae, Rosaceae, Salicaceae, Solanaceae, Arecaceae, lridaceae, Liliaceae, Orchidaceae, Gentianaceae, Labiaceae, Magnoliaceae, Ranunculaceae, Carifolaceae, Rubiaceae, Scrophulariaceae, Ericaceae, Polygonaceae, Violaceae, Juncaceae, Poaceae, perennial grass, fodder crops, vegetables and ornamentals.

Especially preferred are plants selected from the groups of the families Apiaceae, Asteraceae, Brassicaceae, Cucurbitaceae, Fabaceae, Papaveraceae, Rosaceae, Solanaceae, Liliaceae or Poaceae. Especially advantageous are, in particular, crop plants. Accordingly, an advantageous plant preferably belongs to the group of the genus peanut, oilseed rape, canola, sunflower, safflower, olive, sesame, hazelnut, almond, avocado, bay, pumpkin/squash, linseed, soya, pistachio, borage, maize, wheat, rye, oats, sorghum and millet, triticale, rice, barley, cassava, potato, sugarbeet, fodder beet, egg plant, and perennial grasses and forage plants, oil palm, vegetables (brassicas, root vegetables, tuber vegetables, pod vegetables, fruiting vegetables, onion vegetables, leafy vegetables and stem vegetables), buckwheat, Jerusalem artichoke, broad bean, vetches, lentil, alfalfa, dwarf bean, lupin, clover and lucerne.

In one embodiment of the invention host plants are selected from the group comprising corn, soy, oil seed rape (including canola and winter oil seed reap), cotton, wheat and rice.

Further preferred plants are mentioned above.

In order to reduce or repress the activity of a gene product according to the process of the invention by introducing, into a plant the nucleic acid molecule used in the process of the invention, for example an isolated antisense, RNAi, snRNA, dsRNA, siRNA, miRNA, ta-siRNA, cosuppression molecule, or ribozyme molecule or a cosuppression nucleic acid molecule or a viral degradation nucleic acid molecule or a recombination nucleic acid molecule or a mutagenized nucleic acid sequence, advantageously is first transferred into an intermediate host, for example a bacterium or a eukaryotic unicellular cell. The transformation into E. coli, which can be carried out in a manner known per se, for example by means of heat shock or electroporation, has proved itself expedient in this context.

The nucleic acid constructs, which are optionally verified, are subsequently used for the transformation of the plants. To this end, it may first be necessary to obtain the constructs from the intermediate host. For example, the constructs may be obtained as plasmids from bacterial hosts by a method similar to conventional plasmid isolation.

Gene silencing in plants can advantageously achieved by transient transformation technologies, meaning that the nucleic acids are preferably not integrated into the plant genome. Suitable systems for transient plant transformations are for example agrobacterium based and plant virus based systems. Details about virus based transient systems and their use for gene silencing in plants have been described in Lu et al. in Methods 2003, 30(4) 296-303. The use of agrobacterium for the transient expression of nucleic acids in plants have been described for example by Fuentes et al., 2003 in Biotechnol Appl Biochem. 2003 Nov. 21 online: doi:10.1042/BA20030192).

A large number of methods for the transformation of plants are known. Since, in accordance with the invention, a stable integration of heterologous DNA into the genome of plants is advantageous, the T-DNA-mediated transformation has proved expedient in particular. For this purpose, it is first necessary to transform suitable vehicles, in particular agrobacteria, with a gene segment or the corresponding plasmid construct comprising the nucleic acid molecule to be transformed, e.g. a nucleic acid molecule suitable for the process of invention, e.g. as described herein, e.g. an isolated antisense, RNAi, snRNA, dsRNA, siRNA, miRNA, ta-siRNA, cosuppression molecule, or ribozyme molecule or a cosuppression nucleic acid molecule or a viral degradation nucleic acid molecule or a recombination nucleic acid molecule or an other polynucleotide capable to reduce or repress the expression of a gene product as shown in column 5 or 7 of Table II, or in column 5 or 7, Table I, or a homologue thereof.

This can be carried out in a manner known per se. For example, said nucleic acid construct of the invention, or said expression construct or said plasmid construct, which has been generated in accordance with what has been detailed above, can be transformed into competent agrobacteria by means of electroporation or heat shock. In principle, one must differentiate between the formation of cointegrated vectors on the one hand and the transformation with binary vectors on the other hand. In the case of the first alternative, the constructs, which comprise the codogenic gene segment or the nucleic acid molecule for the use according to the process of the invention have no T-DNA sequences, but the formation of the cointegrated vectors or constructs takes place in the agrobacteria by homologous recombination of the construct with T-DNA. The T-DNA is present in the agrobacteria in the form of Ti or Ri plasmids in which exogenous DNA has expediently replaced the oncogenes. If binary vectors are used, they can be transferred to agrobacteria either by bacterial conjugation or by direct transfer. These agrobacteria expediently already comprise the vector bearing the vir genes (currently referred to as helper Ti(Ri) plasmid).

In addition the stable transformation of plastids might be of advantageous in some cases because plastids are inherited maternally in most crops reducing or eliminating the risk of transgene flow through pollen. The process of the transformation of the chloroplast genome is generally achieved by a process which has been schematically displayed in Klaus et al., 2004, Nature Biotechnology 22(2), 225-229). Plastidal transformation might especially advantageously for the repression of plastidal encoded nucleic acids of the invention.

Briefly the sequences to be transformed are cloned together with a selectable marker gene between flanking sequences homologous to the chloroplast genome. These homologous flanking sequences direct site specific intergration into the plastome. Plastidal transformation has been described for many different plant species and an overview can be taken from Bock et al. (2001) Transgenic plastids in basic research and plant biotechnology. J Mol Biol. 2001 Sep. 21; 312(3): 425-38, or Maliga, P , Progress towards commercialization of plastid transformation technology. Trends Biotechnol. 21, 20-28 (2003). Further biotechnological progress has recently been reported in form of marker free plastid transformants, which can be produced by a transient cointegrated maker gene, Klaus et al., 2004, Nature Biotechnology 22(2), 225-229.

One or more markers may expediently also be used together with the nucleic acid construct, or the vector and, if plants or plant cells shall be transformed together with the T-DNA, with the aid of which the isolation or selection of transformed organisms, such as agrobacteria or transformed plant cells, is possible. These marker genes enable the identification of a successful transfer of the nucleic acid molecules according to the invention via a series of different principles, for example via visual identification with the aid of fluorescence, luminescence or in the wavelength range of light which is discernible for the human eye, by a resistance to herbicides or antibiotics, via what are known as nutritive markers (auxotrophism markers) or antinutritive markers, via enzyme assays or via phytohormones. Examples of such markers which may be mentioned are GFP (=green fluorescent protein); the luciferin/luceferase system, the -galactosidase with its colored substrates, for example X-Gal, the herbicide resistances to, for example, imidazolinone, glyphosate, phosphinothricin or sulfonylurea, the antibiotic resistances to, for example, bleomycin, hygromycin, streptomycin, kanamycin, tetracyclin, chloramphenicol, ampicillin, gentamycin, geneticin (G418), spectinomycin or blasticidin, to mention only a few, nutritive markers such as the utilization of mannose or xylose, or antinutritive markers such as the resistance to 2-deoxyglucose or D-amino acids (Erikson et al., 2004, Nature Biotech 22(4), 455-458). This list is a small number of possible markers. The skilled worker is very familiar with such markers. Different markers are preferred, depending on the organism and the selection method.

As a rule, it is desired that the plant nucleic acid constructs are flanked by T-DNA at one or both sides of the gene segment. This is particularly useful when bacteria of the species Agrobacterium tumefaciens or Agrobacterium rhizogenes are used for the transformation. A method, which is preferred in accordance with the invention, is the transformation with the aid of Agrobacterium tumefaciens. However, biolistic methods may also be used advantageously for introducing the sequences in the process according to the invention, and the introduction by means of PEG is also possible. The transformed agrobacteria can be grown in the manner known per se and are thus available for the expedient transformation of the plants. The plants or plant parts to be transformed are grown or provided in the customary manner. The transformed agrobacteria are subsequently allowed to act on the plants or plant parts until a sufficient transformation rate is reached. Allowing the agrobacteria to act on the plants or plant parts can take different forms. For example, a culture of morphogenic plant cells or tissue may be used. After the T-DNA transfer, the bacteria are, as a rule, eliminated by antibiotics, and the regeneration of plant tissue is induced. This is done in particular using suitable plant hormones in order to initially induce callus formation and then to promote shoot development.

The transfer of foreign genes into the genome of a plant is called transformation. In doing this the methods described for the transformation and regeneration of plants from plant tissues or plant cells are utilized for transient or stable transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least the flower primordia. The plant is subsequently grown on until the seeds of the treated plant are obtained (Clough and Bent, Plant J. (1998) 16, 735-743). To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Further advantageous transformation methods, in particular for plants, are known to the skilled worker and are described hereinbelow.

Further advantageous suitable methods are protoplast transformation by poly(ethylene glycol)-induced DNA uptake, the “biolistic” method using the gene cannon—referred to as the particle bombardment method, electroporation, the incubation of dry embryos in DNA solution, microinjection and gene transfer mediated by Agrobacterium. Said methods are described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, in particular of crop plants such as by way of example tobacco plants, for example by bathing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S.D. Kung and R. Wu, Academic Press, 1993, pp. 15-38.

The abovementioned nucleic acid molecules can be cloned into the nucleic acid constructs or vectors according to the invention in combination together with further genes, or else different genes are introduced by transforming several nucleic acid constructs or vectors (including plasmids) into a host cell, advantageously into a plant cell.

In one embodiment, in the process according to the invention, the nucleic acid sequences used in the process according to the invention can be advantageously linked operably to one or more regulatory signals in order to increase gene expression for example if an antisense, RNAi, snRNA, dsRNA, siRNA, miRNA, ta-siRNA, cosuppression molecule, or ribozyme molecule of the invention or the cosuppression nucleic acid molecule or the viral degradation nucleic acid molecule of the invention or encoding a DNA-, RNA- or protein-binding factor against genes, RNA's or proteins, a dominant negative mutant, or an antibody of the invention.

These regulatory sequences are intended to enable the specific expression of nucleic acid molecules, e.g. the genes or gene fragments or of the gene products or the nucleic acid used in the process of the invention. Depending on the host organism for example plant or microorganism, this may mean, for example, that the gene or gene fragment or inhibition constructs is expressed and/or overexpressed after induction only, or that it is expressed and/or overexpressed constitutive. These regulatory sequences are, for example, sequences to which the inductors or repressors bind and which thus regulate the expression of the nucleic acid

Moreover, the gene construct can advantageously also comprise one or more of what are known as enhancer sequences in operable linkage with the promoter, and these enable an increased expression of the nucleic acid sequence. Also, it is possible to insert additional advantageous sequences at the 3′ end of the DNA sequences, such as, for example, further regulatory elements or terminators.

The nucleic acid molecules, which encode proteins according to the invention and nucleic acid molecules, which encode other polypeptides may be present in one nucleic acid construct or vector or in several ones. In one embodiment, only one copy of the nucleic acid molecule for use in the process of the invention or its encoding genes is present in the nucleic acid construct or vector. Several vectors or nucleic acid construct or vector can be expressed together in the host organism. The nucleic acid molecule or the nucleic acid construct or vector according to the invention can be inserted in a vector and be present in the cell in a free form. If a stable transformation is preferred, a vector is used, which is stably duplicated over several generations or which or a part of which is else be inserted into the genome. In the case of plants, integration into the plastid genome or, in particular, into the nuclear genome may have taken place. For the insertion of more than one constructs in the host genome the constructs to be expressed might be present together in one vector, for example in above-described vectors bearing a plurality of constructs.

As a rule, regulatory sequences for the expression rate of a constructs, for example a inhibition constructs like RNAi, miRNA, antisense, cosuppresion constructs are located upstream (5′), within, and/or downstream (3′) relative to the sequence of the nucleic acid molecule to be regulated. They control in particular transcription and/or translation and/or the transcript stability. The expression level is dependent on the conjunction of further cellular regulatory systems, such as the protein biosynthesis and degradation systems of the cell.

Regulatory sequences include transcription and translation regulating sequences or signals, e.g. sequences located upstream (5′), which concern in particular the regulation of transcription or translation initiation, such as promoters or start codons, and sequences located downstream (3′), which concern in particular the regulation of transcription or translation termination and transcript stability, such as polyadenylation signals or stop codons. Regulatory sequences can also be present in transcribed coding regions as well in transcribed non-coding regions, e.g. in introns, as for example splicing sites.

Promoters for the regulation of expression of the nucleic acid molecule according to the invention in a cell and which can be employed are, in principle, all those which are capable of reducing the transcription of the nucleic acid molecules if they replace an endogenous promoter or which can stimulate the transcription of inhibiotory constructs for example an antisense, RNAi, snRNA, dsRNA, siRNA, miRNA, ta-siRNA, cosuppression molecule, or ribozyme molecule of the invention or the cosuppression nucleic acid molecule or the viral degradation nucleic acid molecule of the invention or constructs encoding a DNA-, RNA- or protein-binding factor against genes, RNA's or proteins, a dominant negative mutant, or an antibody of the inventionSuitable promoters, which are functional in these organisms, are generally known. They may take the form of constitutive or inducible promoters. Suitable promoters can enable the development- and/or tissue-specific expression in multi-celled eukaryotes; thus, leaf-, root-, flower-, seed-, stomata-, tuber- or fruit-specific promoters may advantageously be used in plants.

In principle, it is possible to use natural promoters together with their regulatory sequences, such as those mentioned above, for the novel process. It is also possible advantageously to use synthetic promoters, either additionally or alone, in particular when they mediate seed-specific expression such as described in, for example, WO 99/16890.

The expression of the nucleic acid molecules used in the process may be desired alone or in combination with other genes or nucleic acids. Multiple nucleic acid molecules conferring repression or expression of advantageous further genes, depending on the goal to be reached, can be introduced via the simultaneous transformation of several individual suitable nucleic acid constructs, i.e. expression constructs, or, preferably, by combining several expression cassettes on one construct. It is also possible to transform several vectors with in each case several expression cassettes stepwise into the recipient organism.

As described above, the transcription of the genes, which are in addition to the introduced nucleic acid molecules to be expressed or the genes introduced can advantageously be terminated by suitable terminators at the 3′ end of the biosynthesis genes introduced (behind the stop codon). Terminator, which may be used for this purpose are, for example, the OCS1 terminator, the nos3 terminator or the 35S terminator. As is the case with the promoters, different terminator sequences can be used for each gene. Terminators, which are useful in microorganism, are for example the fimA terminator, the txn terminator or the trp terminator. Such terminators can be rho-dependent or rho-independent.

Different plant promoters such as, for example, the USP, the LegB4-, the DC3 promoter or the ubiquitin promoter from parsley or other herein mentioned promoter and different terminators may advantageously be used in the nucleic acid construct useful for the reduction of the nucleic acid molecule shown in column 5 or 7 of Table I or its homologues mentioned herein. Further useful plant promoters are for example the maize ubiquitin promoter, the ScBV (Sugarcaine bacilliform virus) promoter, the Ipt2 or Ipt1-gene promoters from barley (WO 95/15389 and WO 95/23230) or those described in WO 99/16890 (promoters from the barley hordein-gene, the rice glutelin gene, the rice oryzin gene, the rice prolamin gene, the wheat gliadin gene, wheat glutelin gene, the maize zein gene, the oat glutelin gene, the Sorghum kasirin-gene, the rye secalin gene).

In order to ensure the stable integration, into the transgenic plant, of nucleic acid molecules used in the process according to the invention in combination with further biosynthesis genes over a plurality of generations, each of the coding regions used in the process can be expressed under the control of its own, preferably unique, promoter.

The nucleic acid construct is advantageously constructed in such a way that a promoter is followed by a suitable cleavage site for insertion of the nucleic acid to be expressed, advantageously in a polylinker, followed, if appropriate, by a terminator located behind the polylinker. If appropriate, this order is repeated several times so that several genes are combined in one construct and thus can be introduced into the transgenic plant in order to be expressed. The sequence is a for example repeated up to three times. For the expression, the nucleic acid sequences are inserted via the suitable cleavage site, for example in the polylinker behind the promoter. It is advantageous for each nucleic acid sequence to have its own promoter and, if appropriate, its own terminator, as mentioned above. However, it is also possible to insert several nucleic acid sequences behind a promoter and, if appropriate, before a terminator, in particular, if a polycistronic transcription is possible in the host or target cells. In this context, the insertion site, or the sequence of the nucleic acid molecules inserted, in the nucleic acid construct is not decisive, that is to say a nucleic acid molecule can be inserted in the first or last position in the cassette without this having a substantial effect on the expression. However, it is also possible to use only one promoter type in the construct.

Accordingly, in a preferred embodiment, the nucleic acid construct according to the invention confers the reduction or repression of a nucleic acid molecule comprising the polynucleotide as depicted in column 5 or 7 of Table I or an encoded gene product, e.g. a polypeptide as depicted in column 5 or 7 of Table II or encompassing a consensus sequence or a polypeptide motif as depicted in column 7 of Table IV, or a homologue thereof described herein and, optionally further genes, in a plant and comprises one or more plant regulatory elements. Said nucleic acid construct according to the invention advantageously encompasses a plant promoter or a plant terminator or a plant promoter and a plant terminator. It further encodes for example isolated nucleic acid molecule of the invention encoding an antisense, RNAi, snRNA, dsRNA, siRNA, miRNA, ta-siRNA, or ribozyme molecule of the invention or the cosuppression nucleic acid molecule or the viral degradation nucleic acid molecule of the invention or encoding a DNA-, RNA- or protein-binding factor against genes, RNA's or proteins, a dominant negative mutant, or an antibody of the invention or the nucleic acid molecule for a recombination of the invention.

A “plant” promoter comprises regulatory elements, which mediate the expression of a coding sequence segment in plant cells. Accordingly, a plant promoter need not be of plant origin, but may originate from viruses or microorganisms, in particular for example from viruses which attack plant cells.

The plant promoter can also originate from a plant cell, e.g. from the plant, which is transformed with the nucleic acid construct or vector as described herein. This also applies to other “plant” regulatory signals, for example in “plant” terminators.

A nucleic acid construct suitable for plant expression preferably comprises regulatory elements which are capable of controlling the expression of genes in plant cells and which are operably linked so that each sequence can fulfill its function. Accordingly, the nucleic acid construct can also comprise transcription terminators. Examples for transcriptional termination are polyadenylation signals. Preferred polyadenylation signals are those which originate from Agrobacterium tumefaciens T-DNA, such as the gene 3 of the Ti plasmid pTiACH5, which is known as octopine synthase (Gielen et al., EMBO J. 3 (1984) 835 et seq.) or functional equivalents thereof, but all the other terminators which are functionally active in plants are also suitable.

In case a nucleic acid construct suitable for plant expression is used for the expression of a polypeptide preferably it also comprises other operably linked regulatory elements such as translation enhancers, for example the overdrive sequence, which comprises the tobacco mosaic virus 5′-untranslated leader sequence, which increases the protein/RNA ratio (Gallie et al., 1987, Nucl. Acids Research 15:8693-8711).

For expression in plants, the nucleic acid molecule must, as described above, be linked operably to or comprise a suitable promotor which expresses for example the antisense, RNAi, snRNA, dsRNA, siRNA, miRNA, ta-siRNA, cosuppression molecule, or ribozyme molecule of the invention or the cosuppression nucleic acid molecule or the viral degradation nucleic acid molecule of the invention or encoding a DNA-, RNA- or protein-binding factor against genes, RNA's or proteins, a dominant negative mutant, or an antibody of the invention at the right point in time and in a cell- or tissue-specific manner. Usable promoters are constitutive promoters (Benfey et al., EMBO J. 8 (1989) 2195-2202), such as those which originate from plant viruses, such as 35S CAMV (Franck et al., Cell 21 (1980) 285-294), 19S CaMV (see also U.S. Pat. No. 5,352,605 and WO 84/02913), 34S FMV (Sanger et al., Plant. Mol. Biol., 14, 1990: 433-443), the parsley ubiquitin promoter, or plant promoters such as the Rubisco small subunit promoter described in U.S. Pat. No. 4,962,028 or the plant promoters PRP1 [Ward et al., Plant. Mol. Biol. 22 (1993)], SSU, PGEL1, OCS [Leisner (1988) Proc Natl Acad Sci USA 85(5): 2553-2557], lib4, usp, mas [Comai (1990) Plant Mol Biol 15 (3):373-381], STLS1, ScBV (Schenk (1999) Plant Mol Biol 39(6):1221-1230), B33, SAD1 or SAD2 (flax promoters, Jain et al., Crop Science, 39 (6), 1999: 1696-1701) or nos [Shaw et al. (1984) Nucleic Acids Res. 12(20):7831-7846]. Stable, constitutive expression of the antisense, RNAi, snRNA, dsRNA, siRNA, miRNA, ta-siRNA, cosuppression molecule, or the ribozyme molecule of the invention or the cosuppression nucleic acid molecule or the viral degradation nucleic acid molecule of the invention or encoding a DNA-, RNA- or protein-binding factor against genes, RNA's or proteins, a dominant negative mutant, or an antibody of the invention can be advantageous. However, inducible expression of the nucleic acid molecule for the reduction of a nucleic acid molecule usuable for the process of the invention is advantageous, if a late expression before the harvest is of advantage, as metabolic manipulation may lead to plant growth retardation.

The expression of the nucleic acid molecule for the reduction of a nucleic acid molecule usuable for the process of the invention is can also be facilitated as described above via a chemical inducible promoter (for a review, see Gatz 1997, Annu. Rev. Plant Physiol. Plant Mol. Biol., 48:89-108). Chemically inducible promoters are particularly suitable when it is desired to express the gene in a time-specific manner. Examples of such promoters are a salicylic acid inducible promoter (WO 95/19443), and abscisic acid-inducible promoter (EP 335 528), a tetracyclin-inducible promoter (Gatz et al. (1992) Plant J. 2, 397-404), a cyclohexanol- or ethanol-inducible promoter (WO 93/21334) or others as described herein.

Other suitable promoters are those which react to biotic or abiotic stress conditions, for example the pathogen-induced PRP1 gene promoter (Ward et al., Plant. Mol. Biol. 22 (1993) 361-366), the tomato heat-inducible hsp80 promoter (U.S. Pat. No. 5,187,267), the potato chill-inducible alpha-amylase promoter (WO 96/12814) or the wound-inducible pinll promoter (EP-A-0 375 091) or others as described herein.

Preferred promoters are in particular those which bring about gene expression in tissues and organs, in seed cells, such as endosperm cells and cells of the developing embryo.

Suitable promoters are the oilseed rape napin gene promoter (U.S. Pat. No. 5,608,152), the Vicia faba USP promoter (Baeumlein et al., Mol Gen Genet, 1991, 225 (3): 459-67), the Arabidopsis oleosin promoter (WO 98/45461), the Phaseolus vulgaris phaseolin promoter (U.S. Pat. No. 5,504,200), the Brassica Bce4 promoter (WO 91/13980), the bean arc5 promoter, the carrot DcG3 promoter, or the Legumin B4 promoter (LeB4; Baeumlein et al., 1992, Plant Journal, 2 (2): 233-9), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley Ipt2 or Ipt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain [U.S. Pat. No. 5,677,474], Bce4 (oilseed rape) [U.S. Pat. No. 5,530,149], glycinin (soya) [EP 571 741], phosphoenolpyruvate carboxylase (soya) [JP 06/62870], ADR12-2 (soya) [WO 98/08962], isocitrate lyase (oilseed rape) [U.S. Pat. No. 5,689,040] or α-amylase (barley) [EP 781 849]. Other promoters which are available for the expression of genes, e.g. of the nucleic acid molecule used in the process of the invention, in particular for the reduction of a nucleic acid molecule which activity is reduces in the process of the invention is in plants are leaf-specific promoters such as those described in DE-A 19644478 or light-regulated promoters such as, for example, the pea petE promoter.

Further suitable plant promoters are the cytosolic FBPase promoter or the potato ST-LSI promoter (Stockhaus et al., EMBO J. 8, 1989, 2445), the Glycine max phosphoribosylpyrophosphate amidotransferase promoter (GenBank Accession No. U87999) or the node-specific promoter described in EP-A-0 249 676.

Other promoters, which are suitable in specific cases are those which bring about plastid-specific expression. Suitable promoters such as the viral RNA polymerase promoter are described in WO 95/16783 and WO 97/06250, and the Arabidopsis clpP promoter, which is described in WO 99/46394.

Other promoters, which are used for the strong expression of heterologous sequences, e.g. the nucleic acid molecule used in the process of the invention, in particular for the reduction of a nucleic acid molecule which activity is reduced in the process of the invention is in as many tissues as possible, in particular also in leaves, are, in addition to several of the abovementioned viral and bacterial promoters, preferably, plant promoters of actin or ubiquitin genes such as, for example, the rice actin1 promoter. Further examples of constitutive plant promoters are the sugarbeet V-ATPase promoters (WO 01/14572). Examples of synthetic constitutive promoters are the Super promoter (WO 95/14098) and promoters derived from G-boxes (WO 94/12015). If appropriate, chemical inducible promoters may furthermore also be used, compare EP-A 388186, EP-A 335528, WO 97/06268.

Another embodiment of the invention is a nucleic acid construct conferring the expression of for example the antisense, RNAi, snRNA, dsRNA, siRNA, miR-NA, ta-siRNA, cosuppression molecule, or ribozyme molecule of the invention or the cosuppression nucleic acid molecule or the viral degradation nucleic acid molecule of the invention or encoding a DNA-, RNA- or protein-binding factor against genes, RNA's or proteins, a dominant negative mutant, or an antibody of the invention as used in the inventive process, suitable for the expression in plant.

Preferred recipient plants are, as described above, in particular those plants, which can be transformed in a suitable manner. These include monocotyledonous and dicotyledonous plants. Plants which must be mentioned in particular are agriculturally useful plants such as cereals and grasses, for example Triticum spp., Zea mays, Hordeum vulgare, oats, Secale cereale, Oryza sativa, Pennisetum glaucum, Sorghum bicolor, Triticale, Agrostis spp., Cenchrus ciliaris, Dactylis glomerata, Festuca arundinacea, Lolium spp., Medicago spp. and Saccharum spp., legumes and oil crops, for example Brassica juncea, Brassica napus, Glycine max, Arachis hypogaea, Gossypium hirsutum, Cicer arietinum, Helianthus annuus, Lens culinaris, Linum usitatissimum, Sinapis alba, Trifolium repens and Vicia narbonensis, vegetables and fruits, for example bananas, grapes, Lycopersicon esculentum, asparagus, cabbage, watermelons, kiwi fruit, Solanum tuberosum, Beta vulgaris, cassava and chicory, trees, for example Coffea species, Citrus spp., Eucalyptus spp., Picea spp., Pinus spp. and Populus spp., medicinal plants and trees, and flowers.

One embodiment of the present invention also relates to a method for generating a vector, which comprises the insertion, into a vector, of the nucleic acid molecule characterized herein, the nucleic acid molecule according to the invention or the expression cassette according to the invention. The vector can, for example, be introduced into a cell, e.g. a microorganism or a plant cell, as described herein for the nucleic acid construct, or below under transformation or transfection or shown in the examples. A transient or stable transformation of the host or target cell is possible, however, a stable transformation is preferred.

The vector according to the invention is preferably a vector, which is suitable for reducing, repressing, decreasing or deleting of the polypeptide according to the invention in a plant. The method can thus also encompass one or more steps for integrating regulatory signals into the vector, in particular signals, which mediate the reduction, decrease or deletion in an plant.

Accordingly, the present invention also relates to a vector comprising the nucleic acid molecule characterized herein as part of a nucleic acid construct suitable for plant expression or the nucleic acid molecule according to the invention.

A advantageous vector used in the process of the invention, e.g. the vector of the invention, comprises a nucleic acid molecule which encodes a nucleic acid molecule which is used in the process of the invention, or a nucleic acid construct suitable for the expression in plant comprising the nucleic acid molecules usable in the process of the invention as described above.

Accordingly, the recombinant expression vectors which are advantageously used in the process of the invention comprise the nucleic acid molecules used in the process according to the invention or the nucleic acid construct according to the invention in a form which is suitable for repressing the activity of a nucleic acid molecule comprising a polynucleotide as depicted in column 5 or 7 of Table I or of a polypeptide as depicted in column 5 or 7 of Table II, or a homologue thereof and/or in the same time expressing, in a host cell, additional genes, which are accompanied by the nucleic acid molecules according to the invention or described herein. Accordingly, the recombinant expression vectors comprise one or more regulatory signals selected on the basis of the host cells to be used for the expression, in operable linkage with the nucleic acid sequence to be expressed.

In accordance with the invention, the term “vector” refers to a nucleic acid molecule, which is capable of transporting another nucleic acid to which it is linked. One type of vector is a “plasmid”, which means a circular double-stranded DNA loop into which additional DNA segments can be ligated. A further type of vector is a viral vector, it being possible to ligate additional DNA segments into the viral genome. Certain vectors are capable of autonomous replication in a host cell into which they have been introduced (for example bacterial vectors with bacterial replication origin). Other preferred vectors are advantageously completely or partly integrated into the genome of a host cell when they are introduced into the host cell and thus replicate together with the host genome. Moreover, certain vectors are capable of controlling the expression of genes with which they are in operable linkage. In the present context, these vectors are referred to as “expression vectors”. As mentioned above, they are capable of autonomous replication or may be integrated partly or completely into the host genome. Expression vectors, which are suitable for DNA recombination techniques usually, take the form of plasmids. In the present description, “plasmid” and “vector” can be used interchangeably since the plasmid is the most frequently used form of a vector. However, the invention is also intended to encompass these other forms of expression vectors, such as viral vectors, which exert similar functions. The term vector is furthermore also to encompass other vectors which are known to the skilled worker, such as phages, viruses such as SV40, CMV, TMV, transposons, IS elements, phasmids, phagemids, cosmids, and linear or circular DNA.

In a recombinant expression vector, “operable linkage” means that the nucleic acid molecule of interest is linked to the regulatory signals in such a way that expression of the genes is possible: they are linked to one another in such a way that the two sequences fulfill the predicted function assigned to the sequence (for example in an in-vitro transcription/translation system, or in a host cell if the vector is introduced into the host cell).

The term “regulatory sequence” is intended to comprise promoters, enhancers and other expression control elements (for example polyadenylation signals). These regulatory sequences are described, for example, in Goeddel: Gene Expression Technology: Methods in Enzymology 185, Academic Press, San Diego, Calif. (1990), or see: Gruber and Crosby, in: Methods in Plant Molecular Biology and Biotechnolgy, CRC Press, Boca Raton, Fla., Ed.: Glick and Thompson, chapter 7, 89-108, including the references cited therein. Regulatory sequences encompass those, which control the constitutive expression of a nucleotide sequence in many types of host cells and those which control the direct expression of the nucleotide sequence in specific host cells only, and under specific conditions. The skilled worker knows that the design of the expression vector may depend on factors such as the selection of the host cell to be transformed, the extent to which the protein amount is reduced, and the like. A preferred selection of regulatory sequences is described above, for example promoters, terminators, enhancers and the like. The term regulatory sequence is to be considered as being encompassed by the term regulatory signal. Several advantageous regulatory sequences, in particular promoters and terminators are described above. In general, the regulatory sequences described as advantageous for nucleic acid construct suitable for expression are also applicable for vectors.

The recombinant expression vectors used can be designed specifically for the expression, in prokaryotic and/or eukaryotic cells, of nucleic acid molecules used in the process. This is advantageous since intermediate steps of the vector construction are frequently carried out in microorganisms for the sake of simplicity. For example, the genes according to the invention and other genes can be expressed in bacterial cells, insect cells (using baculovirus expression vectors), yeast cells and other fungal cells [Romanos (1992), Yeast 8:423-488; van den Hondel, (1991), in: More Gene Manipulations in Fungi, J. W. Bennet & L. L. Lasure, Ed., pp. 396-428: Academic Press: San Diego; and van den Hondel, C. A. M. J. J. (1991), in: Applied Molecular Genetics of Fungi, Peberdy, J. F., et al., Ed., pp. 1-28, Cambridge University Press: Cambridge], algae [Falciatore et al., 1999, Marine Biotechnology.1, 3:239-251] using vectors and following a transformation method as described in WO 98/01572, and preferably in cells of multi-celled plants [see Schmidt, R. and Willmitzer, L. (1988) Plant Cell Rep.:583-586; Plant Molecular Biology and Biotechnology, C Press, Boca Raton, Fla., chapter 6/7, pp.71-119 (1993); F. F. White, in: Transgenic Plants, Bd. 1, Engineering and Utilization, Ed.: Kung and R. Wu, Academic Press (1993), 128-43; Potrykus, Annu. Rev. Plant Physiol. Plant Molec. Biol. 42 (1991), 205-225 (and references cited therein)]. Suitable host cells are furthermore discussed in Goeddel, Gene Expression Technology: Methods in Enzymology 185, Academic Press, San Diego, Calif. (1990). As an alternative, the sequence of the recombinant expression vector can be transcribed and translated in vitro, for example using T7 promotor-regulatory sequences and T7 polymerase.

In most cases, polynucleotides, as RNA, or polypeptides, or proteins can be expressed in prokaryotes using vectors comprising constitutive or inducible promoters, which control the expression of fusion proteins or nonfusion proteins. Typical fusion expression vectors are, inter alia, pGEX (Pharmacia Biotech Inc; Smith, D. B., and Johnson, K. S. (1988) Gene 67:31-40), pMAL (New England Biolabs, Beverly, Mass.) and pRIT5 (Pharmacia, Piscataway, N.J.), in which glutathione-S-transferase (GST), maltose-E-binding protein or protein A is fused with the recombinant target protein. Examples of suitable inducible nonfusion E. coli expression vectors are, inter alia, pTrc (Amann et al. (1988) Gene 69:301-315) and pET 11d [Studier et al., Gene Expression Technology: Methods in Enzymology 185, Academic Press, San Diego, Calif. (1990) 60-89]. The target gene expression of the pTrc vector is based on the transcription of a hybrid trp-lac fusion promoter by the host RNA polymerase. The target gene expression from the pET 11d vector is based on the transcription of a T7-gn10-lac fusion promoter, which is mediated by a coexpressed viral RNA polymerase (T7 gni). This viral polymerase is provided by the host strains BL21 (DE3) or HMS174 (DE3) by a resident “Symbol”-prophage, which harbors a T7 gn1 gene under the transcriptional control of the lacUV 5 promoter.

Other vectors which are suitable in prokaryotic organisms are known to the skilled worker; these vectors are for example in E. coli pLG338, pACYC184, the pBR series, such as pBR322, the pUC series such as pUC18 or pUC19, the M113mp series, pKC30, pRep4, pHS1, pHS2, pPLc236, pMBL24, pLG200, pUR290, pIN-III¹¹³-B1, “Symborgt11 or pBdCl, in Streptomyces pIJ101, pIJ364, pIJ702 or pIJ361, in Bacillus pUB110, pC194 or pBD214, in Corynebacterium pSA77 or pAJ667.

In a further embodiment, the expression vector is a yeast expression vector. Examples of vectors for expression in the yeasts S. cerevisiae encompass pY-eDesaturasec1 (Baldari et al. (1987) Embo J. 6:229-234), pMFa (Kurjan and Herskowitz (1982) Cell 30:933-943), pJRY88 (Schultz et al. (1987) Gene 54:113-123) and pYES2 (Invitrogen Corporation, San Diego, Calif.). Vectors and methods for the construction of vectors which are suitable for use in other fungi, such as the filamentous fungi, encompass those which are described in detail in: van den Hondel, C. A. M. J. J. [(1991), J. F. Peberdy, Ed., pp. 1-28, Cambridge University Press: Cambridge; or in: More Gene Manipulations in Fungi; J. W. Bennet & L. L. Lasure, Ed., pp. 396-428: Academic Press: San Diego]. Examples of other suitable yeast vectors are 2″Symbol”M, pAG-1, YEp6, YEp13 or pEMBLYe23.

Further vectors, which may be mentioned by way of example, are pALS1, plL2 or pBB116 in fungi or pLGV23, pGHlac⁺, pBIN19, pAK2004 or pDH51 in plants.

As an alternative, the nucleic acid sequences can be expressed in insect cells using baculovirus expression vectors. Baculovirus vectors which are available for expressing proteins in cultured insect cells (for example Sf9 cells) encompass the pAc series (Smith et al. (1983) Mol. Cell Biol. 3:2156-2165) and the pVL series (Lucklow and Summers (1989) Virology 170:31-39).

The abovementioned vectors are only a small overview of potentially suitable vectors. Further plasmids are known to the skilled worker and are described, for example, in: Cloning Vectors (Ed. Pouwels, P. H., et al., Elsevier, Amsterdam-New York-Oxford, 1985, ISBN 0 444 904018). Further suitable expression systems for prokaryotic and eukaryotic cells, see the chapters 16 and 17 by Sambrook, J., Fritsch, E. F., and Maniatis, T., Molecular Cloning: A Laboratory Manual, 2nd Edition, Cold Spring Harbor Laboratory, Cold Spring Harbor Laboratory Press, Cold Spring Harbor, N.Y., 1989.

Accordingly, one embodiment of the invention relates to a vector comprising a nucleic acid molecule for use in the process according to the invention or a nucleic acid construct for use in the process of the invention, e.g. the nucleic acid molecule or the nucleic acid construct of the invention encompassing an isolated nucleic acid molecule encoding an antisense, RNAi, snRNA, dsRNA, siRNA, miRNA, ta-siRNA, cosuppression molecule, or ribozyme molecule of the invention or the cosuppression nucleic acid molecule or the viral degradation nucleic acid molecule of the invention or encoding a DNA-, RNA- or protein-binding factor against genes, RNA's or proteins, a dominant negative mutant, or an antibody of the invention or the nucleic acid molecule for a recombination of the invention, in particular the nucleic acid molecute for a homologous recombination. Said vector is useful for the reduction, repression, decrease or deletion of the polypeptide according to the invention in an organism preferably in a plant. Advantageously said nucleic acid molecule is in an operable linkage with regulatory sequences for the expression in a prokaryotic or eukaryotic, or in a prokaryotic and a eukaryotic host. Furthermore vectors which are suitable for homologous recombination are also within the scope of the invention.

Accordingly, one embodiment of the invention relates to a host cell, which has been transformed stably or transiently with the vector usable in the process of the invention, in particular with the vector according to the invention or the nucleic acid molecule according to the invention or the nucleic acid construct according to the invention. Said host cell may be a microorganism, a non-human animal cell or a plant cell.

In one embodiment, the present invention relates to a polypeptide encoded by the nucleic acid molecule according to the present invention, e.g. encoded by a nucleic acid molecule as depicted in column 5 or 7 of Table IB, this means for example the present invention also relates to a polypeptide as depicted in column 5 or 7 of Table IIB, preferably conferring an increase in the tolerance and/or resistance to environmental stress and in the biomass production as compared to a corresponding nontransformed wild type plant after decreasing or repressing the expression or activity. Advantageously, said polypeptide or a fragment thereof, in particular an epitope or a haptene, which are all comprised by the term “polypeptide of the invention” can be used to produce or generate an antibody against said polypeptide. Advantageously, the antibody inactivates or reduces the activity of a polypeptide, which activity is reduced in the process of the present invention.

The present invention also relates to a process for the production of a polypeptide according to the present invention, the polypeptide being expressed in a host cell according to the invention, preferably in a microorganism, non-human animal cell or a transgenic plant cell.

In one embodiment, the nucleic acid molecule used in the process for the production of the polypeptide is derived from said microorganism, preferably from said prokaryotic or protozoic cell with said eukaryotic organism as host cell. In another embodiment the polypeptide is produced in said plant cell or plant with a nucleic acid molecule derived from a prokaryote or a fungus or an alga or another microorganism but not from plant. In another embodiment the polypeptide is produced in said plant cell or plant with a nucleic acid molecule derived from a plant or algae.

The skilled worker knows that protein and DNA expressed in different organisms differ in many respects and properties, e.g. methylation, degradation and post-translational modification as for example glucosylation, phosphorylation, acetylation, myristoylation, ADP-ribosylation, farnesylation, carboxylation, sulfation, ubiquination, etc. though having the same coding sequence. Preferably, the cellular expression control of the corresponding protein differs accordingly in the control mechanisms controlling the activity and expression of an endogenous protein or another eukaryotic protein. One major difference between proteins expressed in prokaryotic or eukaryotic organism is the amount of glycosylation. For example in E. coli there are no glycosylated proteins. Proteins expressed in yeasts have high mannose content in the glycosylated proteins, whereas in plants the glycosylation pattern is complex.

The polypeptide of the present invention is preferably produced by recombinant DNA techniques. For example, a nucleic acid molecule encoding the protein is cloned into a vector (as described above), the vector is introduced into a host cell (as described above) and said polypeptide is expressed in the host cell. Said polypeptide can then be isolated from the cells by an appropriate purification scheme using standard protein purification techniques. Alternative to recombinant expression, a polypeptide being encoded by a nucleic acid molecule comprising a nucleic acid molecule as depicted in column 5 or 7 of Table I or a homologue thereof, in particular a fragment or a peptide of the present invention can be synthesized chemically using standard peptide synthesis techniques. Moreover, native polypeptides having the same structure and preferably conferring the activity of the protein usable in the process of the invention can be isolated from cells (e.g., endothelial cells), for example using the antibody of the present invention as described below. The antibody can be produced by standard techniques utilizing the polypeptide usable in the process of the present invention or a fragment thereof, i.e., the polypeptide of this invention.

In one embodiment, the present invention relates to a polypeptide having the activity represented by a polypeptide comprising a polypeptide as depicted in column 5 or 7 of Table II or comprising a consensus sequence or a polypeptide motif as depicted in column 7 of Table IV, in particular an activity selected from the group consisting of: 1-phosphatidylinositol 4-kinase, amino acid permease (AAP1), At3g55990-protein, At5g40590-protein, ATP-dependent peptidase/ ATPase/nucleoside-triphosphatase/serine-type endopeptidase, DC1 domain-containing protein/pro-tein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and/or ubiquitin conjugating enzyme/ubiquitin-like activating enzyme. Said polypeptide confers preferably the aforementioned activity, in particular, the polypeptide confers the increase of the tolerance and/or resistance to environmental stress and of the biomass production as compared to a corresponding non-transformed wild type plant after decreasing or repressing the cellular activity, e.g. by decreasing the expression or the specific activity of the polypeptide. In one embodiment, the present invention relates to a polypeptide having the amino acid sequence encoded by a nucleic acid molecule of the invention or obtainable by a process for the production of a polypeptide of the invention.

In one embodiment, said polypeptide distinguishes over the sequence as depicted in column 5 or 7 of Table IIA or B by one or more amino acid. In another embodiment, said polypeptide of the invention does not consist of the sequence as depicted in column 5 or 7 of Table IIA or B. In a further embodiment, said polypeptide of the present invention is less than 100%, 99,999%, 99,99%, 99,9% or 99% identical to column 5 or 7 of Table IIA or B.

Preferably, the sequence of the polypeptide of the invention distinguishes from the sequence as depicted in column 5 or 7 of Table IIA or B by not more than 80% or 70% of the amino acids, preferably not more than 60% or 50%, more preferred not more than 40% or 30%, even more preferred not more than 20% or 10%. In one embodiment, the polypeptide distinguishes form the sequence as depicted in column 5 or 7 of Table IIA or B by more than 5, 6, 7, 8 or 9 amino acids, preferably by more than 10, 15, 20, 25 or 30 amino acids, even more preferred are more than 40, 50, or 60 amino acids. In one embodiment, the polypeptide of the invention originates from a plant cell.

Preferably, the polypeptide is isolated. An “isolated” or “purified” protein or nucleic acid molecule or biologically active portion thereof is substantially free of cellular material when produced by recombinant DNA techniques, or chemical precursors or other chemicals when chemically synthesized.

The language “substantially free of cellular material” includes preparations of the polypeptide in which the protein is separated from cellular components of the cells in which it is naturally or recombinantly produced. In one embodiment, the language “substantially free of cellular material” includes preparations having less than about 30% (by dry weight) of “contaminating protein”, more preferably less than about 20% of “contaminating protein”, still more preferably less than about 10% of “contaminating protein”, and most preferably less than about 5% “contaminating protein”. The term “contaminating protein” relates to polypeptides, which are not polypeptides of the present invention. When the polypeptide of the present invention or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other che-micals” includes preparations in which the polypeptide of the present invention is separated from chemical precursors or other chemicals, which are involved in the synthesis of the protein. The language “substantially free of chemical precursors or other chemicals” includes preparations having less than about 30% (by dry weight) of chemical precursors or other proteins or chemicals which are not identical to the protein, more preferably less than about 20% chemical precursors or other proteins or chemicals, still more preferably less than about 10% chemical precursors or other proteins or chemicals, and most preferably less than about 5% chemical precursors or other proteins or chemicals which are not identical to the protein of the invention. In preferred embodiments, isolated proteins or biologically active portions thereof lack contaminating proteins from the same organism from which the polypeptide of the present invention is derived. Typically, such proteins are produced by recombinant techniques.

A polypeptide of the invention comprises preferably an amino acid sequence which is sufficiently homologous to an amino acid sequence as depicted in column 5 or 7 of Table II or which comprises a consensus sequence or a polypeptide motif as depicted in column 7 of Table IV such that the protein or portion thereof maintains the ability to confer the activity of the present invention. Preferably, the polypeptide has an amino acid sequence identical as depicted in column 5 or 7 of Table II.

Further, the polypeptide of the invention or the polypeptide which activity is to be reduced in the process of the invention can have an amino acid sequence which is encoded by a nucleotide sequence which hybridizes, preferably hybridizes under stringent conditions as described above, to a nucleotide sequence of the nucleic acid molecule of the present invention.

Accordingly, the polypeptide has an amino acid sequence which is encoded by a nucleotide sequence that is at least about 35%, 40%, 45%, 50%, 55%, 60%, 65% or 70%, preferably at least about 75%, 80%, 85% or 90%, and more preferably at least about 91%, 92%, 93%, 94% or 95%, and even more preferably at least about 96%, 97%, 98%, 99% or more homologous to one of the nucleic acid molecules as depicted in column 5 or 7 of Table I. The preferred polypeptide possesses at least one of the activities according to the invention and described herein.

A preferred polypeptide complement the knock out, e.g. an inactivation or a reduction, repression or deletion of a polypeptide comprising a polypeptide as depicted in column 5 or 7 of Table II or comprising a consensus sequence or a polypeptide motif as depicted in column 7 of Table IV, when appropriately expressed in the knock out mutant. Appropriately expressed means in this context, that the polypeptide is produced in a similar quality and quantity and in a same developmental phase, tissue and compartment as the polypeptide inactivated, deleted or reduced in the knock out mutant. A preferred polypeptide of the present invention includes an amino acid sequence encoded by a nucleotide sequence which hybridizes, preferably hybridizes under stringent conditions, to a nucleotide sequence of column 5 or 7 of Table I or which is homologous thereto, as defined above.

Accordingly the polypeptide which activity is to be reduced in the process of the present invention, e.g. the polypeptide of the present invention can vary from the amino acid sequence of a polypeptide as depicted in column 5 or 7 of Table II or comprising a consensus sequence or a polypeptide motif as depicted in column 7 of Table IV in amino acid sequence due to natural variation or mutagenesis, as described in detail herein. Accordingly, the polypeptide comprise an amino acid sequence which is at least about 35%, 40%, 45%, 50%, 55%, 60%, 65% or 70%, preferably at least about 75%, 80%, 85% or 90%, and more preferably at least about 91%, 92%, 93%, mologous to an entire amino acid sequence of a polypeptide as depicted in column 5 or 7 of Table II or comprising a consensus sequence or a polypeptide motif as depicted in column 7 of Table IV.

For the comparison of amino acid sequences the same algorithms as described above or nucleic acid sequences can be used. Results of high quality are reached by using the algorithm of Needleman and Wunsch or Smith and Waterman. Therefore programs based on said algorithms are preferred. Advantageously the comparisons of sequences can be done with the program PileUp (J. Mol. Evolution, 25, 351-360, 1987, Higgins et al., CABIOS, 5 1989: 151-153) or preferably with the programs Gap and BestFit, which are respectively based on the algorithms of Needleman and Wunsch [J. Mol. Biol. 48; 443-453 (1970)] and Smith and Waterman [Adv. Appl. Math. 2; 482-489 (1981)]. Both programs are part of the GCG software-package [Genetics Computer Group, 575 Science Drive, Madison, Wis., USA 53711 (1991); Altschul et al. (1997) Nucleic Acids Res. 25:3389 et seq.]. Therefore preferably the calculations to determine the perentages of sequence homology are done with the program Gap over the whole range of the sequences. The following standard adjustments for the comparison of amino acid sequences were used: gap weight: 8, length weight: 2, average match: 2.912, average mismatch: -2.003.

Biologically active portions of a polypeptide include peptides comprising amino acid sequences derived from the amino acid sequence of the polypeptide disclosed herein, e.g., they comprise the amino acid sequence as depicted in the column 5 or 7 of Table II or the consensus sequence or the polypeptide motifs of column 7 of Table IV or the amino acid sequence of a protein homologous thereto, which include fewer amino acids than a full length protein having the activity of said protein, e.g. as disclosed or a full length protein which is homologous to a protein having the activity of the protein as disclosed or of a polypeptide to be reduced in the process of the present invention as depicted herein, and the repression, reduction or decrease of which lead to an increase of the tolerance and/or resistance to environmental stress and of the biomass production as compared to a corresponding non-transformed wild type plant.

Typically, biologically (or immunologically) active portions i.e. peptides, e.g., peptides which are, for example, 5, 10, 15, 20, 30, 35, 36, 37, 38, 39, 40, 50, 100 or more amino acids in length comprise a domain or motif with at least one activity or epitope of the polypeptide of the present invention. Moreover, other biologically active portions, in which other regions of the polypeptide are deleted, can be prepared by recombinant techniques and evaluated for one or more of the activities described herein.

Any mutagenesis strategies for the polypeptide usable in the process of the invention, in particular, of a polypeptide of the present invention, which result in an increase or in a decrease in the activity disclosed herein are not meant to be limiting; variations on these strategies will be readily apparent to one skilled in the art. Using such strategies, and incorporating the mechanisms disclosed herein, the nucleic acid molecule and polypeptide disclosed herein may be utilized to generate plants or parts thereof, expressing mutated nucleic acid molecule and/or polypeptide molecules still usable in the process of the invention. This desired compound may be any natural product of plants, which includes the final products of biosynthesis pathways and intermediates of naturally-occurring metabolic pathways, as well as molecules which do not naturally occur in the metabolism of said cells, but which are produced by a said cells of the invention.

The invention also provides chimeric or fusion proteins.

As used herein, a “chimeric protein” or “fusion protein” comprises a polypeptide operatively linked to a polypeptide which does not confer above-mentioned activity, in particular, which does confer an increase of the tolerance and/or resistance to environmental stress and of the biomass production as compared to a corresponding non-transformed wild type plant if its expression or activity is decreased.

In one embodiment, a protein (=“polypeptide”) is preferred which confers the increase of the tolerance and/or resistance to environmental stress and of the biomass production as compared to a corresponding non-transformed wild type plant, once its activity is decreased. Said protein refers preferably to a polypeptide having an amino acid sequence corresponding to the polypeptide as disclosed herein, preferably having an amino acid sequence corresponding to the polypeptides as depicted in column 5 or 7 of Table II or comprising a consensus sequence or a polypeptide motif as depicted in column 7 of Table IV, or a homologue thereof.

Within the fusion protein, the term “operatively linked” is intended to indicate that a polypeptide as disclosed herein and an other polypeptide or part thereof are fused to each other so that both sequences fulfil the proposed function addicted to the sequence used. The other polypeptide can be fused to the N-terminus or C-terminus of e.g. a polypeptide which activity is to be reduced in the process of the invention. For example, in one embodiment the fusion protein is a GST fusion protein in which the sequences of the polypeptide are fused to the C-terminus of the GST sequences. Such fusion proteins can facilitate the purification of recombinant polypeptides of the invention.

Preferably, a chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. The fusion gene can be synthesized by conventional techniques including automated DNA synthesizers. Alternatively, PCR amplification of gene fragments can be carried out using anchor primers, which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). The nucleic acid molecule can be cloned into such an expression vector such that the fusion moiety is linked in-frame to the encoded protein.

Furthermore, folding simulations and computer redesign of structural motifs of a protein to be reduced or repressed according to the process of the invention, e.g. of a polypeptide as disclosed herein, can be performed using appropriate computer programs (Olszewski, Proteins 25 (1996), 286-299; Hoffman, Comput. Appl. Biosci. 11 (1995), 675-679). Computer modeling of protein folding can be used for the conformational and energetic analysis of detailed peptide and protein models (Monge, J. Mol. Biol. 247 (1995), 995-1012; Renouf, Adv. Exp. Med. Biol. 376 (1995), 37-45). The appropriate programs can be used for the identification of interactive sites of a polypeptide and its substrates or binding factors or other interacting proteins by computer assistant searches for complementary peptide sequences (Fassina, Immunomethods (1994), 114-120). Further appropriate computer systems for the design of protein and peptides are described in the prior art, for example in Berry, Biochem. Soc. Trans. 22 (1994), 1033-1036; Wodak, Ann. N. Y. Acad. Sci. 501 (1987), 1-13; Pabo, Biochemistry 25 (1986), 5987-5991. The results obtained from the above-described computer analysis can be used for, e.g., the preparation of peptidomimetics of a protein or fragments thereof. Such pseudopeptide analogues of the, natural amino acid sequence of the protein may very efficiently mimic the parent protein (Benkirane, J. Biol. Chem. 271 (1996), 33218-33224). For example, incorporation of easily available achiral Q-amino acid residues into a protein or a fragment thereof results in the substitution of amide bonds by polymethylene units of an aliphatic chain, thereby providing a convenient strategy for constructing a peptidomimetic (Banerjee, Biopolymers 39 (1996), 769-777).

Superactive peptidomimetic analogues of small peptide hormones in other systems are described in the prior art (Zhang, Biochem. Biophys. Res. Commun. 224 (1996), 327-331). Appropriate peptidomimetics of a polypeptide can also be identified by the synthesis of peptidomimetic combinatorial libraries through successive amide alkylation and testing the resulting compounds, e.g., for their binding and immunological properties. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715.

Furthermore, a three-dimensional and/or crystallographic structure of the protein can be used for the design of peptidomimetic inhibitors of the activity of a protein comprising a polypeptide as depicted in column 5 or 7 of Table II or comprising a consensus sequence or a polypeptide motif as depicted in column 7 of Table IV (Rose, Biochemistry 35 (1996), 12933-12944; Rutenber, Bioorg. Med. Chem. 4 (1996), 1545-1558).

Furthermore, a three-dimensional and/or crystallographic structure of a protein described herein and the identification of interactive sites and its substrates or binding factors can be used for design of mutants with modulated binding or turn over activities. For example, the active center of the polypeptide of the present invention can be modelled and amino acid residues participating in the catalytic reaction can be modulated to increase or decrease the binding of the substrate to inactivate the polypeptide. The identification of the active center and the amino acids involved in the catalytic reaction facilitates the screening for mutants having an increased or decreased activity.

One embodiment of the invention also relates to an antibody, which binds specifically to the polypeptide disclosed herein, i.e. specific fragments or epitopes of such a protein.

The term “epitope” relates to specific immunoreactive sites within an antigen, also known as antigenic determinates. These epitopes can be a linear array of monomers in a polymeric composition—such as amino acids in a protein—or consist of or comprise a more complex secondary or tertiary structure. Those of skill will recognize that immunogens (i.e., substances capable of eliciting an immune response) are antigens; however, some antigen, such as haptens, are not immunogens but may be made immunogenic by coupling to a carrier molecule. The term “antigen” includes references to a substance to which an antibody can be generated and/or to which the antibody is specifically immunoreactive.

The antibody preferably confers the reduction, repression or deletion of a protein comprising a polypeptide as depicted in column 5 or 7 of Table II, preferably as depicted in Table IIB, or comprising a consensus sequence or a polypeptide motif as depicted in column 7 of Table of Table IV, or a homologue thereof as described herein, e.g. the antibody inactivates the protein of the invention due to its binding in the organism or a part thereof.

The antibodies of the invention can also be used to identify and isolate a target polypeptide which activity has to be reduces according to the invention. Such antibodies can also be expressed in the suitable host organisms thereby reducing the activity of a gene product disclosed herein, e.g. the polynucleotide or polypeptide disclosed herein, e.g. of a nucleic acid molecule comprising a nucleic acid molecule shown in column 5 or 7 of Table I, e.g. the polypeptide comprising the polypeptide as depicted in column 5 or 7 of Table II, by binding to the expression product leading for example to a steric interferance with their activity.

These antibodies can be monoclonal antibodies, polyclonal antibodies or synthetic antibodies as well as fragments of antibodies, such as Fab, Fv or scFv fragments etc. Monoclonal antibodies can be prepared, for example, by the techniques as originally described in Köhler and Milstein, Nature 256 (1975), 495, and Galfr6, Meth. Enzymol. 73 (1981), 3, which comprise the fusion of mouse myeloma cells to spleen cells derived from immunized mammals.

Furthermore, antibodies or fragments thereof to the aforementioned peptides can be obtained by using methods, which are described, e.g., in Harlow and Lane “Antibodies, A Laboratory Manual”, CSH Press, Cold Spring Harbor, 1988. These antibodies can be used, for example, for the immunoprecipitation and immuno-localization of proteins according to the invention as well as for the monitoring of the synthesis of such proteins, for example, in recombinant organisms, and for the identification of compounds interacting with the protein according to the invention. For example, surface plasmon resonance as employed in the BlAcore system can be used to increase the efficiency of phage antibodies selections, yielding a high increment of affinity from a single library of phage antibodies, which bind to an epitope of the protein of the invention (Schier, Human Antibodies Hybridomas 7 (1996), 97-105; Malmborg, J. Immunol. Methods 183 (1995), 7-13). In many cases, the binding phenomena of antibodies to antigens is equivalent to other ligand/anti-ligand binding.

A further embodiment of the invention also relates to a method for the generation of a transgenic plant cell or a transgenic plant tissue or a transgenic plant, which comprises introducing, into the plant, the plant cell or the plant tissue, the nucleic acid construct according to the invention, the vector according to the invention, or the nucleic acid molecule according to the invention.

A further embodiment of the invention also relates to a method for the transient generation of a transgenic plant cell or a transgenic plant tissue or a transgenic plant, which comprises introducing, into the plant, the plant cell or the plant tissue, the nucleic acid construct according to the invention, the vector according to the invention, the nucleic acid molecule characterized herein as being contained in the nucleic acid construct of the invention or the nucleic acid molecule used in the process according to the invention, whereby the introduced nucleic acid molecules, nucleic acid construct and/or vector is not integrated into the genome of the host or host cell. Therefore the transformants are not stable during the propagation of the host in respect of the introduced nucleic acid molecules, nucleic acid construct and/or vector.

In the process according to the invention, transgenic organisms are also to be understood as meaning—if they take the form of plants—plant cells, plant tissues, plant organs such as root, shoot, stem, seed, flower, tuber or leaf, or intact plants which are grown.

“Growing” is to be understood as meaning for example culturing the transgenic plant cells, plant tissue or plant organs on or in a nutrient medium or the intact plant on or in a substrate, for example in hydroponic culture, potting compost or on a field soil.

In a further advantageous embodiment of the process, the nucleic acid molecules can be expressed in plant cells from higher plants (for example spermatophytes such as crops). Examples of plant expression vectors encompass those which are described in detail herein or in: Becker, D. [(1992) Plant Mol. Biol. 20:1195-1197] and Bevan, M.W. [(1984), Nucl. Acids Res. 12:8711-8721; Vectors for Gene Transfer in Higher Plants; in: Transgenic Plants, Vol. 1, Engineering and Utilization, Ed.: Kung and R. Wu, Academic Press, 1993, pp. 15-38]. An overview of binary vectors and their use is also found in Hellens, R. [(2000), Trends in Plant Science, Vol. 5 No.10, 446-451.

Vector DNA can be introduced into cells via conventional transformation or transfection techniques. The terms “transformation” and “transfection” include conjugation and transduction and, as used in the present context, are intended to encompass a multiplicity of prior-art methods for introducing foreign nucleic acid molecules (for example DNA) into a host cell, including calcium phosphate coprecipitation or calcium chloride coprecipitation, DEAE-dextran-mediated transfection, PEG-mediated transfection, lipofection, natural competence, chemically mediated transfer, electroporation or particle bombardment. Suitable methods for the transformation or transfection of host cells, including plant cells, can be found in Sambrook et al. (Molecular Cloning: A Laboratory Manual., 2^{nd} Ed., Cold Spring Harbor Laboratory, Cold Spring Harbor Laboratory Press, Cold Spring Harbor, N.Y., 1989) and in other laboratory handbooks such as Methods in Molecular Biology, 1995, Vol. 44, Agrobacterium protocols, Ed.: Gartland and Davey, Humana Press, Totowa, N.J.

The above-described methods for the transformation and regeneration of plants from plant tissues or plant cells are exploited for transient or stable transformation of plants. Suitable methods are the transformation of protoplasts by polyethylene-glycol-induced DNA uptake, the biolistic method with the gene gun—known as the particle bombardment method-, electroporation, the incubation of dry embryos in DNA-containing solution, microinjection and the Agrobacterium-mediated gene transfer. The abovementioned methods are described for example in B. Jenes, Techniques for Gene Transfer, in: Transgenic Plants, Vol. 1, Engineering and Utilization, edited by S. D. Kung and R. Wu, Academic Press (1993) 128-143 and in Potrykus Annu. Rev. Plant Physiol. Plant Molec. Biol. 42 (1991) 205-225. The construct to be expressed is preferably cloned into a vector, which is suitable for transforming Agrobacterium tumefaciens, for example pBin19 (Bevan, Nucl. Acids Res. 12 (1984) 8711). Agrobacteria transformed with such a vector can then be used in the known manner for the transformation of plants, in particular crop plants, such as, for example, tobacco plants, for example by bathing scarified leaves or leaf segments in an agrobacterial solution and subsequently culturing them in suitable media. The transformation of plants with Agrobacterium tumefaciens is described for example by Hofgen and Willmitzer in Nucl. Acid Res. (1988) 16, 9877 or known from, inter alia, F. F. White, Vectors for Gene Transfer in Higher Plants; in Transgenic Plants, Vol. 1, Engineering and Utilization, edited by S. D. Kung and R. Wu, Academic Press, 1993, pp. 15-38.

To select for the successful transfer of a nucleic acid molecule, vector or nucleic acid construct into a host organism, it is advantageous to use marker genes as have already been described above in detail. It is known of the stable or transient integration of nucleic acids into plant cells that only a minority of the cells takes up the foreign DNA and, if desired, integrates it into its genome, depending on the expression vector used and the transfection technique used. To identify and select these integrants, a gene encoding for a selectable marker (as described above, for example resistance to antibiotics) is usually introduced into the host cells together with the gene of interest. Preferred selectable markers in plants comprise those, which confer resistance to an herbicide such as glyphosate or gluphosinate. Other suitable markers are, for example, markers, which encode genes involved in biosynthetic pathways of, for example, sugars or amino acids, such as β-galactosidase, ura3 or ilv2. Markers, which encode genes such as luciferase, gfp or other fluorescence genes, are likewise suitable. These markers and the aforementioned markers can be used in mutants in whom these genes are not functional since, for example, they have been deleted by conventional methods. Furthermore, nucleic acid molecules, which encode a selectable marker, can be introduced into a host cell on the same vector as those, which encode the nucleotide acid molecule used in the process or else in a separate vector. Cells which have been transfected stably with the nucleic acid molecule introduced can be identified for example by selection (for example, cells which have integrated the selectable marker survive whereas the other cells die).

Since the marker genes, as a rule specifically the gene for resistance to antibiotics and herbicides, are no longer required or are undesired in the transgenic host cell once the nucleic acids have been introduced successfully, the process according to the invention for introducing the nucleic acids advantageously employs techniques which enable the removal, or excision, of these marker genes. One such a method is what is known as cotransformation. The cotransformation method employs two vectors simultaneously for the transformation, one vector bearing the nucleic acid or nucleic acid construct according to the invention and a second bearing the marker gene(s). A large proportion of transformants receives or, in the case of plants, comprises (up to 40% of the transformants and above), both vectors. The marker genes can subsequently be removed from the transformed plant by performing crosses. In an preferred embodiment, a conditional marker allowing both positive and negative selection is used, in order to first identify the transformation event by the positive selection and later on allowing for the identification of lines which have lost the marker through crossing or segregation by negative selection. Markers which confer resistance against D-amino acids are such preferred conditional markers (Erikson et al., 2004, Nature Biotech 22(4), 455-458). In another method, marker genes integrated into a transposon are used for the transformation together with desired nucleic acid (known as the Ac/Ds technology). In some cases (approx. 10%), the transposon jumps out of the genome of the host cell once transformation has taken place successfully and is lost. In a further number of cases, the transposon jumps to a different location. In these cases, the marker gene must be eliminated by performing crosses. In microbiology, techniques were developed which make possible, or facilitate, the detection of such events. A further advantageous method relies on what are known as recombination systems, whose advantage is that elimination by crossing can be dispensed with. The best-known system of this type is what is known as the Cre/lox system. Crel is a recombinase, which removes the sequences located between the loxP sequence. If the marker gene is integrated between the loxP sequence, it is removed, once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

Agrobacteria transformed with an expression vector according to the invention may also be used in the manner known per se for the transformation of plants such as experimental plants like Arabidopsis or crop plants, such as, for example, cereals, maize, oats, rye, barley, wheat, soya, rice, cotton, sugarbeet, canola, sunflower, flax, hemp, potato, tobacco, tomato, carrot, bell peppers, oilseed rape, tapioca, cassava, arrow root, tagetes, alfalfa, lettuce and the various tree, nut, cotton and grapevine species, in particular oil-containing crop plants such as soya, peanut, castor-oil plant, sunflower, maize, cotton, flax, oilseed rape, coconut, oil palm, safflower (Carthamus tinctorius) or cocoa beans, for example by bathing scarified leaves or leaf segments in an agrobacterial solution and subsequently growing them in suitable media.

In addition to the transformation of somatic cells, which then has to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic (Feldman, K A and Marks M D (1987). Mol Gen Genet 208:274-289; Feldmann K (1992). In: C Koncz, N-H Chua and J Shell, eds, Methods in Arabidopsis Research. Word Scientific, Singapore, pp. 274-289). Alternative methods are based on the repeated removal of the influorescences and incubation of the excision site in the center of the rosette with transformed agrobacteria, whereby transformed seeds can likewise be obtained at a later point in time (Chang (1994). Plant J. 5: 551-558; Katavic (1994) Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension (Bechthold, N (1993). C R Acad Sci Paris Life Sci, 316: 1194-1199), while in the case of the“floral dip” method the developing floral tissue is incubated briefly with a surfactant-treated agrobacterial suspension (Clough, S J and Bent, A F (1998). The Plant J. 16, 735-743). A certain proportion of transgenic seeds are harvested in both cases, and these seeds can be distinguished from nontransgenic seeds by growing under the above-described selective conditions.

The genetically modified plant cells can be regenerated via all methods with which the skilled worker is familiar. Suitable methods can be found in the abovementioned publications by S. D. Kung and R. Wu, Potrykus or Hofgen and Willmitzer.

Accordingly, the present invention thus also relates to a plant cell comprising the nucleic acid construct according to the invention, the nucleic acid molecule according to the invention or the vector according to the invention. Accordingly, the present invention thus also relates to a plant cell produced according to the abovementioned process to produce a plant cell.

Accordingly the present invention relates to any cell, in particular to a plant cell, plant tissue or plant or its progeny, which is transgenic for any nucleic acid molecule or construct disclosed herein, e.g. the nucleic acid molecule's repression or reduction or its gene product activity repression or reduction confers the increased tolerance and/or resistance to environmental stress and increased biomass production as compared to a corresponding non-transformed wild type plant.

Accordingly the present invention relates to any cell transgenic for any nucleic acid molecule comprising the nucleic acid molecule or part of it, which activity is to be reduced or encoding the polypeptide which acitivity is to be reduced in the process of the invention, e.g. the nucleic acid molecule of the invention, the nucleic acid construct of the invention, the antisense molecule of the invention, the vector of the invention or a nucleic acid molecule encoding the polypeptide of the invention, e.g. encoding a polypeptide having activity of the protein of the invention.

Accordingly the present invention relates to any cell transgenic for the the vector, the host cell, the polypeptide, or the antisense, RNAi, snRNA, dsRNA, siR-NA, miRNA, ta-siRNA, cosuppression construct, recombination construct or ribozyme molecule, or the viral nucleic acid molecule, the antibody of the invention, e.g. for the vector, the host cell, the polypeptide, or the antisense, RNAi, snRNA, dsRNA, siRNA, miRNA, ta-siRNA, cosuppression construct, recombination construct or ribozyme molecule, or the viral nucleic acid molecule comprising a fragment of the nucleic acid molecule disclosed herein, the antibody binding to a epitope of the polypeptide disclosed herein.

A naturally occurring expression cassette—for example the naturally occurring combination of the promoter of the protein with the corresponding gene, which codes for the protein of interest—becomes a transgenic expression cassette when it is modified by non-natural, synthetic “artificial” methods such as, for example, mutagenization. Such methods have been described (U.S. Pat. No. 5,565,350; WO 00/15815; also see above).

Further, the plant cell, plant tissue or plant can also be transformed such that further enzymes and proteins are (over)expressed or repressed or reduced for supporting an increased tolerance and/or resistance to environmental stress and increased biomass production as compared to a corresponding non-transformed wild type plant.

With regard to any nucleic acid sequence a nucleic acid construct which contains said nucleic acid sequence or an organism (=transgenic organism) which is transformed with said nucleic acid sequence or said nucleic acid construct, “transgene” means all those constructs which have been brought about by genetic manipulation methods and in which either
a) said nucleic acid sequence or a derivative thereof, or
b) a genetic regulatory element, for example a promoter, which is functionally linked to said nucleic acid sequence or a derivative thereof, or
c) (a) and (b)
   is/are not present in its/their natural genetic environment or has/have been modified by means of genetic manipulation methods, it being possible for the modification to be, by way of example, a substitution, addition, deletion, inversion or insertion of one or more nucleotides or nucleotide radicals.

“Natural genetic environment” means the natural chromosomal locus in the organism of origin or the presence in a genomic library. In the case of a genomic library, the natural, genetic environment of the nucleic acid sequence is preferably at least partially still preserved. The environment flanks the nucleic acid sequence at least on one side and has a sequence length of at least 50 bp, preferably at least 500 bp, particularly preferably at least 1000 bp, very particularly preferably at least 5000 bp.

However, transgenic also means that the nucleic acids according to the invention are located at their natural position in the genome of an organism, but that the sequence has been modified in comparison with the natural sequence and/or that the regulatory sequences of the natural sequences have been modified. Preferably, transgenic/recombinant is to be understood as meaning the expression of the nucleic acids used in the process according to the invention in a non-natural position in the genome, that is to say the expression of the nucleic acids is homologous or, preferably, heterologous. This expression can be transiently or of a sequence integrated stably into the genome.

The use of the nucleic acid sequence described herein in the process of the invention or of the nucleic acid construct or another embodiment according to this invention for the generation of transgenic plants is therefore also subject matter of the invention.

The term “transgenic plants” used in accordance with the invention refers to the progeny of a transgenic plant, for example the T₁, T₂, T₃ and subsequent plant generations or the BC₁, BC₂, BC₃ and subsequent plant generations. Thus, the transgenic plants according to the invention can be raised and selfed or crossed with other individuals in order to obtain further transgenic plants according to the invention. Transgenic plants may also be obtained by propagating transgenic plant cells vegetatively. The present invention also relates to transgenic plant material, which can be derived from a transgenic plant population according to the invention. Such material includes plant cells and certain tissues, organs and parts of plants in all their manifestations, such as seeds, leaves, anthers, fibers, tubers, roots, root hairs, stems, embryo, calli, cotelydons, petioles, harvested material, plant tissue, reproductive tissue and cell cultures, which are derived from the actual transgenic plant and/or can be used for bringing about the transgenic plant.

Any transformed plant obtained according to the invention can be used in a conventional breeding scheme or in in vitro plant propagation to produce more transformed plants with the same characteristics and/or can be used to introduce the same characteristic in other varieties of the same or related species. Such plants are also part of the invention. Seeds obtained from the transformed plants genetically also contain the same characteristic and are part of the invention. As mentioned before, the present invention is in principle applicable to any plant and crop that can be transformed with any of the transformation method known to those skilled in the art. In a specific embodiment the nucleic acid or the polypeptide which activity is reduced according to the process of the invention is mutated or otherwise reduced in its activity in a transformable crop variety. The genes or mutated version of the nucleic acid or the polypeptide conferring the reduction are later on transferred to a elite (commercial relevant) crop variety by for example (marker assisted) crossing, whereby the mutated or otherwise reduced version of the nucleic acid or polypeptide of the invention replace or repress the original or native and active one.

In an especially preferred embodiment, the organism, the host cell, plant cell, plant or plant tissue according to the invention is transgenic.

Accordingly, the invention therefore relates to transgenic organisms transformed with at least one nucleic acid molecule disclosed herein, e.g. the antisense, RNAi, snRNA, dsRNA, siRNA, miRNA, ta-siRNA, cosuppression construct, recombination construct or ribozyme molecule, or the viral nucleic acid molecule, nucleic acid construct or vector according to the invention, and to cells, cell cultures, tissues, parts—such as, for example, in the case of plant organisms, plant tissue, for example leaves, roots and the like—or propagation material derived from such organisms, or intact plants.

Accordingly, the present invention also relates to cells, cell cultures, tissues, parts—such as, for example, in the case of plant organisms, plant tissue, for example leaves, roots and the like—or propagation material derived from such organisms, or intact plants with an increased tolerance and/or resistance to environmental stress and increased biomass production as compared to a corresponding non-transformed wild type plant.

In particular the present invention also relates to cells, cell cultures, tissues, parts—such as, for example, in the case of plant organisms, plant tissue, for example leaves, roots and the like—or propagation material derived from such organisms, or intact plants which have reduced or deleted activity selected from the group consisting of: 1-phosphatidylinositol 4-kinase, amino acid permease (AAP1), At3g55990-protein, At5g40590-protein, ATP-dependent peptidase/ ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1 C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and/or ubiquitin conjugating enzyme/ubiquitin-like activating enzyme.

Further, the present invention also relates to cells, cell cultures, tissues, parts—such as, for example, in the case of plant organisms, plant tissue, for example leaves, roots and the like—or propagation material derived from such organisms, or intact plants comprising a reduced activity or expression of a nucleic acid molecule or polypeptide to be reduced according to the process of the invention.

Accordingly, the present invention in particular relates to cells, cell cultures, tissues, parts—such as, for example, in the case of plant organisms, plant tissue, for example leaves, roots and the like—or propagation material derived from such organisms, or intact plants comprising a reduced activity or expression of nucleic acid molecule comprising a nucleic acid molecule as depicted in column 5 or 7 of Table IA or B or comprising a reduced activity or expression of a polypeptide comprising a polypeptide as depicted in column 5 or 7 of Table IIA or B or comprising a consensus sequence or a polypeptide motif as depicted in column 7 of Table IV.

The terms “recombinant (host)” and “transgenic (host)”are used interchangeably in this context. Naturally, these terms refer not only to the host organism or target cell in question, but also to the progeny, or potential progeny, of these organisms or cells. Since certain modifications may occur in subsequent generations owing to mutation or environmental effects, such progeny is not necessarily identical with the parental cell, but still comes within the scope of the term as used herein.

Suitable organisms for the process according to the invention or as hosts are those as disclosed above. The organisms used as hosts are microorganisms, such as bacteria, fungi, yeasts or algae or plants, such as dicotyledonous or monocotyledonous plants.

In principle all plants can be used as host organism, especially the plants mentioned above as source organism. Preferred transgenic plants are, for example, selected from the families Aceraceae, Anacardiaceae, Apiaceae, Asteraceae, Brassicaceae, Cactaceae, Cucurbitaceae, Euphorbiaceae, Fabaceae, Malvaceae, Nymphaeaceae, Papaveraceae, Rosaceae, Salicaceae, Solanaceae, Arecaceae, Bromeliaceae, Cyperaceae, lridaceae, Liliaceae, Orchidaceae, Gentianaceae, Labiaceae, Magnoliaceae, Ranunculaceae, Carifolaceae, Rubiaceae, Scrophulariaceae, Caryophyllaceae, Ericaceae, Polygonaceae, Violaceae, Juncaceae or Poaceae and preferably from a plant selected from the group of the families Apiaceae, Asteraceae, Brassicaceae, Cucurbitaceae, Fabaceae, Papaveraceae, Rosaceae, Solanaceae, Liliaceae or Poaceae. Preferred are crop plants such as plants advantageously selected from the group of the genus peanut, oilseed rape, canola, sunflower, safflower, olive, sesame, hazelnut, almond, avocado, bay, pumpkin/squash, linseed, soya, pistachio, borage, maize, wheat, rye, oats, sorghum and millet, triticale, rice, barley, cassava, potato, sugarbeet, egg plant, alfalfa, and perennial grasses and forage plants, oil palm, vegetables (brassicas, root vegetables, tuber vegetables, pod vegetables, fruiting vegetables, onion vegetables, leafy vegetables and stem vegetables), buckwheat, Jerusalem artichoke, broad bean, vetches, lentil, dwarf bean, lupin, clover and Lucerne for mentioning only some of them.

Preferred plant cells, plant organs, plant tissues or parts of plants originate from the under source organism mentioned plant families, preferably from the abovementioned plant genus, more preferred from abovementioned plants spezies.

In one embodiment of the invention plant cells, plant organs, plant tissues or parts of plants are selected from the group comprising corn, soy, oil seed rape (including canola and winter oil seed reap), cotton, wheat and rice.

Yet another embodiment of the invention is a composition comprising the protein of the invention, the nucleic acid molecule of the invention, the polypeptide of the invention, the nucleic acid construct or the vector of the invention, the antagonist of the invention, the antibody of the invention and optionally a agricultural acceptable carrier.

In yet another aspect, the invention also relates to harvestable parts and to propagation material of the transgenic plants according to the invention which either contain transgenic plant cells expressing a nucleic acid molecule according to the invention or which contains cells which show a reduced, repressed, decreased or deleted cellular activity selected from the group consisting of: 1-phosphatidylinositol 4-kinase, amino acid permease (AAP1), At3g55990-protein, At5g40590-protein, ATP-dependent peptidase/ATPase/nucleoside- triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and/or ubiquitin conjugating enzyme/ubiquitin-like activating enzyme, e.g. which show a reduced, repressed, decreased or deleted activity of the polypeptide or the nucleic acid molecule to be reduced in the process of the invention, in particular a reduced or deleted activity of a polypeptide comprising a polypeptide as depicted in column 5 or 7 of Table II, preferably as depicted in Table IIB, or comprising a consensus sequence or a polypeptide motif as depicted in column 7 of Table IV, or of a gene product of a nucleic acid molecule comprising the polynucleotide as depicted in column 5 or 7 of Table I, preferably as depicted in Table IB.

Harvestable parts can be in principle any useful parts of a plant, for example, flowers, pollen, seedlings, tubers, leaves, stems, fruit, seeds, roots etc. Propagation material includes, for example, seeds, fruits, cuttings, seedlings, tubers, rootstocks etc. Preferred are seeds, seedlings, tubers or fruits as harvestable or propagation material.

In one embodiment, the present invention relates to a method for the identification of a gene product conferring an increased tolerance and/or resistance to environmental stress and increased biomass production as compared to a corresponding non-transformed wild type plant, comprising the following steps:
a) contacting, e.g. hybridising, the one, some or all nucleic acid molecules of a sample, e.g. cells, tissues, plants or microorganisms or a nucleic acid library , which can contain a candidate gene encoding a gene product conferring an increased tolerance and/or resistance to environmental stress and increased biomass production as compared to a corresponding non-transformed wild type plant after reduction or deletion of its expression, with a nucleic acid molecule as depicted in column 5 or 7 of Table IA or B or a functional homologue thereof;
b) identifying the nucleic acid molecules, which hybridize under relaxed stringent conditions with said nucleic acid molecule, in particular to the nucleic acid molecule sequence as depicted in column 5 or 7 of Table I and, optionally, isolating the full length cDNA clone or complete genomic clone;
c) identifying the candidate nucleic acid molecules or a fragment thereof in host cells, preferably in a plant cell
d) reducing or deletion the expressing of the identified nucleic acid molecules in the host cells;
e) assaying the level of tolerance and/or resistance to environmental stress and biomass production as compared to a corresponding non-transformed wild type plant in the host cells; and
f) identifying the nucleic acid molecule and its gene product which reduction or deletion of expression confers an increased tolerance and/or resistance to environmental stress and increased biomass production as compared to a corresponding non-transformed wild type plant in the host cell after expression compared to the wild type.

Relaxed hybridisation conditions are: After standard hybridisation procedures washing steps can be performed at low to medium stringency conditions usually with washing conditions of 40° -55° C. and salt conditions between 2×SSC and 0,2×SSC with 0,1% SDS in comparison to stringent washing conditions as e.g. 60° to 68° C. with 0.1% SDS. Further examples can be found in the references listed above for the stringend hybridization conditions. Usually washing steps are repeated with increasing stringency and length until a useful signal to noise ratio is detected and depend on many factors as the target, e.g. its purity, GC-content, size etc, the probe, e.g.its length, is it a RNA or a DNA probe, salt conditions, washing or hybridisation temperature, washing or hybridisation time etc.

In another embodiment, the present invention relates to a method for the identification of a gene product the reduction of which confers an increased tolerance and/or resistance to environmental stress and increased biomass production as compared to a corresponding non-transformed wild type plant, comprising the following steps:
a) identifiying a nucleic acid molecule in an organism, which is at least 20%, preferably 25%, more preferably 30%, even more preferred are 35%. 40% or 50%, even more preferred are 60%, 70% or 80%, most preferred are 90% or 95% or more homolog to the nucleic acid molecule encoding a protein comprising the polypeptide molecule as depicted in column 5 or 7 of Table II or comprising a consensus sequence or a polypeptide motif as depicted in column 7 of Table IV or being encoded by a nucleic acid molecule comprising a polynucleotide as depicted in column 5 or 7 of Table I or a homologue thereof as described herein , for example via homology search in a data bank;
b) repressing, reducing or deleting the expression of the identified nucleic acid molecules in the host cells;
c) assaying the level of tolerance and/or resistance to environmental stress and biomass production as compared to a corresponding non-transformed wild type plant; and
d) identifying the host cell, in which the repressing, reducing or deleting of the nucleic acid molecule or its gene product confers an increased tolerance and/or resistance to environmental stress and increased biomass production as compared to a corresponding non-transformed wild type plant.

In another embodiment, the present invention relates to a method for the identification of a gene product the reduction of which confers an increased tolerance and/or resistance to environmental stress and increased biomass production as compared to a corresponding non-transformed wild type plant, comprising the following steps:
a) providing an organism or host cells according to the invention, in which an nucleic acid molecule encoding a protein comprising the polypeptide has been inactivated, deleted or otherwise reduced in its activity;
b) transforming the organism with an cDNA expression or an genomic library or any other nucleic acid library capable of efficiently expressing the encompassed nucleic acid sequence
c) assaying the level of tolerance and/or resistance to environmental stress and biomass production as compared to a corresponding non-transformed wild type plant; and
d) identifying the host cell, in which the introduced nucleic acid sequence reverses the increased tolerance and/or resistance to environmental stress and increased biomass production as compared to a corresponding non-transformed wild type plant, reestablishing the wild type situation.

In one embodiment the different methods for the identification of a gene product the reduction of which confers an increased tolerance and/or resistance to environmental stress and increased biomass production as compared to a corresponding non-transformed wild type plant can be combined in any combination in order to optimize the method.

Furthermore, in one embodiment, the present invention relates to a method for the identification of a compound stimulating the increased tolerance and/or resistance to environmental stress and increased biomass production as compared to a corresponding non-transformed wild type plant to said plant comprising:
a) contacting cells which express the polypeptide as depicted in column 5 or 7 of Table II or being ecoded by a nucleic acid molecule comprising a polynucleotide as depicted in column 5 or 7 of Table I or a homologue thereof as described herein or its mRNA with a candidate compound under cell cultivation conditions;
b) assaying a reduction, decrease or deletion in expression of said polypeptide or said mRNA;
c) comparing the expression level to a standard response made in the absence of said candidate compound; whereby, a reduced, decreased or deleted expression over the standard indicates that the compound is stimulating the increased tolerance and/or resistance to environmental stress and increased biomass production as compared to a corresponding non-transformed wild type plant.

Furthermore, in one embodiment, the present invention relates to a method for the screening for antagonists of the activity of the polypeptide as depicted in column 5 or 7 of Table II or being ecoded by a nucleic acid molecule comprising a polynucleotide as depicted in column 5 or 7 of Table I or a homologue thereof as described herein, e.g. a polypeptide conferring an increased tolerance and/or resistance to environmental stress and increased biomass production as compared to a corresponding non-transformed wild type plant after decreasing its cellular activity, e.g. of the activity of a polypeptide having the activity represented by the protein or nucleic acid molecule to be reduced in the process of the invention or of the polypeptide of the invention comprising:
a) contacting cells, tissues, plants or microorganisms which express the polypeptide according to the invention with a candidate compound or a sample comprising a plurality of compounds under conditions which permit the expression the poly-peptide of the present invention;
b) assaying the tolerance and/or resistance to environmental stress and biomass production level or the polypeptide expression level in the cell, tissue, plant or microorganism or the media the cell, tissue, plant or microorganisms is cultured or maintained in; and
c) identifying an antagonist by comparing the measured tolerance and/or resistance to environmental stress and biomass production level or polypeptide expression level with a standard tolerance and/or resistance to environmental stress and biomass production level or polypeptide expression level measured in the absence of said candidate compound or a sample comprising said plurality of compounds, whereby an increased level of the tolerance and/or resistance to environmental stress and biomass production over the standard indicates that the compound or the sample comprising said plurality of compounds is an antagonist.

Yet another embodiment of the invention relates to a process for the identification of a compound conferring increased tolerance and/or resistance to environmental stress and increased biomass production as compared to a corresponding non-transformed wild type plant in a plant; comprising the following step:
a) culturing or maintaining a plant or animal cell or their tissues or microorganism expressing a polypeptide as depicted in column 5 or 7 of Table II or being encoded by a nucleic acid molecule comprising a polynucleotide as depicted in column 5 or 7 of Table I or a homologue thereof as described herein or a polynucleotide encoding said polypeptide and providing a readout system capable of interacting with the polypeptide under suitable conditions which permit the interaction of the polypeptide with this readout system in the presence of a chemical compound or a sample comprising a plurality of chemical compounds and capable of providing a detectable signal in response to the binding of a chemical compound to said polypeptide under conditions which permit the depression of said readout system and of the protein as depicted in column 5 or 7 of Table II or being ecoded by a nucleic acid molecule comprising a polynucleotide as depicted in column 5 or 7 of Table I or a homologue thereof as described herein; and
b) identifying if the chemical compound is an effective antagonist by detecting the presence or absence or decrease or increase of a signal produced by said readout system.

Said compound may be chemically synthesized or microbiologically produced and/or comprised in, for example, samples, e.g., cell extracts from, e.g., plants, animals or microorganisms, e.g. pathogens. Furthermore, said compound(s) may be known in the art but hitherto not known to be capable of suppressing the poly-peptide of the present invention. The reaction mixture may be a cell free extract or may comprise a cell or tissue culture. Suitable set ups for the process for identification of a compound of the invention are known to the person skilled in the art and are, for example, generally described in Alberts et al., Molecular Biology of the Cell, third edition (1994), in particular Chapter 17. The compounds may be, e.g., added to the reaction mixture, culture medium, injected into the cell or sprayed onto the plant.

If a sample containing a compound is identified in the process, then it is either possible to isolate the compound from the original sample identified as containing the compound capable of increasing tolerance and/or resistance to environmental stress and the biomass production as compared to a corresponding non-transformed wild type plant, or one can further subdivide the original sample, for example, if it consists of a plurality of different compounds, so as to reduce the number of different substances per sample and repeat the method with the subdivisions of the original sample. Depending on the complexity of the samples, the steps described above can be performed several times, preferably until the sample identified according to the said process only comprises a limited number of or only one substance(s). Preferably said sample comprises substances of similar chemical and/or physical properties, and most preferably said substances are identical. Preferably, the compound identified according to the described method above or its derivative is further formulated in a form suitable for the application in plant breeding or plant cell and tissue culture.

The compounds which can be tested and identified according to said process may be expression libraries, e.g., cDNA expression libraries, peptides, proteins, nucleic acids, antibodies, small organic compounds, hormones, peptidomimetics, PNAs or the like (Milner, Nature Medicine 1 (1995), 879-880; Hupp, Cell 83 (1995), 237-245; Gibbs, Cell 79 (1994), 193-198 and references cited supra). Said compounds can also be functional derivatives or analogues of known inhibitors or activators. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art. Furthermore, peptidomimetics and/or computer aided design of appropriate derivatives and analogues can be used, for example, according to the methods described above. The cell or tissue that may be employed in the process preferably is a host cell, plant cell or plant tissue of the invention described in the embodiments hereinbefore.

Thus, in a further embodiment the invention relates to a compound obtained or identified according to the method for identifiying an antagonist of the invention said compound being an antagonist of the polypeptide of the present invention.

Accordingly, in one embodiment, the present invention further relates to a compound identified by the method for identifying a compound of the present invention.

Said compound is, for example, an antagonistic homolog of the poly-peptide of the present invention. Antagonistic homologues of the polypeptide to be reduced in the process of the present invention can be generated by mutagenesis, e.g., discrete point mutation or truncation of the polypeptide of the present invention. As used herein, the term “antagonistic homologue” refers to a variant form of the protein, which acts as an antagonist of the activity of the polypeptide of the present invention. An anatgonist of a protein as depicted in column 5 or 7 of Table II or being ecoded by a nucleic acid molecule comprising a polynucleotide as depicted in column 5 or 7 of Table I or a homologue thereof as described herein, has at least partly lost the biological activities of the polypeptide of the present invention. In particular, said antagonist confers a decrease of the expression level of the polypeptide as depicted in column 5 or 7 of Table II or being ecoded by a nucleic acid molecule comprising a polynucleotide as depicted in column 5 or 7 of Table I or a homologue thereof as described herein and thereby the expression of said antagonist in an organisms or part thereof confers the increased tolerance and/or resistance to environmental stress and increased biomass production as compared to a corresponding non-transformed wild type plant. A typical antagonsist in that sense would be a dominant negative version of the nucleic acid molecule or polypeptide which activity is to be reduced in the process of the invention, for example a protein which still can partizipates in a protein complex, but cannot anymore fulfill its orginal biological, for example enzymatical function, thereby nearly inactivating the complete complex.

In one embodiment, the invention relates to an antibody specifically recognizing the compound or antagonist of the present invention.

The invention also relates to a diagnostic composition comprising at least one of the aforementioned nucleic acid molecules, antisense nucleic acid molecule, RNAi, snRNA, dsRNA, siRNA, miRNA, ta-siRNA, cosuppression molecule, ribozyme, vectors, proteins, antibodies or compounds of the invention and optionally suitable means for detection.

The diagnostic composition of the present invention is suitable for the isolation of mRNA from a cell and contacting the mRNA so obtained with a probe comprising a nucleic acid probe as described above under hybridizing conditions, detecting the presence of mRNA hybridized to the probe, and thereby detecting the expression of the protein in the cell. Further methods of detecting the presence of a protein according to the present invention comprise immunotechniques well known in the art, for example enzyme linked immunoadsorbent assay. Furthermore, it is possible to use the nucleic acid molecules according to the invention as molecular markers or primers in plant breeding. Suitable means for detection are well known to a person skilled in the art, e.g. buffers and solutions for hydridization assays, e.g. the aforementioned solutions and buffers, further and means for Southern-, Western-, Northern etc. -blots, as e.g. described in Sambrook et al. are known. In one embodiment diagnostic composition contain PCR primers designed to specifically detect the presense or the expression level of the nucleic acid molecule to be reduced in the process of the invention, e.g. of the nucleic acid molecule of the invention, or to descriminate between different variants or alleles of the nucleic acid molecule of the invention or which activity is to be reduced in the process of the invention.

In another embodiment, the present invention relates to a kit comprising the nucleic acid molecule, the vector, the host cell, the polypeptide, or the antisense, RNAi, snRNA, dsRNA, siRNA, miRNA, ta-siRNA, cosuppression molecule, or ribozyme molecule, or the viral nucleic acid molecule, the antibody, plant cell, the plant or plant tissue, the harvestable part, the propagation material and/or the compound and/or antagonist identified according to the method of the invention.

The compounds of the kit of the present invention may be packaged in containers such as vials, optionally with/in buffers and/or solution. If appropriate, one or more of said components might be packaged in one and the same container. Additionally or alternatively, one or more of said components might be adsorbed to a solid support as, e.g. a nitrocellulose filter, a glas plate, a chip, or a nylon membrane or to the well of a micro titerplate. The kit can be used for any of the herein described methods and embodiments, e.g. for the production of the host cells, transgenic plants, pharmaceutical compositions, detection of homologous sequences, identification of antagonists or agonists, as food or feed or as a supplement thereof or as supplement for the treating of plants, etc.

Further, the kit can comprise instructions for the use of the kit for any of said embodiments, in particular for the use for producing organisms or part thereof having an increased tolerance and/or resistance to environmental stress and increased biomass production as compared to a corresponding non-transformed wild type plant.

In one embodiment said kit comprises further a nucleic acid molecule encoding one or more of the aforementioned protein, and/or an antibody, a vector, a host cell, an antisense nucleic acid, a plant cell or plant tissue or a plant. In another embodiment said kit comprises PCR primers to detect and discrimante the nucleic acid molecule to be reduced in the process of the invention, e.g. of the nucleic acid molecule of the invention.

In a further embodiment, the present invention relates to a method for the production of an agricultural composition providing the nucleic acid molecule for the use according to the process of the invention, the nucleic acid molecule of the invention, the vector of the invention, the antisense, RNAi, snRNA, dsRNA, siRNA, miRNA, ta-siRNA, cosuppression molecule, ribozyme, or antibody of the invention, the viral nucleic acid molecule of the invention, or the polypeptide of the invention or comprising the steps of the method according to the invention for the identification of said compound or antagonist; and formulating the nucleic acid molecule, the vector or the poly-peptide of the invention or the antagonist, or compound identified according to the methods or processes of the present invention or with use of the subject matters of the present invention in a form applicable as plant agricultural composition.

In another embodiment, the present invention relates to a method for the production of supporting plant culture composition comprising the steps of the method of the present invention; and formulating the compound identified in a form acceptable as agricultural composition.

Under “acceptable as agricultural composition” is understood, that such a composition is in agreement with the laws regulating the content of fungicides, plant nutrients, herbicides, etc. Preferably such a composition is without any harm for the protected plants and the animals (humans included) fed therewith.

The nucleic acid molecules disclosed herein, in particular the nucleic acid as depicted column 5 or 7 of Table IA or B, have a variety of uses. First, they may be used to identify an organism or a close relative thereof. Also, they may be used to identify the presence thereof or a relative thereof in a mixed population of plants. By probing the extracted genomic DNA of a culture of a unique or mixed population of plants under stringent conditions with a probe spanning a region of the gene of the present invention which is unique to this, one can ascertain whether the present invention has been used or whether it or a close relative is present.

Further, the nucleic acid molecule disclosed herein, in particular the nucleic acid molecule as depicted column 5 or 7 of Table IA or B, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule as depicted column 5 or 7 of Table IA or B, or homologous thereof may lead to variation in the activity of the proteins disclosed herein, in particular the proteins comprising polypeptides as depicted in column 5 or 7 of Table IIA or B or comprising the consensus sequence or the polypeptide motif as shown in column 7 of Table IV, and their homolgos and in consequence in natural variation.

In consequence natural variation eventually also exists in form of less active allelic variants leading already to a relative increased tolerance and/or resistance to environmental stress and increased biomass production.

Accordingly, the present invention relates to a method for breeding plants, comprising
a) selecting a first plant variety with increased tolerance and/or resistance to environmental stress and increased biomass production as compared to a corresponding non-transformed wild type plant by reducing, repressing, decreasing or deleting the expression of a polypeptide or nucleic acid molecule which activity is reduced in the process of the present invention, e.g. as disclosed herein, in particular of a nucleic acid molecule comprising a nucleic acid molecule as depicted in column 5 or 7 of Table IA or B or a polypeptide comprising a polypeptide as shown in column 5 or 7 of Table IIA or B or comprising a consensus sequence or a polypeptide motif as depicted in column 7 of Table IV, or a homologue thereof as described herein;
b) associating the increased tolerance and/or resistance to environmental stress and increased biomass production as compared to a corresponding non-transformed wild type plant with the expression level or the genomic structure of a gene encoding said polypeptide or said nucleic acid molecule;
c) crossing the first plant variety with a second plant variety, which significantly differs in its tolerance and/or resistance to environmental stress and its biomass production; and
d) identifying, which of the offspring varieties has got the increased tolerance and/or resistance to environmental stress and increased biomass production as compared to a corresponding non-transformed wild type plant by means of analyzing level of tolerance and/or resistance to environmental stress and biomass production or the expression of said polypeptide or nucleic acid molecule or the genomic structure of the genes encoding said polypeptide or nucleic acid molecule of the invention.
   In one embodiment, the expression level of the gene according to step (b) is reduced.

The nucleic acid molecules of the invention are also useful for evolutionary and protein structural studies. By comparing the sequences, e.g. as depicted in column 5 or 7 of Table I, to those encoding similar enzymes from other organisms, the evolutionary relatedness of the organisms can be assessed. Similarly, such a comparison permits an assessment of which regions of the sequence are conserved and which are not, which may aid in determining those regions of the protein which are essential for the functioning of the enzyme. This type of determination is of value for protein engineering studies and may give an indication of what the protein can tolerate in terms of mutagenesis without losing function.

Accordingly, the nucleic acid molecule disclosed herein, e.g. the nucleic acid molecule which activity is to be reduced according to the process of the invention, e.g. as depicted in column 5 or 7 of Table I, or a homologue thereof, can be used for the identification of other nucleic acids conferring an increased tolerance and/or resistance to environmental stress and increased biomass production as compared to a corresponding non-transformed wild type plant after reduction, repression, decrease or deletion of their expression.

Further, disclosed herein, e.g. the nucleic acid molecule which activity is to be reduced according to the process of the invention, e.g. as depicted in column 5 or 7 of Table I, or a homologue thereof, in particular the nucleic acid molecule of the invention, or a fragment or a gene conferring the expression of the encoded expression product, e.g. the polypeptide of the invention, can be used for marker assisted breeding or association mapping of the tolerance and/or resistance to environmental stress and biomass production related traits.

These and other embodiments are disclosed and encompassed by the description and examples of the present invention.

Further literature concerning any one of the methods, uses and compounds to be employed in accordance with the present invention may be retrieved from public libraries, using for example electronic devices.

For example the public database “Medline” may be utilized which is available on the Internet, for example under hftp://www.ncbi.nlm.nih.gov/PubMed/medline.html. Further databases and addresses, such as hftp://www.ncbi.nlm.nih.gov/, hftp://www.infobiogen. fr/, hftp://www.fmi.ch/biology/research-tools.html, hftp://www.tigr.org/, are known to the person skilled in the art and can also be obtained using, e.g., hftp://www.lycos.com. An overview of patent information in biotechnology and a survey of relevant sources of patent information useful for retrospective searching and for current awareness is given in Berks, TIBTECH 12 (1994), 352-364.

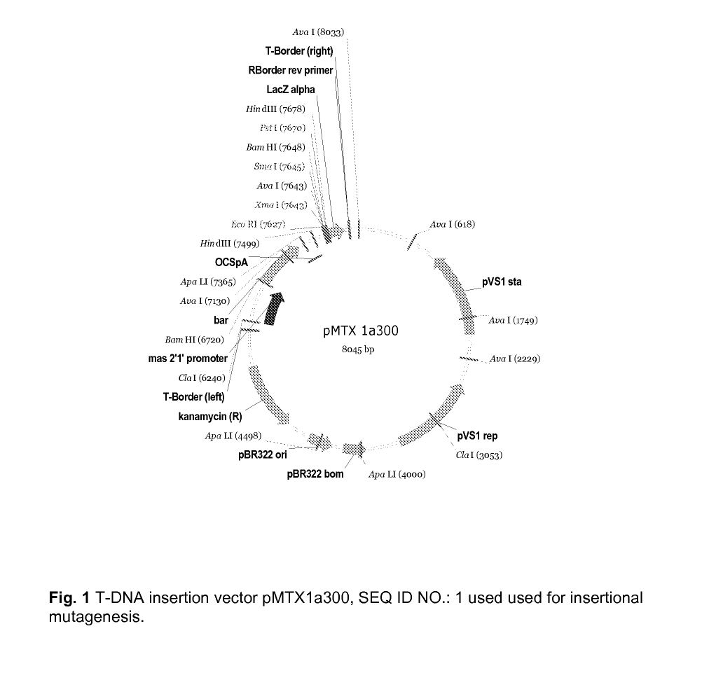

The present invention is illustrated by the examples and the figures (FIG. 1-3), which follow:

Example

Engineering of Arabidopsis plants by inactivation or down-regulation of stress related genes.

Vector Preparation

A binary knock out vector was constructed based on the modified pPZP binary vector backbone (comprising the kanamycin-gene for bacterial selection; Hajdukiewicz, P. et al., 1994, Plant Mol. Biol., 25: 989-994) and the selection marker bargene (De Block et al., 1987, EMBO J. 6, 2513-2518) driven by the mas2′1′ and mas271f promoters (Velten et al., 1984, EMBO J. 3, 2723-2730; Mengiste, Amedeo and Paszkowski, 1997, Plant J., 12, 945-948). The resulting vector, used for insertional mutagenesis, was pMTX1a300 SEQ ID NO.: 1.

Examples of other usable binary vectors for insertional mutagenesis are pBIN19, pBI101, pBinAR, pSun or pGPTV. An overview over binary vectors and their specific features is given in Hellens et al., 2000, Trends in plant Science, 5:446-451and in Guerineau F., Mullineaux P., 1993, Plant transformation and expression vectors in plant molecular biology, LABFAX Series, (Croy R. R. D., ed.) pp. 121-127 Bios Scientific Publishers, Oxford

Transformation of Agrobacteria

The plasmid was transformed into Agrobacterium tumefaciens (GV3101pMP90; Koncz and Schell, 1986 Mol. Gen. Genet. 204:383-396) using heat shock or electroporation protocols. Transformed colonies were grown on YEB medium and selected by respective antibiotics (Rif/Gent/Km) for 2 d at 28 C. These agrobacteria cultures were used for the plant transformation.

Arabidopsis thaliana of the ecotype C24 were grown and transformed according to standard conditions (Bechtold, N., Ellis, J., Pelletier, G. 1993. In planta Agrobacterium mediated gene transfer by infiltration of Arabidopsis thaliana plants, C. R. Acad. Sci. Paris 316:1194-1199; Bent, A. F., Clough, J. C., 1998; Floral dip: a simplified method for Agrobacterium-mediated transformation of Arabidopsis thaliana, PLANT J. 16:735-743).

Transformed plants (F1) were selected by the use of their respective resistance marker. In case of BASTA®-resistance, plantlets were sprayed four times at an interval of 2 to 3 days with 0.02% BASTA® and transformed plants were allowed to set seeds. 50-100 seedlings (F2) were subjected again to marker selection, in case of BASTA-resistance by spaying with 0.1% BASTA® on 4 consecutive days during the plantlet phase. Plants segregating for a single resistance locus (approximately 3:1 resistant seedling to sensitive seedlings) were chosen for further analysis. From these lines three of the resistant seedlings (F2) were again allowed to set seeds and were tested for homozygosis through in-vitro germination of their seeds (F3) on agar medium containing the selection agent (BASTA®, 15 mg/L ammonium glufosinate, Pestanal, Riedel de Haen, Seelze, Germany). Those F2 lines which showed nearly 100% resistant offspring (F3) were considered homozygote and taken for functional analysis.

Measurement of Stress Tolerance and Increased Biomass

Transformed A. thaliana plants were grown individually in pots containing a 4:1 (v/v) mixture of soil and quartz sand in a growth chamber (York Industriekalte GmbH, Mannheim, Germany). To induce germination, sown seeds were kept at 4° C., in the dark, for 3 days. Subsequently conditions were changed for 3 d to 20° C/6° C. day/night temperature with a 16/8 h day-night cycle at 150 μE/m²s. Standard growth conditions were: photoperiod of 16 h light and 8 h dark, 20° C., 60% relative humidity, and a photon flux density of 200 μE. Plants were watered daily until they were approximately 3 weeks old at which time drought was imposed by withholding water. After approximately 12 days of withholding water, most plants showed visual symptoms of injury, such as wilting and leaf browning, whereas tolerant or resistant plants were identified as being visually turgid and healthy green in color. Plants were scored for symptoms of drought symptoms and biomass production comparison to wild type and neighboring plants for 5-6 days in succession.

Three successive experiments were conducted. In the first experiment, one individual of each transformed line was tested.

In the second experiment, the lines that had been scored as drought tolerant or resistant in the first experiment, i.e. survived longer than the wild type control and showed increaed biomass production in comparison to wild type and neighbouring plants, were put through a confirmation screen according to the same experimental procedures. In this experiment, max. 10 plants of each tolerant or resistant line were grown, treated and scored as before.

In the first two experiments, resistance or tolerance biomass production was measured compared to neighboring and wild type plants.

In the third experiment (table 1), 15 replicates of each confirmed tolerant line, i.e. those that had been scored as tolerant or resistant in the second experiment, were grown, treated and scored as before.

In the third experiment, after approximately 10 days of drought, the control (non-transformed Arabidopsis thaliana) and most transformed lines in the test showed extreme visual symptoms of stress including necrosis and cell death. Several transformed plants retained viability as shown by their turgid appearance and maintenance of green color.

Table 1:

Table 1: Duration of survival and biomass production of transformed Arabidopsis thaliana after imposition of drought stress on 3-week-old plants. Drought tolerance and biomass production was measured visually at daily intervals. Average performance is the average of transgenic plants that survived longer than the wild type control. Maximum performance is the longest period that any single transformed plant survived longer than the wild type control. Average biomass is the average of days of transgenic plants plants increase in biomass in comparison to the wild type control and neighbouring plants. Maximum biomass is the longest period that any single transformed plant showed increase in biomass in comparison to the wild type control and neighbouring plants.

TABLE 1

| | | Average | | | |
|---|---|---|---|---|---|
| | | Perfor- | Maximum | Average | Maximum |
| SeqID | Locus | mance | Performance | Biomass | Biomass |
| 1418 | At5g50870 | 2.7 | 5 | 0.6 | 2 |
| 1025 | At4g31120 | 4.7 | 5 | 0.5 | 5 |
| 729 | At3g14230 | 1.8 | 4 | 1.2 | 3 |
| 27 | At1g12110 | 3 | 5 | 1.8 | 3 |
| 104 | At1g13270 | 2.9 | 5 | 1.4 | 3 |
| 190 | At1g27080 | 2.9 | 5 | 1.3 | 2 |
| 512 | AT1G58360 | 2.5 | 4 | 1 | 2 |
| 1464 | At5g60780 | 2.4 | 5 | 1.3 | 3 |
| 813 | At3g54920 | 2.7 | 4 | 1 | 3 |
| 673 | AT2G03670 | 2.8 | 5 | 1.5 | 4 |
| 27 | At1g12110 | 2.7 | 5 | 1.7 | 4 |
| 512 | AT1G58360 | 2.9 | 5 | 1.4 | 2 |
| 1385 | At5g40590 | 2.5 | 5 | 0.9 | 2 |
| 410 | At1g33760 | 2.7 | 5 | 1.2 | 4 |
| 923 | At4g13430 | 2.5 | 5 | 1.3 | 4 |
| 1593 | At5g66160 | 4 | 5 | 0.1 | 0.1 |
| 1083 | At5g02330 | 2.2 | 4 | 1 | 3 |
| 1551 | At5g64070 | 2.3 | 4 | 0.7 | 2 |
| 1650 | At3g55990 | 4.5 | 5 | 2.2 | 4 |

Analysis of the Selected Stress Resistant Lines

Since the lines were preselected for single insertion loci and a homozygous situation of the resistance marker, the disruption (or mutation) of single genes through the integration of the T-DNA were expected to have lead to the stress-resistant phenotype. Lines which showed a consistent phenotype were chosen for molecular analysis.

Genomic DNA was purified from approximately 100 mg of leaf tissue from these lines using standard procedures (either spins columns from Qiagen, Hilden, Germany or the Nucleon Phytopure Kit from Amersham Biosciences, Freiburg, Germany). The amplification of the insertion side of the T-DNA was achieved using two different methods. Either by an adaptor PCR-method according to Spertini D, Béliveau C. and Bellemare G., 1999, Biotechniques, 27, 308-314 using T-DNA specific primers LB1 (5′-TGA CGC CAT TTC GCC TTT TCA-3′; SEQ ID NO: 4) or RB 1-2 (5′-CAA CTT AAT CGC CTT GCA GCA CA-3′; SEQ ID NO: 5) for the first and LB2 (5′-CAG AAA TGG ATA AAT AGC CTT GCT TCC-3′; SEQ ID NO: 6) or RB4-2 (5′-AGC TGG CGT AAT AGC GAA GAG-3′; SEQ ID NO: 7) for the second PCR respectively. Alternatively TAIL-PCR (Liu Y-G, Mitsukawa N, Oosumi T and Whittier R F, 1995, Plant J. 8, 457-463) was performed. In this case for the first PCR LB1 (5′-TGA CGC CAT TTC GCC TTT TCA-3′, SEQ ID NO: 4) or RB1-2 (5′-CAA CTT AAT CGC CTT GCA GCA CA-3′; SEQ ID NO: 5), for the second PCR LB2 (5′ -CAG AAA TGG ATA AAT AGC CTT GCT TCC-3′; SEQ ID NO: 6) or RB4-2 (5′-AGC TGG CGT AAT AGC GAA GAG- 3′, SEQ ID NO: 7) and for the last PCR LB3 (5′-CCA ATA CAT TAC ACT AGC ATC TG-3′; SEQ ID NO: 8) or RB5 (5′-AAT GCT AGA GCA GCT TGA-3′; SEQ ID NO: 9) were used as T-DNA specific primers for left or right T-DNA borders respectively.

Appropriate PCR-products were identified on agarose gels and purified using columns and standard procedures (Qiagen, Hilden, Germany). PCR-products were sequenced with additional T-DNA-specific primers located towards the borders relative to the primers used for amplification. For adaptor PCR products containing left border sequences primer LBseq (5′-CAA TAC ATT ACA CTA GCA TCT G-3′; SEQ ID NO: 10) and for sequences containing right border sequences primer RBseq (5′-AGA GGC CCG CAC CGA TCG-3′; SEQ ID NO: 11) were used for sequencing reactions. For TAIL PCR products containing left border sequences primer LBseq2 (5′-CTA GCA TCT GAA TTT CAT AAC C-3′; SEQ ID NO: 12) and for PCR products containing right border sequences primer RBseq2 (5′-GCT TGA GCT TGG ATC AGA TTG-3′; SEQ ID NO: 13) were used for sequencing reactions. The resulting sequences were taken for comparison with the available Arabidopsis genome sequence from Genbank using the blast algorithm (Alt-schul et al., 1990. J Mol Biol, 215:403-410).

Further details on PCR products used to identify the genomic locus are given in table 2 below. Indicated are the identified annotated open reading frame in the Arabidopsis genome, the estimated size of the obtained PCR product (in base pairs), the T-DNA border (LB: left border, RB: right border) for which the amplification was achieved, the method which resulted in the indicated PCR product (explanation see text above), the respective restriction enzymes in case of adaptor PCR, and the degenerated primer in the case of TAIL PCR. Routinely degenerated primers
ADP2 (5′-NGT CGA SWG ANA WGA A-3′; SEQ ID NO: 14),
ADP3 (5′-WGTGNAGWANCANAGA-3′; SEQ ID NO: 15),
ADP5 (5′-STT GNT AST NCT NTG C-3′; SEQ ID NO: 16),
ADP6 (5′-AGWGNAGWANCANAGA-3′; SEQ ID NO: 17),
ADP8 (5′-NTGCGASWGANWAGAA-3′; SEQ ID NO: 18),
ADP9 (5′-NTG CGA SWG ANT AGA A-3′; SEQ ID NO: 19) and
ADP11 (5′-SST GGS TAN ATW ATW CT-3′; SEQ ID NO: 20) were used.

The identification of the insertion locus in each case was confirmed by a control PCR, using one of the above mentioned T-DNA-specific primers and a primer deduced from the identified genomic locus, near to the insertion side. The amplification of a PCR-product of the expected size from the insertion line using these two primers proved the disruption of the identified locus by the T-DNA integration.

Table 2: Details on PCR products used to identify the down-regulated gene in lines showing increased tolerance and/or resistance to environmental stress and increased biomass production as compared to a corresponding non-transformed wild type plant. The down regulated gene is defined by its TAIR Locus (Locus).

TABLE 2
PCR-products

| | | | | Restriction enzyme |
|---|---|---|---|---|
| | | | | or deg. |
| SEQ ID | Locus | Border | Method | primer |
| 1418 | At5g50870 | RB | Adapter | MunI |
| 1025 | At4g31120 | LB | Adapter | SpeI |
| 729 | At3g14230 | LB | Adapter | BgllI |
| 27 | At1g12110 | LB | Adapter | BgllI |
| 104 | At1g13270 | LB | Adapter | MunI |
| 190 | At1g27080 | RB | Adapter | Psp1406I/Bsp119I |
| 512 | AT1G58360 | RB | Adapter | MunI |
| 1464 | At5g60780 | LB | Adapter | Psp1406I/Bsp119I |
| 813 | At3g54920 | LB | Adapter | BgllI |
| 673 | AT2G03670 | LB | Adapter | BgllI |
| 27 | At1g12110 | LB | Adapter | BgllI |
| 512 | AT1G58360 | RB | Adapter | MunI |
| 1385 | At5g40590 | LB | Adapter | SpeI |
| 410 | At1g33760 | LB | Adapter | SpeI |
| 923 | At4g13430 | LB | Adapter | Psp1406I/Bsp119I |
| 1593 | At5g66160 | RB | Adapter | SpeI |
| 1083 | At5g02330 | LB | Adapter | MunI |
| 1551 | At5g64070 | RB | Adapter | SpeI |
| 1650 | At3g55990 | LB | Adapter | Psp1406I/Bsp119I |

Column 1 refers to the SEQ ID NO.: of the gene which has been knocked out, column 2 refers to the TAIR Locus of the knocked out gene (Locus), column 3 refers to the T-DNA border for which the PCR product was amplified, column 4 refers to the PCR method for amplification and column 5 refers to restriction enzyme of degenerate primer used in the PCR method (for detailed examplation to columns 4 and 5 see text above; APX means either primer AP2, primer AP5, primer AP6, primer AP9 or primer AP11)

Construction of antisense constructs for repression of the activity or expression of a gene, e.g. a gene comprising SEQ ID NO.: 1025

Construction of antisense constructs for repression of the activity or expression of a gene, e.g. a gene comprising SEQ ID NO.: 104

Construction of antisense constructs for repression of the activity or expression of a gene, e.g. a gene comprising SEQ ID NO.: 190

Construction of antisense constructs for repression of the activity or expression of a gene, e.g. a gene comprising SEQ ID NO.: 410

Construction of antisense constructs for repression of the activity or expression of a gene, e.g. a gene comprising SEQ ID NO.: 512

Construction of antisense constructs for repression of the activity or expression of a gene, e.g. a gene comprising SEQ ID NO.: 673

Construction of antisense constructs for repression of the activity or expression of a gene, e.g. a gene comprising SEQ ID NO.: 729

Construction of antisense constructs for repression of the activity or expression of a gene, e.g. a gene comprising SEQ ID NO.: 27

Construction of antisense constructs for repression of the activity or expression of a gene, e.g. a gene comprising SEQ ID NO.: 923

Construction of antisense constructs for repression of the activity or expression of a gene, e.g. a gene comprising SEQ ID NO.: 1083

Construction of antisense constructs for repression of the activity or expression of a gene, e.g. a gene comprising SEQ ID NO.: 1385

Construction of antisense constructs for repression of the activity or expression of a gene, e.g. a gene comprising SEQ ID NO.: 1418

Construction of antisense constructs for repression of the activity or expression of a gene, e.g. a gene comprising SEQ ID NO.: 1464

Construction of antisense constructs for repression of the activity or expression of a gene, e.g. a gene comprising SEQ ID NO.: 1551

Construction of antisense constructs for repression of the activity or expression of a gene, e.g. a gene comprising SEQ ID NO.: 1593

Construction of antisense constructs for repression of the activity or expression of a gene, e.g. a gene comprising SEQ ID NO.: 813

Construction of antisense constructs for repression of the activity or expression of a gene, e.g. a gene comprising SEQ ID NO.: 1650

A fragment of SEQ ID NO: 1025 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 104 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 190 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 410 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 512 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 673 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 729 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 27 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 923 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 1083 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 1385 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 1418 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 1464 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 1551 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 1593 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 813 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

A fragment of SEQ ID NO: 1650 is amplified by PCR. To enable cloning of the PCR product, restriction sites may be added to the primers used for the amplification. Alternatively recombination sites may be added to the primers to enable a recombination reaction. The PCR fragment is either cloned or recombined into a binary vector, preferently under control of a strong constitutive, tissue or developmental specific promoters in a way, that the orientation to the direction of the gene is opposite of the direction the gene has in its original genomic position.

The amplification of a fragment of a sequences indicated in a line of column 5 of Table III can be performed using those primers which are indicated in column 7 in the respective same line in the same Table III, comprising the extensions 5′-ATACCCGGG-3′ (SEQ ID NO.: 21) or 5′-ATAGAGCTC-3′ (SEQ ID NO.: 22). The extensions 5′-ATACCCGGG-3_{—} (SEQ ID NO.: 21) or 5′-ATAGAGCTC (SEQ ID NO.: 22) contain the Xmal and Sacl restriction enzyme recognition sides, respectively, for cloning purposes.

The Oligonucleotides are solved in water to give a concentration of 20 μM. The PCR reaction contains 5 μl Herculase buffer (Stratagene), 0.4 μl dNTPs (25 mM each) (Amersham), 0.5 μl of each primer, 0.5 μl Herculase (Stratagene), 0.5 μl gDNA and 42.6 μl water. The PCR is performed on MJ-Cycler Tetrad (BioZym) with the following programm:

4 min 94° C., followed by 30 cycles of 1 min 94° C., 1 min 50° C., 2 min 72° C. followed by 10 min 72° C. and cooling to 25° C.

The PCR product can be purified using a Kit from Qiagen. The DNA is subsequently digested with Xmal/Sacl at 37° C. over night. The fragment can then be cloned into the vector 1bxPcUbicolic SEQ ID NO.: 2, which is digested with Xmal/Sacl.

Construction of RNAi constructs for repression of the activity or expression of a gene, e.g. of a gene comprising SEQ ID NO.: 1025

Construction of RNAi constructs for repression of the activity or expression of a gene, e.g. of a gene comprising SEQ ID NO.: 104

Construction of RNAi constructs for repression of the activity or expression of a gene, e.g. of a gene comprising SEQ ID NO.: 190

Construction of RNAi constructs for repression of the activity or expression of a gene, e.g. of a gene comprising SEQ ID NO.: 410

Construction of RNAi constructs for repression of the activity or expression of a gene, e.g. of a gene comprising SEQ ID NO.: 512

Construction of RNAi constructs for repression of the activity or expression of a gene, e.g. of a gene comprising SEQ ID NO.: 673

Construction of RNAi constructs for repression of the activity or expression of a gene, e.g. of a gene comprising SEQ ID NO.: 729

Construction of RNAi constructs for repression of the activity or expression of a gene, e.g. of a gene comprising SEQ ID NO.: 27

Construction of RNAi constructs for repression of the activity or expression of a gene, e.g. of a gene comprising SEQ ID NO.: 923

Construction of RNAi constructs for repression of the activity or expression of a gene, e.g. of a gene comprising SEQ ID NO.: 1083

Construction of RNAi constructs for repression of the activity or expression of a gene, e.g. of a gene comprising SEQ ID NO.: 1385

Construction of RNAi constructs for repression of the activity or expression of a gene, e.g. of a gene comprising SEQ ID NO.: 1418

Construction of RNAi constructs for repression of the activity or expression of a gene, e.g. of a gene comprising SEQ ID NO.: 1464

Construction of RNAi constructs for repression of the activity or expression of a gene, e.g. of a gene comprising SEQ ID NO.: 1551

Construction of RNAi constructs for repression of the activity or expression of a gene, e.g. of a gene comprising SEQ ID NO.: 1593

Construction of RNAi constructs for repression of the activity or expression of a gene, e.g. of a gene comprising SEQ ID NO.: 813

Construction of RNAi constructs for repression of the activity or expression of a gene, e.g. of a gene comprising SEQ ID NO.: 1650

A fragment of SEQ ID NO: 1025 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 104 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 190 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 410 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 512 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 673 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 729 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 27 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 923 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 1083 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 1385 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 1418 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 1464 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 1551 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 1593 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 813 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

A fragment of SEQ ID NO: 1650 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the fragment is introduced twice in the vector as an inverted repeat, the repeats separated by a DNA spacer.

The amplification of a fragment of a sequence indicated in a line of column 5 of Table III can be performed using those primers which are indicated in column 7 in the respective same line in the same Table III comprising the extensions 5′-ATAGGTACC-3′ (SEQ ID NO.: 23) or 5′-ATAGTCGAC-3′(SEQ ID NO.: 24). The extensions 5′-ATAGGTACC-3′ (SEQ ID NO.: 23) or 5′-ATAGTCGAC-3′(SEQ ID NO.: 24) contain the Asp718 and Sall restriction enzyme recognition sides respectively for cloning purposes.

The Oligonucleotides are solved in water to give a concentration of 20 μM. The PCR reaction contains 5 μl Herculase buffer (Stratagene), 0.4 μl dNTPs (25 mM each) (Amersham), 0.5 μl of each primer, 0.5 μl Herculase (Stratagene), 0.5 μl gDNA and 42.6 μl water. The PCR is performed on MJ-Cycler Tetrad (BioZym) with the following programm:

4 min 94° C., followed by 30 cycles of 1 min 94° C., 1 min 50° C., 2 min 72° C. followed by 10 min 72° C. and cooling to 25° C.

The PCR product can be purified using a Kit from Qiagen. The DNA is subsequently digested with Asp718/Sall at 37° C. over night. The fragment can then be cloned into the vector 10xPcUbispacer SEQ ID NO: 3 which is digested with Asp718/Sall. The resulting construct is digested with Xhol/BsrGl and the same Asp718/Sall digested PCR fragment is ligated into this vector. Subsequently, the expression cassette giving rise to BASTA resistance is ligated as Xbal fragment into this vector that is opened with Xbal and dephosphorilized before.

Construction of Cosuppression constructs for repression of the activity or expression of a gene, e.g. of a gene comprising SEQ ID NO.: 1025

Construction of Cosuppression constructs for repression of the activity or expression of a gene, e.g. of a gene comprising SEQ ID NO.: 104

Construction of Cosuppression constructs for repression of the activity or expression of a gene, e.g. of a gene comprising SEQ ID NO.: 190

Construction of Cosuppression constructs for repression of the activity or expression of a gene, e.g. of a gene comprising SEQ ID NO.: 410

Construction of Cosuppression constructs for repression of the activity or expression of a gene, e.g. of a gene comprising SEQ ID NO.: 512

Construction of Cosuppression constructs for repression of the activity or expression of a gene, e.g. of a gene comprising SEQ ID NO.: 673

Construction of Cosuppression constructs for repression of the activity or expression of a gene, e.g. of a gene comprising SEQ ID NO.: 729

Construction of Cosuppression constructs for repression of the activity or expression of a gene, e.g. of a gene comprising SEQ ID NO.: 27

Construction of Cosuppression constructs for repression of the activity or expression of a gene, e.g. of a gene comprising SEQ ID NO.: 923

Construction of Cosuppression constructs for repression of the activity or expression of a gene, e.g. of a gene comprising SEQ ID NO.: 1083

Construction of Cosuppression constructs for repression of the activity or expression of a gene, e.g. of a gene comprising SEQ ID NO.: 1385

Construction of Cosuppression constructs for repression of the activity or expression of a gene, e.g. of a gene comprising SEQ ID NO.: 1418

Construction of Cosuppression constructs for repression of the activity or expression of a gene, e.g. of a gene comprising SEQ ID NO.: 1464

Construction of Cosuppression constructs for repression of the activity or expression of a gene, e.g. of a gene comprising SEQ ID NO.: 1551

Construction of Cosuppression constructs for repression of the activity or expression of a gene, e.g. of a gene comprising SEQ ID NO.: 1593

Construction of Cosuppression constructs for repression of the activity or expression of a gene, e.g. of a gene comprising SEQ ID NO.: 813

Construction of Cosuppression constructs for repression of the activity or expression of a gene, e.g. of a gene comprising SEQ ID NO.: 1650

A fragment of SEQ ID NO: 1025 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 104 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 190 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 410 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 512 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 673 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 729 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 27 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 923 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 1083 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 1385 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 1418 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 1464 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 1551 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 1593 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 813 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

A fragment of SEQ ID NO: 1650 is amplified by PCR. To enable cloning of the PCR product, restriction sites may be added to the primers used for the amplification. Alternatively recombinationsites may be added to the primers to enable a recombination reaction. The PCR fragment is either cloned or recombined into a binary vector, preferently under control of a strong constitutive, tissue or developmental specific promoters in a way, that the orientation to the promoter is identical to the direction the gen has in its original genomic position.

The amplification of the fragment of the sequences indicated in a line of column 5 of Table III is performed using those primers, which are indicated in column 7 of the respective same line in the same Table III, which comprises the extensions 5′-ATACCATGG-3′ (SEQ ID NO.: 25) or 5′-ATATTAATTAA-3′ (SEQ ID NO.: 26). The extensions 5′-ATACCATGG-3′ (SEQ ID NO.: 25) or 5′-ATATTAATTAA-3′ (SEQ ID NO.: 26), contain the Ncol and Pacl restriction enzyme recognition sides respectively for cloning purposes

The Oligonucleotides are solved in water to give a concentration of 20 μM. The PCR reaction contains 5 μl Herculase buffer (Stratagene), 0.4 μl dNTPs (25 mM each) (Amersham), 0.5 μl of each p, 0.5 μl Herculase (Stratagene), 0.5 μl gDNA and 42.6 μl water. The PCR is performed on MJ-Cycler Tetrad (BioZym) with the following programm:

4 min 94° C., followed by 30 cycles of 1 min 94° C., 1 min 50° C., 2 min 72° C. followed by 10 min 72° C. and cooling to 25° C.

The PCR product is purified using a Kit from Qiagen. The DNA is subsequently digested with Ncol/Pacl at 37° C. over night. The fragment can then be cloned into the vector 1bxPcUbicolic SEQ ID NO: 2 which is digested with Ncol/Pacl.

Reducing the activity or expression of a gene, e.g. of a gene comprising SEQ ID NO.: 1025 by artificial transcription factors

Reducing the activity or expression of a gene, e.g. of a gene comprising SEQ ID NO.: 104 by artificial transcription factors

Reducing the activity or expression of a gene, e.g. of a gene comprising SEQ ID NO.: 190 by artificial transcription factors

Reducing the activity or expression of a gene, e.g. of a gene comprising SEQ ID NO.: 410 by artificial transcription factors

Reducing the activity or expression of a gene, e.g. of a gene comprising SEQ ID NO.: 512 by artificial transcription factors

Reducing the activity or expression of a gene, e.g. of a gene comprising SEQ ID NO.: 673 by artificial transcription factors

Reducing the activity or expression of a gene, e.g. of a gene comprising SEQ ID NO.: 729 by artificial transcription factors

Reducing the activity or expression of a gene, e.g. of a gene comprising SEQ ID NO.: 27 by artificial transcription factors

Reducing the activity or expression of a gene, e.g. of a gene comprising SEQ ID NO.: 923 by artificial transcription factors

Reducing the activity or expression of a gene, e.g. of a gene comprising SEQ ID NO.: 1083 by artificial transcription factors

Reducing the activity or expression of a gene, e.g. of a gene comprising SEQ ID NO.: 1385 by artificial transcription factors

Reducing the activity or expression of a gene, e.g. of a gene comprising SEQ ID NO.: 1418 by artificial transcription factors

Reducing the activity or expression of a gene, e.g. of a gene comprising SEQ ID NO.: 1464 by artificial transcription factors

Reducing the activity or expression of a gene, e.g. of a gene comprising SEQ ID NO.: 1551 by artificial transcription factors

Reducing the activity or expression of a gene, e.g. of a gene comprising SEQ ID NO.: 1593 by artificial transcription factors

Reducing the activity or expression of a gene, e.g. of a gene comprising SEQ ID NO.: 813 by artificial transcription factors

Reducing the activity or expression of a gene, e.g. of a gene comprising SEQ ID NO.: 1650 by artificial transcription factors

A gene and its homologous ORFs in other species may also be down regulated by introducing a synthetic specific repressor. For this purpose, a gene for a chimeric zinc finger protein, which binds to a specific region in the regulatory or coding region of the genes of interests or its homologs in other species is constructed. The artificial zinc finger protein comprises a specific DNA-binding domain consting for example of zinc finger and optional an repression like the EAR domain (Hiratsu et al., 2003. Plant J. 34(5), 733-739 Dominant repression of target genes by chimeric repressors that include the EAR motif, a repression domain, in Arabidopsis.)

Expression of this chimeric repressor for example in plants then results in specific repression of the target gene or of its homologs in other plant species lead to increased metabolite production. The experimental details expecially about the desing and construction of specific zinc finger domains may be carried out as described, or WO 01/52620 or Ordiz M I, (Proc. Natl. Acad. Sci. USA, 2002, Vol. 99, Issue 20, 13290) or Guan, (Proc. Natl. Acad. Sci. USA, 2002, Vol. 99, Issue 20, 13296).

Engineering ryegrass plants by repressing the activity or expression of a gene, e.g. of a gene homolog to a gene comprising SEQ ID NO.: 1025 in ryegrass

Engineering ryegrass plants by repressing the activity or expression of a gene, e.g. of a gene homolog to a gene comprising SEQ ID NO.: 104 in ryegrass

Engineering ryegrass plants by repressing the activity or expression of a gene, e.g. of a gene homolog to a gene comprising SEQ ID NO.: 190 in ryegrass

Engineering ryegrass plants by repressing the activity or expression of a gene, e.g. of a gene homolog to a gene comprising SEQ ID NO.: 410 in ryegrass

Engineering ryegrass plants by repressing the activity or expression of a gene, e.g. of a gene homolog to a gene comprising SEQ ID NO.: 512 in ryegrass

Engineering ryegrass plants by repressing the activity or expression of a gene, e.g. of a gene homolog to a gene comprising SEQ ID NO.: 673 in ryegrass

Engineering ryegrass plants by repressing the activity or expression of a gene, e.g. of a gene homolog to a gene comprising SEQ ID NO.: 729 in ryegrass

Engineering ryegrass plants by repressing the activity or expression of a gene, e.g. of a gene homolog to a gene comprising SEQ ID NO.: 27 in ryegrass

Engineering ryegrass plants by repressing the activity or expression of a gene, e.g. of a gene homolog to a gene comprising SEQ ID NO.: 923 in ryegrass

Engineering ryegrass plants by repressing the activity or expression of a gene, e.g. of a gene homolog to a gene comprising SEQ ID NO.: 1083 in ryegrass

Engineering ryegrass plants by repressing the activity or expression of a gene, e.g. of a gene homolog to a gene comprising SEQ ID NO.: 1385 in ryegrass

Engineering ryegrass plants by repressing the activity or expression of a gene, e.g. of a gene homolog to a gene comprising SEQ ID NO.: 1418 in ryegrass

Engineering ryegrass plants by repressing the activity or expression of a gene, e.g. of a gene homolog to a gene comprising SEQ ID NO.: 1464 in ryegrass

Engineering ryegrass plants by repressing the activity or expression of a gene, e.g. of a gene homolog to a gene comprising SEQ ID NO.: 1551 in ryegrass

Engineering ryegrass plants by repressing the activity or expression of a gene, e.g. of a gene homolog to a gene comprising SEQ ID NO.: 1593 in ryegrass

Engineering ryegrass plants by repressing the activity or expression of a gene, e.g. of a gene homolog to a gene comprising SEQ ID NO.: 813 in ryegrass

Engineering ryegrass plants by repressing the activity or expression of a gene, e.g. of a gene homolog to a gene comprising SEQ ID NO.: 1650 in ryegrass

Seeds of several different ryegrass varieties can be used as explant sources for transformation, including the commercial variety Gunne available from Svalof Weibull Seed Company or the variety Affinity. Seeds are surface-sterilized sequentially with 1% Tween-20 for 1 minute, 100% bleach for 60 minutes, 3 rinses with 5 minutes each with de-ionized and distilled H₂O, and then germinated for 3-4 days on moist, sterile filter paper in the dark. Seedlings are further sterilized for 1 minute with 1% Tween-20, 5 minutes with 75% bleach, and rinsed 3 times with ddH₂O, 5 min each.

Surface-sterilized seeds are placed on the callus induction medium containing Murashige and Skoog basal salts and vitamins, 20 g/l sucrose, 150 mg/l asparagine, 500 mg/l casein hydrolysate, 3 g/l Phytagel, 10 mg/l BAP, and 5 mg/l dicamba. Plates are incubated in the dark at 25° C. for 4 weeks for seed germination and embryogenic callus induction.

After 4 weeks on the callus induction medium, the shoots and roots of the seedlings are trimmed away, the callus is transferred to fresh media, is maintained in culture for another 4 weeks, and is then transferred to MSO medium in light for 2 weeks. Several pieces of callus (11-17 weeks old) are either strained through a 10 mesh sieve and put onto callus induction medium, or are cultured in 100 ml of liquid ryegrass callus induction media (same medium as for callus induction with agar) in a 250 ml flask. The flask is wrapped in foil and shaken at 175 rpm in the dark at 23° C. for 1 week. Sieving the liquid culture with a 40-mesh sieve is collected the cells. The fraction collected on the sieve is plated and is cultured on solid ryegrass callus induction medium for 1 week in the dark at 25° C. The callus is then transferred to and is cultured on MS medium containing 1% sucrose for 2 weeks.

Transformation can be accomplished with either Agrobacterium or with particle bombardment methods. An expression vector is created containing a constitutive or otherwise appropiate plant promoter and the antisense, RNAi, snRNA, dsRNA, siRNA, miRNA, ta-siRNA, cosuppression construct, recombination construct or ribozyme molecule, or the viral nucleic acid molecule, nucleic acid construct in a pUC vector. The plasmid DNA is prepared from E. coli cells using with Qiagen kit according to manufacturer's instruction. Approximately 2 g of embryogenic callus is spread in the center of a sterile filter paper in a Petri dish. An aliquot of liquid MSO with 10 g/l sucrose is added to the filter paper. Gold particles (1.0 μm in size) are coated with plasmid DNA according to method of Sanford et al., 1993 and are delivered to the embryogenic callus with the following parameters: 500 μg particles and 2 μg DNA per shot, 1300 psi and a target distance of 8.5 cm from stopping plate to plate of callus and 1 shot per plate of callus.

After the bombardment, calli are transferred back to the fresh callus development medium and maintained in the dark at room temperature for a 1-week period. The callus is then transferred to growth conditions in the light at 25° C. to initiate embryo differentiation with the appropriate selection agent, e.g. 250 nM Arsenal, 5 mg/l PPT or 50 mg/L Kanamycin. Shoots resistant to the selection agent are appearing and once rooted are transferred to soil.

Samples of the primary transgenic plants (T0) are analyzed by PCR to confirm the presence of T-DNA. These results are confirmed by Southern hybridization in which DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labelled probe by PCR, and used as recommended by the manufacturer. Furthermore the primary transgenic plants (T0) are analyzed for repressed expression of the gene to be repressed by standard methods such as Northern blots or quantitative RTPCR.

Transgenic T0 ryegrass plants are propagated vegetatively by excising tillers. The transplanted tillers are maintained in the greenhouse for 2 months until well established. The shoots are defoliated and allowed to grow for 2 weeks.

Engineering soybean plants by repressing the activity or expression of a gene, e.g. of a gene homolog to a gene comprising SEQ ID NO.: 1025 in soybean

Engineering soybean plants by repressing the activity or expression of a gene, e.g. of a gene homolog to a gene comprising SEQ ID NO.: 104 in soybean

Engineering soybean plants by repressing the activity or expression of a gene, e.g. of a gene homolog to a gene comprising SEQ ID NO.: 190 in soybean

Engineering soybean plants by repressing the activity or expression of a gene, e.g. of a gene homolog to a gene comprising SEQ ID NO.: 410 in soybean

Engineering soybean plants by repressing the activity or expression of a gene, e.g. of a gene homolog to a gene comprising SEQ ID NO.: 512 in soybean

Engineering soybean plants by repressing the activity or expression of a gene, e.g. of a gene homolog to a gene comprising SEQ ID NO.: 673 in soybean

Engineering soybean plants by repressing the activity or expression of a gene, e.g. of a gene homolog to a gene comprising SEQ ID NO.: 729 in soybean

Engineering soybean plants by repressing the activity or expression of a gene, e.g. of a gene homolog to a gene comprising SEQ ID NO.: 27 in soybean

Engineering soybean plants by repressing the activity or expression of a gene, e.g. of a gene homolog to a gene comprising SEQ ID NO.: 923 in soybean

Engineering soybean plants by repressing the activity or expression of a gene, e.g. of a gene homolog to a gene comprising SEQ ID NO.: 1083 in soybean

Engineering soybean plants by repressing the activity or expression of a gene, e.g. of a gene homolog to a gene comprising SEQ ID NO.: 1385 in soybean

Engineering soybean plants by repressing the activity or expression of a gene, e.g. of a gene homolog to a gene comprising SEQ ID NO.: 1418 in soybean

Engineering soybean plants by repressing the activity or expression of a gene, e.g. of a gene homolog to a gene comprising SEQ ID NO.: 1464 in soybean

Engineering soybean plants by repressing the activity or expression of a gene, e.g. of a gene homolog to a gene comprising SEQ ID NO.: 1551 in soybean

Engineering soybean plants by repressing the activity or expression of a gene, e.g. of a gene homolog to a gene comprising SEQ ID NO.: 1593 in soybean

Engineering soybean plants by repressing the activity or expression of a gene, e.g. of a gene homolog to a gene comprising SEQ ID NO.: 813 in soybean

Engineering soybean plants by repressing the activity or expression of a gene, e.g. of a gene homolog to a gene comprising SEQ ID NO.: 1650 in soybean

Soybean can be transformed according to the following modification of the method described in the Texas A&M patent U.S. Pat. No. 5,164,310. Several commercial soybean varieties are amenable to transformation by this method. The cultivar Jack (available from the Illinois Seed Foundation) is commonly used for transformation. Seeds are sterilized by immersion in 70% (v/v) ethanol for 6 min and in 25% commercial bleach (NaOCl) supplemented with 0.1% (v/v) Tween for 20 min, followed by rinsing 4 times with sterile double distilled water. Removing the radicle, hypocotyl and one cotyledon from each seedling propagates seven-day seedlings. Then, the epicotyl with one cotyledon is transferred to fresh germination media in petri dishes and incubated at 25° C. under a 16-hr photoperiod (approx. 100 μE-m-2s-1) for three weeks. Axillary nodes (approx. 4 mm in length) are cut from 3 to 4 week-old plants. Axillary nodes are excised and incubated in Agrobacterium LBA4404 culture.

Many different binary vector systems have been described for plant transformation (e.g. An, G. in Agrobacterium Protocols. Methods in Molecular Biology vol 44, pp 47-62, Gartland K M A and M R Davey eds. Humana Press, Totowa, N.J.). Many are based on the vector pBIN19 described by Bevan (Nucleic Acid Research. 1984. 12:8711-8721) that includes a plant gene expression cassette flanked by the left and right border sequences from the Ti plasmid of Agrobacterium tumefaciens. A plant gene expression cassette consists of at least two genes—a selection marker gene and a plant promoter regulating the transcription of the antisense, RNAi, snRNA, dsRNA, siRNA, miRNA, ta-siRNA, cosuppression construct, or ribozyme molecule, or the viral nucleic acid molecule. Various selection marker genes can be used as described above, including the Arabidopsis gene encoding a mutated acetohydroxy acid synthase (AHAS) enzyme (U.S. Pat. Nos. 5,767,366 and 6,225,105). Similarly, various promoters can be used to regulate the repression cassette to provide constitutive, developmental, tissue or environmental repression of gene transcription as described above. In this example, the 34S promoter (GenBank Accession numbers M59930 and X16673) is used to provide constitutive repression of the repression cassette.

After the co-cultivation treatment, the explants are washed and transferred to selection media supplemented with 500 mg/L timentin. Shoots are excised and placed on a shoot elongation medium. Shoots longer than 1 cm are placed on rooting medium for two to four weeks prior to transplanting to soil.

The primary transgenic plants (TO) are analyzed by PCR to confirm the presence of T-DNA. These results are confirmed by Southern hybridization in which DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labelled probe by PCR, and is used as recommended by the manufacturer. Furthermore the primary transgenic plants (T0) are analyzed for repressed expression of the gene to be repressed by standard methods such as Northern blots or quantitative RTPCR.

Engineering corn plants by repressing the activity or expression of a gene, e.g. of a gene homolog to a gene comprising SEQ ID NO.: 1025 in corn

Engineering corn plants by repressing the activity or expression of a gene, e.g. of a gene homolog to a gene comprising SEQ ID NO.: 104 in corn

Engineering corn plants by repressing the activity or expression of a gene, e.g. of a gene homolog to a gene comprising SEQ ID NO.: 190 in corn

Engineering corn plants by repressing the activity or expression of a gene, e.g. of a gene homolog to a gene comprising SEQ ID NO.: 410 in corn

Engineering corn plants by repressing the activity or expression of a gene, e.g. of a gene homolog to a gene comprising SEQ ID NO.: 512 in corn

Engineering corn plants by repressing the activity or expression of a gene, e.g. of a gene homolog to a gene comprising SEQ ID NO.: 673 in corn

Engineering corn plants by repressing the activity or expression of a gene, e.g. of a gene homolog to a gene comprising SEQ ID NO.: 729 in corn

Engineering corn plants by repressing the activity or expression of a gene, e.g. of a gene homolog to a gene comprising SEQ ID NO.: 27 in corn

Engineering corn plants by repressing the activity or expression of a gene, e.g. of a gene homolog to a gene comprising SEQ ID NO.: 923 in corn

Engineering corn plants by repressing the activity or expression of a gene, e.g. of a gene homolog to a gene comprising SEQ ID NO.: 1083 in corn

Engineering corn plants by repressing the activity or expression of a gene, e.g. of a gene homolog to a gene comprising SEQ ID NO.: 1385 in corn

Engineering corn plants by repressing the activity or expression of a gene, e.g. of a gene homolog to a gene comprising SEQ ID NO.: 1418 in corn

Engineering corn plants by repressing the activity or expression of a gene, e.g. of a gene homolog to a gene comprising SEQ ID NO.: 1464 in corn

Engineering corn plants by repressing the activity or expression of a gene, e.g. of a gene homolog to a gene comprising SEQ ID NO.: 1551 in corn

Engineering corn plants by repressing the activity or expression of a gene, e.g. of a gene homolog to a gene comprising SEQ ID NO.: 1593 in corn

Engineering corn plants by repressing the activity or expression of a gene, e.g. of a gene homolog to a gene comprising SEQ ID NO.: 813 in corn

Engineering corn plants by repressing the activity or expression of a gene, e.g. of a gene homolog to a gene comprising SEQ ID NO.: 1650 in corn

Transformation of maize (Zea Mays L.) is performed with a modification of the method described by Ishida et al. (1996. Nature Biotech 14745-50). Transformation is genotype-dependent in corn and only specific genotypes are amenable to transformation and regeneration. The inbred line A188 (University of Minnesota) or hybrids with A188 as a parent are good sources of donor material for transformation (Fromm et al. 1990 Biotech 8:833-839), but other genotypes can be used successfully as well. Ears are harvested from corn plants at approximately 11 days after pollination (DAP) when the length of immature embryos is about 1 to 1.2 mm. Immature embryos are cocultivated with Agrobacterium tumefaciens that carry “super binary” vectors and transgenic plants are recovered through organogenesis. The super binary vector system of Japan Tobacco is described in WO patents WO94/00977 and WO95/06722. Vectors can be constructed as described. Various selection marker genes can be used including the maize gene encoding a mutated acetohydroxy acid synthase (AHAS) enzyme (U.S. Pat. No. 6,025,541). Similarly, various promoters can be used to regulate the repression cassette to provide constitutive, developmental, tissue or environmental expression of the antisense, RNAi, snRNA, dsRNA, siRNA, miRNA, ta-siRNA, cosuppression construct, or ribozyme molecule, or the viral nucleic acid molecule,. In this example, the 34S promoter (GenBank Accession numbers M59930 and X16673) is used to provide constitutive expression of the repression cassette.

Excised embryos are grown on callus induction medium, then maize regeneration medium, containing imidazolinone as a selection agent. The Petri plates are incubated in the light at 25° C. for 2 to 3 weeks, or until shoots develop. The green shoots are transferred from each embryo to maize rooting medium and incubated at 25° C. for 2 to 3 weeks, until roots develop. The rooted shoots are transplanted to soil in the greenhouse. T1 seeds are produced from plants that exhibit tolerance to the imidazolinone herbicides and which show repressed expression of the gene to be repressed. Such analysis can be done by standard methods such as Northern blots or quantitative RTPCR.

The T1 generation of single locus insertions of the T-DNA can segregate for the transgene in a 3:1 ratio. Those progeny containing one or two copies of the transgene are tolerant of the imidazolinone herbicide. Homozygous T2 plants can exhibit similar phenotypes as the T1 plants. Hybrid plants (F1 progeny) of homozygous transgenic plants and non-transgenic plants can also exhibited increased similar phenotyps.

Engineering wheat plants by repressing the activity or expression of a gene, e.g. of a gene homolog to a gene comprising SEQ ID NO.: 1025 in wheat

Engineering wheat plants by repressing the activity or expression of a gene, e.g. of a gene homolog to a gene comprising SEQ ID NO.: 104 in wheat

Engineering wheat plants by repressing the activity or expression of a gene, e.g. of a gene homolog to a gene comprising SEQ ID NO.: 190 in wheat

Engineering wheat plants by repressing the activity or expression of a gene, e.g. of a gene homolog to a gene comprising SEQ ID NO.: 410 in wheat

Engineering wheat plants by repressing the activity or expression of a gene, e.g. of a gene homolog to a gene comprising SEQ ID NO.: 512 in wheat

Engineering wheat plants by repressing the activity or expression of a gene, e.g. of a gene homolog to a gene comprising SEQ ID NO.: 673 in wheat

Engineering wheat plants by repressing the activity or expression of a gene, e.g. of a gene homolog to a gene comprising SEQ ID NO.: 729 in wheat

Engineering wheat plants by repressing the activity or expression of a gene, e.g. of a gene homolog to a gene comprising SEQ ID NO.: 27 in wheat

Engineering wheat plants by repressing the activity or expression of a gene, e.g. of a gene homolog to a gene comprising SEQ ID NO.: 923 in wheat

Engineering wheat plants by repressing the activity or expression of a gene, e.g. of a gene homolog to a gene comprising SEQ ID NO.: 1083 in wheat

Engineering wheat plants by repressing the activity or expression of a gene, e.g. of a gene homolog to a gene comprising SEQ ID NO.: 1385 in wheat

Engineering wheat plants by repressing the activity or expression of a gene, e.g. of a gene homolog to a gene comprising SEQ ID NO.: 1418 in wheat

Engineering wheat plants by repressing the activity or expression of a gene, e.g.

of a gene homolog to a gene comprising SEQ ID NO.: 1464 in wheat

Engineering wheat plants by repressing the activity or expression of a gene, e.g. of a gene homolog to a gene comprising SEQ ID NO.: 1551 in wheat

Engineering wheat plants by repressing the activity or expression of a gene, e.g. of a gene homolog to a gene comprising SEQ ID NO.: 1593 in wheat

Engineering wheat plants by repressing the activity or expression of a gene, e.g. of a gene homolog to a gene comprising SEQ ID NO.: 813 in wheat

Engineering wheat plants by repressing the activity or expression of a gene, e.g. of a gene homolog to a gene comprising SEQ ID NO.: 1650 in wheat

Transformation of wheat is performed with the method described by Ishida et al. (1996 Nature Biotech. 14745-50). The cultivar Bobwhite (available from CYMMIT, Mexico) is commonly used in transformation. Immature embryos are cocultivated with Agrobacterium tumefaciens that carry “super binary” vectors, and transgenic plants are recovered through organogenesis. The super binary vector system of Japan Tobacco is described in WO patents WO94/00977 and WO95/06722. Vectors were constructed as described. Various selection marker genes can be used including the maize gene encoding a mutated acetohydroxy acid synthase (AHAS) enzyme (U.S. Pat. No. 6,025,541). Similarly, various promoters can be used to regulate the repression cassette to provide constitutive, developmental, tissue or environmental regulation of the antisense, RNAi, snRNA, dsRNA, siRNA, miRNA, ta-siRNA, cosuppression construct, ribozyme molecule, or the viral nucleic acid molecule. In this example, the 34S promoter (GenBank Accession numbers M59930 and X16673) can be used to provide constitutive expression of the repression cassette.

After incubation with Agrobacterium, the embryos are grown on callus induction medium, then regeneration medium, containing imidazolinone as a selection agent. The Petri plates are incubated in the light at 25° C. for 2 to 3 weeks, or until shoots develop. The green shoots are transferred from each embryo to rooting medium and incubated at 25° C. for 2 to 3 weeks, until roots develop. The rooted shoots are transplanted to soil in the greenhouse. T1 seeds are produced from plants that exhibit tolerance to the imidazolinone herbicides and which show repressed expression of the gene to be repressed. Such analysis can be done by standard methods such as Northern blots or quantitative RTPCR.

The T1 generation of single locus insertions of the T-DNA can segregate for the transgene in a 3:1 ratio. Those progeny containing one or two copies of the transgene are tolerant of the imidazolinone herbicide. Homozygous T2 plants exhibited similar phenotypes.

Engineering Rapeseed/Canola plants by repressing the activity or expression of a gene, e.g. of a gene homolog to a gene comprising SEQ ID NO.: 1025 in rapeseed/canola plants

Engineering Rapeseed/Canola plants by repressing the activity or expression of a gene, e.g. of a gene homolog to a gene comprising SEQ ID NO.: 104 in rapeseed/canola plants

Engineering Rapeseed/Canola plants by repressing the activity or expression of a gene, e.g. of a gene homolog to a gene comprising SEQ ID NO.: 190 in rapeseed/canola plants

Engineering Rapeseed/Canola plants by repressing the activity or expression of a gene, e.g. of a gene homolog to a gene comprising SEQ ID NO.: 410 in rapeseed/canola plants

Engineering Rapeseed/Canola plants by repressing the activity or expression of a gene, e.g. of a gene homolog to a gene comprising SEQ ID NO.: 512 in rapeseed/canola plants

Engineering Rapeseed/Canola plants by repressing the activity or expression of a gene, e.g. of a gene homolog to a gene comprising SEQ ID NO.: 673 in rapeseed/canola plants

Engineering Rapeseed/Canola plants by repressing the activity or expression of a gene, e.g. of a gene homolog to a gene comprising SEQ ID NO.: 729 in rapeseed/canola plants

Engineering Rapeseed/Canola plants by repressing the activity or expression of a gene, e.g. of a gene homolog to a gene comprising SEQ ID NO.: 27 in rapeseed/canola plants

Engineering Rapeseed/Canola plants by repressing the activity or expression of a gene, e.g. of a gene homolog to a gene comprising SEQ ID NO.: 923 in rapeseed/canola plants

Engineering Rapeseed/Canola plants by repressing the activity or expression of a gene, e.g. of a gene homolog to a gene comprising SEQ ID NO.: 1083 in rapeseed/canola plants

Engineering Rapeseed/Canola plants by repressing the activity or expression of a gene, e.g. of a gene homolog to a gene comprising SEQ ID NO.: 1385 in rapeseed/canola plants

Engineering Rapeseed/Canola plants by repressing the activity or expression of a gene, e.g. of a gene homolog to a gene comprising SEQ ID NO.: 1418 in rapeseed/canola plants

Engineering Rapeseed/Canola plants by repressing the activity or expression of a gene, e.g. of a gene homolog to a gene comprising SEQ ID NO.: 1464 in rapeseed/canola plants

Engineering Rapeseed/Canola plants by repressing the activity or expression of a gene, e.g. of a gene homolog to a gene comprising SEQ ID NO.: 1551 in rapeseed/canola plants

Engineering Rapeseed/Canola plants by repressing the activity or expression of a gene, e.g. of a gene homolog to a gene comprising SEQ ID NO.: 1593 in rapeseed/canola plants

Engineering Rapeseed/Canola plants by repressing the activity or expression of a gene, e.g. of a gene homolog to a gene comprising SEQ ID NO.: 813 in rapeseed/canola plants

Engineering Rapeseed/Canola plants by repressing the activity or expression of a gene, e.g. of a gene homolog to a gene comprising SEQ ID NO.: 1650 in rapeseed/canola plants

Cotyledonary petioles and hypocotyls of 5-6 day-old young seedlings are used as explants for tissue culture and transformed according to Babic et al. (1998, Plant Cell Rep 17: 183-188). The commercial cultivar Westar (Agriculture Canada) is the standard variety used for transformation, but other varieties can be used.

Agrobacterium tumefaciens LBA4404 containing a binary vector are used for canola transformation. Many different binary vector systems have been described for plant transformation (e.g. An, G. in Agrobacterium Protocols. Methods in Molecular Biology vol 44, pp 47-62, Gartland K M A and M R Davey eds. Humana Press, Totowa, N.J.). Many are based on the vector pBIN19 described by Bevan (Nucleic Acid Research. 1984. 12:8711-8721) that includes a plant gene expression cassette flanked by the left and right border sequences from the Ti plasmid of Agrobacterium tumefaciens. A plant gene expression cassette consists of at least two genes—a selection marker gene and a plant promoter regulating the transcription of the repression cassette of the trait gene. Various selection marker genes can be used including the Arabidopsis gene encoding a mutated acetohydroxy acid synthase (AHAS) enzyme (U.S. Pat. Nos. 5,767,3666 and 6,225,105). Similarly, various promoters can be used to regulate the repression cassette to provide constitutive, developmental, tissue or environmental regulation of the antisense, RNAi, snRNA, dsRNA, siRNA, miRNA, ta-siRNA, cosuppression construct, ribozyme molecule, or the viral nucleic acid molecule. In this example, the 34S promoter (GenBank Accession numbers M59930 and X16673) can be used to provide constitutive expression of the repression cassette.

Canola seeds are surface-sterilized in 70% ethanol for 2 min., and then in 30% Clorox with a drop of Tween-20 for 10 min, followed by three rinses with sterilized distilled water. Seeds are then germinated in vitro 5 days on half strength MS medium without hormones, 1% sucrose, 0.7% Phytagar at 23° C., 16 hr. light. The cotyledon petiole explants with the cotyledon attached are excised from the in vitro seedlings, and are inoculated with Agrobacterium by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5 to 10 mm in length, they are cut and transferred to shoot elongation medium (MSBAP-0.5, containing 0.5 mg/l BAP). Shoots of about 2 cm in length are transferred to the rooting medium (MS0) for root induction.

Samples of the primary transgenic plants (T0) are analyzed by PCR to confirm the presence of T-DNA. These results are confirmed by Southern hybridization in which DNA is electrophoresed on a 1% agarose gel and are transferred to a positively charged nylon membrane (Roche Diagnostics). The PCR DIG Probe Synthesis Kit (Roche Diagnostics) is used to prepare a digoxigenin-labelled probe by PCR, and used as recommended by the manufacturer. Furthermore the primary transgenic plants (T0) are analyzed for repressed expression of the gene to be repressed by standard methods such as Northern blots or quantitative RTPCR.

Engineering alfalfa plants by repressing the activity or expression of a gene, e.g. of a gene homolog to a gene comprising SEQ ID NO.: 1025 in alfalfa

Engineering alfalfa plants by repressing the activity or expression of a gene, e.g. of a gene homolog to a gene comprising SEQ ID NO.: 104 in alfalfa

Engineering alfalfa plants by repressing the activity or expression of a gene, e.g. of a gene homolog to a gene comprising SEQ ID NO.: 190 in alfalfa

Engineering alfalfa plants by repressing the activity or expression of a gene, e.g. of a gene homolog to a gene comprising SEQ ID NO.: 410 in alfalfa

Engineering alfalfa plants by repressing the activity or expression of a gene, e.g. of a gene homolog to a gene comprising SEQ ID NO.: 512 in alfalfa

Engineering alfalfa plants by repressing the activity or expression of a gene, e.g. of a gene homolog to a gene comprising SEQ ID NO.: 673 in alfalfa

Engineering alfalfa plants by repressing the activity or expression of a gene, e.g. of a gene homolog to a gene comprising SEQ ID NO.: 729 in alfalfa

Engineering alfalfa plants by repressing the activity or expression of a gene, e.g. of a gene homolog to a gene comprising SEQ ID NO.: 27 in alfalfa

Engineering alfalfa plants by repressing the activity or expression of a gene, e.g. of a gene homolog to a gene comprising SEQ ID NO.: 923 in alfalfa

Engineering alfalfa plants by repressing the activity or expression of a gene, e.g. of a gene homolog to a gene comprising SEQ ID NO.: 1083 in alfalfa

Engineering alfalfa plants by repressing the activity or expression of a gene, e.g. of a gene homolog to a gene comprising SEQ ID NO.: 1385 in alfalfa

Engineering alfalfa plants by repressing the activity or expression of a gene, e.g. of a gene homolog to a gene comprising SEQ ID NO.: 1418 in alfalfa

Engineering alfalfa plants by repressing the activity or expression of a gene, e.g. of a gene homolog to a gene comprising SEQ ID NO.: 1464 in alfalfa

Engineering alfalfa plants by repressing the activity or expression of a gene, e.g. of a gene homolog to a gene comprising SEQ ID NO.: 1551 in alfalfa

Engineering alfalfa plants by repressing the activity or expression of a gene, e.g.

of a gene homolog to a gene comprising SEQ ID NO.: 1593 in alfalfa

Engineering alfalfa plants by repressing the activity or expression of a gene, e.g. of a gene homolog to a gene comprising SEQ ID NO.: 813 in alfalfa

Engineering alfalfa plants by repressing the activity or expression of a gene, e.g. of a gene homolog to a gene comprising SEQ ID NO.: 1650 in alfalfa

A regenerating clone of alfalfa (Medicago sativa) is transformed using the method of McKersie et al., 1999 Plant Physiol 119: 839-847. Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown D C W and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659).

Petiole explants are cocultivated with an overnight culture of Agrobacterium tumefaciens C58C1 pMP90 (McKersie et al., 1999 Plant Physiol 119: 839-847) or LBA4404 containing a binary vector. Many different binary vector systems have been described for plant transformation (e.g. An, G. in Agrobacterium Protocols. Methods in Molecular Biology vol 44, pp 47-62, Gartland K M A and M R Davey eds. Humana Press, Totowa, N.J.). Many are based on the vector pBIN19 described by Bevan (Nucleic Acid Research. 1984. 12:8711-8721) that includes a plant gene expression cassette flanked by the left and right border sequences from the Ti plasmid of Agrobacterium tumefaciens. A plant gene expression cassette consists of at least two genes—a selection marker gene and a plant promoter regulating the transcription of the antisense, RNAi, snRNA, dsRNA, siRNA, miRNA, ta-siRNA, cosuppression construct, ribozyme molecule, or the viral nucleic acid molecule. Various selection marker genes can be used including the Arabidopsis gene encoding a mutated acetohydroxy acid synthase (AHAS) enzyme (U.S. Pat. Nos. 5,7673,666 and 6,225,105). Similarly, various promoters can be used to regulate the repression cassette that provides constitutive, developmental, tissue or environmental regulation of gene repression. In this example, the 34S promoter (GenBank Accession numbers M59930 and X16673) can be used to provide constitutive expression of the repression cassette.

The explants are cocultivated for 3 d in the dark on SH induction medium containing 288 mg/L Pro, 53 mg/L thioproline, 4.35 g/L K₂SO₄, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings are transplanted into pots and grown in a greenhouse.

The T0 transgenic plants are propagated by node cuttings and rooted in Turface growth medium. The plants are defoliated and grown to a height of about 10 cm (approximately 2 weeks after defoliation). Furthermore the primary transgenic plants (T0) are analyzed for repressed expression of the gene to be repressed by standard methods such as Northern blots or quantitative RTPCR.

Tolerant plants according to [0412.1.1.1] (ryegrass plants), [0420.1.1.1] (soybean plants), [0425.1.1.1] (corn plants), [0429.1.1.1] (wheat plants), [0433.1.1.1] (Rapeseed/Canola) or [0438.1.1.1] (alfalfa plants) have higher survival rates and biomass production including seed yield, photosynthesis and dry matter production than susceptible plants.

Knock out of a gene by homologous recombination, e.g. of a gene comprising the sequence shown in SEQ ID NO.: 1025

Knock out of a gene by homologous recombination, e.g. of a gene comprising the sequence shown in SEQ ID NO.: 104

Knock out of a gene by homologous recombination, e.g. of a gene comprising the sequence shown in SEQ ID NO.: 190

Knock out of a gene by homologous recombination, e.g. of a gene comprising the sequence shown in SEQ ID NO.: 410

Knock out of a gene by homologous recombination, e.g. of a gene comprising the sequence shown in SEQ ID NO.: 512

Knock out of a gene by homologous recombination, e.g. of a gene comprising the sequence shown in SEQ ID NO.: 673

Knock out of a gene by homologous recombination, e.g. of a gene comprising the sequence shown in SEQ ID NO.: 729

Knock out of a gene by homologous recombination, e.g. of a gene comprising the sequence shown in SEQ ID NO.: 27

Knock out of a gene by homologous recombination, e.g. of a gene comprising the sequence shown in SEQ ID NO.: 923

Knock out of a gene by homologous recombination, e.g. of a gene comprising the sequence shown in SEQ ID NO.: 1083

Knock out of a gene by homologous recombination, e.g. of a gene comprising the sequence shown in SEQ ID NO.: 1385

Knock out of a gene by homologous recombination, e.g. of a gene comprising the sequence shown in SEQ ID NO.: 1418

Knock out of a gene by homologous recombination, e.g. of a gene comprising the sequence shown in SEQ ID NO.: 1464

Knock out of a gene by homologous recombination, e.g. of a gene comprising the sequence shown in SEQ ID NO.: 1551

Knock out of a gene by homologous recombination, e.g. of a gene comprising the sequence shown in SEQ ID NO.: 1593

Knock out of a gene by homologous recombination, e.g. of a gene comprising the sequence shown in SEQ ID NO.: 813

Knock out of a gene by homologous recombination, e.g. of a gene comprising the sequence shown in SEQ ID NO.: 1650

1. Identifying Mutations in the Gene in Random Mutagenized Populations:

a) In Chemically or Radiation Mutated Population

Production of chemically or radiation mutated populations is a common technique and known to the skilled worker. Methods are described by Koorneef et al. 1982 and the citations therein and by Lightner and Caspar in “Methods in Molecular Biology” Vol 82. These techniques usually induce pointmutations that can be identified in any known gene using methods such as TILLING (Colbert et al. 2001).

b) In T-DNA or Transposon Mutated Lopulation by Reserve Genetics

Reverse genetic strategies to identify insertion mutants in genes of interest have been described for various cases eg. Krysan et al., 1999 (Plant Cell 1999, 11, 2283-2290); Sessions et al., 2002 (Plant Cell 2002, 14, 2985-2994); Young et al., 2001, (Plant Physiol. 2001, 125, 513-518); Koprek et al., 2000 (Plant J. 2000, 24, 253-263) ; Jeon et al., 2000 (Plant J. 2000, 22, 561-570) ; Tissier et al., 1999 (Plant Cell 1999, 11, 1841-1852); Speulmann et al., 1999 (Plant Cell 1999,11 , 1853-1866). Briefly material from all plants of a large T-DNA or transposon mutagenized plant population is harvested and genomic DNA prepared. Then the genomic DNA is pooled following specific architectures as described for example in Krysan et al., 1999 (Plant Cell 1999, 11, 2283-2290). Pools of genomics DNAs are then screened by specific multiplex PCR reactions detecting the combination of the insertional mutagen (eg T-DNA or Transposon) and the gene of interest. Therefore PCR reactions are run on the DNA pools with specific combinations of T-DNA or transposon border primers and gene specific primers. General rules for primer design can again be taken from Krysan et al., 1999 (Plant Cell 1999, 11, 2283-2290) Rescreening of lower levels DNA pools lead to the identifcation of individual plants in which the gene of interest is disrupted by the insertional mutagen.

Plant Screening for Growth Under Low Temperature Conditions

In a standard experiment soil was prepared as 3.5:1 (v/v) mixture of nutrient rich soil (GS90, Tantau, Wansdorf, Germany) and sand. Pots were filled with soil mixture and placed into trays. Water was added to the trays to let the soil mixture take up appropriate amount of water for the sowing procedure. The seeds for transgenic A. thaliana plants were sown in pots (6 cm diameter). Pots were collected until they filled a tray for the growth chamber. Then the filled tray was covered with a transparent lid and transferred into the shelf system of the precooled (4° C.-5° C.) growth chamber. Stratification was established for a period of 2-3 days in the dark at 4° C.-5° C. Germination of seeds and growth was initiated at a growth condition of 20° C., 60% relative humidity, 16 h photoperiod and illumination with fluorescent light at 200 μmol/m2s. Covers were removed 7 days after sowing. BASTA selection was done at day 9 after sowing by spraying pots with plantlets from the top. Therefore, a 0.07% (v/v) solution of BASTA concentrate (183 g/l glufosinate-ammonium) in tap water was sprayed. Transgenic events and wild-type control plants were distributed randomly over the chamber. The location of the trays inside the chambers was changed on working days from day 7 after sowing. Watering was carried out every two days after covers were removed from the trays. Plants were individualized 12-13 days after sowing by removing the surplus of seedlings leaving one seedling in a pot. Cold (chilling to 11° C.-12° C.) was applied 14 days after sowing until the end of the experiment. For measuring biomass performance, plant fresh weight was determined at harvest time (29-30 days after sowing) by cutting shoots and weighing them. Beside weighing, phenotypic information was added in case of plants that differ from the wild type control. Plants were in the stage prior to flowering and prior to growth of inflorescence when harvested. Significance values for the statistical significance of the biomass changes were calculated by applying the ‘student's’ t test (parameters: two-sided, unequal variance).

Three successive experiments were conducted. In the first experiment, one individual of each transformed line was tested.

In the second experiment, the event that had been determined as chilling tolerant or resistant in the first experiment, i.e. showed increased yield, in this case increased biomass production, in comparison to wild type, were put through a confirmation screen according to the same experimental procedures. In this experiment, max. 10 plants of each tolerant or resistant event were grown, treated and measured as before.

In the first two experiments, chilling tolerance or tolerance and biomass production was compared to wild type plants.

In the third experiment up to 20 replicates of each confirmed tolerant event, i.e. those that had been scored as tolerant or resistant in the second experiment, were grown, treated and scored as before. The results thereof are summarized in table VIII.

Table 3: Biomass production of transgenic A. thaliana after imposition of chilling stress.

Biomass production was measured by weighing plant rosettes. Biomass increase was calculated as ratio of average weight for trangenic plants compared to average weight of wild type control plants. The minimum and maximum biomass increase seen within the group of transgenic events is given for a locus with all events showing a significance value ≦0.1 and a biomass increase ≧1.1.

TABLE 3

| | | Biomass | Biomass |
|---|---|---|---|
| SeqID | Locus | Increase min | Increase max |
| 1385 | At5g40590 KO | 1.233 | 1.233 |

Equivalents

Those of ordinary skill in the art will recognize, or will be able to ascertain using no more than routine experimentation, many equivalents to the specific embodiments of the invention described herein. Such equivalents are intended to be encompassed by the following claims.

FIG. 1 T-DNA insertion vector pMTX1a300, SEQ ID NO.: 1 used used for insertional mutagenesis.

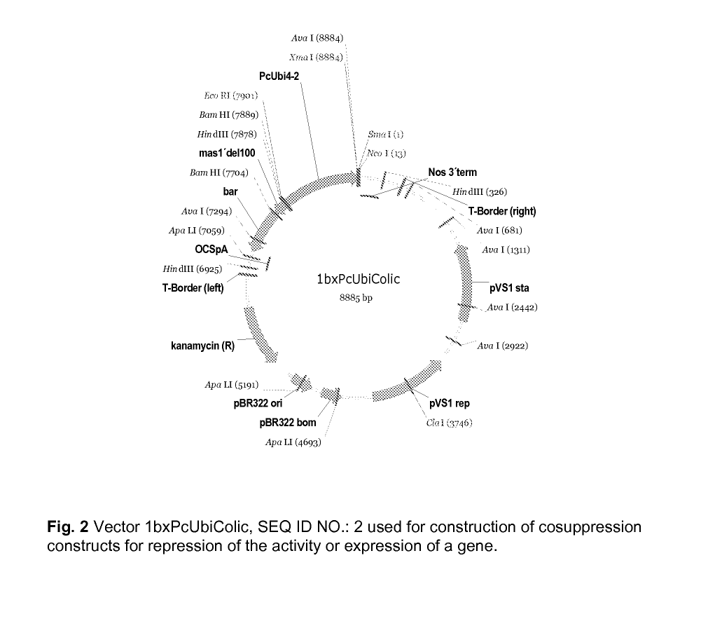

FIG. 2 Vector 1bxPcUbiColic, SEQ ID NO.: 2 used for construction of cosuppression constructs for repression of the activity or expression of a gene.

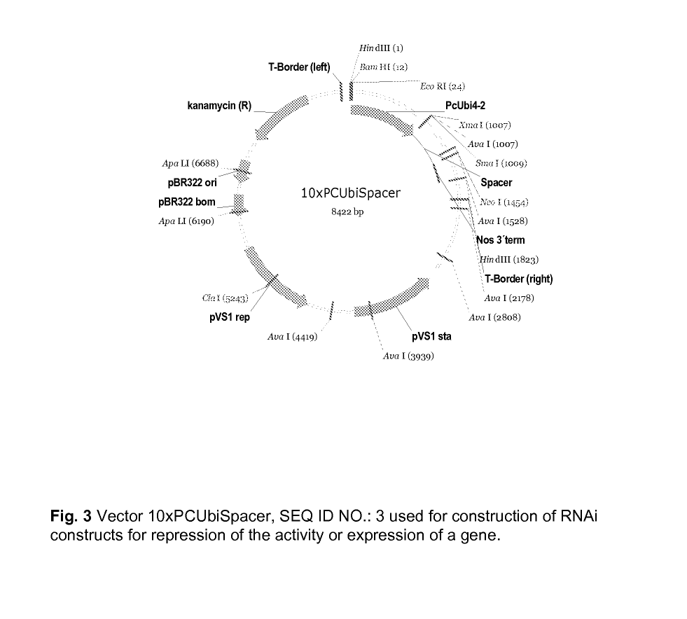

FIG. 3 Vector 10xPCUbiSpacer, SEQ ID NO.: 3 used for construction of RNAi constructs for repression of the activity or expression of a gene.

TABLE IA
Nucleic acid sequence ID numbers

| | | | | | 5. | | |
|---|---|---|---|---|---|---|---|
| | 1. | 2. | 3. | 4. | Lead | 6. | 7. |
| Application | Hit | Project | Locus | Organism | SEQ ID | Target | SEQ IDs of Nucleic Acid Homologs |
| 1 | 1 | LW1_KO_PCT | At5g50870 | A. th. | 1418 | — | 1420, 1422, 1424, 1426, 1428, 1430 |
| 1 | 2 | LW1_KO_PCT | At4g31120 | A. th. | 1025 | — | 1027, 1029, 1031, 1033, 1035, 1037, 1039, 1041, 1043 |
| 1 | 3 | LW1_KO_PCT | At3g14230 | A. th. | 729 | — | 731, 733, 735, 737, 739, 741, 743, 745, 747, 749, 751, 753, 755, |
| | | | | | | | 757, 759, 761, 763, 765, 767, 769, 771, 773 |
| 1 | 4 | LW1_KO_PCT | At1g12110 | A. th. | 27 | — | 29, 31, 33, 35, 37, 39, 41, 43, 45, 47, 49, 51, 53, 55, 57, 59, 61, |
| | | | | | | | 63, 65, 67, 69 |
| 1 | 5 | LW1_KO_PCT | At1g13270 | A. th. | 104 | — | 106, 108, 110, 112, 114, 116, 118, 120, 122, 124, 126, 128, 130, |
| | | | | | | | 132, 134, 136, 138, 140, 142, 144, 146, 148, 150, 152 |
| 1 | 6 | LW1_KO_PCT | At1g27080 | A. th. | 190 | — | 192, 194, 196, 198, 200, 202, 204, 206, 208, 210, 212, 214, 216, |
| | | | | | | | 218, 220, 222, 224, 226, 228, 230, 232, 234, 236, 238, 240, 242, |
| | | | | | | | 244, 246, 248, 250, 252, 254, 256, 258, 260, 262, 264, 266, 268, |
| | | | | | | | 270, 272, 274, 276, 278, 280, 282, 284, 286, 288, 290, 292, 294, |
| | | | | | | | 296, 298, 300, 302, 304, 306, 308, 310, 312, 314, 316, 318, 320, |
| | | | | | | | 322, 324, 326, 328, 330, 332, 334, 336, 338, 340, 342, 344, 346, |
| | | | | | | | 348, 350, 352, 354 |
| 1 | 7 | LW1_KO_PCT | At1g58360 | A. th. | 512 | — | 514, 516, 518, 520, 522, 524, 526, 528, 530, 532, 534, 536, 538, |
| | | | | | | | 540, 542, 544, 546, 548, 550, 552, 554, 556, 558, 560, 562, 564, |
| | | | | | | | 566, 568, 570, 572, 574, 576, 578, 580, 582, 584, 586, 588, 590, |
| | | | | | | | 592, 594, 596, 598, 600, 602, 604, 606, 608, 610, 612, 614, 616, |
| | | | | | | | 618, 620, 622, 624, 626, 628, 630, 632, 634, 636 |
| 1 | 8 | LW1_KO_PCT | At5g60780 | A. th. | 1464 | — | 1466, 1468, 1470, 1472, 1474, 1476, 1478, 1480, 1482, 1484, |
| | | | | | | | 1486, 1488, 1490, 1492, 1494, 1496, 1498, 1500, 1502, 1504, |
| | | | | | | | 1506, 1508, 1510, 1512, 1514, 1516, 1518, 1520, 1522, 1524 |
| 1 | 9 | LW1_KO_PCT | At3g54920 | A. th. | 813 | — | 815, 817, 819, 821, 823, 825, 827, 829, 831, 833, 835, 837, 839, |
| | | | | | | | 841, 843, 845, 847, 849, 851, 853, 855, 857, 859, 861, 863, 865, |
| | | | | | | | 867, 869, 871, 873, 875, 877, 879, 881, 883, 885, 887, 889, 891, |
| | | | | | | | 893, 895, 897, 899, 901, 903 |
| 1 | 10 | LW1_KO_PCT | At2g03670 | A. th. | 673 | — | 675, 677, 679, 681, 683, 685, 687, 689, 691, 693, 695, 697, 699, |
| | | | | | | | 701, 703, 705, 707, 709 |
| 1 | 11 | LW1_KO_PCT | At1g12110 | A. th. | 27 | — | 29, 31, 33, 35, 37, 39, 41, 43, 45, 47, 49, 51, 53, 55, 57, 59, 61, |
| | | | | | | | 63, 65, 67, 69 |
| 1 | 12 | LW1_KO_PCT | At1g58360 | A. th. | 512 | — | 514, 516, 518, 520, 522, 524, 526, 528, 530, 532, 534, 536, 538, |
| | | | | | | | 540, 542, 544, 546, 548, 550, 552, 554, 556, 558, 560, 562, 564, |
| | | | | | | | 566, 568, 570, 572, 574, 576, 578, 580, 582, 584, 586, 588, 590, |
| | | | | | | | 592, 594, 596, 598, 600, 602, 604, 606, 608, 610, 612, 614, 616, |
| | | | | | | | 618, 620, 622, 624, 626, 628, 630, 632, 634, 636 |
| 1 | 13 | LW1_KO_PCT | At5g40590 | A. th. | 1385 | — | 1387, 1389, 1391, 1393, 1395, 1397, 1399, 1401 |
| 1 | 14 | LW1_KO_PCT | At1g33760 | A. th. | 410 | — | 412, 414, 416, 418, 420, 422, 424, 426, 428, 430, 432, 434, 436, |
| | | | | | | | 438, 440, 442, 444, 446, 448, 450, 452, 454, 456, 458 |
| 1 | 15 | LW1_KO_PCT | At4g13430 | A. th. | 923 | — | 925, 927, 929, 931, 933, 935, 937, 939, 941, 943, 945, 947, 949, |
| | | | | | | | 951, 953, 955, 957, 959, 961, 963, 965, 967, 969, 971, 973, 975, |
| | | | | | | | 977, 979, 981, 983, 985, 987, 989, 991 |
| 1 | 16 | LW1_KO_PCT | At5g66160 | A. th. | 1593 | — | 1595, 1597, 1599, 1601, 1603, 1605, 1607, 1609, 1611, 1613, |
| | | | | | | | 1615, 1617, 1619, 1621, 1623, 1625, 1627, 1629, 1631, 1633 |
| 1 | 17 | LW1_KO_PCT | At3g55990 | A. th. | 1650 | — | 1652, 1654, 1656, 1658, 1660, 1662, 1664, 1666, 1668, 1670, |
| | | | | | | | 1672, 1674, 1676, 1678, 1680, 1682, 1684, 1686 |
| 1 | 18 | LW1_KO_PCT | At5g02330 | A. th. | 1083 | — | 1085, 1087, 1089, 1091, 1093, 1095, 1097, 1099, 1101, 1103, |
| | | | | | | | 1105, 1107, 1109, 1111, 1113, 1115, 1117, 1119, 1121, 1123, |
| | | | | | | | 1125, 1127, 1129, 1131, 1133, 1135, 1137, 1139, 1141, 1143, |
| | | | | | | | 1145, 1147, 1149, 1151, 1153, 1155, 1157, 1159, 1161, 1163, |
| | | | | | | | 1165, 1167, 1169, 1171, 1173, 1175, 1177, 1179, 1181, 1183, |
| | | | | | | | 1185, 1187, 1189, 1191, 1193, 1195, 1197, 1199, 1201, 1203, |
| | | | | | | | 1205, 1207, 1209, 1211, 1213, 1215, 1217, 1219, 1221, 1223, |
| | | | | | | | 1225, 1227, 1229, 1231, 1233, 1235, 1237, 1239, 1241, 1243, |
| | | | | | | | 1245, 1247, 1249, 1251, 1253, 1255, 1257, 1259, 1261, 1263, |
| | | | | | | | 1265, 1267, 1269, 1271, 1273, 1275, 1277, 1279, 1281, 1283, |
| | | | | | | | 1285, 1287, 1289, 1291, 1293, 1295, 1297, 1299, 1301, 1303, |
| | | | | | | | 1305, 1307, 1309, 1311, 1313, 1315, 1317, 1319, 1321, 1323, |
| | | | | | | | 1325, 1327, 1329, 1331, 1333, 1335, 1337, 1339, 1341, 1343, |
| | | | | | | | 1345, 1347, 1349, 1351, 1353, 1355, 1357, 1359, 1361, 1363, |
| | | | | | | | 1365, 1367, 1369, 1371 |
| 1 | 19 | LW1_KO_PCT | At5g64070 | A. th. | 1551 | — | 1553, 1555, 1557, 1559, 1561, 1563, 1565 |

TABLE IB
Nucleic acid sequence ID numbers

| | | | | | 5. | | |
|---|---|---|---|---|---|---|---|
| | 1. | 2. | 3. | 4. | Lead | 6. | 7. |
| Application | Hit | Project | Locus | Organism | SEQ ID | Target | SEQ IDs of Nucleic Acid Homologs |
| 1 | 1 | LW1_KO_PCT | At5g50870 | A. th. | 1418 | — | 1432, 1434, 1436, 1438, 1440, 1442, 1444, 1446, 1448, 1859, |
| | | | | | | | 1861, 1863, 1865, 1867, 1869, 1871, 1873, 1875, 1877, 1879, |
| | | | | | | | 1881, 1883, 1885, 1887, 1889, 1891, 1893, 1895, 1897 |
| 1 | 2 | LW1_KO_PCT | At4g31120 | A. th. | 1025 | — | 1045, 1047, 1049, 1051, 1053, 1055, 1057, 1059, 1849 |
| 1 | 3 | LW1_KO_PCT | At3g14230 | A. th. | 729 | — | 775, 777, 779, 781, 783, 785, 787, 789, 791, 793, 795, 797, |
| | | | | | | | 1799, 1801, 1803 |
| 1 | 4 | LW1_KO_PCT | At1g12110 | A. th. | 27 | — | 71, 73, 75, 77, 79, 81, 83, 1715, 1717, 1719, 1721, 1723, 1725, |
| | | | | | | | 1727 |
| 1 | 5 | LW1_KO_PCT | At1g13270 | A. th. | 104 | — | 154, 156, 158, 160, 162, 164, 166, 168, 170, 172, 174, 1731, |
| | | | | | | | 1733 |
| 1 | 6 | LW1_KO_PCT | At1g27080 | A. th. | 190 | — | 356, 358, 360, 362, 364, 366, 368, 370, 372, 374, 376, 378, 380, |
| | | | | | | | 382, 384, 386, 388, 390, 392, 394, 396, 1737, 1739, 1741, 1743, |
| | | | | | | | 1745, 1747, 1749, 1751, 1753, 1755, 1757, 1759, 1761 |
| 1 | 7 | LW1_KO_PCT | At1g58360 | A. th. | 512 | — | 638, 640, 642, 644, 646, 648, 650, 652, 1773, 1775, 1777, 1779, |
| | | | | | | | 1781, 1783, 1785, 1787, 1789 |
| 1 | 8 | LW1_KO_PCT | At5g60780 | A. th. | 1464 | — | 1526, 1528, 1530, 1532 |
| 1 | 9 | LW1_KO_PCT | At3g54920 | A. th. | 813 | — | 905, 1807, 1809, 1811, 1813, 1815, 1817, 1819, 1821, 1823, |
| | | | | | | | 1825, 1827, 1829, 1831 |
| 1 | 10 | LW1_KO_PCT | At2g03670 | A. th. | 673 | — | 711, 1793, 1795 |
| 1 | 11 | LW1_KO_PCT | At1g12110 | A. th. | 27 | — | 71, 73, 75, 77, 79, 81, 83, 1715, 1717, 1719, 1721, 1723, 1725, |
| | | | | | | | 1727 |
| 1 | 12 | LW1_KO_PCT | At1g58360 | A. th. | 512 | — | 638, 640, 642, 644, 646, 648, 650, 652, 1773, 1775, 1777, 1779, |
| | | | | | | | 1781, 1783, 1785, 1787, 1789 |
| 1 | 13 | LW1_KO_PCT | At5g40590 | A. th. | 1385 | — | 1853, 1855 |
| 1 | 14 | LW1_KO_PCT | At1g33760 | A. th. | 410 | — | 460, 462, 464, 466, 468, 470, 472, 474, 476, 478, 480, 482, 484, |
| | | | | | | | 486, 488, 490, 492, 494, 496, 498, 500, 1765, 1767, 1769 |
| 1 | 15 | LW1_KO_PCT | At4g13430 | A. th. | 923 | — | 993, 995, 997, 999, 1001, 1003, 1005, 1841, 1843, 1845 |
| 1 | 16 | LW1_KO_PCT | At5g66160 | A. th. | 1593 | — | 1635 |
| 1 | 17 | LW1_KO_PCT | At3g55990 | A. th. | 1650 | — | 1688, 1690, 1692, 1835, 1837 |
| 1 | 18 | LW1_KO_PCT | At5g02330 | A. th. | 1083 | — | — |
| 1 | 19 | LW1_KO_PCT | At5g64070 | A. th. | 1551 | — | 1567 |

TABLE IIA
Amino acid sequence ID numbers

| | | | | | 5. | | |
|---|---|---|---|---|---|---|---|
| | 1. | 2. | 3. | 4. | Lead | 6. | 7. |
| Application | Hit | Project | Locus | Organism | SEQ ID | Target | SEQ IDs of Polypeptide Homologs |
| 1 | 1 | LW1_KO_PCT | At5g50870 | A. th. | 1419 | — | 1421, 1423, 1425, 1427, 1429, 1431 |
| 1 | 2 | LW1_KO_PCT | At4g31120 | A. th. | 1026 | — | 1028, 1030, 1032, 1034, 1036, 1038, 1040, 1042, 1044 |
| 1 | 3 | LW1_KO_PCT | At3g14230 | A. th. | 730 | — | 732, 734, 736, 738, 740, 742, 744, 746, 748, 750, 752, 754, 756, |
| | | | | | | | 758, 760, 762, 764, 766, 768, 770, 772, 774 |
| 1 | 4 | LW1_KO_PCT | At1g12110 | A. th. | 28 | — | 30, 32, 34, 36, 38, 40, 42, 44, 46, 48, 50, 52, 54, 56, 58, 60, 62, |
| | | | | | | | 64, 66, 68, 70 |
| 1 | 5 | LW1_KO_PCT | At1g13270 | A. th. | 105 | — | 107, 109, 111, 113, 115, 117, 119, 121, 123, 125, 127, 129, 131, |
| | | | | | | | 133, 135, 137, 139, 141, 143, 145, 147, 149, 151, 153 |
| 1 | 6 | LW1_KO_PCT | At1g27080 | A. th. | 191 | — | 193, 195, 197, 199, 201, 203, 205, 207, 209, 211, 213, 215, 217, |
| | | | | | | | 219, 221, 223, 225, 227, 229, 231, 233, 235, 237, 239, 241, 243, |
| | | | | | | | 245, 247, 249, 251, 253, 255, 257, 259, 261, 263, 265, 267, 269, |
| | | | | | | | 271, 273, 275, 277, 279, 281, 283, 285, 287, 289, 291, 293, 295, |
| | | | | | | | 297, 299, 301, 303, 305, 307, 309, 311, 313, 315, 317, 319, 321, |
| | | | | | | | 323, 325, 327, 329, 331, 333, 335, 337, 339, 341, 343, 345, 347, |
| | | | | | | | 349, 351, 353, 355 |
| 1 | 7 | LW1_KO_PCT | At1g58360 | A. th. | 513 | — | 515, 517, 519, 521, 523, 525, 527, 529, 531, 533, 535, 537, 539, |
| | | | | | | | 541, 543, 545, 547, 549, 551, 553, 555, 557, 559, 561, 563, 565, |
| | | | | | | | 567, 569, 571, 573, 575, 577, 579, 581, 583, 585, 587, 589, 591, |
| | | | | | | | 593, 595, 597, 599, 601, 603, 605, 607, 609, 611, 613, 615, 617, |
| | | | | | | | 619, 621, 623, 625, 627, 629, 631, 633, 635, 637 |
| 1 | 8 | LW1_KO_PCT | At5g60780 | A. th. | 1465 | — | 1467, 1469, 1471, 1473, 1475, 1477, 1479, 1481, 1483, 1485, |
| | | | | | | | 1487, 1489, 1491, 1493, 1495, 1497, 1499, 1501, 1503, 1505, |
| | | | | | | | 1507, 1509, 1511, 1513, 1515, 1517, 1519, 1521, 1523, 1525 |
| 1 | 9 | LW1_KO_PCT | At3g54920 | A. th. | 814 | — | 816, 818, 820, 822, 824, 826, 828, 830, 832, 834, 836, 838, 840, |
| | | | | | | | 842, 844, 846, 848, 850, 852, 854, 856, 858, 860, 862, 864, 866, |
| | | | | | | | 868, 870, 872, 874, 876, 878, 880, 882, 884, 886, 888, 890, 892, |
| | | | | | | | 894, 896, 898, 900, 902, 904 |
| 1 | 10 | LW1_KO_PCT | At2g03670 | A. th. | 674 | — | 676, 678, 680, 682, 684, 686, 688, 690, 692, 694, 696, 698, 700, |
| | | | | | | | 702, 704, 706, 708, 710 |
| 1 | 11 | LW1_KO_PCT | At1g12110 | A. th. | 28 | — | 30, 32, 34, 36, 38, 40, 42, 44, 46, 48, 50, 52, 54, 56, 58, 60, 62, |
| | | | | | | | 64, 66, 68, 70 |
| 1 | 12 | LW1_KO_PCT | At1g58360 | A. th. | 513 | — | 515, 517, 519, 521, 523, 525, 527, 529, 531, 533, 535, 537, 539, |
| | | | | | | | 541, 543, 545, 547, 549, 551, 553, 555, 557, 559, 561, 563, 565, |
| | | | | | | | 567, 569, 571, 573, 575, 577, 579, 581, 583, 585, 587, 589, 591, |
| | | | | | | | 593, 595, 597, 599, 601, 603, 605, 607, 609, 611, 613, 615, 617, |
| | | | | | | | 619, 621, 623, 625, 627, 629, 631, 633, 635, 637 |
| 1 | 13 | LW1_KO_PCT | At5g40590 | A. th. | 1386 | — | 1388, 1390, 1392, 1394, 1396, 1398, 1400, 1402 |
| 1 | 14 | LW1_KO_PCT | At1g33760 | A. th. | 411 | — | 413, 415, 417, 419, 421, 423, 425, 427, 429, 431, 433, 435, 437, |
| | | | | | | | 439, 441, 443, 445, 447, 449, 451, 453, 455, 457, 459 |
| 1 | 15 | LW1_KO_PCT | At4g13430 | A. th. | 924 | — | 926, 928, 930, 932, 934, 936, 938, 940, 942, 944, 946, 948, 950, |
| | | | | | | | 952, 954, 956, 958, 960, 962, 964, 966, 968, 970, 972, 974, 976, |
| | | | | | | | 978, 980, 982, 984, 986, 988, 990, 992 |
| 1 | 16 | LW1_KO_PCT | At5g66160 | A. th. | 1594 | — | 1596, 1598, 1600, 1602, 1604, 1606, 1608, 1610, 1612, 1614, |
| | | | | | | | 1616, 1618, 1620, 1622, 1624, 1626, 1628, 1630, 1632, 1634 |
| 1 | 17 | LW1_KO_PCT | At3g55990 | A. th. | 1651 | — | 1653, 1655, 1657, 1659, 1661, 1663, 1665, 1667, 1669, 1671, |
| | | | | | | | 1673, 1675, 1677, 1679, 1681, 1683, 1685, 1687 |
| 1 | 18 | LW1_KO_PCT | At5g02330 | A. th. | 1084 | — | 1086, 1088, 1090, 1092, 1094, 1096, 1098, 1100, 1102, 1104, |
| | | | | | | | 1106, 1108, 1110, 1112, 1114, 1116, 1118, 1120, 1122, 1124, |
| | | | | | | | 1126, 1128, 1130, 1132, 1134, 1136, 1138, 1140, 1142, 1144, |
| | | | | | | | 1146, 1148, 1150, 1152, 1154, 1156, 1158, 1160, 1162, 1164, |
| | | | | | | | 1166, 1168, 1170, 1172, 1174, 1176, 1178, 1180, 1182, 1184, |
| | | | | | | | 1186, 1188, 1190, 1192, 1194, 1196, 1198, 1200, 1202, 1204, |
| | | | | | | | 1206, 1208, 1210, 1212, 1214, 1216, 1218, 1220, 1222, 1224, |
| | | | | | | | 1226, 1228, 1230, 1232, 1234, 1236, 1238, 1240, 1242, 1244, |
| | | | | | | | 1246, 1248, 1250, 1252, 1254, 1256, 1258, 1260, 1262, 1264, |
| | | | | | | | 1266, 1268, 1270, 1272, 1274, 1276, 1278, 1280, 1282, 1284, |
| | | | | | | | 1286, 1288, 1290, 1292, 1294, 1296, 1298, 1300, 1302, 1304, |
| | | | | | | | 1306, 1308, 1310, 1312, 1314, 1316, 1318, 1320, 1322, 1324, |
| | | | | | | | 1326, 1328, 1330, 1332, 1334, 1336, 1338, 1340, 1342, 1344, |
| | | | | | | | 1346, 1348, 1350, 1352, 1354, 1356, 1358, 1360, 1362, 1364, |
| | | | | | | | 1366, 1368, 1370, 1372 |
| 1 | 19 | LW1_KO_PCT | At5g64070 | A. th. | 1552 | — | 1554, 1556, 1558, 1560, 1562, 1564, 1566 |

TABLE IIB
Amino acid sequence ID numbers

| | | | | | 5. | | |
|---|---|---|---|---|---|---|---|
| | 1. | 2. | 3. | 4. | Lead | 6. | 7. |
| Application | Hit | Project | Locus | Organism | SEQ ID | Target | SEQ IDs of Polypeptide Homologs |
| 1 | 1 | LW1_KO_PCT | At5g50870 | A. th. | 1419 | — | 1433, 1435, 1437, 1439, 1441, 1443, 1445, 1447, 1449, 1860, |
| | | | | | | | 1862, 1864, 1866, 1868, 1870, 1872, 1874, 1876, 1878, 1880, |
| | | | | | | | 1882, 1884, 1886, 1888, 1890, 1892, 1894, 1896, 1898 |
| 1 | 2 | LW1_KO_PCT | At4g31120 | A. th. | 1026 | — | 1046, 1048, 1050, 1052, 1054, 1056, 1058, 1060, 1850 |
| 1 | 3 | LW1_KO_PCT | At3g14230 | A. th. | 730 | — | 776, 778, 780, 782, 784, 786, 788, 790, 792, 794, 796, 798, |
| | | | | | | | 1800, 1802, 1804 |
| 1 | 4 | LW1_KO_PCT | At1g12110 | A. th. | 28 | — | 72, 74, 76, 78, 80, 82, 84, 1716, 1718, 1720, 1722, 1724, 1726, |
| | | | | | | | 1728 |
| 1 | 5 | LW1_KO_PCT | At1g13270 | A. th. | 105 | — | 155, 157, 159, 161, 163, 165, 167, 169, 171, 173, 175, 1732, |
| | | | | | | | 1734 |
| 1 | 6 | LW1_KO_PCT | At1g27080 | A. th. | 191 | — | 357, 359, 361, 363, 365, 367, 369, 371, 373, 375, 377, 379, 381, |
| | | | | | | | 383, 385, 387, 389, 391, 393, 395, 397, 1738, 1740, 1742, 1744, |
| | | | | | | | 1746, 1748, 1750, 1752, 1754, 1756, 1758, 1760, 1762 |
| 1 | 7 | LW1_KO_PCT | At1g58360 | A. th. | 513 | — | 639, 641, 643, 645, 647, 649, 651, 653, 1774, 1776, 1778, 1780, |
| | | | | | | | 1782, 1784, 1786, 1788, 1790 |
| 1 | 8 | LW1_KO_PCT | At5g60780 | A. th. | 1465 | — | 1527, 1529, 1531, 1533 |
| 1 | 9 | LW1_KO_PCT | At3g54920 | A. th. | 814 | — | 906, 1808, 1810, 1812, 1814, 1816, 1818, 1820, 1822, 1824, |
| | | | | | | | 1826, 1828, 1830, 1832 |
| 1 | 10 | LW1_KO_PCT | At2g03670 | A. th. | 674 | — | 712, 1794, 1796 |
| 1 | 11 | LW1_KO_PCT | At1g12110 | A. th. | 28 | — | 72, 74, 76, 78, 80, 82, 84, 1716, 1718, 1720, 1722, 1724, 1726, |
| | | | | | | | 1728 |
| 1 | 12 | LW1_KO_PCT | At1g58360 | A. th. | 513 | — | 639, 641, 643, 645, 647, 649, 651, 653, 1774, 1776, 1778, 1780, |
| | | | | | | | 1782, 1784, 1786, 1788, 1790 |
| 1 | 13 | LW1_KO_PCT | At5g40590 | A. th. | 1386 | — | 1854, 1856 |
| 1 | 14 | LW1_KO_PCT | At1g33760 | A. th. | 411 | — | 461, 463, 465, 467, 469, 471, 473, 475, 477, 479, 481, 483, 485, |
| | | | | | | | 487, 489, 491, 493, 495, 497, 499, 501, 1766, 1768, 1770 |
| 1 | 15 | LW1_KO_PCT | At4g13430 | A. th. | 924 | — | 994, 996, 998, 1000, 1002, 1004, 1006, 1842, 1844, 1846 |
| 1 | 16 | LW1_KO_PCT | At5g66160 | A. th. | 1594 | — | 1636 |
| 1 | 17 | LW1_KO_PCT | At3g55990 | A. th. | 1651 | — | 1689, 1691, 1693, 1836, 1838 |
| 1 | 18 | LW1_KO_PCT | At5g02330 | A. th. | 1084 | — | — |
| 1 | 19 | LW1_KO_PCT | At5g64070 | A. th. | 1552 | — | 1568 |

TABLE III
Primer nucleic acid sequence ID numbers

| | | | | | 5. | | |
|---|---|---|---|---|---|---|---|
| | 1. | 2. | 3. | 4. | Lead | 6. | 7. |
| Application | Hit | Project | Locus | Organism | SEQ ID | Target | SEQ IDs of Primers |
| 1 | 1 | LW1_KO_PCT | At5g50870 | A. th. | 1418 | — | 1450, 1451, 1452, 1453, 1454, 1455, 1456, 1457 |
| 1 | 2 | LW1_KO_PCT | At4g31120 | A. th. | 1025 | — | 1061, 1062, 1063, 1064, 1065, 1066, 1067, 1068 |
| 1 | 3 | LW1_KO_PCT | At3g14230 | A. th. | 729 | — | 799, 800, 801, 802, 803, 804, 805, 806 |
| 1 | 4 | LW1_KO_PCT | At1g12110 | A. th. | 27 | — | 85, 86, 87, 88, 89, 90, 91, 92 |
| 1 | 5 | LW1_KO_PCT | At1g13270 | A. th. | 104 | — | 176, 177, 178, 179, 180, 181, 182, 183 |
| 1 | 6 | LW1_KO_PCT | At1g27080 | A. th. | 190 | — | 398, 399, 400, 401, 402, 403, 404, 405 |
| 1 | 7 | LW1_KO_PCT | At1g58360 | A. th. | 512 | — | 654, 655, 656, 657, 658, 659, 660, 661 |
| 1 | 8 | LW1_KO_PCT | At5g60780 | A. th. | 1464 | — | 1534, 1535, 1536, 1537, 1538, 1539, 1540, 1541 |
| 1 | 9 | LW1_KO_PCT | At3g54920 | A. th. | 813 | — | 907, 908, 909, 910, 911, 912, 913, 914 |
| 1 | 10 | LW1_KO_PCT | At2g03670 | A. th. | 673 | — | 713, 714, 715, 716, 717, 718, 719, 720 |
| 1 | 11 | LW1_KO_PCT | At1g12110 | A. th. | 27 | — | 85, 86, 87, 88, 89, 90, 91, 92 |
| 1 | 12 | LW1_KO_PCT | At1g58360 | A. th. | 512 | — | 654, 655, 656, 657, 658, 659, 660, 661 |
| 1 | 13 | LW1_KO_PCT | At5g40590 | A. th. | 1385 | — | 1403, 1404, 1405, 1406, 1407, 1408, 1409, 1410 |
| 1 | 14 | LW1_KO_PCT | At1g33760 | A. th. | 410 | — | 502, 503, 504, 505, 506, 507, 508, 509 |
| 1 | 15 | LW1_KO_PCT | At4g13430 | A. th. | 923 | — | 1007, 1008, 1009, 1010, 1011, 1012, 1013, 1014 |
| 1 | 16 | LW1_KO_PCT | At5g66160 | A. th. | 1593 | — | 1637, 1638, 1639, 1640, 1641, 1642, 1643, 1644 |
| 1 | 17 | LW1_KO_PCT | At3g55990 | A. th. | 1650 | — | 1694, 1695, 1696, 1697, 1698, 1699, 1700, 1701 |
| 1 | 18 | LW1_KO_PCT | At5g02330 | A. th. | 1083 | — | 1373, 1374, 1375, 1376, 1377, 1378, 1379, 1380 |
| 1 | 19 | LW1_KO_PCT | At5g64070 | A. th. | 1551 | — | 1569, 1570, 1571, 1572, 1573, 1574, 1575, 1576 |

TABLE IV
Consensus amino acid sequence ID numbers

| | | | | | 5. | | |
|---|---|---|---|---|---|---|---|
| | 1. | 2. | 3. | 4. | Lead SEQ | 6. | 7. |
| Application | Hit | Project | Locus | Organism | ID | Target | SEQ IDs of Consensus/Pattern Sequences |
| 1 | 1 | LW1_KO_PCT | At5g50870 | A. th. | 1419 | — | 1458, 1459, 1460, 1461, 1462, 1463 |
| 1 | 2 | LW1_KO_PCT | At4g31120 | A. th. | 1026 | — | 1069, 1070, 1071, 1072, 1073, 1074, 1075, 1076, 1077, 1078, |
| | | | | | | | 1079, 1080, 1081, 1082 |
| 1 | 3 | LW1_KO_PCT | At3g14230 | A. th. | 730 | — | 807, 808, 809, 810, 811, 812 |
| 1 | 4 | LW1_KO_PCT | At1g12110 | A. th. | 28 | — | 93, 94, 95, 96, 97, 98, 99, 100, 101, 102, 103 |
| 1 | 5 | LW1_KO_PCT | At1g13270 | A. th. | 105 | — | 184, 185, 186, 187, 188, 189 |
| 1 | 6 | LW1_KO_PCT | At1g27080 | A. th. | 191 | — | 406, 407, 408, 409 |
| 1 | 7 | LW1_KO_PCT | At1g58360 | A. th. | 513 | — | 662, 663, 664, 665, 666, 667, 668, 669, 670, 671, 672 |
| 1 | 8 | LW1_KO_PCT | At5g60780 | A. th. | 1465 | — | 1542, 1543, 1544, 1545, 1546, 1547, 1548, 1549, 1550 |
| 1 | 9 | LW1_KO_PCT | At3g54920 | A. th. | 814 | — | 915, 916, 917, 918, 919, 920, 921, 922 |
| 1 | 10 | LW1_KO_PCT | At2g03670 | A. th. | 674 | — | 721, 722, 723, 724, 725, 726, 727, 728 |
| 1 | 11 | LW1_KO_PCT | At1g12110 | A. th. | 28 | — | 93, 94, 95, 96, 97, 98, 99, 100, 101, 102, 103 |
| 1 | 12 | LW1_KO_PCT | At1g58360 | A. th. | 513 | — | 662, 663, 664, 665, 666, 667, 668, 669, 670, 671, 672 |
| 1 | 13 | LW1_KO_PCT | At5g40590 | A. th. | 1386 | — | 1411, 1412, 1413, 1414, 1415, 1416, 1417 |
| 1 | 14 | LW1_KO_PCT | At1g33760 | A. th. | 411 | — | 510, 511 |
| 1 | 15 | LW1_KO_PCT | At4g13430 | A. th. | 924 | — | 1015, 1016, 1017, 1018, 1019, 1020, 1021, 1022, 1023, 1024 |
| 1 | 16 | LW1_KO_PCT | At5g66160 | A. th. | 1594 | — | 1645, 1646, 1647, 1648, 1649 |
| 1 | 17 | LW1_KO_PCT | At3g55990 | A. th. | 1651 | — | 1702, 1703, 1704, 1705, 1706, 1707, 1708, 1709, 1710, 1711, |
| | | | | | | | 1712 |
| 1 | 18 | LW1_KO_PCT | At5g02330 | A. th. | 1084 | — | 1381, 1382, 1383, 1384 |
| 1 | 19 | LW1_KO_PCT | At5g64070 | A. th. | 1552 | — | 1577, 1578, 1579, 1580, 1581, 1582, 1583, 1584, 1585, 1586, |
| | | | | | | | 1587, 1588, 1589, 1590, 1591, 1592 |

1. A method for producing a transgenic plant with increased tolerance and/or resistance to environmental stress and increased biomass production as compared to a corresponding non-transformed wild type plant, which comprises:
a) reducing, repressing or deleting one or more activities selected from the group consisting of: 1-phosphatidylinositol 4-kinase, amino acid permease (AAP1), At3g55990-protein, At5g40590-protein, ATP-dependent peptidase/ATPase/nucleoside-triphosphatase/serine-type endopeptidase, DC1 domain-containing protein/protein-binding protein/zinc ion binding protein, DNA binding protein/transcription factor, hydro-lyase/aconitate hydratase, metalloexopeptidase (MAP1C), methyltransferase, nitrate transporter (ATNRT2.3), nitrate/chlorate transporter (NRT1.1), pectate lyase protein/powdery mildew susceptibility protein (PMR6), peptidase/ubiquitin-protein ligase/zinc ion binding protein (JR700), proton-dependent oligopeptide transport protein, transcription factor, and ubiquitin conjugating enzyme/ubiquitin-like activating enzyme, in a plant cell, a plant or a part thereof, and
b) generating a transformed plant with increased tolerance and/or resistance to environmental stress and increased biomass production as compared to a corresponding non-transformed wild type plant and growing under conditions which permit the development of the plant.

2. A method for producing a transgenic plant with increased tolerance and/or resistance to environmental stress and increased biomass production as compared to a corresponding non-transformed wild type plant, which comprises:
a) reducing, repressing, or deleting the activity of
(i) a polypeptide comprising a polypeptide, a consensus sequence or at least one polypeptide motif as depicted in column 5 or 7 of Table II or of Table IV, respectively; or
(ii) an expression product of a nucleic acid molecule comprising a polynucleotide as depicted in column 5 or 7 of Table I,
(iii) or a functional equivalent of (i) or (ii);
in a plant cell, a plant or a part thereof, and
b) generating a transformed plant with increased tolerance and/or resistance to environmental stress and increased biomass production as compared to a corresponding non-transformed wild type plant and growing under conditions which permit the development of the plant.

3. The method of claim 2, comprising reducing, decreasing or deleting the expression or activity of at least one nucleic acid molecule having or encoding the activity of at least one nucleic acid molecule represented by the nucleic acid molecule as depicted in column 5 of Table I, and comprising a nucleic acid molecule which is selected from the group consisting of:
a) a nucleic acid molecule encoding the polypeptide shown in column 5 or 7 of Table II;
b) a nucleic acid molecule shown in column 5 or 7 of Table I;
c) a nucleic acid molecule, which, as a result of the degeneracy of the genetic code, can be derived from a polypeptide sequence depicted in column 5 or 7 of Table II;
d) a nucleic acid molecule having at least 30% identity with the nucleic acid molecule sequence of a polynucleotide comprising the nucleic acid molecule shown in column 5 or 7 of Table I;
e) a nucleic acid molecule encoding a polypeptide having at least 30% identity with the amino acid sequence of the polypeptide encoded by the nucleic acid molecule of (a) to (c) and having the activity represented by a nucleic acid molecule comprising a polynucleotide as depicted in column 5 of Table I;
f) a nucleic acid molecule encoding a polypeptide which can be isolated with the aid of monoclonal or polyclonal antibodies made against a polypeptide encoded by one of the nucleic acid molecules of (a) to (e) and having the activity represented by the nucleic acid molecule comprising a polynucleotide as depicted in column 5 of Table I;
g) a nucleic acid molecule encoding a polypeptide comprising the consensus sequence or one or more polypeptide motifs as shown in column 7 of Table IV and having the activity represented by a nucleic acid molecule comprising a polynucleotide as depicted in column 5 of Table II or IV;
h) a nucleic acid molecule encoding a polypeptide having the activity represented by a protein as depicted in column 5 of Table II;
i) a nucleic acid molecule which comprises a polynucleotide, which is obtained by amplifying a cDNA library or a genomic library using the primers in column 7 of Table III which do not start at their 5′-end with the nucleotides ATA and having the activity represented by a nucleic acid molecule comprising a polynucleotide as depicted in column 5 of Table II or IV;
j) a nucleic acid molecule encoding a polypeptide, the polypeptide being derived by substituting, deleting and/or adding one or more amino acids of the amino acid sequence of the polypeptide encoded by the nucleic acid molecules (a) to (d); and
k) a nucleic acid molecule which is obtainable by screening a suitable nucleic acid library under stringent hybridization conditions with a probe comprising a complementary sequence of a nucleic acid molecule of (a) or (b) or with a fragment thereof, having at least 15 nt of a nucleic acid molecule complementary to a nucleic acid molecule sequence characterized in (a) to (d) and encoding a polypeptide having the activity represented by a protein comprising a polypeptide as depicted in column 5 of Table II;
or which comprises a sequence which is complementary thereto;
or reducing, repressing, decreasing or deleting an expression product of a nucleic acid molecule comprising a nucleic acid molecule as depicted in (a) to (k) or a protein encoded by said nucleic acid molecule.

4. The method of claim 3, wherein the activity or expression of a polypeptide comprising a polypeptide encoded by the nucleic acid molecule characterized in claim 3 is reduced in a plant cell, a plant or a part thereof.

5. The method of claim 3, whereby the process comprises at least one step selected from the group consisting of:
(a) introducing of a nucleic acid molecule encoding a ribonucleic acid sequence, which is able to form a double-stranded ribonucleic acid molecule, whereby a fragment of at least 17 nt of said double-stranded ribonucleic acid molecule has a homology of at least 50% to a nucleic acid molecule selected from the group of
(aa) a nucleic acid molecule as characterized in claim 3;
(ab) a nucleic acid molecule as depicted in column 5 or 7 of Table I or encoding a polypeptide as depicted in column 5 or 7 of Table II, and
(ac) a nucleic acid molecule encoding a polypeptide having the activity of polypeptide depicted in column 5 of Table II or encoding the expression product of a polynucleotide comprising a nucleic acid molecule as depicted in column 5 or 7 of Table I;
(b) introducing an RNAi, snRNA, dsRNA, siRNA, miRNA, ta-siRNA, cosuppression molecule, ribozyme, or antisense nucleic acid molecule, whereby the RNAi, snRNA, dsRNA, siRNA, miRNA, ta-siRNA, cosuppression molecule, ribozyme, or antisense nucleic acid molecule comprises a fragment of at least 17 nt with a a homology of at least 50% to a nucleic acid molecule selected from a group defined in section (a) of this claim.
(c) introducing of a ribozyme which specifically cleaves a nucleic acid molecule selected from the group defined in section (a) of this claim;
(d) introducing of the RNAi, snRNA, dsRNA, siRNA, miRNA, ta-siRNA, cosuppression molecule, ribozyme, or antisense nucleic acid molecule characterized in (b) and the ribozyme characterized in (c);
(e) introducing of a sense nucleic acid molecule conferring the expression of a nucleic acid molecule comprising a nucleic acid molecule selected from the group defined in claim 3 or defined in section (ab) or (ac) of this claim or a nucleic acid molecule encoding a polypeptide having at least 50% identity with the amino acid sequence of the polypeptide encoded by the nucleic acid molecule of claim 3 (a) to (c) and having the activity represented by a protein comprising a polypeptide depicted in column 5 of Table II for inducing a co-suppression of the endogenous expression product;
(f) introducing a nucleic acid molecule conferring the expression of a dominant-negative mutant of a protein having the activity of a protein as depicted in column 5 or 7 of Table II or comprising a polypeptide being encoded by a nucleic acid molecule of claim 3;
(g) introducing a nucleic acid molecule encoding a factor, which binds to a nucleic acid molecule comprising a nucleic acid molecule selected from the group defined in claim 3 or defined in section (ab) or (ac) of this claim conferring the expression of a protein having the activity of a protein encoded by a nucleic acid molecule as characterized in claim 3;
(h) introducing a viral nucleic acid molecule conferring the decline of a RNA molecule comprising a nucleic acid molecule selected from the group defined in claim 3 or defined in section (ab) or (ac) of this claim conferring the expression of a protein encoded by a nucleic acid molecule as characterized in claim 3;
(i) introducing a nucleic acid construct capable to recombine with and silence, inactivate, repress or reduces the activity of an endogenous gene comprising a nucleic acid molecule selected from the group defined in claim 3 or defined in section (ab) or (ac) of this claim conferring the expression of a protein encoded by a nucleic acid molecule as characterized in claim 3;
(j) introducing a non-silent mutation in a endogenous gene comprising a nucleic acid molecule selected from the group defined in claim 3 or defined in section (ab) or (ac) of this claim; and
(k) introducing an expression construct conferring the expression of nucleic acid molecule characterized in any one of (a) to (i).

6. The method of claim 3, wherein a fragment of at least 17 by of a 3′- or 5′-nucleic acid sequence of a sequence comprising a nucleic acid molecule selected from the group defined in claim 3; or a nucleic acid with an identity of at least 50% comprising a nucleic acid molecule as depicted in column 5 or 7 of Table I or encoding a polypeptide as depicted in column 5 or 7 of Table II, or a nucleic acid molecule encoding a polypeptide having the activity of polypeptide depicted in column 5 of Table II or encoding the expression product of a polynucleotide comprising a nucleic acid molecule as depicted in column 5 or 7 of Table I, is used for the reduction of the nucleic acid molecule of claim 3 or the polypeptide encoded by said nucleic acid molecule.

7. The method of claim 1, wherein the reduction or deletion is caused by applying a chemical compound to the plant cell, the plant or a part thereof.

8. The method of claim 1, wherein the plant is selected from the group consisting of Anacardiaceae, Asteraceae, Apiaceae, Betulaceae, Boraginaceae, Brassicaceae, Bromeliaceae, Caricaceae, Cannabaceae, Convolvulaceae, Chenopodiaceae, Cucurbitaceae, Elaeagnaceae, Ericaceae, Euphorbiaceae, Fabaceae, Geraniaceae, Gramineae, Juglandaceae, Lauraceae, Leguminosae, Linaceae, perennial grass, fodder crops, vegetables and ornamentals.

9. The method of claim 3 comprising a RNAi, snRNA, dsRNA, siRNA, miRNA, ta-siRNA, cosuppression molecule, ribozyme, antibody and/or antisense nucleic that has been designed to target the expression product of a gene comprising the nucleic acid molecule as characterized in claim 3 to induce a breakdown of the mRNA of the said gene of interest and thereby silence the gene expression, or of an expression cassette ensuring the expression of the former.

10. An isolated nucleic acid molecule which comprises a nucleic acid molecule selected from the group consisting of:
a) a nucleic acid molecule which encodes a polypeptide comprising the polypeptide shown in column 5 or 7 of Table IIB;
b) a nucleic acid molecule which comprising a polynucleotide shown in column 5 or 7 of Table IB;
c) a nucleic acid molecule comprising a nucleic acid sequence, which, as a result of the degeneracy of the genetic code, can be derived from a polypeptide sequence depicted in column 5 or 7 of Table IIB and having the activity represented by the protein depicted in column 5 of Table II;
d) a nucleic acid molecule encoding a polypeptide having at least 50% identity with the amino acid sequence of a polypeptide encoded by the nucleic acid molecule of (a) or (c) and having the activity represented by the protein depicted in column 5 of Table II;
e) a nucleic acid molecule encoding a polypeptide, which is isolated with the aid of monoclonal antibodies against a polypeptide encoded by one of the nucleic acid molecules of (a) to (c) and having the activity represented by the protein depicted in column 5 of Table II;
f) a nucleic acid molecule encoding a polypeptide comprising the consensus sequence or a polypeptide motif shown in column 7 of Table IV and having the biological activity represented by the protein depicted in column 5 of Table II;
g) a nucleic acid molecule encoding a polypeptide having the activity represented by a protein as depicted in column 5 of Table II;
h) a nucleic acid molecule which comprises a polynucleotide, which is obtained by amplifying a cDNA library or a genomic library using the primers in column 7 of Table III which do not start at their 5′-end with the nucleotides ATA; and
i) a nucleic acid molecule which is obtainable by screening a suitable library under stringent hybridization conditions with a probe comprising one of the sequences of the nucleic acid molecule of (a) to (c) or with a fragment of at least 17 nt of the nucleic acid molecule characterized in any one of (a) to (h) and encoding a polypeptide having the activity represented by the protein depicted in column 5 of Table II;
or which comprises a sequence which is complementary thereto;
whereby the nucleic acid molecule according to (a) to (i) is at least in one or more nucleotides different from the sequence depicted in column 5 or 7 of Table IA and which encodes a protein which differs at least in one or more amino acids from the protein sequences depicted in column 5 or 7 of Table IIA.

11. A RNAi, snRNA, dsRNA, siRNA, miRNA, ta-siRNA, cosuppression molecule, ribozyme, antibody or antisense nucleic acid molecule for the reduction of the activity or expression of the nucleic acid molecule of claim 10 or a polypeptide encoded by said nucleic acid molecule.

12. A RNAi, snRNA, dsRNA, siRNA, miRNA, ta-siRNA, cosuppression molecule, ribozyme, or antisense nucleic acid molecule comprising a fragment of at least 17 nt of the nucleic acid molecule of claim 10.

13. A double-stranded RNA (dsRNA), RNAi, snRNA, siRNA, miRNA, antisense or to-siRNA molecule or ribozyme, which is able to form a double-stranded ribonucleic acid molecule, whereby a fragment of at least 17 at of said double-stranded ribonucleic acid molecule has a homology of at least 50% to a nucleic acid molecule selected from the group of
(aa) the nucleic acid molecule of claim 10;
(ab) a nucleic acid molecule as depicted in column 5 or 7 of Table I or encoding a polypeptide as depicted in column 5 or 7 of Table II, and
(ac) a nucleic acid molecule encoding a polypeptide having the activity of polypeptide depicted in column 5 or 7 of Table II or encoding the expression product of a polynucleotide comprising a nucleic acid molecule as depicted in column 5 or 7 of Table I.

14. The dsRNA molecule of claim 11, whereby the sense strand and the antisense strand are covalently bound to each other and the antisense strand is essentially the complement of the “sense”-RNA strand.

15. A viral nucleic acid molecule conferring the decline of the activity or expression of a nucleic acid molecule as characterized in claim 10 or a polypeptide encoded by said nucleic acid molecule.

16. A TILLING primer for the identification of a knock out of a gene comprising a nucleic acid sequence of a nucleic acid molecule as depicted in any one column 5 or 7 of Table I.

17. A dominant-negative mutant of a polypeptide comprising a polypeptide as shown in column 5 or 7 of Table II.

18. A nucleic acid molecule encoding the dominant negative mutant of claim 17.

19. The TILLING primer of claim 16 comprising a fragment of a nucleic acid sequence as depicted in column 5 or 7 of Table I or complementary fragment thereof.

20. A nucleic acid construct conferring the expression of the nucleic acid molecule of claim 10, of a RNAi, snRNA, dsRNA, siRNA, miRNA, ta-siRNA, cosuppression molecule, ribozyme, antibody or antisense nucleic acid molecule for the reduction of the activity or expression of the nucleic acid molecule or a polypeptide encoded by said nucleic acid molecule, of as viral nucleic acid molecule conferring the decline of the activity or expression of the nucleic acid molecule or a polypeptide encoded by said nucleic acid molecule.

21. A nucleic acid construct comprising the isolated nucleic acid molecule as claimed in claim 10 or a RNAi, snRNA, dsRNA, siRNA, miRNA, ta-siRNA, cosuppression molecule, ribozyme, or antisense nucleic acid molecule for the reduction of the activity or expression of the nucleic acid molecule or a polypeptide encoded by said nucleic acid molecule, or a viral nucleic acid molecule conferring the decline of the activity or expression of the nucleic acid molecule or a polypeptide encoded by said nucleic acid molecule, wherein the nucleic acid molecule is functionally linked to one or more regulatory signals.

22. A vector comprising the nucleic acid molecule claimed in claim 10 or a RNAi, snRNA, dsRNA, siRNA, miRNA, ta-siRNA, cosuppression molecule, ribozyme, or antisense nucleic acid molecule for the reduction of the activity or expression of the nucleic acid molecule or a polypeptide encoded by said nucleic acid molecule, or a viral nucleic acid molecule conferring the decline of the activity or expression of the nucleic acid molecule or a polypeptide encoded by said nucleic acid molecule, or a nucleic acid construct comprising the nucleic acid molecule or the RNAi, snRNA, dsRNA, siRNA, miRNA, ta-siRNA, cosuppression molecule, ribozyme, or antisense nucleic acid molecule.

23. The vector as claimed in claim 22, wherein nucleic acid molecule is in operable linkage with regulatory sequences for the expression in a plant cell, a plant or a part thereof.

24. A transgenic plant cell, plant or a part thereof which has been transformed stably or transiently with the nucleic acid molecule as claimed in claim 10; or with a nucleic acid construct comprising the nucleic acid molecule or a RNAi, snRNA, dsRNA, siRNA, miRNA, ta-siRNA, cosuppression molecule, ribozyme, or antisense nucleic acid molecule for the reduction of the activity or expression of the nucleic acid molecule or a polypeptide encoded by said nucleic acid molecule or a viral nucleic acid molecule conferring the decline of the activity or expression of the nucleic acid molecule or a polypeptide encoded by said nucleic acid molecule; or with a vector comprising the nucleic acid molecule or the nucleic acid construct.

25. A transgenic plant cell, plant or a part thereof wherein the activity of a protein comprising a polypeptide, a consensus sequence or a polypeptide motif as depicted in column 5 or 7 of Table II or IV or a nucleic acid molecule comprising a nucleic acid molecule as depicted in column 5 or 7 of Table I is reduced.

26. The transgenic plant cell, a plant or a part thereof of claim 24 derived from a monocotyledonous plant.

27. The transgenic plant cell, a plant or a part thereof of claim 24 derived from a dicotyledonous plant.

28. The transgenic plant cell, a plant or a part thereof of claim 24 wherein the plant is selected from the group consisting of maize (corn), wheat, rye, oat, triticale, rice, barley, soy, peanut, cotton, oil seed rape, including canola and winter oil seed rape, manihot, pepper, sunflower, flax, borage, safflower, linseed, primrose, rapeseed, turnip rape, tagetes, solanaceous plants, potato, tobacco, eggplant, tomato, Vicia species, pea, alfalfa, coffee, cacao, tea, Salix species, oil palm, coconut, perennial grass, forage crops and Arabidopsis thaliana.

29. The transgenic plant cell, a plant or a part thereof of claim 24, derived from a gymnosperm plant.

30. An isolated polypeptide encoded by the nucleic acid molecule as claimed in claim 10 or comprising the polypeptide as depicted in column 7 of Table IIB.

31. An antibody, which specifically binds to the polypeptide as claimed in claim 30.

32. A plant tissue, plant, harvested plant material or propagation material of a plant comprising the plant cell as claimed in claim 24.

33. A process for producing a polypeptide encoded by the nucleic acid sequence as claimed in claim 10, comprising expressing the polypeptide in a host cell comprising the nucleic acid sequence.

34. The transgenic plant cell, a plant or a part thereof of claims 24, wherein the transgenic plant cell, plant, or part thereof has increased tolerance and/or resistance to environmental stress and increased biomass production as compared to a corresponding non-transformed wild type plant cell, plant, or part thereof, wherein the environmental stress is selected from the group comprised of salinity, drought, temperature, metal, chemical, pathogenic and oxidative stresses, or combinations thereof.

35. The transgenic plant cell, a plant or a part thereof of claim 34, wherein the environmental stress is drought and/or desiccation.

36. A transgenic plant cell, a plant or a part thereof of claim 24 that has
i) an increased biomass production under conditions where water would be limiting for growth for a non-transformed wild type plant cell, a plant or part thereof
ii) an increased biomass production under conditions of drought and/or desiccation where said conditions would be limiting for growth for a non-transformed wild type plant cell, a plant or part thereof and/or
iii) an increased biomass production under conditions of low humidity where said conditions would be limiting for growth for a non-transformed wild type plant cell, a plant or part thereof.

37. A method for screening for an antagonists of the activity being represented by the polypeptide encoded by the nucleic acid molecule characterized in claim 2:
i) contacting an organism, its cells, tissues or parts, which express the polypeptide with a chemical compound or a sample comprising a plurality of chemical compounds under conditions which permit the reduction or deletion of the expression of the nucleic acid molecule encoding the activity represented by the protein or which permit the reduction or deletion of the activity of the protein;
ii) assaying the level of the activity of the protein or the polypeptide expression level in the plant, its cells, tissues or parts thereof; and
iii) identifying an antagonist by comparing the measured level of the activity of the protein or the polypeptide expression level with a standard level of the activity of the protein or the polypeptide expression level measured in the absence of said chemical compound or a sample comprising said plurality of chemical compounds, whereby an decreased level in comparison to the standard indicates that the chemical compound or the sample comprising said plurality of chemical compounds is an antagonist.

38. A process for the identification of a compound conferring increased tolerance and/or resistance to environmental stress and increased biomass production as compared to a corresponding non-transformed wild type plant in a plant; comprising the steps:
i) culturing or maintaining a plant or a part thereof expressing the polypeptide encoded by the nucleic acid molecule characterized in claim 2 or a polynucleotide encoding said polypeptide and a readout system capable of interacting with the polypeptide under suitable conditions which permit the interaction of the polypeptide with this readout system in the presence of a chemical compound or a sample comprising a plurality of chemical compounds and capable of providing a detectable signal in response to the binding of a chemical compound to said polypeptide under conditions which permit the depression of said readout system and of said polypeptide; and
ii) identifying if the chemical compound is an effective antagonist by detecting the presence or absence or decrease or increase of a signal produced by said readout system.

39. A composition comprising the nucleic acid molecule of claim 10; a protein encoded by the nucleic acid molecule; a nucleic acid construct comprising the nucleic acid molecule or a RNAi, snRNA, dsRNA, siRNA, miRNA, ta-siRNA, cosuppression molecule, ribozyme, or antisense nucleic acid molecule for the reduction of the activity or expression of the nucleic acid molecule or a polypeptide encoded by said nucleic acid molecule or a viral nucleic acid molecule conferring the decline of the activity or expression of the nucleic acid molecule or a polypeptide encoded by said nucleic acid molecule; a vector comprising the nucleic acid molecule or the nucleic acid construct; a plant comprising the nucleic acid molecule; or a RNAi, snRNA, dsRNA, siRNA, miRNA, ta-siRNA, cosuppression molecule, ribozyme, or antisense nucleic acid molecule for the reduction of the activity or expression of the nucleic acid molecule or a polypeptide encoded by said nucleic acid molecule; and optionally a agricultural acceptable carrier.

40. A food or feed composition comprising the nucleic acid molecule of claim 10; a protein encoded by the nucleic acid molecule; a nucleic acid construct comprising the nucleic acid molecule or a RNAi, snRNA, dsRNA, siRNA, miRNA, ta-siRNA, cosuppression molecule, ribozyme, or antisense nucleic acid molecule for the reduction of the activity or expression of the nucleic acid molecule or a polypeptide encoded by said nucleic acid molecule or a viral nucleic acid molecule conferring the decline of the activity or expression of the nucleic acid molecule or a polypeptide encoded by said nucleic acid molecule; a vector comprising the nucleic acid molecule or the nucleic acid construct; a RNAi, snRNA, dsRNA, siRNA, miRNA, ta-siRNA, cosuppression molecule, ribozyme, or antisense nucleic acid molecule for the reduction of the activity or expression of the nucleic acid molecule or a polypeptide encoded by said nucleic acid molecule; a plant, plant tissue, harvested plant material or propagation material of said plant comprising the nucleic acid molecule.

41. (canceled)
